(19) United States
(12) Patent Application Publication
(10) Pub. No.: US 2024/0417365 A1
(43) Pub. Date: Dec. 19, 2024

(54) CARBOXYLATE, CARBOXYLIC ACID GENERATOR, RESIN, RESIST COMPOSITION AND METHOD FOR PRODUCING RESIST PATTERN

(71) Applicant: SUMITOMO CHEMICAL COMPANY, LIMITED, Tokyo (JP)

(72) Inventors: Shohei TERAHIGASHI, Osaka-shi (JP); Yoshitaka AONO, Osaka-shi (JP); Koji ICHIKAWA, Osaka-shi (JP)

(73) Assignee: SUMITOMO CHEMICAL COMPANY, LIMITED, Tokyo (JP)

(21) Appl. No.: 18/655,757

(22) Filed: May 6, 2024

(30) Foreign Application Priority Data
May 9, 2023 (JP) 2023-077329

(51) Int. Cl. C07C 67/08; G03F 7/004

(52) U.S. Cl. CPC C07C 67/08; G03F 7/0045

(57) ABSTRACT

Disclosed are a carboxylate represented by formula (I), a carboxylic acid generator and a resist composition:

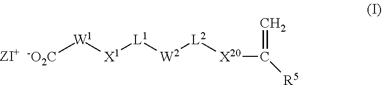

wherein W¹ and W² each represent a substituted/unsubstituted cyclic hydrocarbon group, —CH₂— in the group may be replaced by —O—, —S—, —CO— or —SO₂—, X¹ represents —CO—O—, —O—CO—, etc., L¹ and L² each independently represent a single bond or a substituted/unsubstituted hydrocarbon group, X²⁰ represents single bond, *—O—**, *—CO—O—**, *—O—CO—O—**, etc., R⁵ represents a H atom, a halogen atom, or an alkyl group which may have a halogen atom, and ZI⁺ represents an organic cation.

### BACKGROUND OF THE INVENTION

### Field of the Invention

The present invention relates to a carboxylate for acid generator which is used for fine processing of semiconductors, a carboxylic acid generator including the carboxylate, a resin, a resist composition and a method for producing a resist pattern.

### Description of the Related Art

JP 2011-037834 A mentions a resist composition comprising a resin including a structural unit derived from a carboxylate represented by the following formula, respectively.

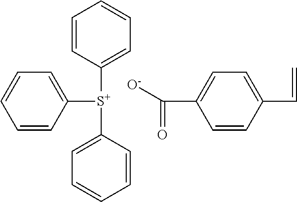

### SUMMARY OF THE INVENTION

The present invention provides a carboxylate capable of producing a resist pattern with CD uniformity (CDU) which is better than a resist pattern formed from the above resist composition comprising a resin including a structural unit derived from a carboxylate.

The present invention includes the following inventions.

[1] A carboxylate represented by formula (I):

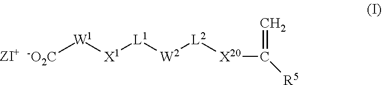

wherein, in formula (I),
W¹ and W² each independently represent a cyclic hydrocarbon group having 3 to 36 carbon atoms which may have a substituent, and —CH₂— included in the cyclic hydrocarbon group may be replaced by —O—, —S—, —CO— or —SO₂—,
X¹ represents —CO—O—, —O—CO—, —O—CO—O— or —O—,
L¹ and L² each independently represent a single bond or a hydrocarbon group having 1 to 28 carbon atoms which may have a substituent, and —CH₂— included in the hydrocarbon group may be replaced by —O—, —S—, —SO₂— or —CO—,
X²⁰ represents a single bond, *—O—**, *—CO—O—**, *—O—CO—O—** or *-Ax-Ph-Ay-**,
Ph represents a phenylene group which may have a substituent,
Ax and Ay each independently represent one or more bond species selected from the group consisting of a single bond, an ether bond, a thioether bond, an ester bond, an amide bond and a carbonic acid ester bond,
* and ** represent a bonding site, and * represents a bonding site to carbon atoms to which R⁵ is bonded,
R⁵ represents a hydrogen atom, a halogen atom, or an alkyl group having 1 to 6 carbon atoms which may have a halogen atom, and
ZI⁺ represents an organic cation.
   [2] The carboxylate according to [1], wherein X²⁰ is a single bond or a group represented by any one of formula (X²⁰-1) to formula (X²⁰-10)

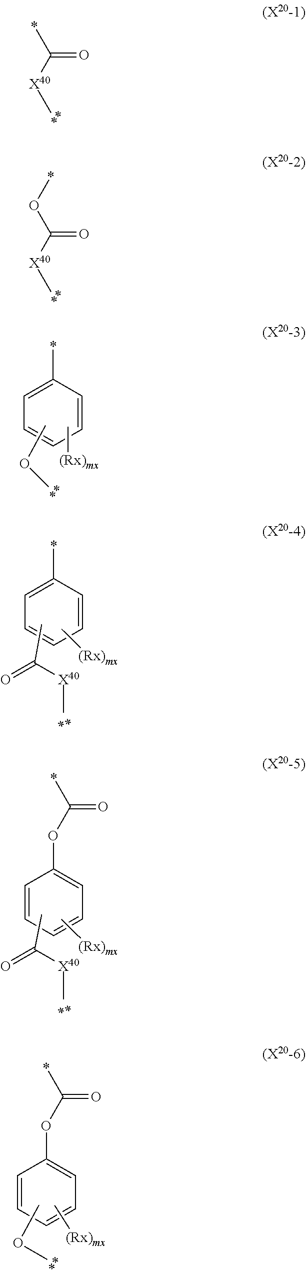

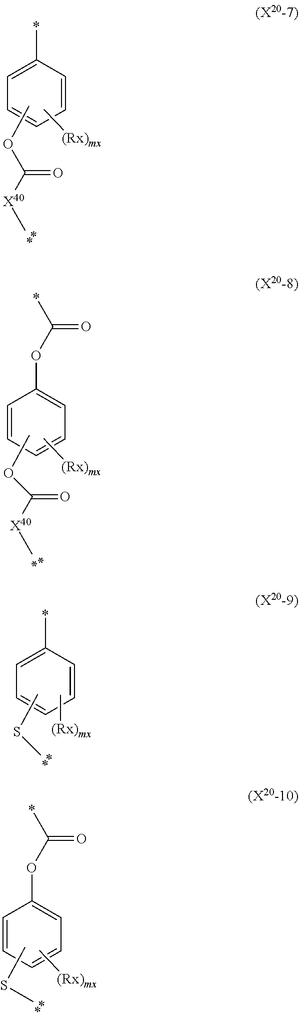

wherein, in formula (X²⁰-1) to formula (X²⁰-10),
* and ** are bonding sites, and * represents a bonding site to carbon atoms to which R⁵ is bonded,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms,
mx represents an integer of 0 to 4,
X⁴⁰ represents —O— or —NR³—, and
R³ represents a hydrogen atom or an alkyl group having 1 to 6 carbon atoms.
   [3] The carboxylate according to [1] or [2], wherein W¹ is a cyclic hydrocarbon group represented by formula (W-1):

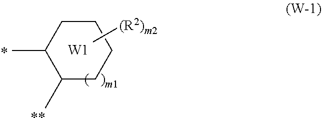

wherein, in formula (W-1),
W¹ represents a cyclic hydrocarbon group having (5+ ml) carbon atoms, a carbon atom included in the cyclic hydrocarbon group may form a double bond between two adjacent carbon atoms, —CH₂— included in the cyclic hydrocarbon group may be replaced by —O—, —S—, —CO— or —SO₂—, and a hydrogen atom included in the cyclic hydrocarbon group may be substituted with a substituent,
R² represents a halogen atom, a haloalkyl group having 1 to 6 carbon atoms or an alkyl group having 1 to 12 carbon atoms, —CH₂— included in the alkyl group may be replaced by —O—, —CO—, —S— or —SO₂—, the alkyl group may be bonded to two carbon atoms included in W¹ to form an alkanediyl bridge, —CH₂— included in the alkanediyl bridge may be replaced by —O—, —CO—, —S— or —SO₂—, and the alkanediyl bridge may form a double bond between two adjacent carbon atoms,
m1 represents an integer of 0 to 3,
m2 represents an integer of 0 to 3, and when m2 is 2 or more, a plurality of R² may be the same or different from each other, and
* represents a bonding site to carbon atoms included in COO—, and ** represents a bonding site to X¹.
   [4] The carboxylate according to any one of [1] to [3], wherein X¹ is *—CO—O— or *—O— (in which * represents a bonding site to W¹).
   [5] The carboxylate according to any one of [1] to [4], wherein L¹ and L² each independently represent a single bond or an alkanediyl group having 1 to 8 carbon atoms (—CH₂—included in the alkanediyl group may be replaced by —O— or —CO—).
   [6] The carboxylate according to any one of [1] to [5], wherein W² is an aromatic hydrocarbon group having 6 to 10 carbon atoms which may have a halogen atom, a haloalkyl group having 1 to 6 carbon atoms or an alkyl group having 1 to 12 carbon atoms (—CH₂— included in the alkyl group may be replaced by —O—, —CO—, —S— or —SO₂—).
   [7] A carboxylic acid generator comprising the carboxylate according to any one of [1] to [6] or a structural unit derived from the carboxylate according to any one of [1] to [6].
   [8] A resin including a structural unit derived from the carboxylate according to any one of [1] to [6].
   [9] A resist composition comprising the carboxylic acid generator according to [7], and an acid generator other than the carboxylic acid generator.
   [10] The resist composition according to [9], wherein
the carboxylic acid generator is a carboxylate represented by formula (I), and
a resin including a structural unit having an acid-labile group is further included, or
the carboxylic acid generator is a resin including a structural unit derived from a carboxylate represented by formula (I), and
the resin further includes a structural unit having an acid-labile group.
   [11] The resist composition according to [10], wherein the structural unit having an acid-labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5) and a structural unit represented by formula (a1-6)

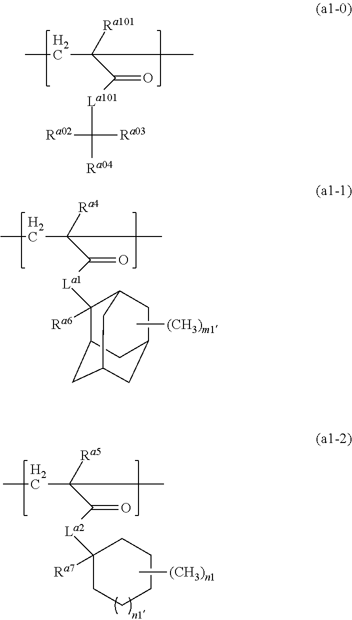

wherein, in formula (a1-0), formula (a1-1) and formula (a1-2),
L^{a01}, L^{a1} and L^{a2} each independently represent —O— or *—O—(CH₂)ₖ₁—CO—O—, k1 represents an integer of 1 to 7, and * represents a bonding site to —CO—,
R^{a01}, R^{a4} and R^{a5} each independently represent a hydrogen atom, a halogen atom, or an alkyl group having 1 to 6 carbon atoms which may have a halogen atom,
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups formed by combining these groups, and the alkyl group, the alkenyl group, the alicyclic hydrocarbon group and the aromatic hydrocarbon group may have a halogen atom,
m1′ represents an integer of 0 to 14,
n1 represents an integer of 0 to 10, and
n1′ represents an integer of 0 to 3:

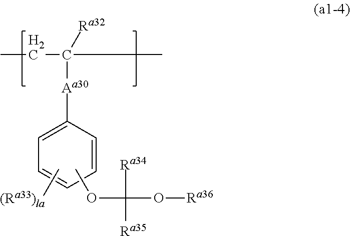

wherein, in formula (a1-4),
R^{a32} represents a hydrogen atom, a halogen atom, or an alkyl group having 1 to 6 carbon atoms which may have a halogen atom,
R^{a33} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a30} represents a single bond or *—X^{a31}-(A^{a32}-X^{a32})_{nc}—, and * represents a bonding site to carbon atoms to which —R^{a32} is bonded,
A^{a32} represents an alkanediyl group having 1 to 8 carbon atoms,
X^{a31} and X^{a32} each independently represent —O—, —CO—O—or —O—CO—,
nc represents 0 or 1,
1a represents an integer of 0 to 4, and when 1a is an integer of 2 or more, a plurality of R^{a33} may be the same or different from each other, and
R^{a34} and R^{a35} each independently represent a hydrogen atom or a hydrocarbon group having 1 to 12 carbon atoms, R^{a36} represents a hydrocarbon group having 1 to 20 carbon atoms, or R^{a35} and R^{a36} may be bonded to each other to form a divalent hydrocarbon group having 2 to 20 carbon atoms together with —C—O— to which R^{a35} and R^{a36} are bonded, and —CH₂— included in the hydrocarbon group and the divalent hydrocarbon group may be replaced by —O— or —S—:

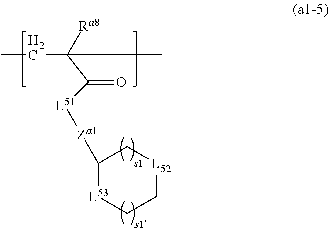

wherein, in formula (a1-5),
R^{aB} represents an alkyl group having 1 to 6 carbon atoms which may have a halogen atom, a hydrogen atom or a halogen atom,
Z^{a1} represents a single bond or *—(CH₂)ₕ₃—CO-L⁵⁴-, h3 represents an integer of 1 to 4, and * represents a bonding site to L⁵¹,
L⁵¹, L⁵², L⁵³ and L⁵⁴ each independently represent —O— or —S—,
s1 represents an integer of 1 to 3, and
s1′ represents an integer of 0 to 3, and:

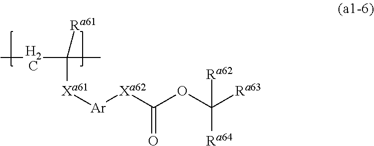

wherein, in formula (a1-6),
R^{a61} represents a hydrogen atom, a halogen atom, or an alkyl group having 1 to 6 carbon atoms which may have a halogen atom,
R^{a62}, R^{a63} and R^{a64} each independently represent an alkyl group having 1 to 6 carbon atoms or a cyclic hydrocarbon group having 3 to 18 carbon atoms which may have a substituent, or R^{a62} and R^{a63} may be bonded to each other to form a ring having 3 to 20 carbon atoms together with carbon atoms to which R^{a62} and R^{a63} are bonded,
X^{a61} represents a single bond, —CO—O—* or —CO—NR⁵—*, * represents a bonding site to —Ar, and R⁵ represents a hydrogen atom or an alkyl group having 1 to 4 carbon atoms,
X^{a62} represents a single bond, *—O-L^{a61}- or *—CO—O— L^{a62}-, * represents a bonding site to —Ar, and L^{a61} and L^{a62} each independently represent an alkanediyl group having 1 to 4 carbon atoms, and
Ar represents an aromatic hydrocarbon group having 6 to 20 carbon atoms which may have a substituent.
   [12] The resist composition according to [10] or [11], wherein the resin including a structural unit having an acid-labile group includes a structural unit represented by formula (a2-A):

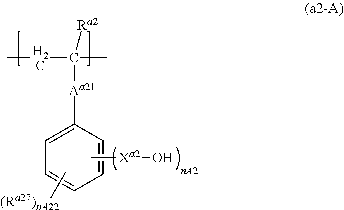

wherein, in formula (a2-A),
R^{a2} represents a hydrogen atom, a halogen atom, or an alkyl group having 1 to 6 carbon atoms which may have a halogen atom,
A^{a21} represents a single bond or an alkanediyl group having 1 to 12 carbon atoms, and —CH₂— included in the alkanediyl group may be replaced by —O—, —CO— or —NR^{a2}—,
R^{a28} represents a hydrogen atom or an alkyl group having 1 to 4 carbon atoms,
X^{a2} represents a single bond or —CO—,
R^{a27} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group,
nA2 represents an integer of 1 to 5, and when nA2 is 2 or more, a plurality of X^{a2} may be the same or different from each other, and
nA22 represents an integer of 0 to 4, and when nA22 is 2 or more, a plurality of R^{a27} may be the same or different from each other.
   [13] The resist composition according to any one of [9] to [12], wherein the acid generator includes a salt represented by formula (B1):

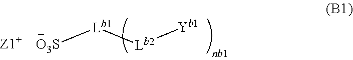

wherein, in formula (B1),
L^{b1} represents a single bond or a (nb1+1)-valent hydrocarbon group which may have a substituent, and —CH₂— included in the hydrocarbon group may be replaced by —O—, —S—, —CO— or —SO₂—,
L^{b2} represents a single bond or a divalent hydrocarbon group having 1 to 24 carbon atoms which may have a substituent, and —CH₂— included in the hydrocarbon group may be replaced by —O—, —S—, —CO— or —SO₂—,
Y^{b1} represents a methyl group which may have a substituent, or a cyclic hydrocarbon group having 3 to 24 carbon atoms which may have a substituent, and —CH₂— included in the cyclic hydrocarbon group may be replaced by —O—, —S—, —CO— or —SO₂—,
nb1 represents an integer of 1 to 6, and when nb1 is 2 or more, a plurality of groups in parentheses may be the same or different from each other, and
Z1⁺ represents an organic cation.
   [14] A method for producing a resist pattern, which comprises:
(1) a step of applying the resist composition according to any one of [9] to [13] on a substrate,
(2) a step of drying the applied composition to form a composition layer,
(3) a step of exposing the composition layer,
(4) a step of heating the exposed composition layer, and
(5) a step of developing the heated composition layer.

It is possible to produce a resist pattern with satisfactory CD uniformity (CDU) by using a resist composition of the present invention.

### DETAILED DESCRIPTION OF THE PREFERRED EMBODIMENTS

As used herein, “(meth)acrylic monomer” means “at least one of acrylic monomer and methacrylic monomer”. Notations such as “(meth)acrylate” and “(meth)acrylic acid” mean the same thing. In groups mentioned herein, regarding groups capable of having both a linear structure and a branched structure, they may have either the linear or branched structure. When —CH₂— included in the hydrocarbon group or the like is replaced by —O—, —S—, —CO— or —SO₂—, the same examples shall apply for each group, and the number of carbon atoms before replacement is taken as the number of carbon atoms of the hydrocarbon group or the like. “Combined group” means a group in which two or more exemplified groups are bonded, and valences of those groups may be appropriately varied by bonding forms. “Derived” or “Induced” means that a polymerizable C═C bond included in the molecule becomes a —C—C-group (single bond) by polymerization. When stereoisomers exist, all stereoisomers are included. The “acid-labile group” means a group having a leaving group which is eliminated by contact with an acid (e.g., trifluoromethanesulfonic acid, etc.), thus forming a hydrophilic group (e.g., a hydroxy group or a carboxy group). The “base-labile group” means a group having a leaving group which is eliminated by contact with a base (e.g., trimethylamine, etc.), thus forming a hydrophilic group (e.g., a carboxy group or a hydroxy group). Hydrogen atoms at any position and any number of hydrogen atoms included in each group may be sometimes replaced by a bond depending on the number of substituents or the like. The number of carbon atoms in the substituents is not included in the number of carbon atoms in the group to be substituted.

As used herein, “solid component of the resist composition” means the total amount of components in which the below-mentioned solvent (E) is removed from the total amount of the resist composition.

### [Carboxylate Represented by Formula (I)]

The present invention relates to a carboxylate represented by formula (I) (hereinafter sometimes referred to as “salt (I)” or “carboxylate (I)”).

Of the salt (I), the side having positive charge is sometimes referred to as “cation (I)”, and the side having negative charge is sometimes referred to as “anion (I)”:

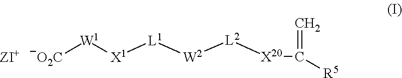

wherein all symbols are the same as defined above.

In formula (I), the cyclic hydrocarbon group having 3 to 36 carbon atoms as for W¹ and W² (hereinafter sometimes referred to as “ring W¹” and “ring W²”) may be either saturated or unsaturated, or may be either monocyclic or polycyclic, and the polycyclic cyclic hydrocarbon group may be a bridged ring or may form spiro. Examples of the cyclic hydrocarbon group include an alicyclic hydrocarbon group (—CH₂— included in the alicyclic hydrocarbon group may be replaced by —O—, —S—, —CO— or —SO₂—) or an aromatic hydrocarbon group. The carbon atom included in the alicyclic hydrocarbon group may form a double bond between two adjacent carbon atoms. The number of carbon atoms of the cyclic hydrocarbon group is preferably 3 to 30, more preferably 3 to 24, still more preferably 3 to 20.

Examples of the alicyclic hydrocarbon group include the following alicyclic hydrocarbon groups and the like. The bonding site can be any position.

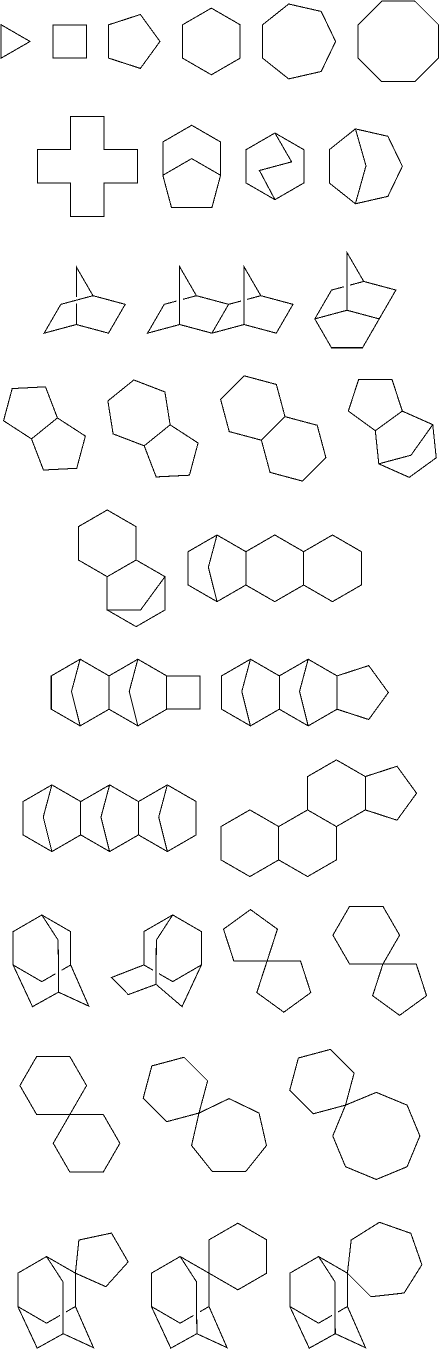

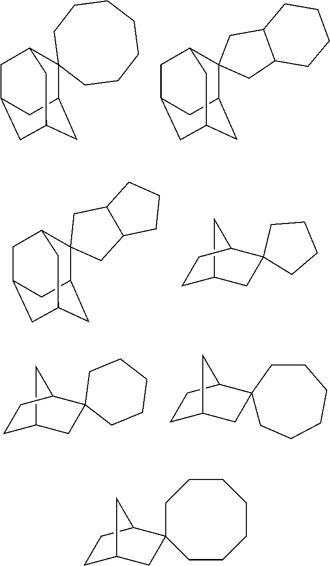

Specific examples of the monocyclic alicyclic hydrocarbon group include monocyclic cycloalkanediyl groups such as a cyclopropanediyl group, a cyclobutanediyl group, a cyclopentanediyl group, a cyclohexanediyl group, a cycloheptanediyl group, a cyclooctanediyl group and a cyclodecanediyl group. Examples of the polycyclic alicyclic hydrocarbon group include polycyclic cycloalkanediyl groups such as a decahydronaphthanediyl group, an adamantanediyl group, a norbornanediyl group, a norbornenediyl group and a bicyclo[3.3.0]octanediyl group.

Examples of the cyclic hydrocarbon group which forms a double bond between two adjacent carbon atoms included in the cyclic hydrocarbon group include the following cyclic hydrocarbon groups and the like.

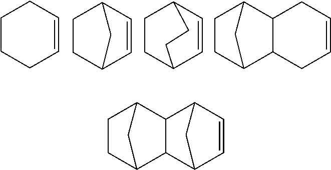

Examples of the group in which —CH₂— included in the alicyclic hydrocarbon group is replaced by —O—, —S—, —CO— or —SO₂— include the following groups and the like. Examples thereof also include groups in which —O— of the following groups is replaced by —S—, and —CO— thereof is replaced by —SO₂—, respectively. The bonding site can be any position.

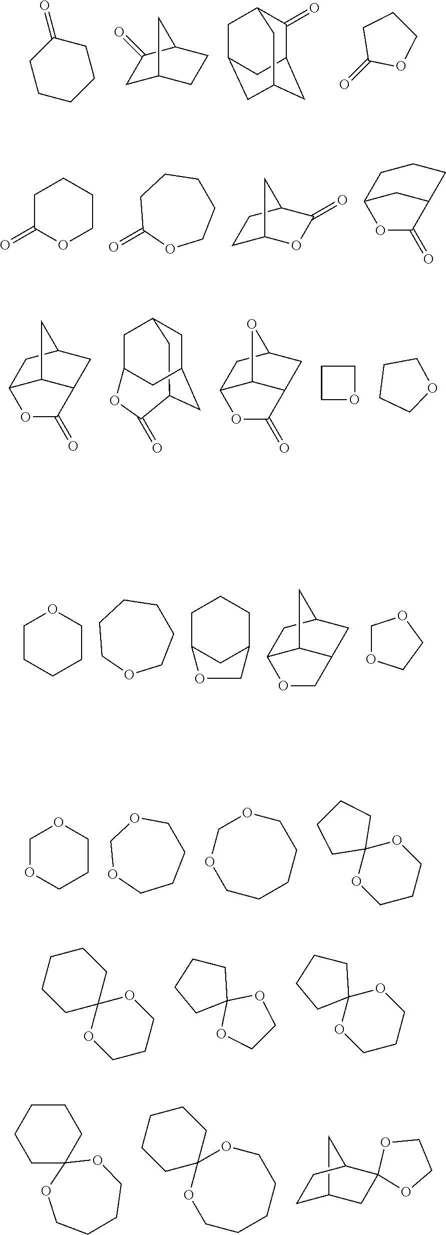

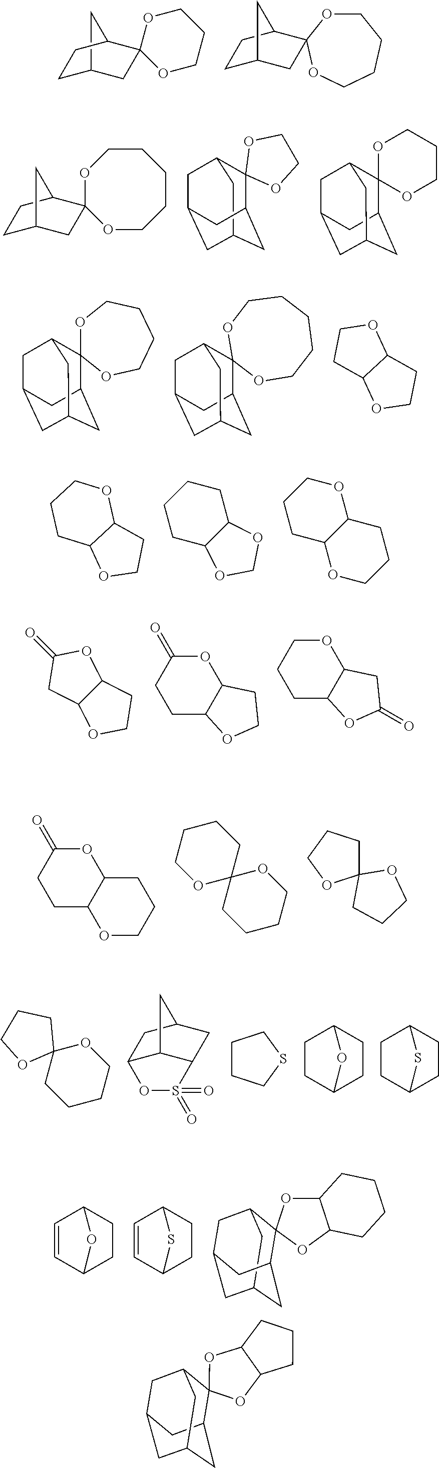

The number of carbon atoms of the alicyclic hydrocarbon group is preferably 3 to 24, more preferably 3 to 18, still more preferably 3 to 16, and yet more preferably 3 to 12.

Examples of the aromatic hydrocarbon group include aromatic hydrocarbon groups, for example, arylene groups such as a phenylene group, a naphthylene group, an anthrylene group, a biphenylene group and a phenanthrene group. The number of carbon atoms of the aromatic hydrocarbon group is preferably 6 to 24, more preferably 6 to 18, still more preferably 6 to 14, and yet more preferably 6 to 10.

The cyclic hydrocarbon group is preferably a cyclic hydrocarbon group having 3 to 18 carbon atoms (a hydrogen atom included in the cyclic hydrocarbon group may be substituted with a substituent, and —CH₂— included in the cyclic hydrocarbon group may be replaced by —O—, —S—, —CO— or —SO₂—), more preferably an alicyclic hydrocarbon group having 3 to 18 carbon atoms or an aromatic hydrocarbon group having 6 to 18 carbon atoms (a hydrogen atom included in the alicyclic hydrocarbon group and the aromatic hydrocarbon group may be substituted with a substituent, and —CH₂— included in the alicyclic hydrocarbon group may be replaced by —O—, —S—, —CO— or —SO₂—), and still more preferably a group represented by any one of formula (w1-1) to formula (w1-32):

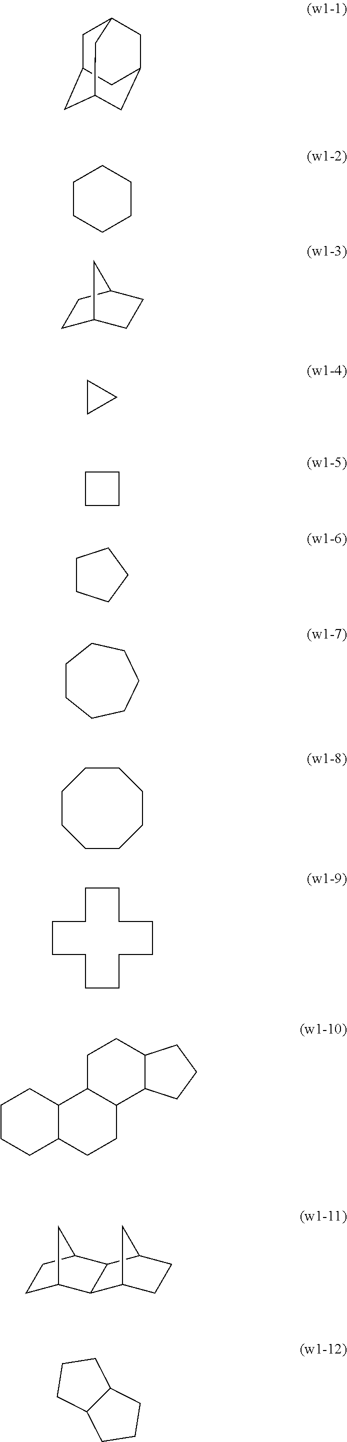

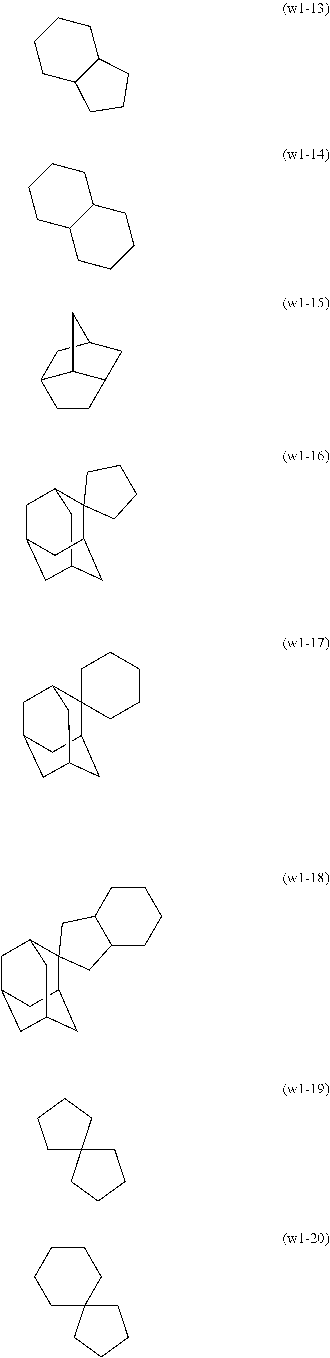

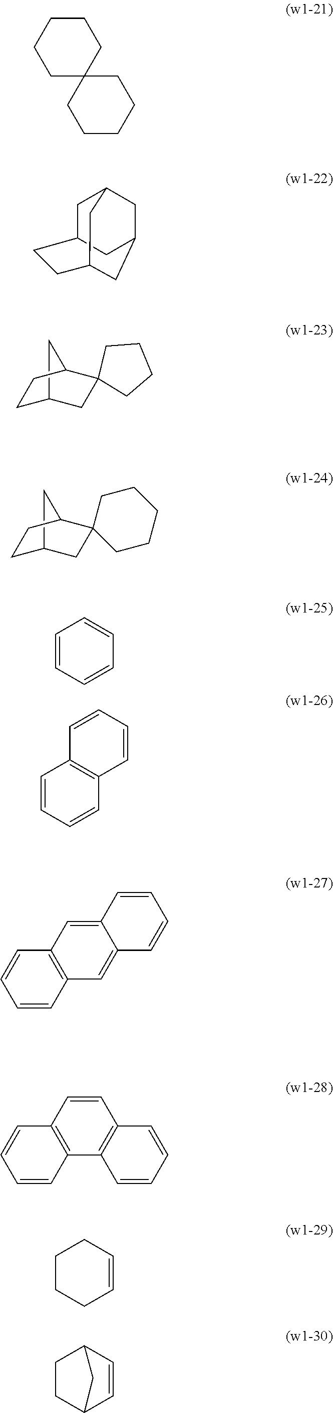

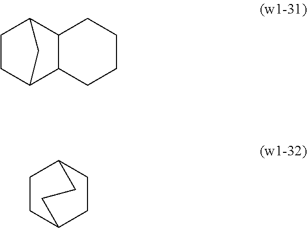

wherein, in formula (w1-1) to formula (w1-32),

a hydrogen atom included in the group may be substituted with a substituent, and —CH₂— included in the group may be replaced by —O—, —S—, —CO— or —SO₂—. The bonding site is any position.

Examples of the substituent which may be substituted with a hydrogen atom included in the ring W¹ and the ring W² include a halogen atom, a haloalkyl group having 1 to 6 carbon atoms, and an alkyl group having 1 to 12 carbon atoms (—CH₂— included in the alkyl group may be replaced by —O— or —CO—).

Examples of the halogen atom include a fluorine atom, a chlorine atom, a bromine atom and an iodine atom.

Examples of the haloalkyl group having 1 to 6 carbon atoms include an alkyl fluoride group having 1 to 6 carbon atoms, an alkyl chloride group having 1 to 6 carbon atoms, an alkyl bromide group having 1 to 6 carbon atoms, an alkyl iodide group having 1 to 6 carbon atoms and the like. Examples of the haloalkyl group include a perfluoroalkyl group having 1 to 6 carbon atoms (a trifluoromethyl group, a pentafluoroethyl group, a heptafluoropropyl group, a nonafluorobutyl group, etc.), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group, a fluoromethyl group, a difluoromethyl group and the like. The number of carbon atoms of the haloalkyl group is preferably 1 to 4, and more preferably 1 to 3.

Examples of the alkyl group having 1 to 12 carbon atoms include alkyl groups such as a methyl group, an ethyl group, a propyl group, an isopropyl group, a butyl group, an isobutyl group, a tert-butyl group, a pentyl group, a hexyl group, an octyl group, a nonyl group and the like. The number of carbon atoms of the alkyl group is preferably 1 to 9, more preferably 1 to 6, and still more preferably 1 to 4.

When —CH₂— included in the alkyl group is replaced by —O— or —CO—, the number of carbon atoms before replacement is taken as the total number of carbon atoms of the alkyl group.

Examples of the replaced group include a hydroxy group (a group in which —CH₂— included in the methyl group is replaced by —O—), a carboxy group (a group in which —CH₂—CH₂—included in the ethyl group is replaced by —O—CO—), an oxy group (a group in which —CH₂— included in the methylene group is replaced by —O—), a carbonyl group (a group in which —CH₂—included in the methylene group is replaced by —CO—), an alkoxy group (a group in which —CH₂— at any position included in the alkyl group is replaced by —O—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—) and the like.

Examples of the alkoxy group include alkoxy groups having 1 to 11 carbon atoms, for example, a methoxy group, an ethoxy group, a propoxy group, a butoxy group, a pentyloxy group, a hexyloxy group, an octyloxy group, a 2-ethylhexyloxy group, a nonyloxy group, a decyloxy group, an undecyloxy group and the like. The number of carbon atoms of the alkoxy group is preferably 1 to 6, more preferably 1 to 4, and still more preferably 1 to 3.

Examples of the alkoxycarbonyl group include alkoxycarbonyl groups having 2 to 11 carbon atoms, for example, a methoxycarbonyl group, an ethoxycarbonyl group, a butoxycarbonyl group and the like. Examples of the alkylcarbonyl group include alkylcarbonyl groups having 2 to 12 carbon atoms, for example, an acetyl group, a propionyl group and a butyryl group. Examples of the alkylcarbonyloxy group include alkylcarbonyloxy groups having 2 to 11 carbon atoms, for example, an acetyloxy group, a propionyloxy group, a butyryloxy group and the like. The number of carbon atoms of the alkoxycarbonyl group, the alkylcarbonyl group and the alkylcarbonyloxy group is preferably 2 to 6, more preferably 2 to 4, and still more preferably 2 or 3, respectively.

The substituent which may be possessed by the ring W¹ and the ring W² is preferably a halogen atom, a haloalkyl group having 1 to 3 carbon atoms or an alkyl group having 1 to 6 carbon atoms (—CH₂— included in the alkyl group may be replaced by —O— or —CO—), more preferably a fluorine atom, an iodine atom, an alkyl fluoride group having 1 to 3 carbon atoms, an alkyl group having 1 to 3 carbon atoms, a hydroxy group, an alkoxy group having 1 to 3 carbon atoms or a carboxy group, and still more preferably a fluorine atom, an iodine atom, a trifluoromethyl group, a methyl group, an ethyl group, a hydroxy group, a methoxy group, an ethoxy group or a carboxy group.

The number of the substituent which may be possessed by the ring W¹ and the ring W² is not particularly limited and is preferably 0, 1 or 2.

The ring W¹ is preferably a cyclic hydrocarbon group having 3 to 18 carbon atoms (a hydrogen atom included in the cyclic hydrocarbon group may be substituted with a substituent, and —CH₂— included in the cyclic hydrocarbon group may be replaced by —O—, —S—, —CO— or —SO₂—), more preferably a cyclic hydrocarbon group represented by formula (W-0) (hereinafter sometimes referred to as “ring W-0”), and still more preferably a cyclic hydrocarbon group represented by formula (W-1) (hereinafter sometimes referred to as “ring W-1”):

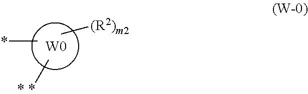

wherein, in formula (W-0),
W0 represents a cyclic hydrocarbon group having 3 to carbon atoms, a carbon atom included in the cyclic hydrocarbon group may form a double bond between two adjacent carbon atoms, —CH₂— included in the cyclic hydrocarbon group may be replaced by —O—, —S—, —CO— or —SO₂—, and a hydrogen atom included in the cyclic hydrocarbon group may be substituted with a substituent,
R² represents a halogen atom, a haloalkyl group having 1 to 6 carbon atoms or an alkyl group having 1 to 12 carbon atoms, —CH₂— included in the alkyl group may be replaced by —O—, —CO—, —S— or —SO₂—, the alkyl group may be bonded to two carbon atoms included in W¹ to form an alkanediyl bridge, —CH₂— included in the alkanediyl bridge may be replaced by —O—, —CO—, —S— or —SO₂—, and the alkanediyl bridge may form a double bond between two adjacent carbon atoms,
m2 represents an integer of 0 to 3, and when m2 is 2 or more, a plurality of R² may be the same or different from each other,
* represents a bonding site to carbon atoms included in COO—, and
** represents a bonding site to X¹:

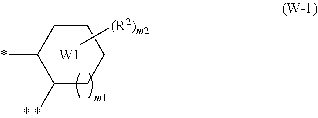

wherein, in formula (W-1),
W¹ represents a cyclic hydrocarbon group having (5+m1) carbon atoms, a carbon atom included in the cyclic hydrocarbon group may form a double bond between two adjacent carbon atoms, —CH₂— included in the cyclic hydrocarbon group may be replaced by —O—, —S—, —CO— or —SO₂—, and a hydrogen atom included in the cyclic hydrocarbon group may be substituted with a substituent,
R² represents a halogen atom, a haloalkyl group having 1 to 6 carbon atoms or an alkyl group having 1 to 12 carbon atoms, —CH₂— included in the alkyl group may be replaced by —O—, —CO—, —S— or —SO₂—, the alkyl group may be bonded to two carbon atoms included in W¹ to form an alkanediyl bridge, —CH₂— included in the alkanediyl bridge may be replaced by —O—, —CO—, —S— or —SO₂—, and the alkanediyl bridge may form a double bond between two adjacent carbon atoms,
m1 represents an integer of 0 to 3,
m2 represents an integer of 0 to 3, and when m2 is 2 or more, a plurality of R² may be the same or different from each other,
* represents a bonding site to carbon atoms included in COO⁻, and ** represents a bonding site to X¹.

In the ring W-0 or ring W-1, a hydrogen atom included in the ring W-0 or ring W-1 may be substituted with a substituent.

In the ring W-0 or the ring W-1, a carbon atom included in the ring W-0 or the ring W-1 may form a double bond between two adjacent carbon atoms. The number of double bonds may be 1, 2, or 3 or more, and an aromatic ring may be formed.

Examples of the cyclic hydrocarbon group having 3 to carbon atoms as for W0 or the cyclic hydrocarbon group having (5+m1) carbon atoms ((5+m1)-membered ring) as for W1 include the same group as mentioned above as long as the upper limit of the number of carbon atoms permits.

The group in which an alkyl group as for R² is bonded to two carbon atoms included in W¹ to form an alkanediyl bridge is a linear or branched alkanediyl group, and examples thereof include a methylene group, an ethylene group, a propanediyl group, a butanediyl group, a pentanediyl group, a hexanediyl group, a 2-methylene-propanediyl group and the like. The number of carbon atoms of R² is preferably 1 to 6, more preferably 1 to 4, and still more preferably 1 to 3.

The alkanediyl group is bonded to two carbon atoms included in the ring W¹, and in this case, the alkanediyl group may be bonded to carbon atoms constituting the cyclic hydrocarbon group of W0 in the ring W-0 or W¹ in the ring W-1 to form an alkanediyl bridge, or two or more alkanediyl groups are present and carbon atoms constituting one alkanediyl group may be bonded to carbon atoms constituting the other alkanediyl group to form an alkanediyl bridge. The substituents mentioned above may be bonded to the alkanediyl bridge. When —CH₂— included in the alkanediyl group is replaced by —O—, —CO—, —S— or —SO₂—, —O— or —S— is preferable.

m1 is preferably an integer of 0 to 2, more preferably 0 or 1, and still more preferably 1.

m2 is preferably an integer of 0 to 2, and more preferably 0 or 1.

The cyclic hydrocarbon group represented by formula (W-0) or formula (W-1) is preferably cyclic hydrocarbon groups mentioned below. In the following cyclic hydrocarbon groups, a bonding site to the carbon atom included in COO— and X¹ can be any position as long as they are adjacent carbon atoms.

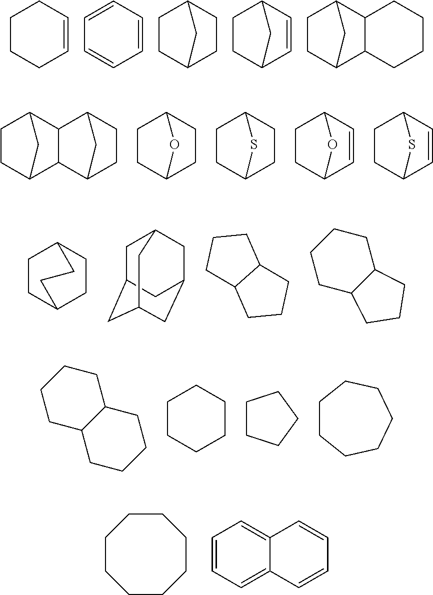

Of these, preferred are cyclic hydrocarbon groups mentioned below.

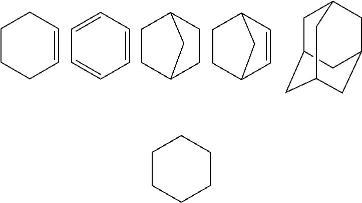

The substituent which may be possessed by the ring W¹ is preferably a halogen atom, a haloalkyl group having 1 to 3 carbon atoms or an alkyl group having 1 to 6 carbon atoms (—CH₂— included in the alkyl group may be replaced by —O— or —CO—), more preferably a fluorine atom, an iodine atom, an alkyl fluoride group having 1 to 3 carbon atoms, an alkyl group having 1 to 3 carbon atoms, a hydroxy group, an alkoxy group having 1 to 3 carbon atoms or a carboxy group, and still more preferably a fluorine atom, an iodine atom, a trifluoromethyl group, a methyl group, an ethyl group, a hydroxy group, a methoxy group, an ethoxy group or a carboxy group.

The ring W² is preferably an aromatic hydrocarbon group having 6 to 10 carbon atoms which may have a halogen atom, a haloalkyl group having 1 to 6 carbon atoms or an alkyl group having 1 to 12 carbon atoms (—CH₂— included in the alkyl group may be replaced by —O—, —CO—, —S— or —SO₂—), and more preferably a phenylene group which may have an iodine atom, a fluorine atom, a trifluoromethyl group, a hydroxy group or a methoxy group.

X¹ is preferably *—CO—O—, *—O—CO— or *—O— (* represents a bonding site to W¹), and more preferably *—CO—O— or *—O— (* represents a bonding site to W¹).

Examples of the hydrocarbon group in L¹ and L² include divalent chain hydrocarbon groups such as an alkanediyl group, divalent cyclic hydrocarbon groups such as a monocyclic or polycyclic (including a spiro ring, a fused ring or a bridged ring) divalent alicyclic hydrocarbon group and a divalent aromatic hydrocarbon group, or the hydrocarbon group may be groups obtained by combining two or more of these groups (e.g., a hydrocarbon group formed from an alicyclic hydrocarbon group and an alkanediyl group).

Examples of the alkanediyl group include linear alkanediyl groups such as a methylene group, an ethylene group, a propane-1,3-diyl group, a butane-1,4-diyl group, a pentane-1,5-diyl group, a hexane-1,6-diyl group, a heptane-1,7-diyl group, a octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group, and

branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The end of the branched alkanediyl group may be an alkyl group such as a methyl group.

The number of carbon atoms of the chain hydrocarbon group is preferably 1 to 18, more preferably 1 to 12, still more preferably 1 to 9, yet more preferably 1 to 6, and further preferably 1 to 4.

Examples of the monocyclic or polycyclic divalent alicyclic hydrocarbon group include the following groups and the like. The bonding site can be any position.

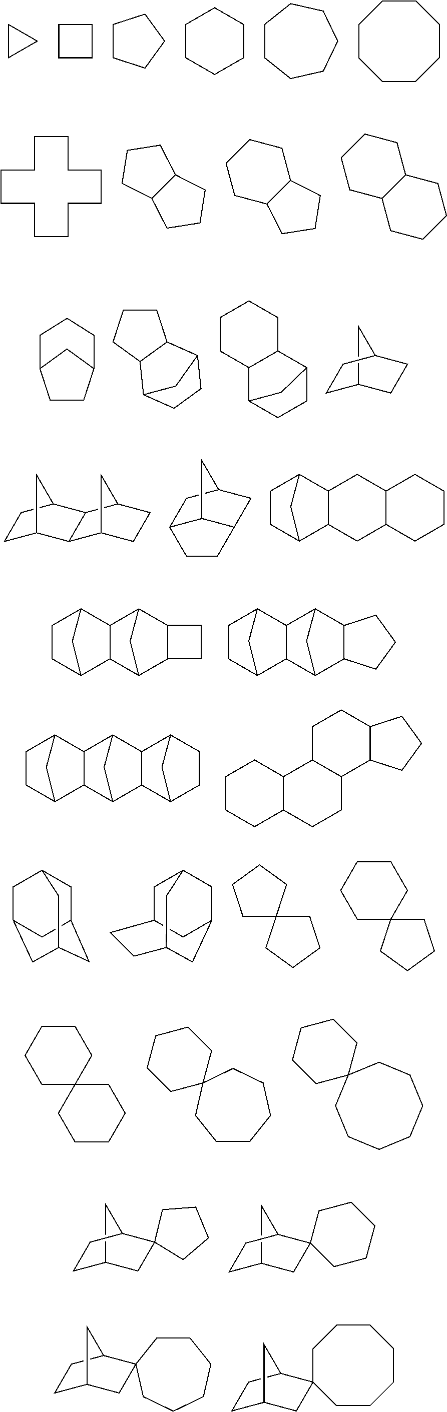

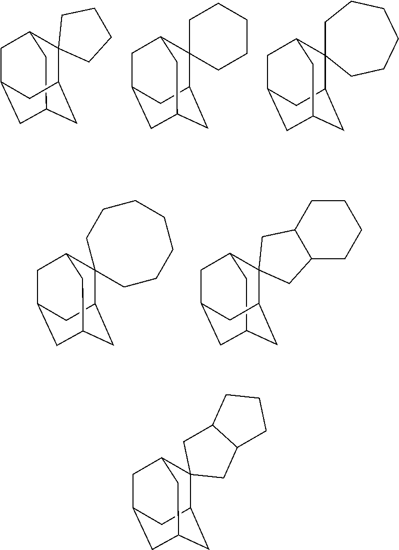

Specifically, examples of the alicyclic hydrocarbon group include monocyclic divalent alicyclic hydrocarbon groups which are monocyclic cycloalkanediyl groups, such as a cyclobutane-1,3-diyl group, a cyclopentane-1,3-diyl group, a cyclohexane-1,4-diyl group and a cyclooctane-1,5-diyl group, and

polycyclic divalent alicyclic hydrocarbon groups which are polycyclic cycloalkanediyl groups, such as a norbornane-1,4-diyl group, a norbornane-2,5-diyl group, an adamantane-1,5-diyl group, an adamantane-2,6-diyl group, spiro rings having a cycloalkyl group, a norbornyl group or an adamantyl group, and a cycloalkyl group spiro-bonded to each group, such as a spirocyclohexane-1,2′-cyclopentane group and a spiroadamantane-2,3′-cyclopentane group. The number of carbon atoms of the alicyclic hydrocarbon group is preferably 3 to 18, more preferably 3 to 16, and still more preferably 3 to 12.

Examples of the divalent aromatic hydrocarbon group include aromatic hydrocarbon groups, for example, arylene groups such as a phenylene group, a naphthylene group, an anthrylene group, a biphenylene group and a phenanthrene group. The number of carbon atoms of the aromatic hydrocarbon group is preferably 6 to 18, more preferably 6 to 14, and still more preferably 6 to 10.

Examples of the groups obtained by combining two or more groups include a group obtained by combining an alicyclic hydrocarbon group with an alkanediyl group, a group obtained by combining an aromatic hydrocarbon group with an alkanediyl group, and a group obtained by combining an alicyclic hydrocarbon group with an aromatic hydrocarbon group. In combination, two or more of alicyclic hydrocarbon groups, aromatic hydrocarbon groups and chain hydrocarbon groups may be respectively combined. Any group may also be bonded to X¹ or W².

Examples of the group obtained by combining an alicyclic hydrocarbon group with an alkanediyl group include a -divalent alicyclic hydrocarbon group-alkanediyl group-, an -alkanediyl group-divalent alicyclic hydrocarbon group-alkanediyl group-, an -alkanediyl group-divalent alicyclic hydrocarbon group- and the like.

Examples of the group obtained by combining an aromatic hydrocarbon group with an alkanediyl group include a -divalent aromatic hydrocarbon group-alkanediyl group-, an -alkanediyl group-divalent aromatic hydrocarbon group-alkanediyl group-, an -alkanediyl group-divalent aromatic hydrocarbon group- and the like.

Examples of the group obtained by combining an alicyclic hydrocarbon group with an aromatic hydrocarbon group include an -aromatic hydrocarbon group-alicyclic hydrocarbon group-, an -alicyclic hydrocarbon group-aromatic hydrocarbon group-alicyclic hydrocarbon group- and the like.

—CH₂— included in the hydrocarbon group having 1 to 28 carbon atoms as for L¹ and L² may be replaced by —O—, —S—, —SO₂— or —CO—.

When —CH₂— included in the hydrocarbon group having 1 to 28 carbon atoms as for L¹ and L² is replaced by —O—, —S—, —SO₂— or —CO—, the number of carbon atoms before replacement is taken as the number of carbon atoms of the hydrocarbon group.

Examples of the group in which —CH₂— included in the hydrocarbon group is replaced by —O—, —S—, —SO₂— or —CO— include a hydroxy group (a group in which —CH₂— included in the methyl group is replaced by —O—), a carboxy group (a group in which —CH₂—CH₂— included in the ethyl group is replaced by —O—CO—), a thiol group (a group in which —CH₂—included in the methyl group is replaced by —S—), an oxy group (a group in which —CH₂— included in the methylene group is replaced by —O—), a carbonyl group (a group in which —CH₂—included in the methylene group is replaced by —CO—), a thio group (a group in which —CH₂— included in the methylene group is replaced by —S—), a sulfonyl group (a group in which —CH₂—included in the methylene group is replaced by —SO₂—), an alkoxy group (a group in which —CH₂— at any position included in the alkyl group is replaced by —O—), an alkylthio group (a group in which —CH₂— at any position included in the alkyl group is replaced by —S—), an alkylsulfonyl group (a group in which —CH₂— at any position included in the alkyl group is replaced by —SO₂—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), an alkanediylsulfonyl group (a group in which —CH₂— at any position included in alkanediyl group is replaced by —SO₂—), an alkanediylthio group (a group in which —CH₂— at any position included in the alkanediyl group is replaced by —S—), a cycloalkoxy group, a cycloalkylalkoxy group, an alkoxycarbonyloxy group, an aromatic hydrocarbon group-carbonyloxy group, an aromatic hydrocarbon group-carbonyl group, an aromatic hydrocarbon group-oxy group, a haloalkoxy group (a group in which —CH₂—at any position included in the haloalkyl group is replaced by —O—), a haloalkoxycarbonyl group (a group in which —CH₂—CH₂— at any position included in the haloalkyl group is replaced by —O—CO—), a haloalkylcarbonyl group (a group in which —CH₂— at any position included in the haloalkyl group is replaced by —CO—), a haloalkylcarbonyloxy group (a group in which —CH₂—CH₂— at any position included in the haloalkyl group is replaced by —CO—O—), and groups obtained by combining two or more of these groups.

Examples of the alkoxy group include alkoxy groups having 1 to 27 carbon atoms, for example, a methoxy group, an ethoxy group, a propoxy group, a butoxy group, a pentyloxy group, a hexyloxy group, an octyloxy group, a 2-ethylhexyloxy group, a nonyloxy group, a decyloxy group, an undecyloxy group and the like. The number of carbon atoms of the alkoxy group may be 1 to 17, and is preferably 1 to 11, more preferably 1 to 6, still more preferably 1 to 4, and yet more preferably 1 to 3.

Examples of the alkylthio group include alkylthio groups having 1 to 27 carbon atoms, for example, a methylthio group, an ethylthio group, a propylthio group, a butylthio group, a pentylthio group, a hexylthio group, an octylthio group, a 2-ethylhexylthio group, a nonylthio group, a decylthio group, an undecylthio group and the like. The number of carbon atoms of the alkylthio group may be 1 to 17, and is preferably 1 to 11, more preferably 1 to 6, still more preferably 1 to 4, and yet more preferably 1 to 3.

The alkoxycarbonyl group, the alkylcarbonyl group and the alkylcarbonyloxy group represent a group in which a carbonyl group or a carbonyloxy group is bonded to the above-mentioned alkyl group or alkoxy group.

Examples of the alkoxycarbonyl group include alkoxycarbonyl groups having 2 to 27 carbon atoms, for example, a methoxycarbonyl group, an ethoxycarbonyl group, a butoxycarbonyl group and the like. Examples of the alkylcarbonyl group include alkylcarbonyl groups having 2 to 28 carbon atoms, for example, an acetyl group, a propionyl group and a butyryl group. Examples of the alkylcarbonyloxy group include alkylcarbonyloxy groups having 2 to 27 carbon atoms, for example, an acetyloxy group, a propionyloxy group, a butyryloxy group and the like. The number of carbon atoms of the alkoxycarbonyl group may be 2 to 17, and is preferably 2 to 11, more preferably 2 to 6, still more preferably 2 to 4, and yet more preferably 2 or 3. The number of carbon atoms of the alkylcarbonyl group may be 2 to 18, and is preferably 2 to 12, more preferably 2 to 6, still more preferably 2 to 4, and yet more preferably 2 or 3. The number of carbon atoms of the alkylcarbonyloxy group may be 2 to 17, and is preferably 2 to 11, more preferably 2 to 6, still more preferably 2 to 4, and yet more preferably 2 or 3.

Examples of the alkylsulfonyl group include alkylsulfonyl groups having 1 to 27 carbon atoms, for example, a methylsulfonyl group, an ethylsulfonyl group, a propylsulfonyl group, a butylsulfonyl group, a pentylsulfonyl group, a hexylsulfonyl group, an octylsulfonyl group, a 2-ethylhexylsulfonyl group, a nonylsulfonyl group, a decylsulfonyl group, an undecylsulfonyl group and the like. The number of carbon atoms of the alkylsulfonyl group may be 1 to 17, and is preferably 1 to 11, more preferably 1 to 6, still more preferably 1 to 4, and yet more preferably 1 to 3.

Examples of the alkanediyloxy group include alkanediyloxy groups having 1 to 27 carbon atoms, for example, a methyleneoxy group, an ethyleneoxy group, a propanediyloxy group, a butanediyloxy group, a pentanediyloxy group and the like. The number of carbon atoms of the alkanediyloxy group may be 1 to 17, and is preferably 1 to 11, more preferably 1 to 6, still more preferably 1 to 4, and yet more preferably 1 to 3.

Examples of the alkanediyloxycarbonyl group include alkanediyloxycarbonyl groups having 2 to 27 carbon atoms, for example, a methyleneoxycarbonyl group, an ethyleneoxycarbonyl group, a propanediyloxycarbonyl group, a butanediyloxycarbonyl group and the like. Examples of the alkanediylcarbonyl group include alkanediylcarbonyl groups having 2 to 28 carbon atoms, for example, a methylenecarbonyl group, an ethylenecarbonyl group, a propanediylcarbonyl group, a butanediylcarbonyl group, a pentanediylcarbonyl group and the like. Examples of the alkanediylcarbonyloxy group include alkanediylcarbonyloxy groups having 2 to 27 carbon atoms, for example, a methylenecarbonyloxy group, an ethylenecarbonyloxy group, a propanediylcarbonyloxy group, a butanediylcarbonyloxy group and the like. The number of carbon atoms of the alkanediyloxycarbonyl group may be 2 to 17, and is preferably 2 to 11, more preferably 2 to 6, still more preferably 2 to 4, and yet more preferably 2 or 3. The number of carbon atoms of the alkanediylcarbonyl group may be 2 to 18, and is preferably 2 to 12, more preferably 2 to 6, still more preferably 2 to 4, and yet more preferably 2 or 3. The number of carbon atoms of the alkanediylcarbonyloxy group may be 2 to 17, and is preferably 2 to 11, more preferably 2 to 6, still more preferably 2 to 4, and yet more preferably 2 or 3.

Examples of the alkanediylsulfonyl group include alkanediylsulfonyl groups having 1 to 27 carbon atoms, for example, a methylenesulfonyl group, an ethylenesulfonyl group, a propylenesulfonyl group and the like. The number of carbon atoms of the alkanediylsulfonyl group may be 1 to 17, and is preferably 1 to 11, more preferably 1 to 6, still more preferably 1 to 4, and yet more preferably 1 to 3.

Examples of the alkanediylthio group include alkanediylthio groups having 1 to 27 carbon atoms, for example, a methylenethio group, an ethylenethio group, a propylenethio group and the like. The number of carbon atoms of the alkanediylthio group may be 1 to 17, and is preferably 1 to 11, more preferably 1 to 6, still more preferably 1 to 4, and yet more preferably 1 to 3.

Examples of the cycloalkoxy group include cycloalkoxy groups having 3 to 27 carbon atoms, for example, a cyclohexyloxy group and the like. Examples of the cycloalkylalkoxy group include cycloalkylalkoxy groups having 4 to 27 carbon atoms, for example, a cyclohexylmethoxy group and the like. Examples of the alkoxycarbonyloxy group include alkoxycarbonyloxy groups having 2 to 26 carbon atoms, for example, a butoxycarbonyloxy group and the like. Examples of the aromatic hydrocarbon group-carbonyloxy group include aromatic hydrocarbon group-carbonyloxy group having 7 to 27 carbon atoms, for example, a benzoyloxy group and the like. Examples of the aromatic hydrocarbon group-carbonyl group include aromatic hydrocarbon group-carbonyl groups having 7 to 28 carbon atoms, for example, a benzoyl group and the like. Examples of the aromatic hydrocarbon group-oxy group include aromatic hydrocarbon group-oxy groups having 6 to 27 carbon atoms, for example, a phenyloxy group and the like.

Examples of the group in which —CH₂— included in the alicyclic hydrocarbon group is replaced by —O—, —S—, —CO— or —SO₂— include the following groups and the like. —O— or —CO— shown below may be respectively replaced by —S— or —SO₂—. The bonding site can be any position.

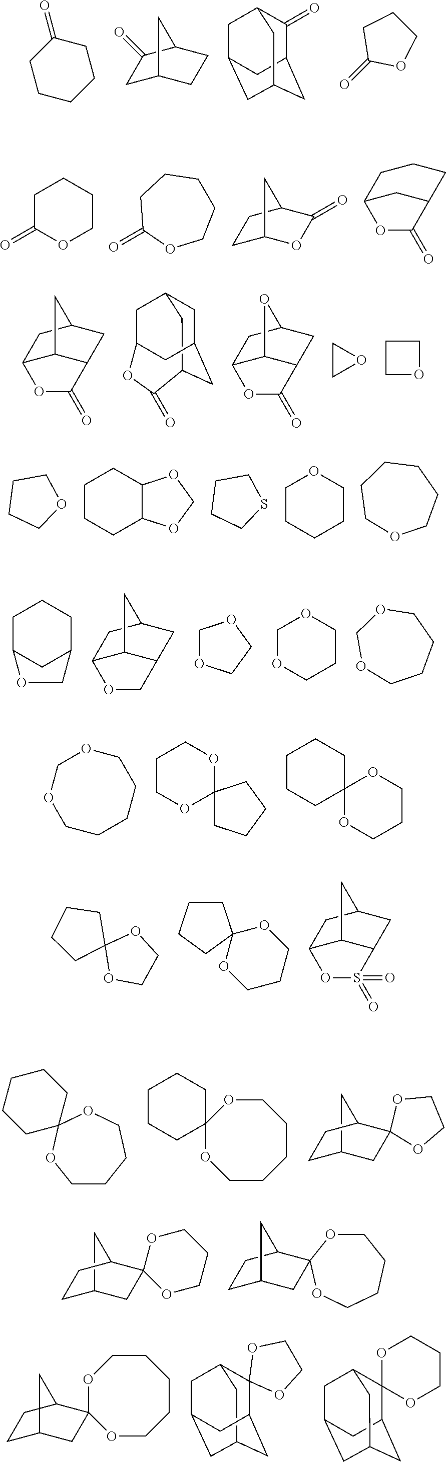

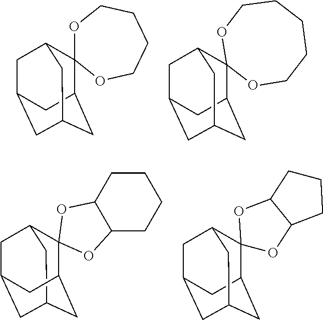

Examples of the substituent which may be possessed by L¹ and L² include a halogen atom, a cyano group, a nitro group and the like. By replacing —CH₂— included in L¹ and L² by —O—, —S—, —CO— or —SO₂—, L¹ and L² can substantially have a substituent such as a hydroxy group, a carboxy group, an alkoxy group, an alkoxycarbonyl group, an alkylcarbonyl group, an alkylcarbonyloxy group, a thiol group or a sulfonyl group. When L¹ and L² are groups obtained by combining an alicyclic hydrocarbon group or an aromatic hydrocarbon group with an alkyl group, the alkyl group can serve as the substituent of the alicyclic hydrocarbon group or the aromatic hydrocarbon group.

Examples of the halogen atom include a fluorine atom, a chlorine atom, a bromine atom and an iodine atom.

The hydrocarbon group as for L¹ and L² may have one substituent or a plurality of substituents.

The substituent in L¹ and L² is preferably an alkyl group having 1 to 4 carbon atoms, a hydroxy group or a halogen atom, more preferably an alkyl group having 1 to 4 carbon atoms or a halogen atom, and still more preferably a methyl group or a fluorine atom.

Preferably, L¹ and L² are each independently a single bond, an alkanediyl group having 1 to 10 carbon atoms (—CH₂—included in the alkanediyl group may be replaced by —O— or —CO—), or a group obtained by combining an alkanediyl group having 1 to 4 carbon atoms with an alicyclic hydrocarbon group having 3 to 12 carbon atoms (—CH₂— included in the alkanediyl group and the alicyclic hydrocarbon group may be replaced by —O— or —CO—), more preferably a single bond or an alkanediyl group having 1 to 8 carbon atoms (—CH₂— included in the alkanediyl group may be replaced by —O— or —CO—), and still more preferably a single bond or a methylene group.

In formula (I), when X²⁰ is a group represented by *-Ax-Ph-Ay-**, preferred is a linking group represented by the following formula (X20):

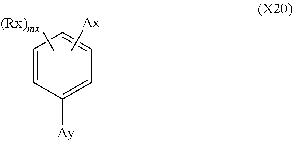

wherein, in formula (X20),
Ax represents bond species bonded to carbon atoms to which R⁵ is bonded, and represents one or more bond species selected from the group consisting of a single bond, an ether bond, a thioether bond, an ester bond, an amide bond and a carbonic acid ester bond,
Ay represents bond species bonded to L², and represents one or more bond species selected from the group consisting of a single bond, an ether bond, a thioether bond, an ester bond, an amide bond and a carbonic acid ester bond,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms, and
mx represents an integer of 0 to 4, and when mx is an integer of 2 or more, a plurality of Rx may be the same or different from each other.

When one of Ax and Ay is a single bond, the other is preferably one selected from the group consisting of an ether bond, a thioether bond, an ester bond, an amide bond and a carbonic acid ester bond.

When either Ax or Ay is an amide bond, a bond represented by —CO—NR³— is preferable.

mx is preferably 0, 1 or 2.

The bonding site of Ay in the phenylene group is preferably the m-position or the p-position, and more preferably the p-position, with respect to the bonding site of Ax.

Rx is preferably a fluorine atom, an iodine atom, a trifluoromethyl group, a methyl group or an ethyl group.

Examples of X²⁰ include groups represented by the following formula (X²⁰-1) to formula (X²⁰-10) * represents a bonding site to carbon atoms to which —R⁵ is bonded. ** represents a bonding site to L² or a ring W²:

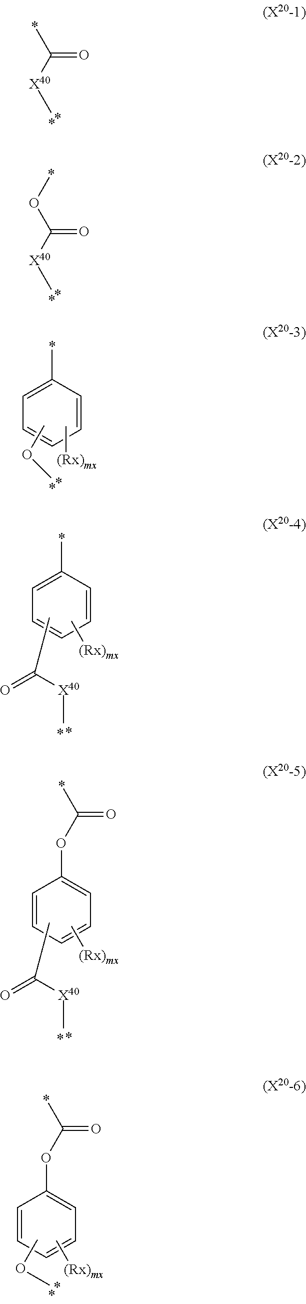

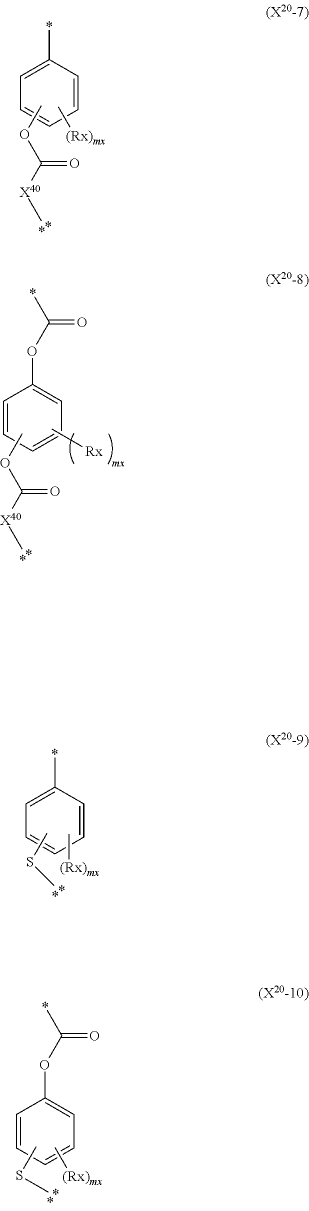

wherein, in formula (X²⁰-1) to formula (X²⁰-10),
* and ** are bonding sites, * represents a bonding site to carbon atoms to which R⁵ is bonded,
Rx represents a halogen atom, a hydroxy group, an alkyl fluoride group having 1 to 6 carbon atoms, an alkyl group having 1 to 18 carbon atoms or an alkoxy group having 1 to 6 carbon atoms,
mx represents an integer of 0 to 4,
X⁴⁰ represents —O— or —NR³—, and
R³ represents a hydrogen atom or an alkyl group having 1 to 6 carbon atoms.

Examples of the alkyl group as for R³ include the same group as mentioned as for the alkyl group as for R⁵.

Specific examples of the groups represented by formula (X²⁰-1) to formula (X²⁰-10) include the following groups.

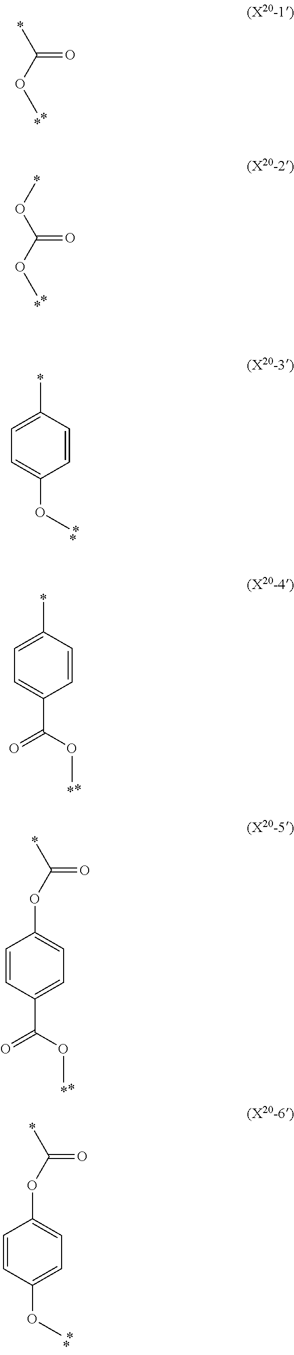

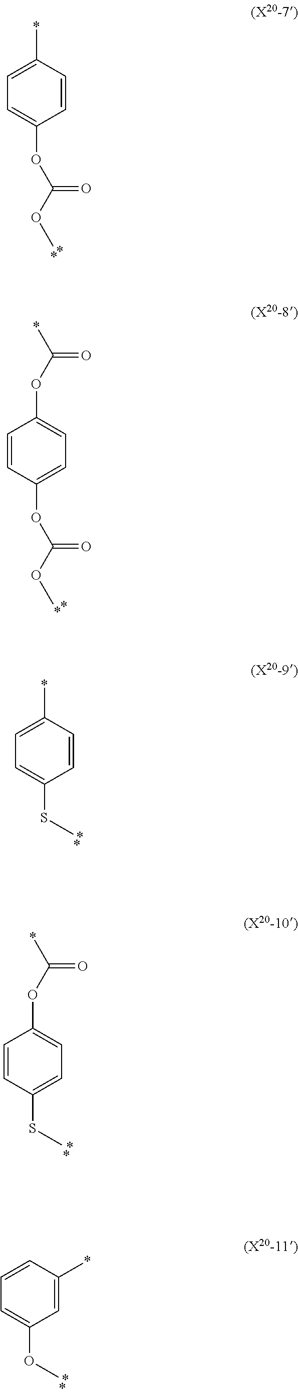

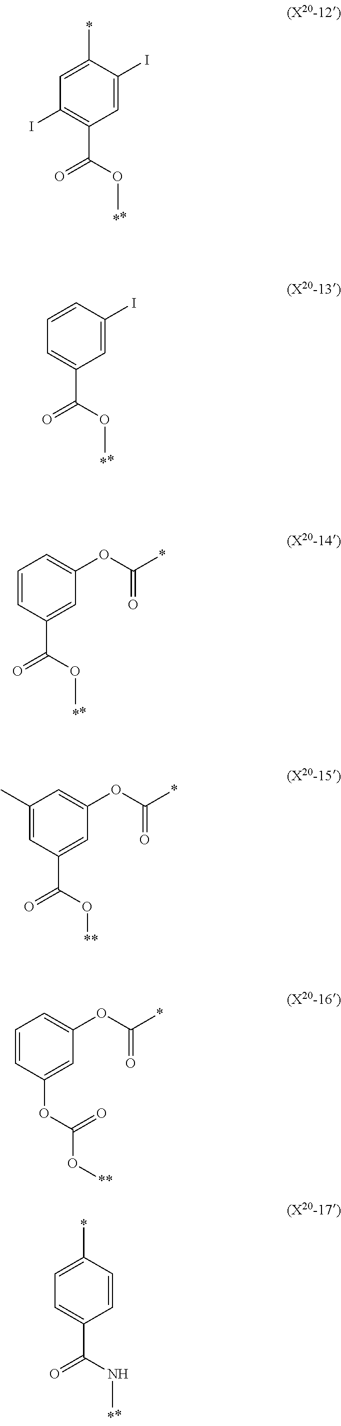

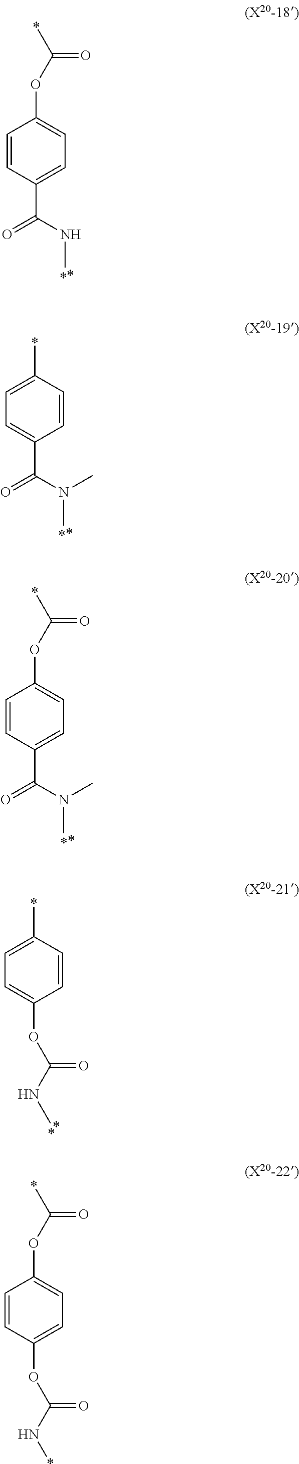 [SMILES: *C(=O)Oc1ccc(C(=O)NC)cc1 ; *c1ccc(C(=O)N(C)C)cc1 ; *C(=O)Oc1ccc(C(=O)N(C)C)cc1 ; *c1ccc(OC(=O)NC)cc1 ; *NC(=O)Oc1ccc(OC(*)=O)cc1]

Particularly, X²⁰ is preferably a single bond or a group represented by any one of formula (X²⁰-1′) and formula (X²⁰-3′) to formula (X²⁰-11′), more preferably a single bond or a group represented by any one of formula (X²⁰-1′), formula (X²⁰-4′), formula (X²⁰-5′), formula (X²⁰-6′), formula (X²⁰-7′) and formula (X²⁰-11′), still more preferably a single bond, a group represented by formula (X²⁰-1′), a group represented by formula (X²⁰-5′) or a group represented by formula (X²⁰-6′), and yet more preferably a single bond or a group represented by formula (X²⁰-1′).

Examples of the halogen atom as for R⁵ include a fluorine atom, a chlorine atom, a bromine atom and an iodine atom.

The alkyl group having 1 to 6 carbon atoms in R⁵ is a linear or branched alkyl group, and examples thereof include alkyl groups such as a methyl group, an ethyl group, a propyl group, an isopropyl group, a butyl group, an isobutyl group, a tert-butyl group, a pentyl group and a hexyl group. The alkyl group as for R⁵ may be a haloalkyl group which is an alkyl group having a halogen atom, and examples of the haloalkyl group include an alkyl fluoride group having 1 to 6 carbon atoms, an alkyl chloride group having 1 to 6 carbon atoms, an alkyl bromide group having 1 to 6 carbon atoms, an alkyl iodide group having 1 to 6 carbon atoms and the like. Examples of the haloalkyl group include a perfluoroalkyl group having 1 to 6 carbon atoms (a trifluoromethyl group, a pentafluoroethyl group, etc.), a chloromethyl group, a bromomethyl group, an iodomethyl group and the like.

R⁵ is preferably a hydrogen atom or an alkyl group having 1 to 3 carbon atoms, and more preferably a hydrogen atom or a methyl group.

Examples of the anion (I) include the following anions. Of these, anions represented by formula (Ia-1) to formula (Ia-8), formula (Ia-18) to formula (Ia-20), formula (Ia-27) to formula (Ia-50) are preferable, and anions represented by formula (Ia-1) to formula (Ia-5), formula (Ia-18) to formula (Ia-20) and formula (Ia-27) to formula (Ia-44) are preferable.

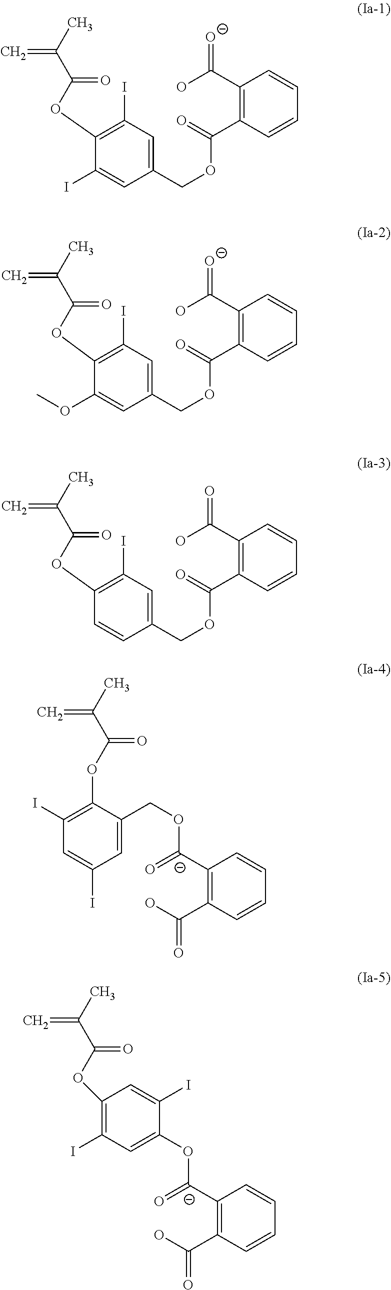

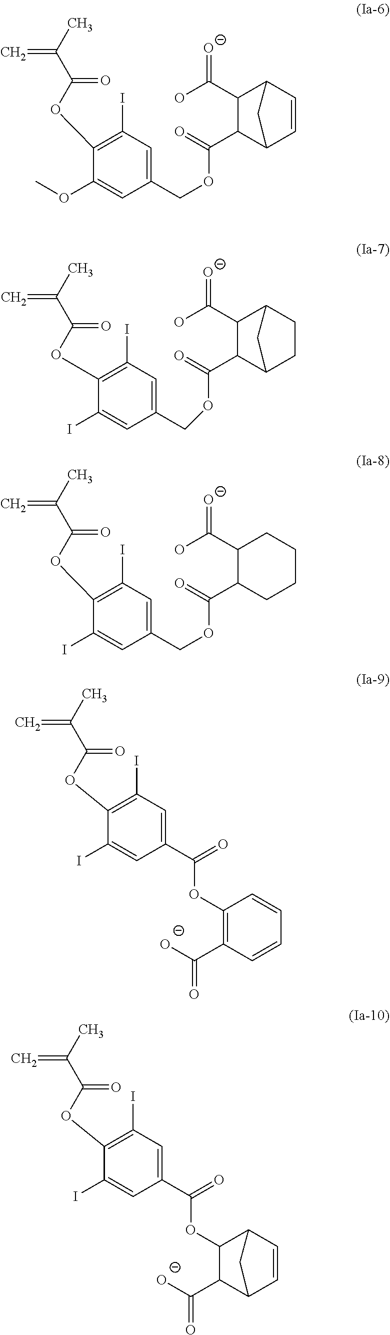

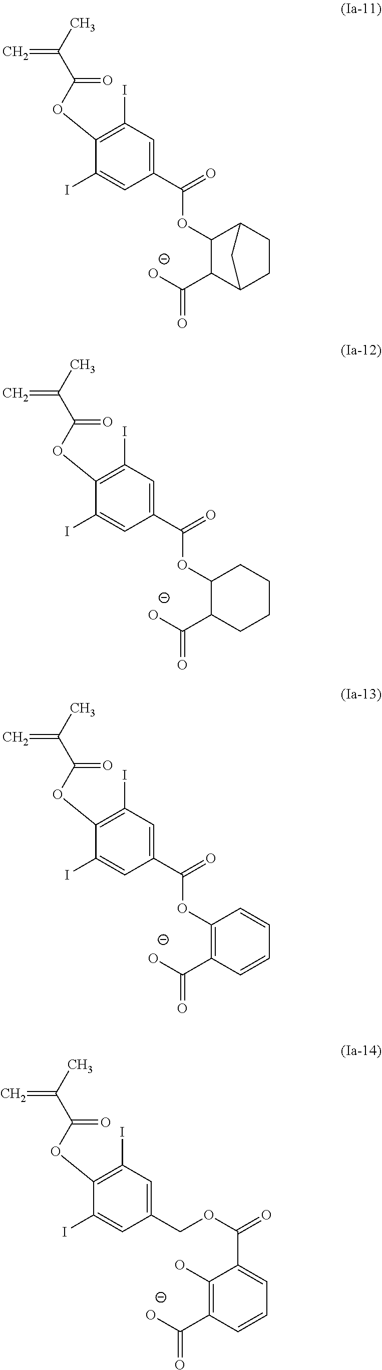 [SMILES: C=C(C)C(=O)Oc1c(I)cc(C(=O)OC2C3CCC(C3)C2C(=O)[O-])cc1I ; C=C(C)C(=O)Oc1c(I)cc(C(=O)OC2CCCCC2C(=O)[O-])cc1I ; C=C(C)C(=O)Oc1c(I)cc(C(=O)Oc2ccccc2C(=O)[O-])cc1I ; C=C(C)C(=O)Oc1c(I)cc(COC(=O)c2cccc(C(=O)[O-])c2O)cc1I]

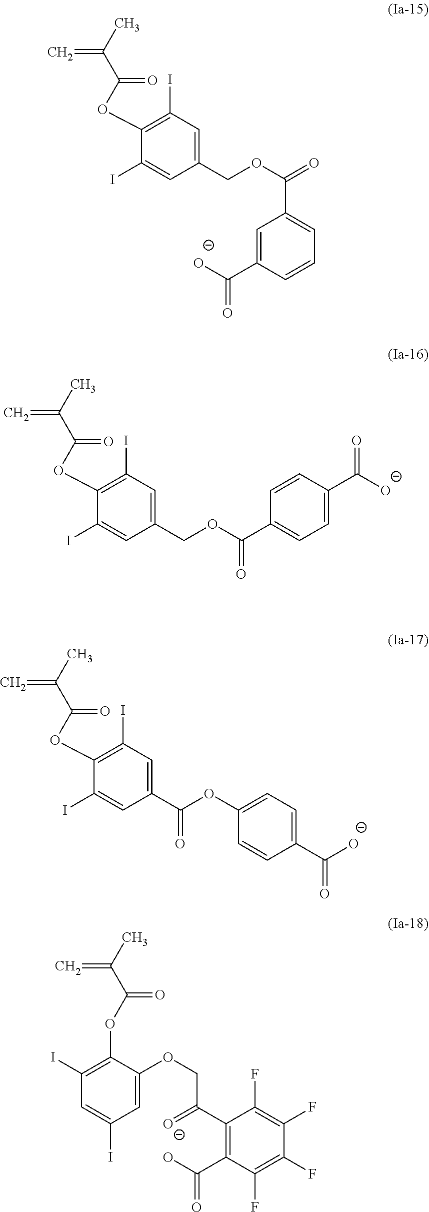

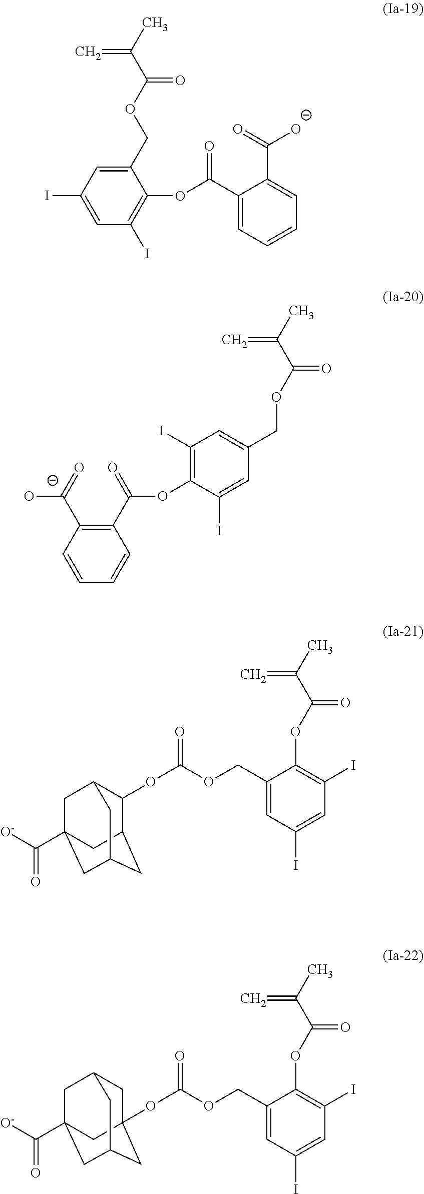 [SMILES: C=C(C)C(=O)OCc1cc(I)cc(I)c1OC(=O)c1ccccc1C(=O)[O-] ; C=C(C)C(=O)OCc1cc(I)c(OC(=O)c2ccccc2C(=O)O)c(I)c1 ; C=C(C)C(=O)Oc1c(I)cc(I)cc1COC(=O)OC1C2CC3CC1CC(C(=O)[O-])(C3)C2 ; C=C(C)C(=O)Oc1c(I)cc(I)cc1COC(=O)OC12CC3CC(C1)CC(C(=O)[O-])(C3)C2]

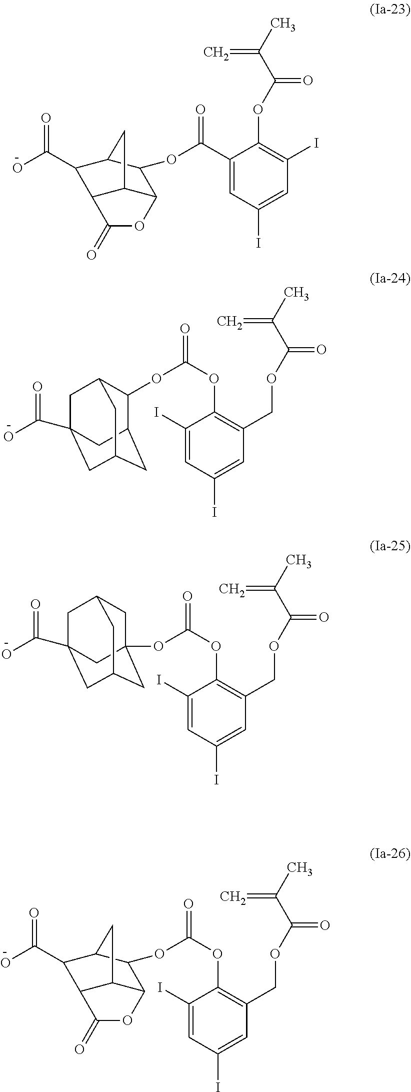 [SMILES: C=C(C)C(=O)Oc1c(I)cc(I)cc1C(=O)OC1C2CC3C1OC(=O)C3C2C(=O)O ; C=C(C)C(=O)OCc1cc(I)cc(I)c1OC(=O)OC1C2CC3CC1CC(C(=O)O)(C3)C2 ; C=C(C)C(=O)OCc1cc(I)cc(I)c1OC(=O)OC12CC3CC(C1)CC(C(=O)O)(C3)C2 ; C=C(C)C(=O)OCc1cc(I)cc(I)c1OC(=O)OC1C2CC3C1OC(=O)C3C2C(=O)O]

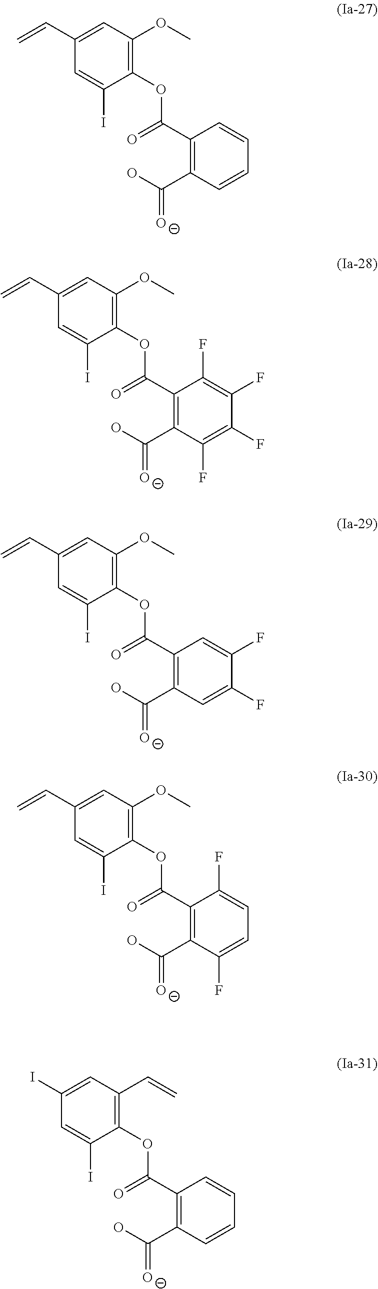

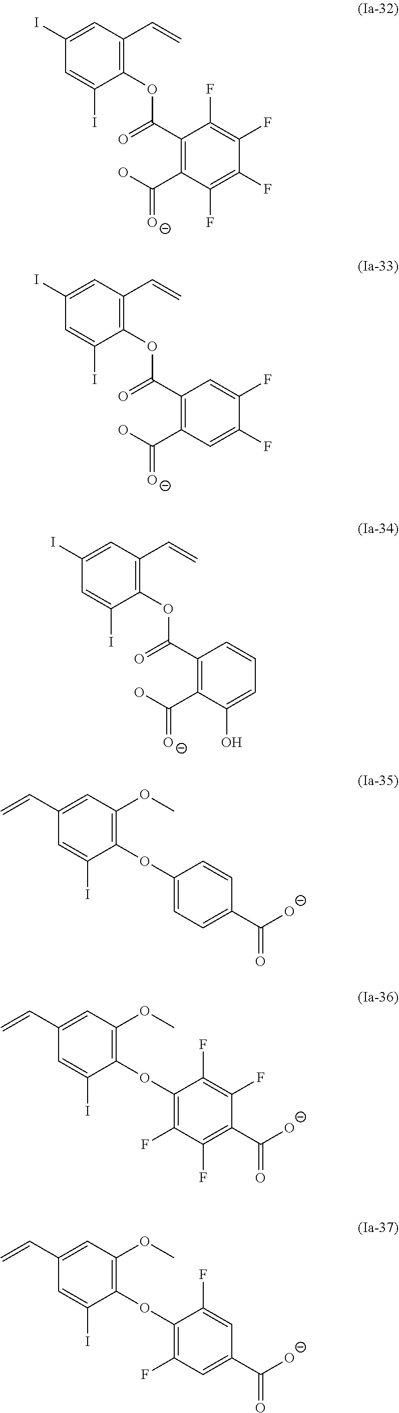

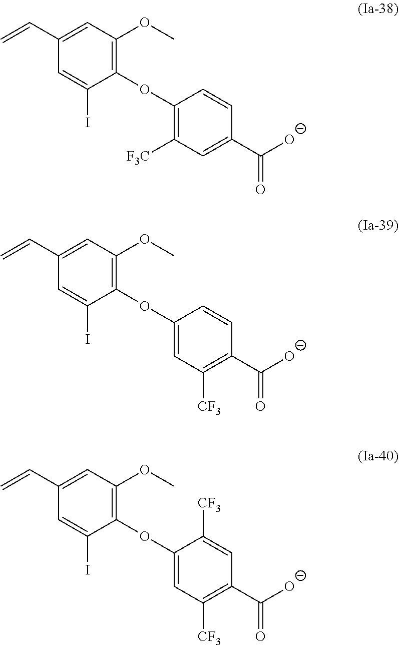 [SMILES: C=Cc1cc(I)c(Oc2ccc(C(=O)[O-])cc2C(F)(F)F)c(OC)c1 ; C=Cc1cc(I)c(Oc2ccc(C(=O)[O-])c(C(F)(F)F)c2)c(OC)c1 ; C=Cc1cc(I)c(Oc2cc(C(F)(F)F)c(C(=O)[O-])cc2C(F)(F)F)c(OC)c1]

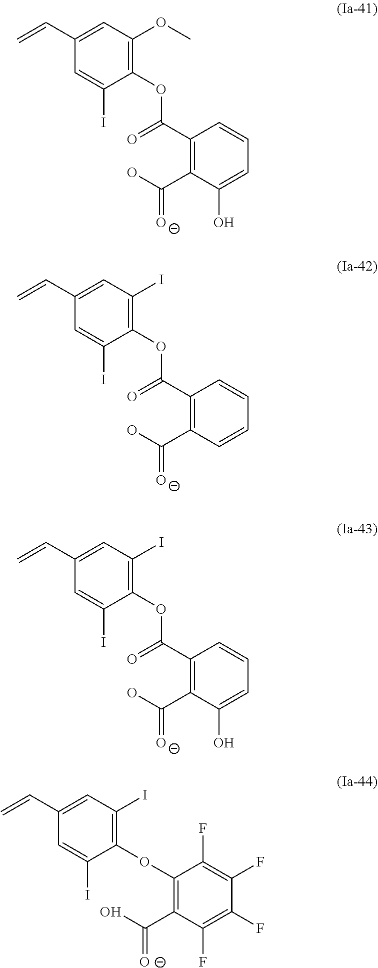

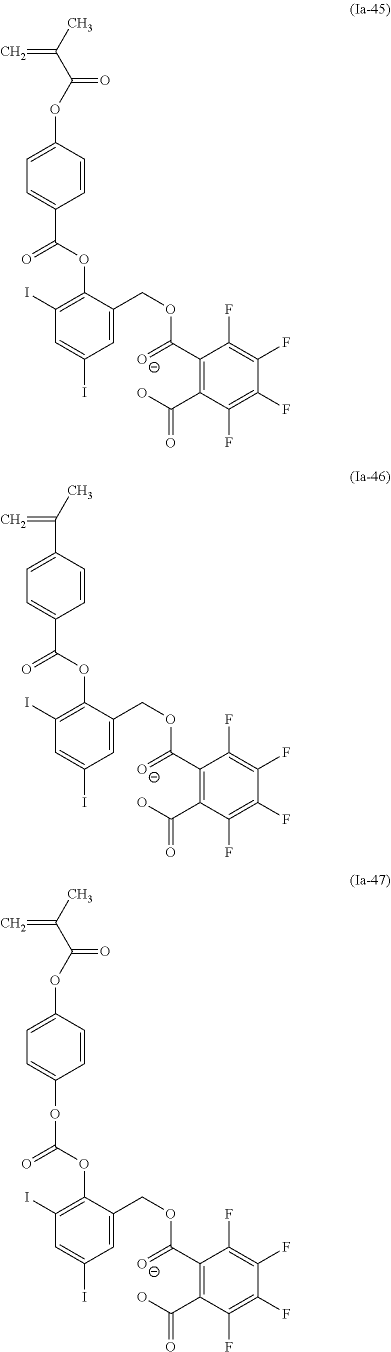

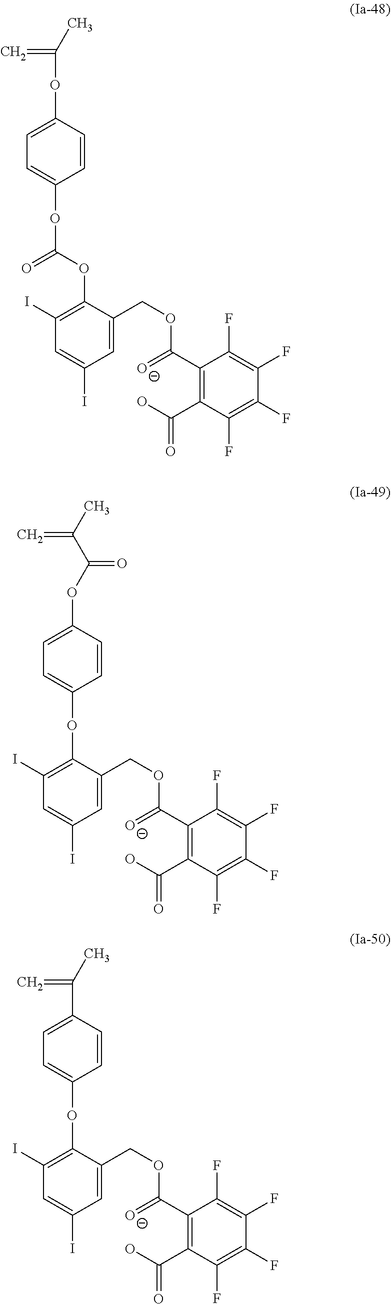

### [Cation (I)]

Examples of the organic cation as for ZI⁺ include an organic onium cation, an organic sulfonium cation, an organic iodonium cation, an organic ammonium cation, a benzothiazolium cation and an organic phosphonium cation. Of these, an organic sulfonium cation and an organic iodonium cation are preferable, and an arylsulfonium cation is more preferable. Specific examples thereof include a cation represented by any one of formula (b2-1) to formula (b2-5) (hereinafter sometimes referred to as “cation (b2-1)” or the like according to the number of formula):

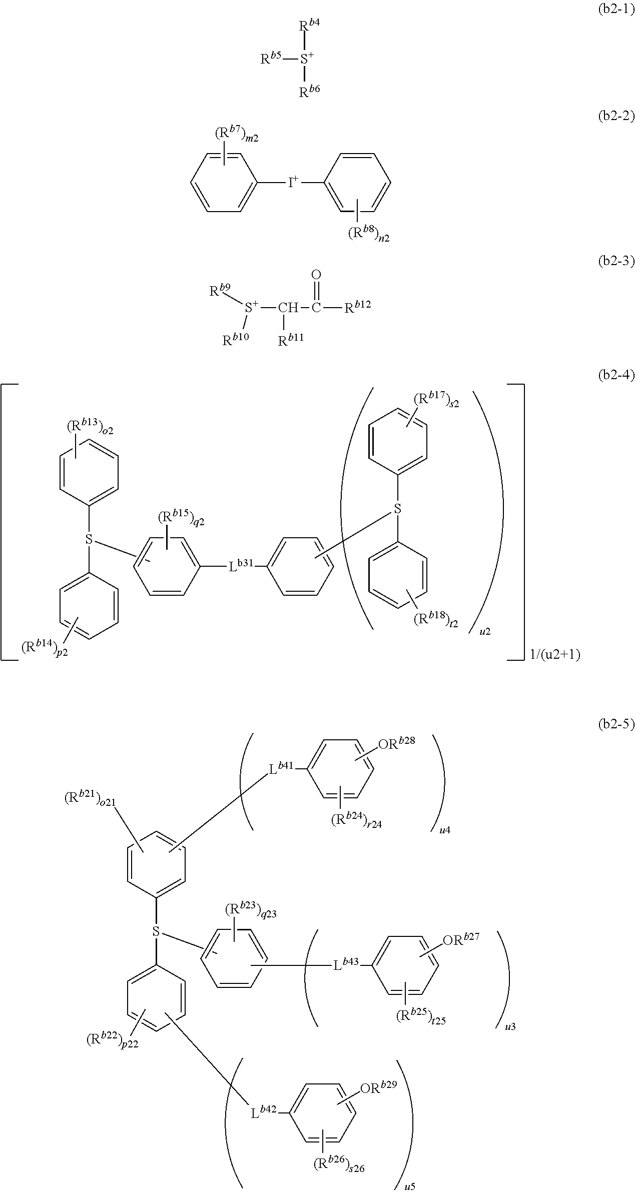

wherein, in formula (b2-1) to formula (b2-5),
R^{b4} to R^{b6} each independently represent a chain hydrocarbon group having 1 to 30 carbon atoms, an alicyclic hydrocarbon group having 3 to 36 carbon atoms or an aromatic hydrocarbon group having 6 to 36 carbon atoms, a hydrogen atom included in the chain hydrocarbon group may be substituted with a hydroxy group, an alkoxy group having 1 to 12 carbon atoms, an alicyclic hydrocarbon group having 3 to 12 carbon atoms or an aromatic hydrocarbon group having 6 to 18 carbon atoms, a hydrogen atom included in the alicyclic hydrocarbon group may be substituted with a halogen atom, an aliphatic hydrocarbon group having 1 to 18 carbon atoms, an alkylcarbonyl group having 2 to 4 carbon atoms or a glycidyloxy group, and a hydrogen atom included in the aromatic hydrocarbon group may be substituted with a halogen atom, a hydroxy group, an aliphatic hydrocarbon group having 1 to 18 carbon atoms, an alkyl fluoride group having 1 to 12 carbon atoms or an alkoxy group having 1 to 12 carbon atoms,
R^{b4} and R^{b5} may be bonded to each other to form a ring together with sulfur atoms to which R^{b4} and R^{b5} are bonded, and —CH₂— included in the ring may be replaced by —O—, —S— or —CO—,
R^{b7} and R^{b8} each independently represent a halogen atom, a hydroxy group, an aliphatic hydrocarbon group having 1 to 12 carbon atoms, an alkyl fluoride group having 1 to 12 carbon atoms or an alkoxy group having 1 to 12 carbon atoms,
m2 and n2 each independently represent an integer of 0 to 5,
when m2 is 2 or more, a plurality of R^{b7} may be the same or different, and when n2 is 2 or more, a plurality of R^{b8} may be the same or different,
R^{b9} and R^{b10} each independently represent a chain hydrocarbon group having 1 to 36 carbon atoms or an alicyclic hydrocarbon group having 3 to 36 carbon atoms,
R^{b9} and R^{b10} may be bonded to each other to form a ring together with sulfur atoms to which R^{b9} and R^{b10} are bonded, and —CH₂— included in the ring may be replaced by —O—, —S— or —CO—,
R^{b11} represents a hydrogen atom, a chain hydrocarbon group having 1 to 36 carbon atoms, an alicyclic hydrocarbon group having 3 to 36 carbon atoms or an aromatic hydrocarbon group having 6 to 18 carbon atoms,
R^{b12} represents a chain hydrocarbon group having 1 to 12 carbon atoms, an alicyclic hydrocarbon group having 3 to 18 carbon atoms or an aromatic hydrocarbon group having 6 to 18 carbon atoms, a hydrogen atom included in the chain hydrocarbon group may be substituted with an aromatic hydrocarbon group having 6 to 18 carbon atoms, a hydrogen atom included in the aromatic hydrocarbon group may be substituted with an alkoxy group having 1 to 12 carbon atoms or an alkylcarbonyloxy group having 1 to 12 carbon atoms,
R^{b11} and R^{b12} may be bonded to each other to form a ring, including —CH—CO— to which R^{b11} and R^{b12} are bonded, and —CH₂— included in the ring may be replaced by —O—, —S— or —CO—,
R^{b13} to R^{b18} and R^{b21} to R^{b26} each independently represent a halogen atom, a hydroxy group, an aliphatic hydrocarbon group having 1 to 12 carbon atoms, an alkyl fluoride group having 1 to 12 carbon atoms or an alkoxy group having 1 to 12 carbon atoms,
R^{b13} and R^{b14}, and R^{b21} and R^{b22} may be bonded to each other to form a ring having a sulfur atom together with the benzene ring to which R^{b13} and R^{b14} are bonded, and —CH₂— included in the ring may be replaced by —O—, —S— or —CO—,
R^{b27} to R^{b29} each independently represent a hydrogen atom or a group having an acid-labile group,
L^{b31} and L^{b4}1 to L^{b43} each independently represent a sulfur atom or an oxygen atom,
o2, p2, s2 and t2 each independently represent an integer of 0 to 5,
q2 and r2 each independently represent an integer of 0 to 4,
u2 represents 0 or 1,
u3 represents an integer of 1 to 3, and satisfies: 1≤u3+q23≤5,
u4 and u5 each independently represent an integer of 0 to 3, and satisfy: 0≤u4+o21≤5 and 0≤u5+p22≤5,
o21, p22, q23, r24, t25 and s26 each independently represent an integer of 0 to 4,
when o2 is 2 or more, a plurality of R^{b13} are the same or different, when p2 is 2 or more, a plurality of R^{b14} are the same or different, when q2 is 2 or more, a plurality of R^{b15} are the same or different, when r2 is 2 or more, a plurality of R^{b16} are the same or different, when s2 is 2 or more, a plurality of R^{b17} are the same or different, and when t2 is 2 or more, a plurality of R^{b18} are the same or different,
when u3, u4 and u5 are each 2 or more, a plurality of groups in parentheses are each the same or different, and
when o21, p22, q23, r24, t25 and s26 are each 2 or more, a plurality of R^{b21} to R^{b26} are each the same or different.

When u2 is 0, any one of o2, p2, q2 and r2 is preferably 1 or more and at least one of R^{b13} to R^{b16} is preferably a halogen atom, and when u2 is 1, any one of o2, p2, s2, t2, q2 and r2 is preferably 1 or more and at least one of R^{b13} to R^{b18} is preferably a halogen atom.

Further, when u2 is 0, r2 is preferably 1 or more, and more preferably 1. When u2 is 0 and r2 is 1 or more, R^{b16} is preferably a halogen atom.

The aliphatic hydrocarbon group represents a chain hydrocarbon group and an alicyclic hydrocarbon group.

Examples of the chain hydrocarbon group include alkyl groups such as a methyl group, an ethyl group, a propyl group, an isopropyl group, a butyl group, a sec-butyl group, a tert-butyl group, a pentyl group, a hexyl group, an octyl group and a 2-ethylhexyl group.

Particularly, the chain hydrocarbon group of R^{b9} to R^{b12} preferably has 1 to 12 carbon atoms.

The alicyclic hydrocarbon group may be either monocyclic or polycyclic, and examples of the monocyclic alicyclic hydrocarbon group include cycloalkyl groups such as a cyclopropyl group, a cyclobutyl group, a cyclopentyl group, a cyclohexyl group, a cycloheptyl group, a cyclooctyl group and a cyclodecyl group. Examples of the polycyclic alicyclic hydrocarbon group include a decahydronaphthyl group, an adamantyl group, a norbornyl group and the following groups.

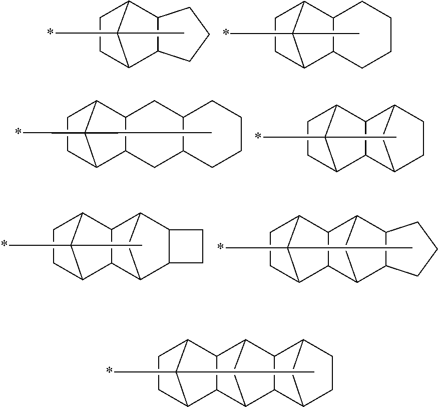

Particularly, the alicyclic hydrocarbon group of R^{b9} to R^{b12} preferably has 3 to 18 carbon atoms, and more preferably 4 to 12 carbon atoms.

Examples of the alicyclic hydrocarbon group in which a hydrogen atom is substituted with an aliphatic hydrocarbon group include a methylcyclohexyl group, a dimethylcyclohexyl group, a 2-methyladamantan-2-yl group, a 2-ethyladamantan-2-yl group, a 2-isopropyladamantan-2-yl group, a methylnorbornyl group, an isobornyl group and the like. In the alicyclic hydrocarbon group in which a hydrogen atom is substituted with an aliphatic hydrocarbon group, the total number of carbon atoms of the alicyclic hydrocarbon group and the aliphatic hydrocarbon group is preferably 20 or less.

The alkyl fluoride group represents an alkyl group having 1 to 12 carbon atoms which has a fluorine atom, and examples thereof include a fluoromethyl group, a difluoromethyl group, a trifluoromethyl group, a perfluorobutyl and the like. The number of carbon atoms of the alkyl fluoride group is preferably 1 to 9, more preferably 1 to 6, still more preferably 1 to 4.

Examples of the aromatic hydrocarbon group include aryl groups such as a phenyl group, a biphenyl group, a naphthyl group and a phenanthryl group. The aromatic hydrocarbon group may have a chain hydrocarbon group or an alicyclic hydrocarbon group, and examples thereof include aromatic hydrocarbon groups which have a chain hydrocarbon group having 1 to 18 carbon atoms (a tolyl group, a xylyl group, a cumenyl group, a mesityl group, a p-ethylphenyl group, a p-tert-butylphenyl group, a 2,6-diethylphenyl group, a 2-methyl-6-ethylphenyl group, etc.) and aromatic hydrocarbon groups which have an alicyclic hydrocarbon group having 3 to 18 carbon atoms (a p-cyclohexylphenyl group, a p-adamantylphenyl group, etc.) and the like. When the aromatic hydrocarbon group has a chain hydrocarbon group or an alicyclic hydrocarbon group, a chain hydrocarbon group having 1 to 18 carbon atoms and an alicyclic hydrocarbon group having 3 to 18 carbon atoms are preferable.

Examples of the aromatic hydrocarbon group in which a hydrogen atom is substituted with an alkoxy group include a p-methoxyphenyl group and the like.

Examples of the chain hydrocarbon group in which a hydrogen atom is substituted with an aromatic hydrocarbon group include aralkyl groups such as a benzyl group, a phenethyl group, a phenylpropyl group, a trityl group, a naphthylmethyl group and a naphthylethyl group.

Examples of the alkoxy group include a methoxy group, an ethoxy group, a propoxy group, a butoxy group, a pentyloxy group, a hexyloxy group, a heptyloxy group, an octyloxy group, a decyloxy group and a dodecyloxy group.

Examples of the alkylcarbonyl group include an acetyl group, a propionyl group and a butyryl group.

Examples of the halogen atom include a fluorine atom, a chlorine atom, a bromine atom and an iodine atom.

Examples of the alkylcarbonyloxy group include a methylcarbonyloxy group, an ethylcarbonyloxy group, a propylcarbonyloxy group, an isopropylcarbonyloxy group, a butylcarbonyloxy group, a sec-butylcarbonyloxy group, a tert-butylcarbonyloxy group, a pentylcarbonyloxy group, a hexylcarbonyloxy group, an octylcarbonyloxy group and a 2-ethylhexylcarbonyloxy group.

The ring formed by bonding R^{b4} and R^{b5} each other, together with sulfur atoms to which R^{b4} and R^{b5} are bonded, may be a monocyclic, polycyclic, aromatic, nonaromatic, saturated or unsaturated ring. This ring includes a ring having 3 to 18 carbon atoms and is preferably a ring having 4 to 18 carbon atoms. The ring having a sulfur atom includes a 3-membered to 12-membered ring and is preferably a 3-membered to 7-membered ring and includes, for example, the following rings and the like. * represents a bonding site.

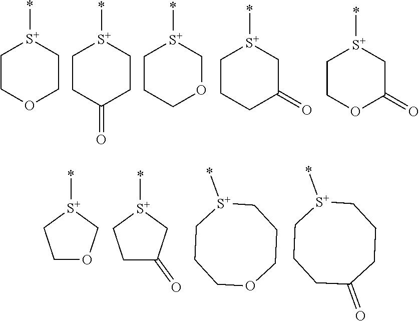

The ring formed by combining R^{b9} and R^{b10} together may be a monocyclic, polycyclic, aromatic, nonaromatic, saturated or unsaturated ring. This ring includes a 3-membered to 12-membered ring and is preferably a 3-membered to 7-membered ring. The ring includes, for example, a thiolan-1-ium ring (tetrahydrothiophenium ring), a thian-1-ium ring, a 1,4-oxathian-4-ium ring and the like.

The ring formed by combining R^{b1} and R^{b12} together may be a monocyclic, polycyclic, aromatic, nonaromatic, saturated or unsaturated ring. This ring includes a 3-membered to 12-membered ring and is preferably a 3-membered to 7-membered ring. Examples thereof include an oxocycloheptane ring, an oxocyclohexane ring, an oxonorbornane ring, an oxoadamantane ring and the like.

Examples of the group having an acid-labile group as for R^{b27} to R^{b29} include a group represented by —R^{c30}, —CO—O—R^{c30} or -L^{c10}-CO—O—R^{c30} (L^{c10} represents an alkanediyl group having 1 to 6 carbon atoms, and R^{c30} represents an acid-labile group) and the like.

Examples of the acid-labile group as for R^{b27} to R^{b29} include groups represented by formula (1) or formula (2) mentioned above.

Of cation (b2-1) to cation (b2-5), a cation (b2-1), a cation (b2-4) and a cation (b2-5) are preferable.

Examples of the cation (b2-1) include the following cations.

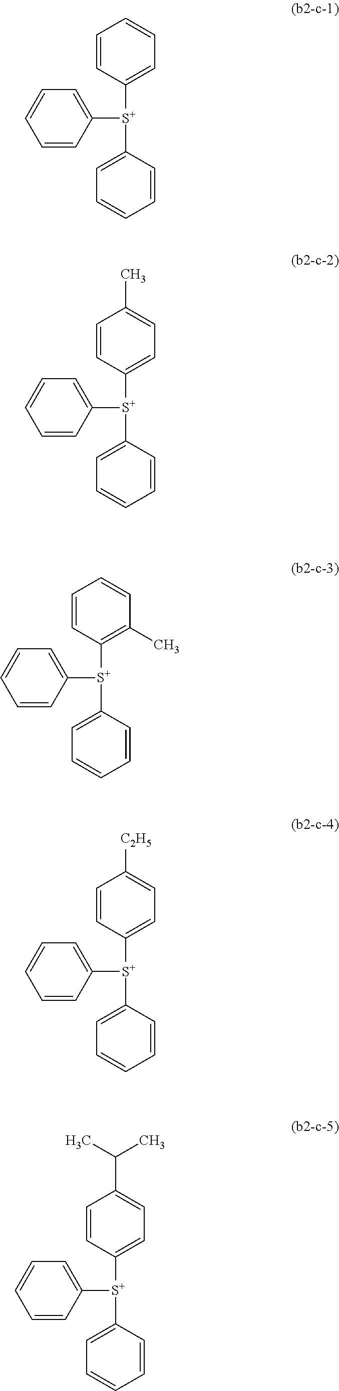 [SMILES: c1ccc([S+](c2ccccc2)c2ccccc2)cc1 ; Cc1ccc([S+](c2ccccc2)c2ccccc2)cc1 ; Cc1ccccc1[S+](c1ccccc1)c1ccccc1 ; CCc1ccc([S+](c2ccccc2)c2ccccc2)cc1 ; CC(C)c1ccc([S+](c2ccccc2)c2ccccc2)cc1]

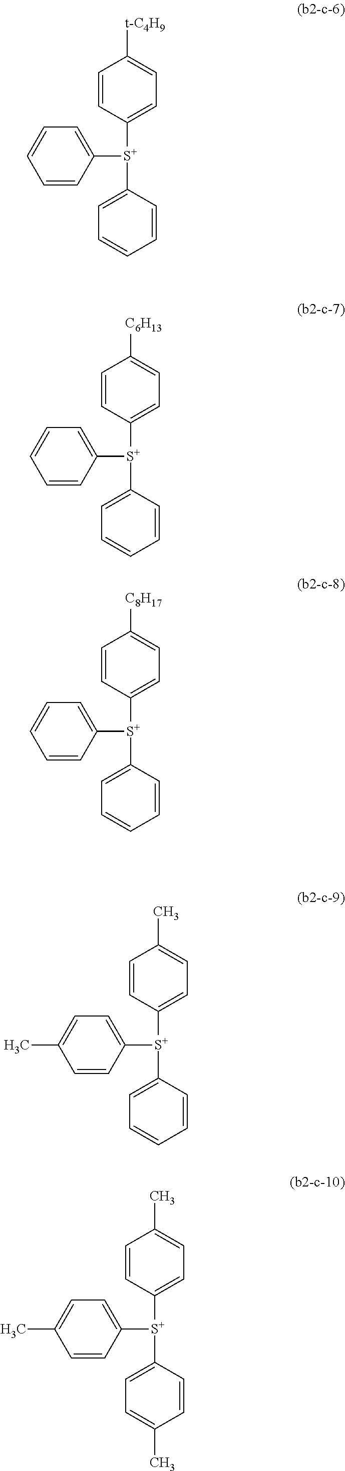

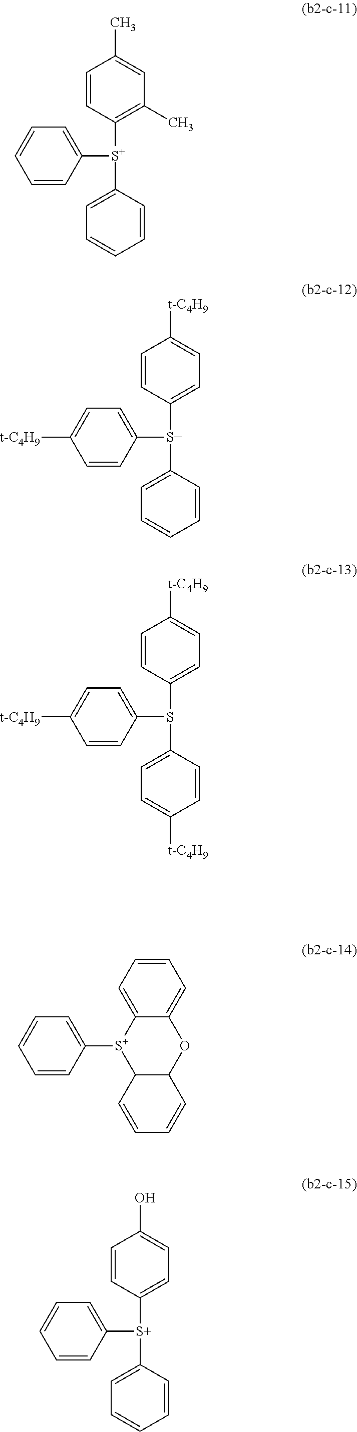

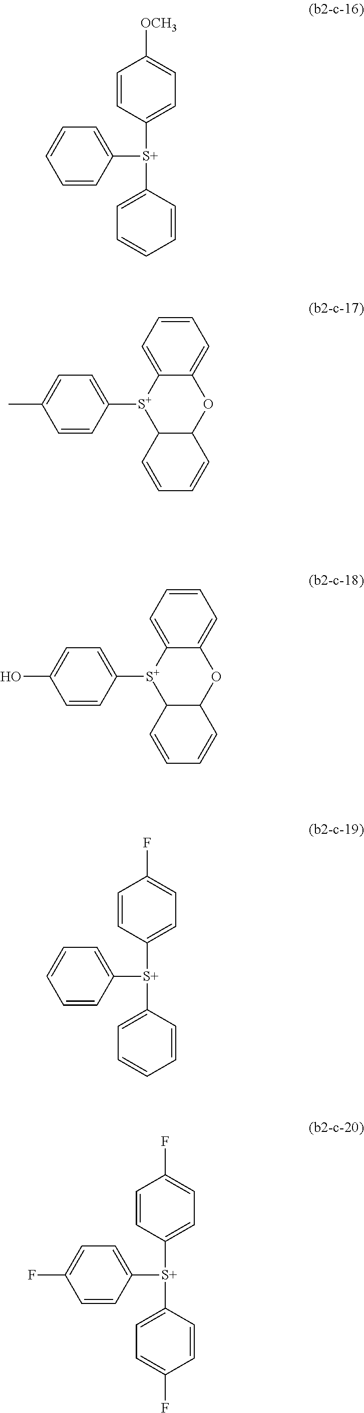 [SMILES: COc1ccc([S+](c2ccccc2)c2ccccc2)cc1 ; Cc1ccc([S+]2c3ccccc3OC3C=CC=CC32)cc1 ; Oc1ccc([S+]2c3ccccc3OC3C=CC=CC32)cc1 ; Fc1ccc([S+](c2ccccc2)c2ccccc2)cc1 ; Fc1ccc([S+](c2ccc(F)cc2)c2ccc(F)cc2)cc1]

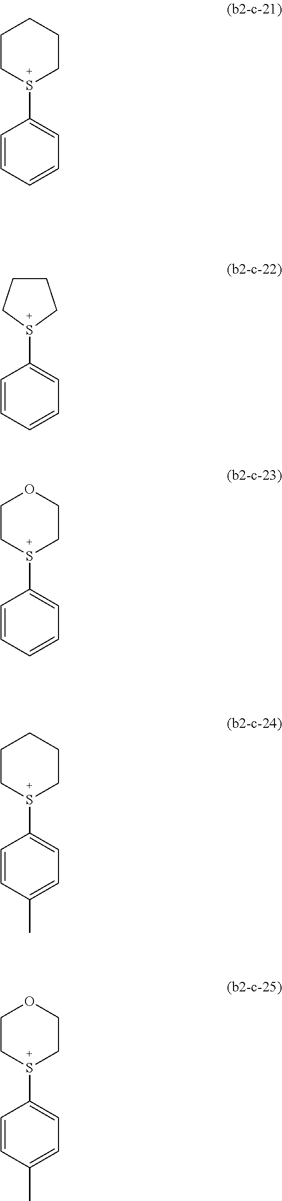

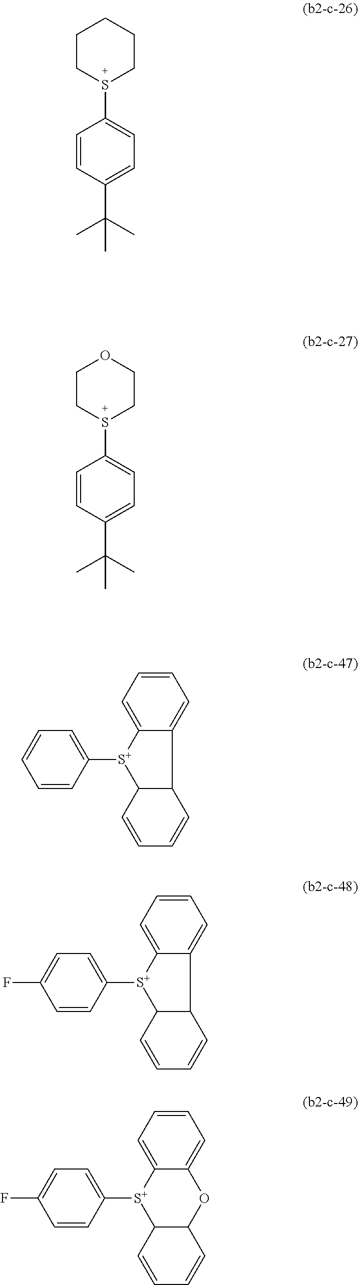

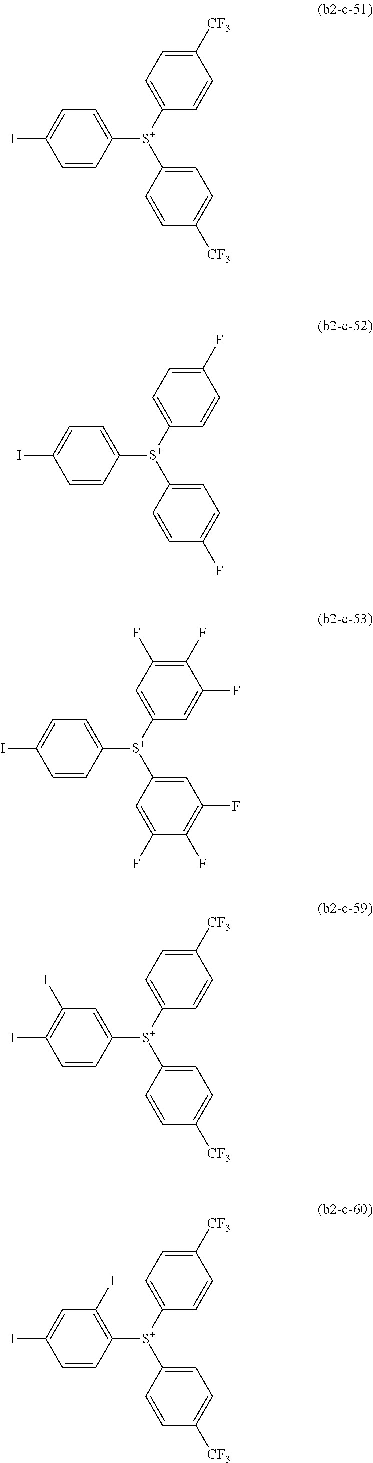 [SMILES: FC(F)(F)c1ccc([S+](c2ccc(I)cc2)c2ccc(C(F)(F)F)cc2)cc1 ; Fc1ccc([S+](c2ccc(F)cc2)c2ccc(I)cc2)cc1 ; Fc1cc([S+](c2ccc(I)cc2)c2cc(F)c(F)c(F)c2)cc(F)c1F ; FC(F)(F)c1ccc([S+](c2ccc(C(F)(F)F)cc2)c2ccc(I)c(I)c2)cc1 ; FC(F)(F)c1ccc([S+](c2ccc(C(F)(F)F)cc2)c2ccc(I)cc2I)cc1]

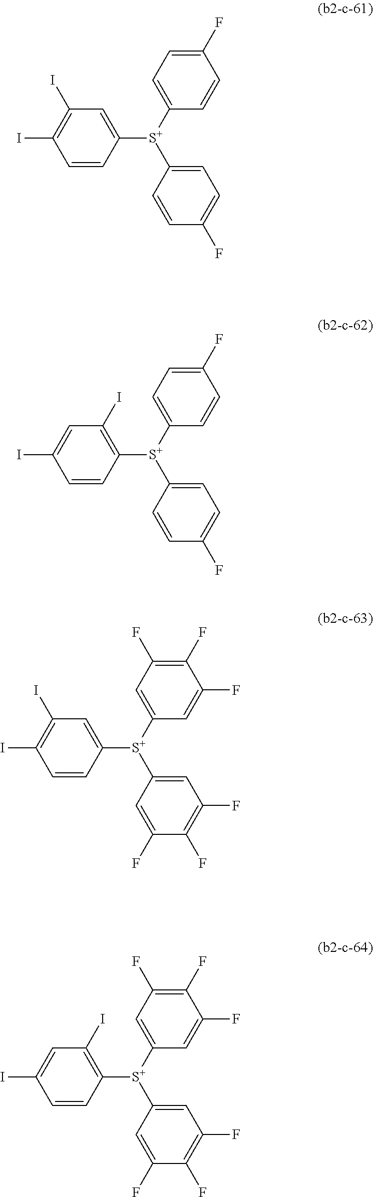 [SMILES: Fc1ccc([S+](c2ccc(F)cc2)c2ccc(I)c(I)c2)cc1 ; Fc1ccc([S+](c2ccc(F)cc2)c2ccc(I)cc2I)cc1 ; Fc1cc([S+](c2cc(F)c(F)c(F)c2)c2ccc(I)c(I)c2)cc(F)c1F ; Fc1cc([S+](c2cc(F)c(F)c(F)c2)c2ccc(I)cc2I)cc(F)c1F]

Examples of the cation (b2-2) include the following cations.

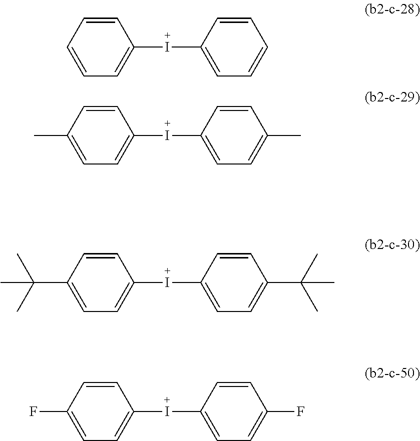

Examples of the cation (b2-3) include the following cations.

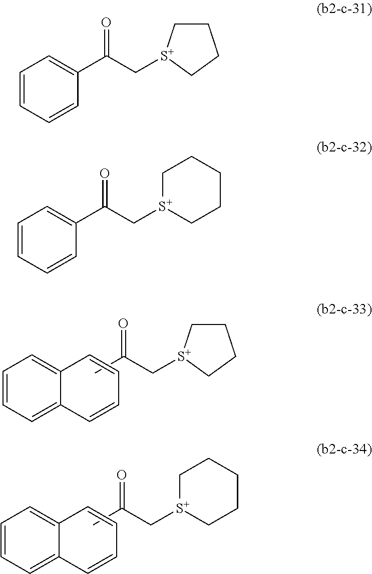

Examples of the cation (b2-4) include the following cations.

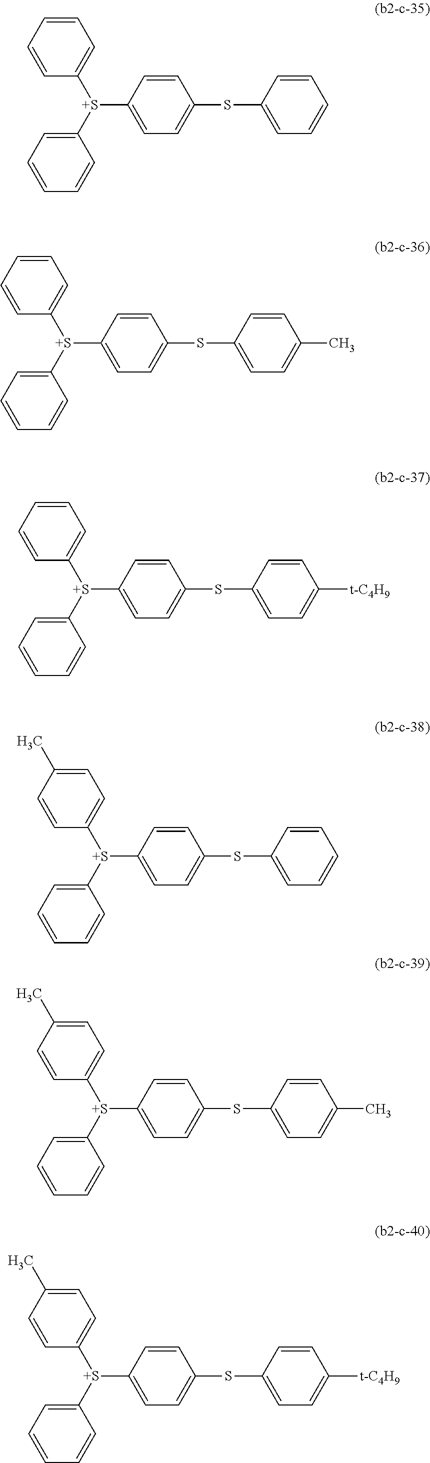

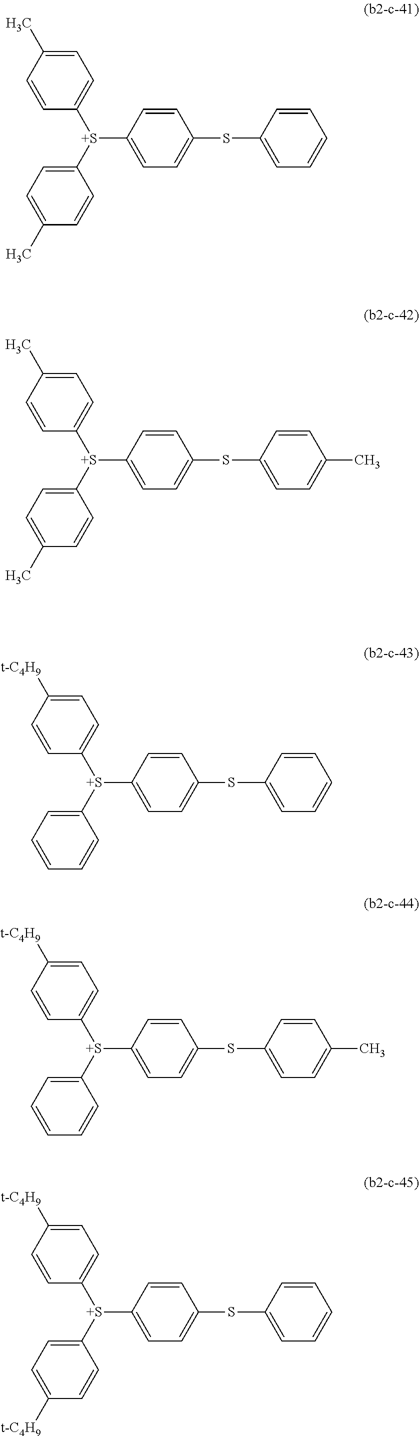

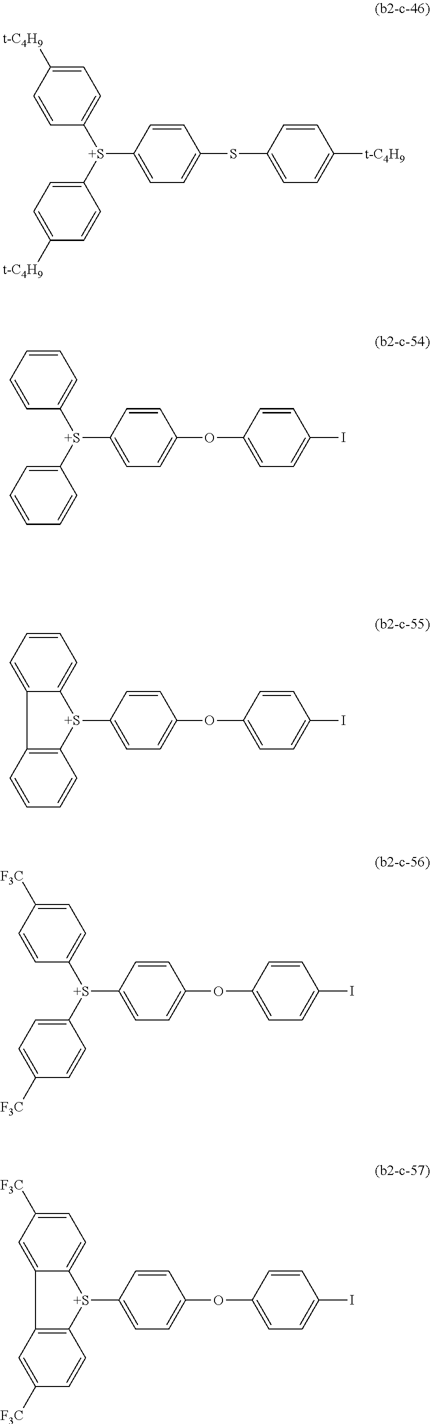

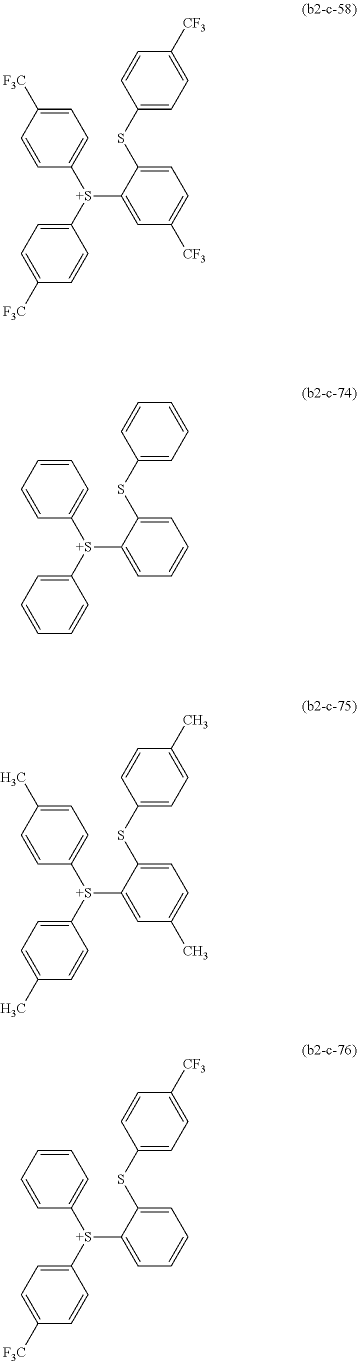 [SMILES: FC(F)(F)c1ccc(Sc2ccc(C(F)(F)F)cc2[S+](c2ccc(C(F)(F)F)cc2)c2ccc(C(F)(F)F)cc2)cc1 ; c1ccc(Sc2ccccc2[S+](c2ccccc2)c2ccccc2)cc1 ; Cc1ccc(Sc2ccc(C)cc2[S+](c2ccc(C)cc2)c2ccc(C)cc2)cc1 ; FC(F)(F)c1ccc(Sc2ccccc2[S+](c2ccccc2)c2ccc(C(F)(F)F)cc2)cc1]

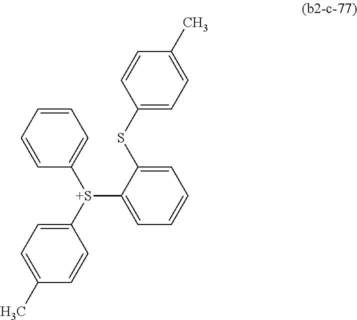 [SMILES: Cc1ccc(Sc2ccccc2[S+](c2ccccc2)c2ccc(C)cc2)cc1]

Examples of the cation (b2-5) include the following cations.

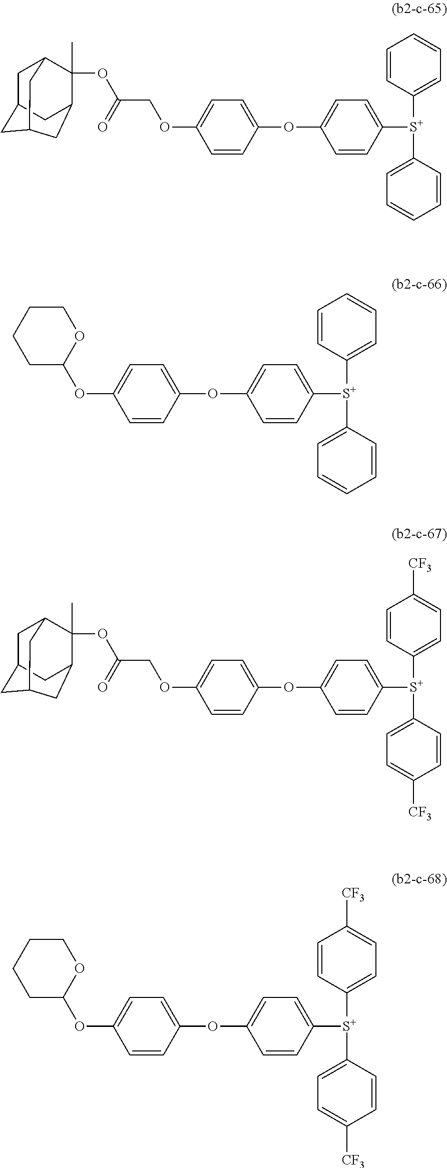 [SMILES: CC1(OC(=O)COc2ccc(Oc3ccc([S+](c4ccccc4)c4ccccc4)cc3)cc2)C2CC3CC(C2)CC1C3 ; c1ccc([S+](c2ccccc2)c2ccc(Oc3ccc(OC4CCCCO4)cc3)cc2)cc1 ; CC1(OC(=O)COc2ccc(Oc3ccc([S+](c4ccc(C(F)(F)F)cc4)c4ccc(C(F)(F)F)cc4)cc3)cc2)C2CC3CC(C2)CC1C3 ; FC(F)(F)c1ccc([S+](c2ccc(Oc3ccc(OC4CCCCO4)cc3)cc2)c2ccc(C(F)(F)F)cc2)cc1]

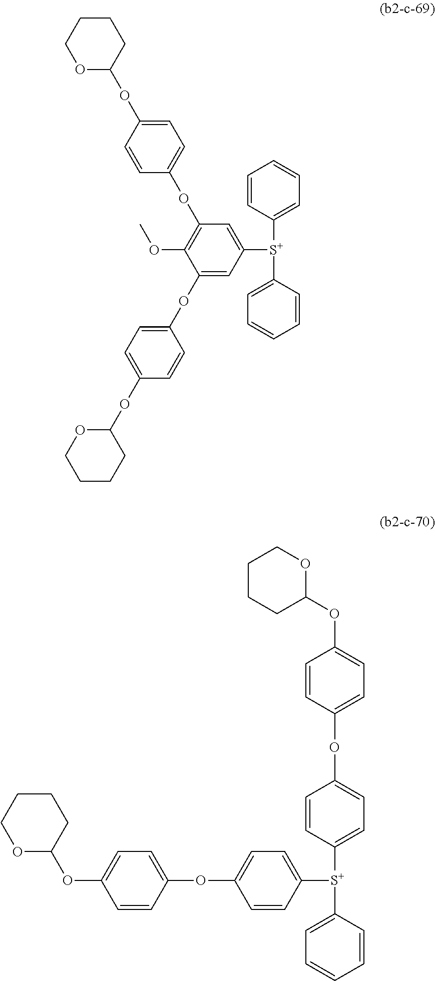 [SMILES: COc1c(Oc2ccc(OC3CCCCO3)cc2)cc([S+](c2ccccc2)c2ccccc2)cc1Oc1ccc(OC2CCCCO2)cc1 ; c1ccc([S+](c2ccc(Oc3ccc(OC4CCCCO4)cc3)cc2)c2ccc(Oc3ccc(OC4CCCCO4)cc3)cc2)cc1]

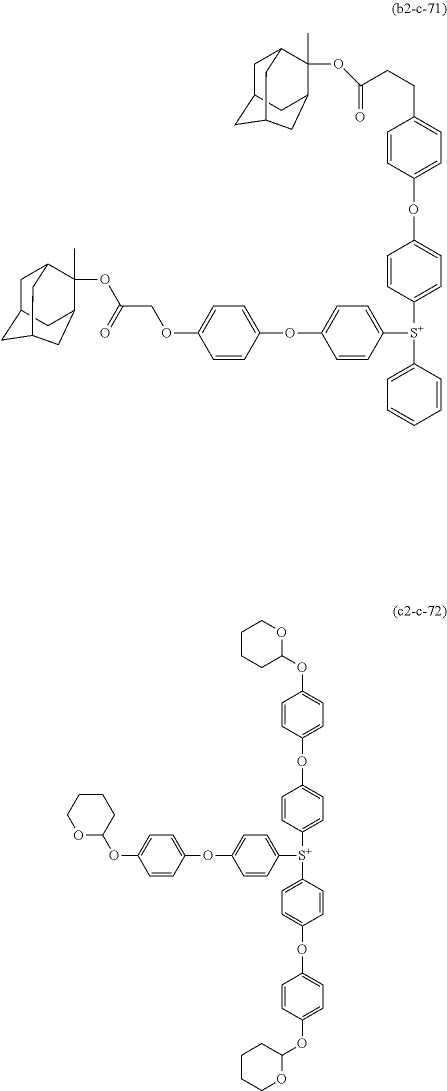 [SMILES: CC1(OC(=O)CCc2ccc(Oc3ccc([S+](c4ccccc4)c4ccc(Oc5ccc(OCC(=O)OC6(C)C7CC8CC(C7)CC6C8)cc5)cc4)cc3)cc2)C2CC3CC(C2)CC1C3 ; c1cc(OC2CCCCO2)ccc1Oc1ccc([S+](c2ccc(Oc3ccc(OC4CCCCO4)cc3)cc2)c2ccc(Oc3ccc(OC4CCCCO4)cc3)cc2)cc1]

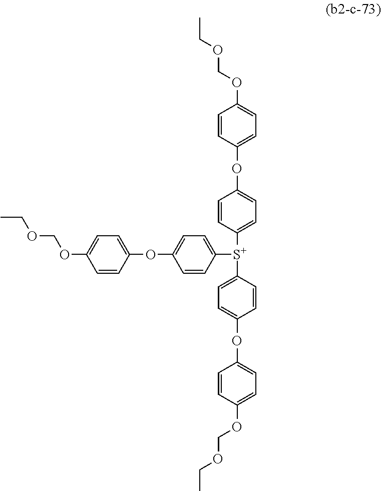 [SMILES: CCOCOc1ccc(Oc2ccc([S+](c3ccc(Oc4ccc(OCOCC)cc4)cc3)c3ccc(Oc4ccc(OCOCC)cc4)cc3)cc2)cc1]

The salt (I) is a combination of the anion mentioned above and the organic cation mentioned above, and these can be optionally combined. Examples of the salt (I) preferably include a combination of an anion represented by any one of formula (Ia-1) to formula (Ia-8), formula (Ia-18) to formula (Ia-20), formula (Ia-27) to formula (Ia-50) with a cation (b2-1), a cation (b2-2), a cation (b2-3), a cation (b2-4) or a cation (b2-5)

Examples of the salt (I) include salts shown in Table 1. In the following table, the respective symbols represent symbols imparted to structures showing the above-mentioned anions and cations. For example, the salt (I-1) means a salt composed of an anion represented by formula (Ia-1) and a cation represented by formula (b2-c-1), and represents salts shown below.

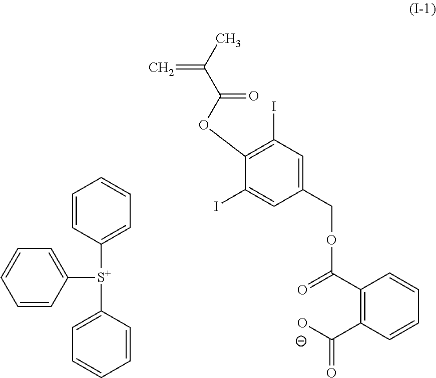 [SMILES: c1ccc([S+](c2ccccc2)c2ccccc2)cc1 ; C=C(C)C(=O)Oc1c(I)cc(COC(=O)c2ccccc2C(=O)[O-])cc1I]

TABLE 1

| Salt (I) | Anion (I) | Cation (I) |
|---|---|---|
| (I-1) to (I-20) | (Ia-1) to (Ia-20) | (b2-c-1) |
| (I-21) to (I-40) | (Ia-1) to (Ia-20) | (b2-c-10) |
| (I-41) to (I-60) | (Ia-1) to (Ia-20) | (b2-c-14) |
| (I-61) to (I-80) | (Ia-1) to (Ia-20) | (b2-c-17) |
| (I-81) to (I-100) | (Ia-1) to (Ia-20) | (b2-c-18) |
| (I-101) to (I-120) | (Ia-1) to (Ia-20) | (b2-c-19) |
| (I-121) to (I-140) | (Ia-1) to (Ia-20) | (b2-c-20) |
| (I-141) to (I-160) | (Ia-1) to (Ia-20) | (b2-c-27) |
| (I-161) to (I-180) | (Ia-1) to (Ia-20) | (b2-c-30) |
| (I-181) to (I-200) | (Ia-1) to (Ia-20) | (b2-c-31) |
| (I-201) to (I-220) | (Ia-1) to (Ia-20) | (b2-c-50) |
| (I-221) to (I-240) | (Ia-1) to (Ia-20) | (b2-c-51) |
| (I-241) to (I-260) | (Ia-1) to (Ia-20) | (b2-c-54) |
| (I-261) to (I-280) | (Ia-1) to (Ia-20) | (b2-c-55) |
| (I-181) to (I-300) | (Ia-1) to (Ia-20) | (b2-c-56) |
| (I-301) to (I-320) | (Ia-1) to (Ia-20) | (b2-c-57) |
| (I-321) to (I-340) | (Ia-1) to (Ia-20) | (b2-c-58) |
| (I-341) to (I-360) | (Ia-1) to (Ia-20) | (b2-c-59) |
| (I-361) to (I-380) | (Ia-1) to (Ia-20) | (b2-c-60) |
| (I-381) to (I-400) | (Ia-1) to (Ia-20) | (b2-c-61) |
| (I-401) to (I-420) | (Ia-1) to (Ia-20) | (b2-c-62) |
| (I-421) to (I-440) | (Ia-1) to (Ia-20) | (b2-c-63) |
| (I-441) to (I-460) | (Ia-1) to (Ia-20) | (b2-c-64) |
| (I-461) to (I-480) | (Ia-1) to (Ia-20) | (b2-c-65) |
| (I-481) to (I-500) | (Ia-1) to (Ia-20) | (b2-c-66) |
| (I-501) to (I-520) | (Ia-1) to (Ia-20) | (b2-c-67) |
| (I-521) to (I-540) | (Ia-1) to (Ia-20) | (b2-c-68) |
| (I-541) to (I-560) | (Ia-1) to (Ia-20) | (b2-c-69) |
| (I-561) to (I-580) | (Ia-1) to (Ia-20) | (b2-c-70) |
| (I-581) to (I-600) | (Ia-1) to (Ia-20) | (b2-c-71) |
| (I-601) to (I-620) | (Ia-1) to (Ia-20) | (b2-c-72) |
| (I-621) to (I-640) | (Ia-1) to (Ia-20) | (b2-c-73) |
| (I-641) to (I-660) | (Ia-1) to (Ia-20) | (b2-c-74) |
| (I-661) to (I-680) | (Ia-1) to (Ia-20) | (b2-c-75) |
| (I-681) to (I-700) | (Ia-1) to (Ia-20) | (b2-c-76) |
| (I-701) to (I-720) | (Ia-1) to (Ia-20) | (b2-c-77) |
| (I-721) to (I-750) | (Ia-21) to (Ia-50) | (b2-c-1) |
| (I-751) to (I-780) | (Ia-21) to (Ia-50) | (b2-c-10) |
| (I-781) to (I-810) | (Ia-21) to (Ia-50) | (b2-c-14) |
| (I-811) to (I-840) | (Ia-21) to (Ia-50) | (b2-c-17) |
| (I-841) to (I-870) | (Ia-21) to (Ia-50) | (b2-c-18) |
| (I-871) to (I-900) | (Ia-21) to (Ia-50) | (b2-c-19) |
| (I-901) to (I-930) | (Ia-21) to (Ia-50) | (b2-c-20) |
| (I-931) to (I-960) | (Ia-21) to (Ia-50) | (b2-c-27) |
| (I-961) to (I-990) | (Ia-21) to (Ia-50) | (b2-c-30) |
| (I-991) to (I-1020) | (Ia-21) to (Ia-50) | (b2-c-31) |
| (I-1021) to (I-1050) | (Ia-21) to (Ia-50) | (b2-c-50) |
| (I-1051) to (I-1080) | (Ia-21) to (Ia-50) | (b2-c-51) |
| (I-1081) to (I-1110) | (Ia-21) to (Ia-50) | (b2-c-54) |
| (I-1111) to (I-1140) | (Ia-21) to (Ia-50) | (b2-c-55) |
| (I-1141) to (I-1170) | (Ia-21) to (Ia-50) | (b2-c-56) |
| (I-1171) to (I-1200) | (Ia-21) to (Ia-50) | (b2-c-57) |
| (I-1201) to (I-1230) | (Ia-21) to (Ia-50) | (b2-c-58) |
| (I-1231) to (I-1260) | (Ia-21) to (Ia-50) | (b2-c-59) |
| (I-1261) to (I-1290) | (Ia-21) to (Ia-50) | (b2-c-60) |
| (I-1291) to (I-1320) | (Ia-21) to (Ia-50) | (b2-c-61) |
| (I-1321) to (I-1350) | (Ia-21) to (Ia-50) | (b2-c-62) |
| (I-1351) to (I-1380) | (Ia-21) to (Ia-50) | (b2-c-63) |
| (I-1381) to (I-1410) | (Ia-21) to (Ia-50) | (b2-c-64) |
| (I-1411) to (I-1440) | (Ia-21) to (Ia-50) | (b2-c-65) |
| (I-1441) to (I-1470) | (Ia-21) to (Ia-50) | (b2-c-66) |
| (I-1471) to (I-1500) | (Ia-21) to (Ia-50) | (b2-c-67) |
| (I-1501) to (I-1530) | (Ia-21) to (Ia-50) | (b2-c-68) |
| (I-1531) to (I-1560) | (Ia-21) to (Ia-50) | (b2-c-69) |
| (I-1561) to (I-1590) | (Ia-21) to (Ia-50) | (b2-c-70) |
| (I-1591) to (I-1620) | (Ia-21) to (Ia-50) | (b2-c-71) |
| (I-1621) to (I-1650) | (Ia-21) to (Ia-50) | (b2-c-72) |
| (I-1651) to (I-1680) | (Ia-21) to (Ia-50) | (b2-c-73) |
| (I-1681) to (I-1710) | (Ia-21) to (Ia-50) | (b2-c-74) |
| (I-1711) to (I-1740) | (Ia-21) to (Ia-50) | (b2-c-75) |
| (I-1741) to (I-1770) | (Ia-21) to (Ia-50) | (b2-c-76) |
| (I-1771) to (I-1800) | (Ia-21) to (Ia-50) | (b2-c-77) |

Of these, carboxylate (I) is preferably a salt obtained by combining an anion represented by any one of formula (Ia-1) to formula (Ia-8), formula (Ia-18) to formula (Ia-20), formula (Ia-27) to formula (Ia-50) with a cation represented by any one of formula (b2-c-1), formula (b2-c-10), formula (b2-c-13), formula (b2-c-14), formula (b2-c-18) to formula (b2-c-20), formula (b2-c-27), formula (b2-c-30), formula (b2-c-31), formula (b2-c-50), formula (b2-c-51) and formula (b2-c-54) to formula (b2-c-77).

### <Method for Producing Carboxylate (I)>

It is possible to produce a carboxylate in which X²⁰ is a group represented by any one of formula (X²⁰-1), formula (X²⁰-2), formula (X²⁰-4) and formula (X²⁰-5) in a carboxylate (I) (hereinafter sometimes referred to as carboxylate (I1)), for example, by reacting a compound represented by formula (I1-b) or a compound represented by formula (I1-a) with carbonyldiimidazole in a solvent, followed by a reaction with a carboxylate represented by formula (I-b).

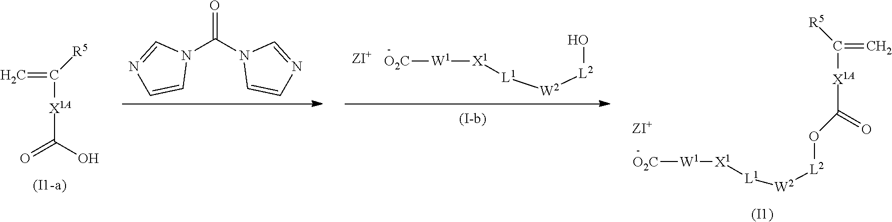

wherein all symbols are the same as defined above, respectively, X^{1A} represents a single bond, —O—, —Ar¹— or —CO—O—Ar¹—*, Ar¹ represents a 1,4-phenylene group, and * represents a bonding site to carbon atoms of a carbonyl group.

Examples of the solvent include tetrahydrofuran, chloroform and acetonitrile.

The reaction temperature is usually 0° C. to 80° C., and the reaction time is usually 0.5 hour to 24 hours.

Examples of the compound represented by formula (I1-a) include compounds represented by the following formulas and the like, which are easily available on the market.

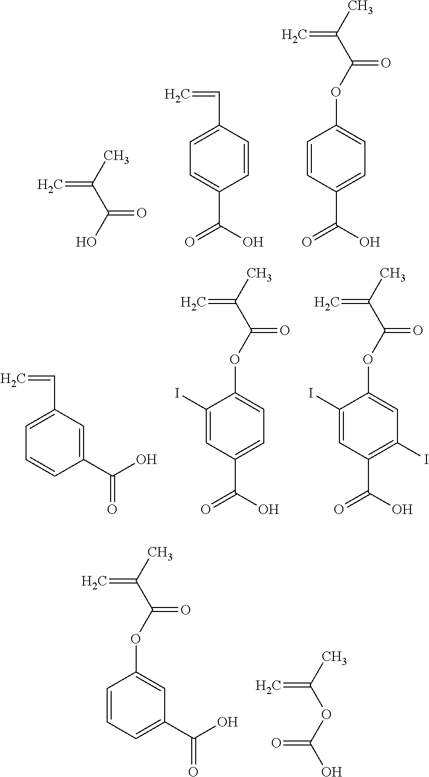

Examples of the carboxylate represented by formula (I-b) include compounds represented by the following formulas and the like, which are easily available on the market, and can also be easily produced by a known production method.

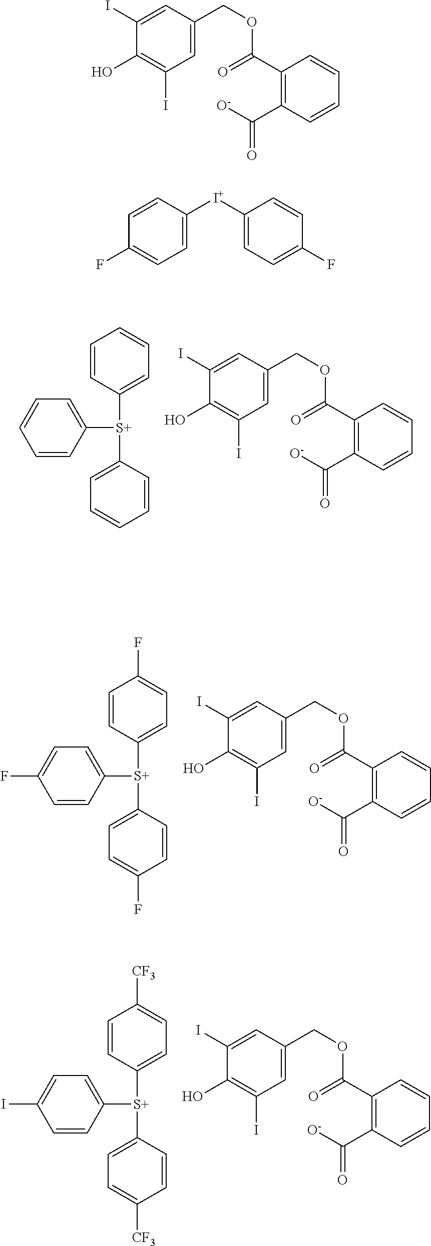

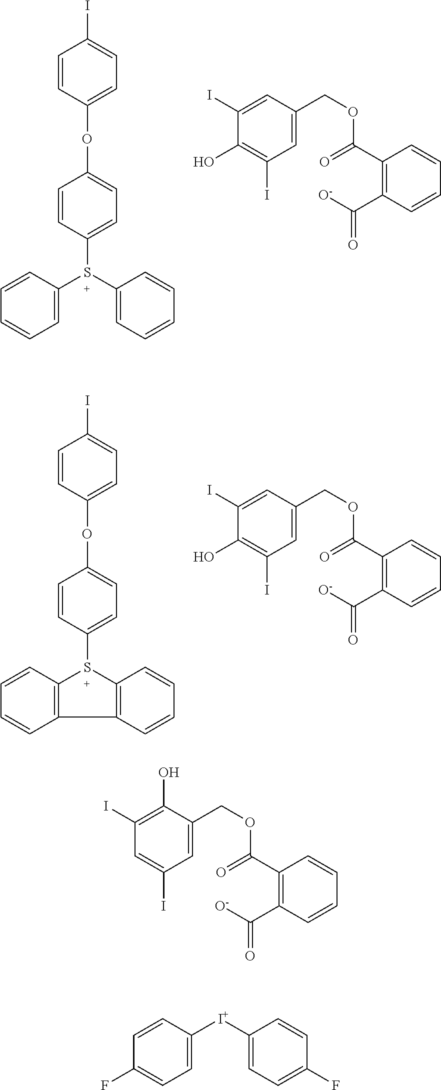

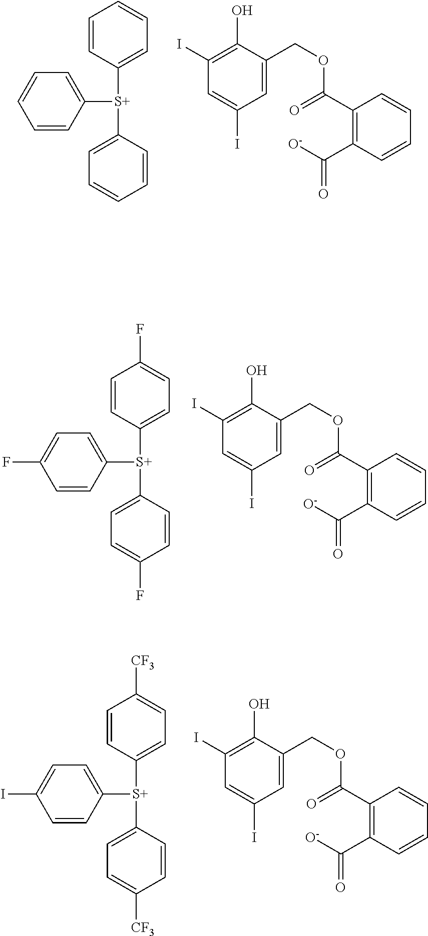 [SMILES: c1ccc([S+](c2ccccc2)c2ccccc2)cc1 ; O=C([O-])c1ccccc1C(=O)OCc1cc(I)cc(I)c1O ; Fc1ccc([S+](c2ccc(F)cc2)c2ccc(F)cc2)cc1 ; O=C([O-])c1ccccc1C(=O)OCc1cc(I)cc(I)c1O ; FC(F)(F)c1ccc([S+](c2ccc(I)cc2)c2ccc(C(F)(F)F)cc2)cc1 ; O=C([O-])c1ccccc1C(=O)OCc1cc(I)cc(I)c1O]

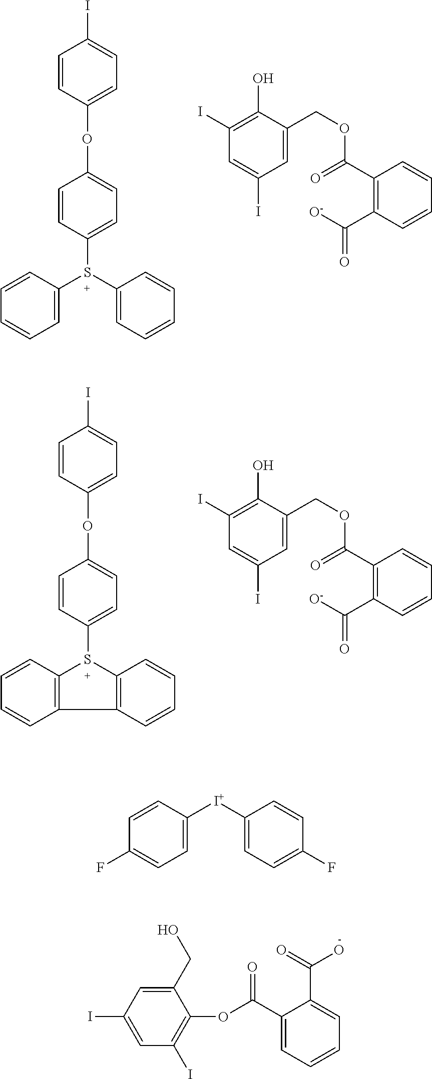

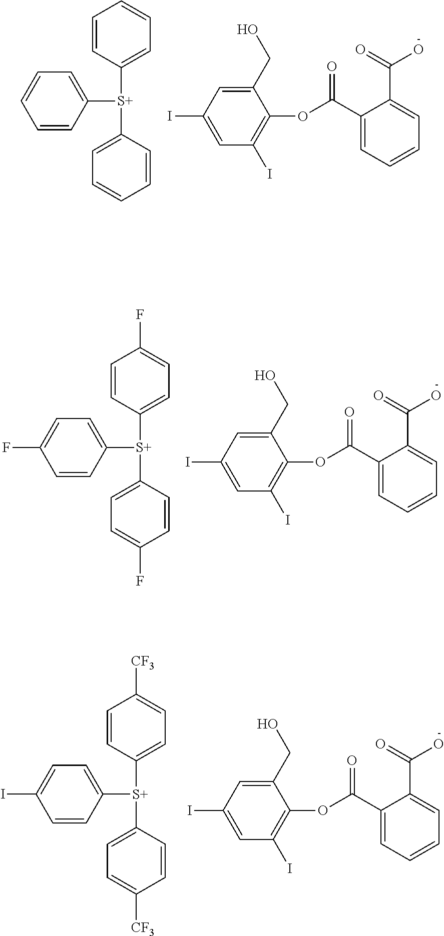 [SMILES: c1ccc([S+](c2ccccc2)c2ccccc2)cc1 ; O=C(O)c1ccccc1C(=O)Oc1c(I)cc(I)cc1CO ; Fc1ccc([S+](c2ccc(F)cc2)c2ccc(F)cc2)cc1 ; O=C(O)c1ccccc1C(=O)Oc1c(I)cc(I)cc1CO ; FC(F)(F)c1ccc([S+](c2ccc(I)cc2)c2ccc(C(F)(F)F)cc2)cc1 ; O=C(O)c1ccccc1C(=O)Oc1c(I)cc(I)cc1CO]

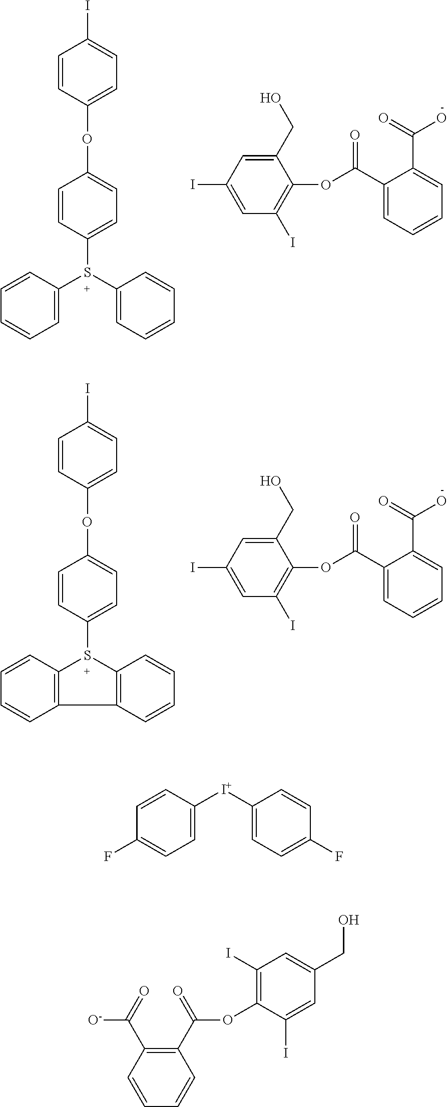

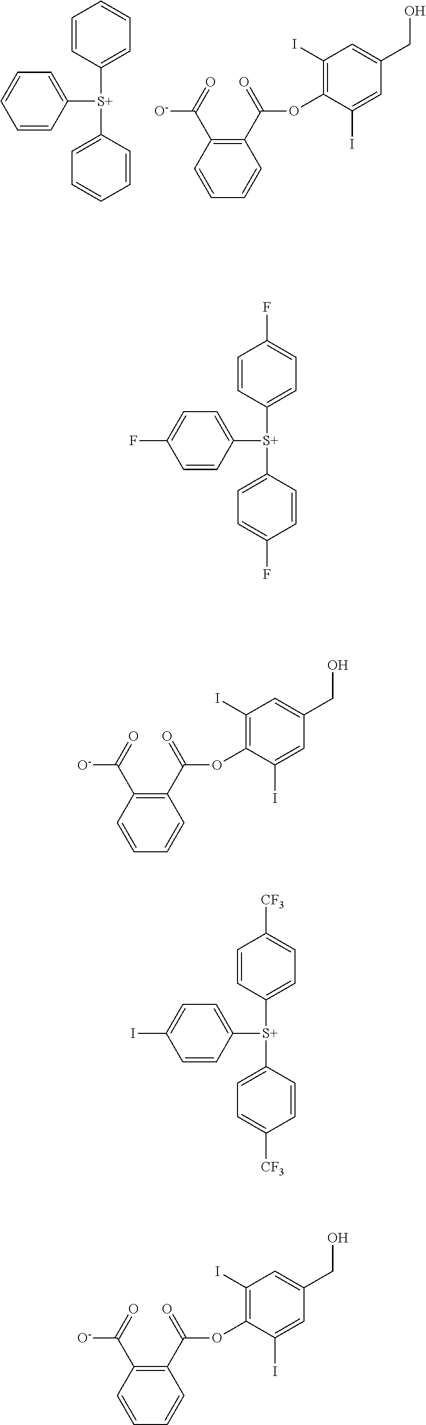 [SMILES: c1ccc([S+](c2ccccc2)c2ccccc2)cc1 ; O=C([O-])c1ccccc1C(=O)Oc1c(I)cc(CO)cc1I ; Fc1ccc([S+](c2ccc(F)cc2)c2ccc(F)cc2)cc1 ; O=C([O-])c1ccccc1C(=O)Oc1c(I)cc(CO)cc1I ; FC(F)(F)c1ccc([S+](c2ccc(I)cc2)c2ccc(C(F)(F)F)cc2)cc1 ; O=C([O-])c1ccccc1C(=O)Oc1c(I)cc(CO)cc1I]

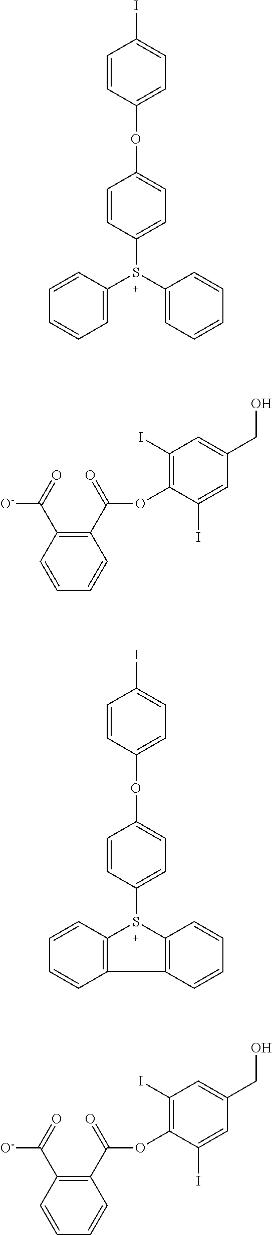

The carboxylate (I1) can also be produced, for example, by reacting a compound represented by formula (I1-a′) with a carboxylate represented by formula (I-b) in the presence of a base in a solvent:

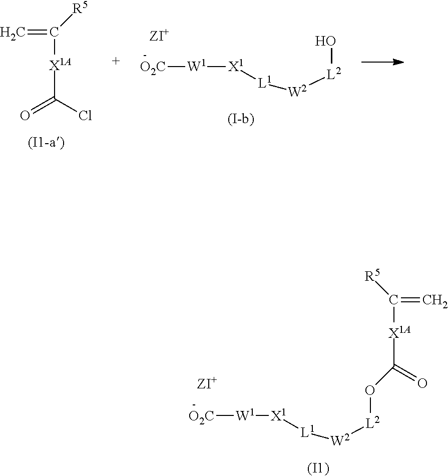

wherein all symbols are the same as defined above, respectively.

Examples of the base catalyst in this reaction include triethylamine, diisopropylethylamine, pyridine, dimethylaminopyridine and the like.

Examples of the solvent in this reaction include chloroform, acetonitrile and the like.

The reaction temperature is usually 5° C. to 80° C., and the reaction time is usually 0.5 hour to 24 hours.

Examples of the compound represented by formula (I1-a) include compounds represented by the following formulas and the like, which are easily available on the market, and can also be easily produced by a known production method.

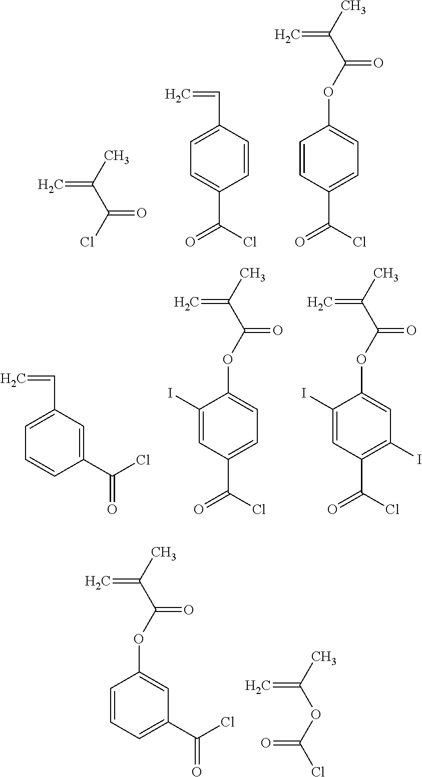

It is possible to produce a carboxylate in which X²⁰ is —O—, a group represented by formula (X²⁰-3) or formula (X²⁰-6), formula (X²⁰-9) or formula (X²⁰-10) in a carboxylate (I) (carboxylate represented by formula (I2)) by reacting a compound represented by formula (I2-a) with a carboxylate represented by formula (I-b) in the presence of a base in a solvent:

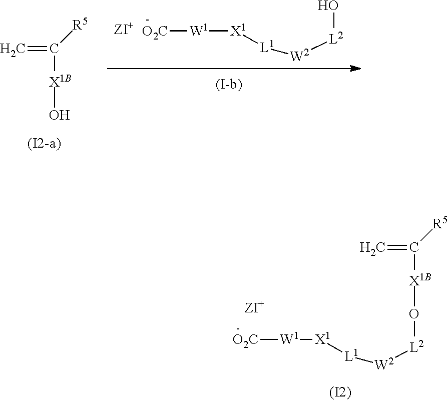

wherein all symbols are the same as defined above, respectively, X^{1B} represents a single bond, —Ar¹— or —CO—O—Ar¹—*, and * represents a bonding site to the oxygen atom.

Examples of the solvent include tetrahydrofuran, chloroform and acetonitrile.

Examples of the base include potassium hydroxide and the like.

The reaction temperature is usually 0° C. to 80° C., and the reaction time is usually 0.5 hour to 24 hours.

Examples of the compound represented by formula (I2-a) include compounds represented by the following formulas and the like, which are easily available on the market.

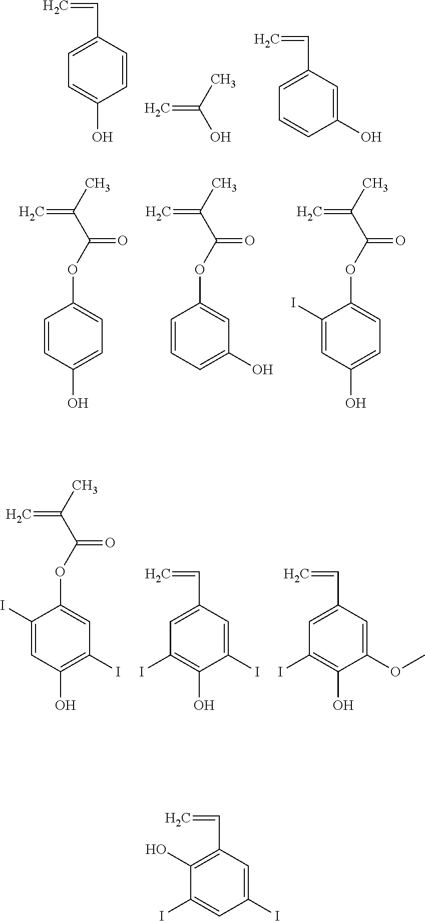

It is possible to produce a carboxylate in which X²⁰ is a group represented by formula (X¹-7′) or formula (X¹-8′) in a carboxylate (I) (carboxylate represented by formula (I3)) by reacting a compound represented by formula (I2-a) with carbonyldiimidazole in a solvent, followed by a reaction with a carboxylate represented by formula (I-b):

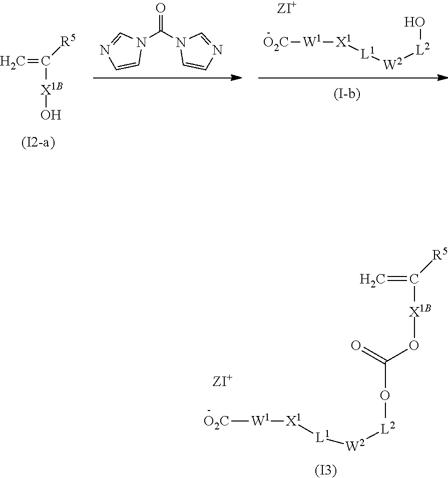

wherein all symbols are the same as defined above, respectively, X^{1B} represents —Ar¹— or —CO—O—Ar¹—*, and * represents a bonding site to the oxygen atom.

Examples of the solvent include tetrahydrofuran, chloroform and acetonitrile.

The reaction temperature is usually 0° C. to 80° C., and the reaction time is usually 0.5 hour to 24 hours.

It is possible to produce a carboxylate in which X²⁰ is a single bond, L² is a single bond, and X¹ is *—CO—O— (in which * represents a bonding site to W¹) in a carboxylate (I) (carboxylate represented by formula (I4)), for example, by reacting a compound represented by formula (14-b) or a compound represented by formula (I4-b′) with a compound represented by formula (I4-a) in the presence of a base catalyst in a solvent:

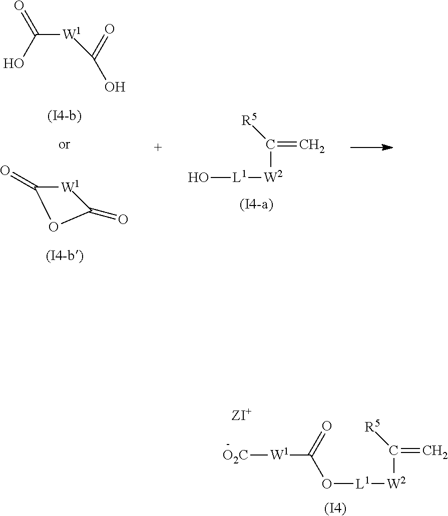

wherein all symbols are the same as defined above, respectively.

Examples of the base in this reaction include dimethylaminopyridine, pyridine, triethylamine and the like.

Examples of the solvent in this reaction include chloroform, acetonitrile, dimethylformamide and the like.

The reaction temperature is usually 5° C. to 80° C., and the reaction time is usually 0.5 hour to 24 hours.

Examples of the compound represented by formula (I4-a) include compounds shown below and the like, which are easily available on the market, and can also be easily produced by a known production method.

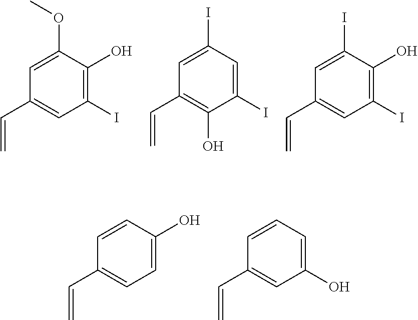

Examples of the compound represented by formula (I4-b) include compounds shown below and the like, which are easily available on the market.

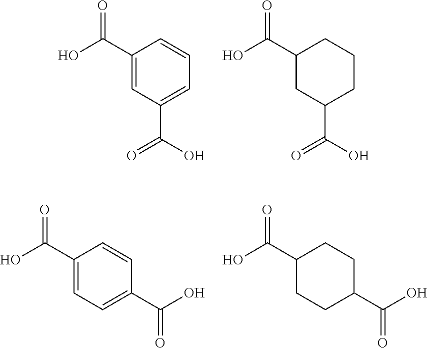 [SMILES: O=C(O)c1cccc(C(=O)O)c1 ; O=C(O)C1CCCC(C(=O)O)C1 ; O=C(O)c1ccc(C(=O)O)cc1 ; O=C(O)C1CCC(C(=O)O)CC1]

Examples of the compound represented by formula (I4-b′) include compounds shown below and the like, which are easily available on the market.

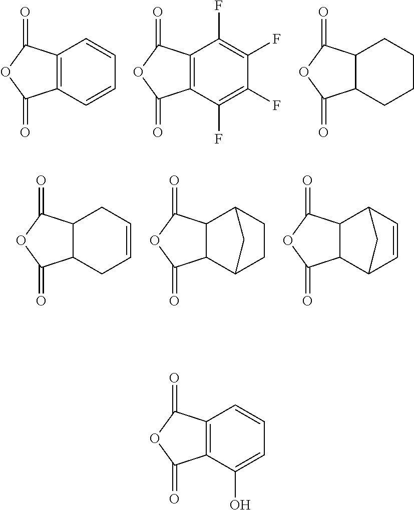

It is possible to produce a carboxylate in which X²⁰ is a single bond, L² is a single bond, and X¹ is *—O—CO— (in which * represents a bonding site to W¹) in a carboxylate (I) (carboxylate represented by formula (I5)) by reacting a compound represented by formula (I5-a) with carbonyldiimidazole in a solvent, followed by a reaction with a salt represented by formula (I5-b):

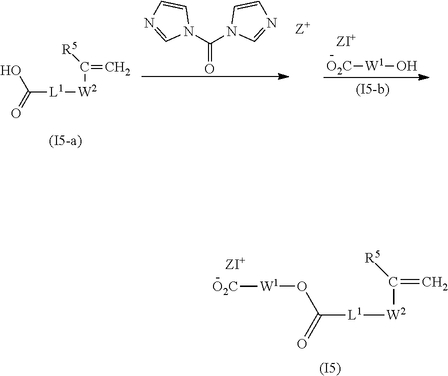

wherein all symbols are the same as defined above, respectively.

Examples of the solvent in this reaction include chloroform, acetonitrile and the like.

The reaction temperature is usually 5° C. to 80° C., and the reaction time is usually 0.5 hour to 24 hours.

Examples of the compound represented by formula (I5-a) include compounds shown below, which are easily available on the market, and can also be easily produced by a known production method.

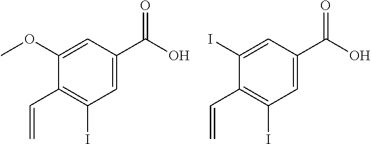

Examples of the salt represented by formula (I5-b) include salts shown below, which are easily available on the market, and can also be easily produced by a known production method.

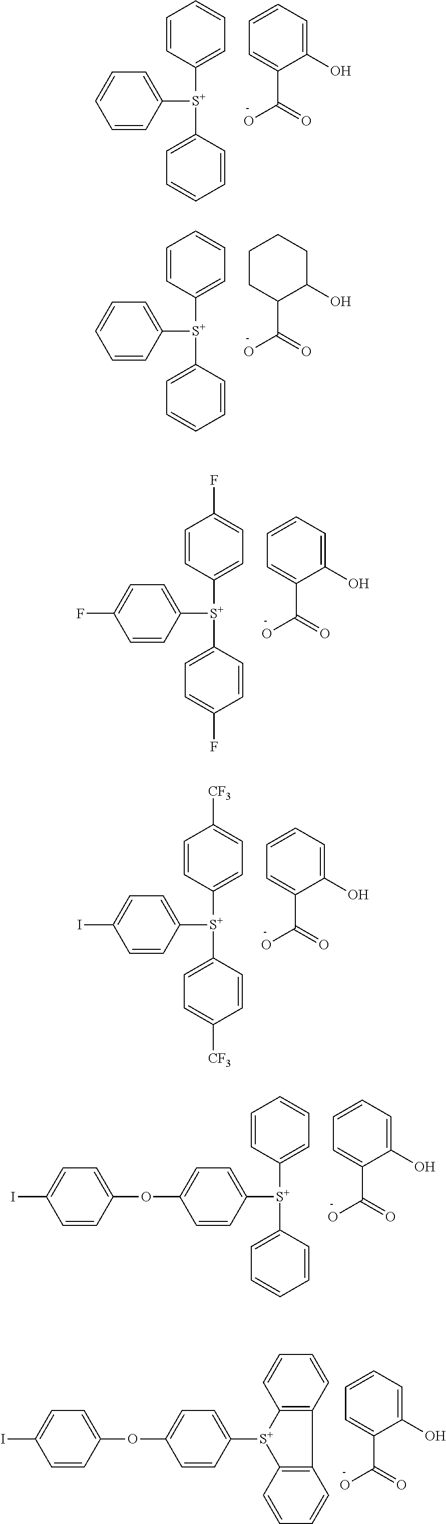

It is possible to produce a carboxylate in which X²⁰ is a single bond, L² is a single bond, and X¹ is *—O—CO—O— (in which * represents a bonding site to W¹) in a carboxylate (I) (carboxylate represented by formula (I6)), for example, by reacting a compound represented by formula (I4-a) with a carbonyldiimidazole in a solvent, followed by a reaction with a salt represented by formula (I5-b).

Alternatively, a carboxylate (carboxylate represented by formula (I6)) can be produced, for example, by reacting a salt represented by formula (I5-b) with carbonyldiimidazole in a solvent, followed by a reaction with a compound represented by formula (I4-a):

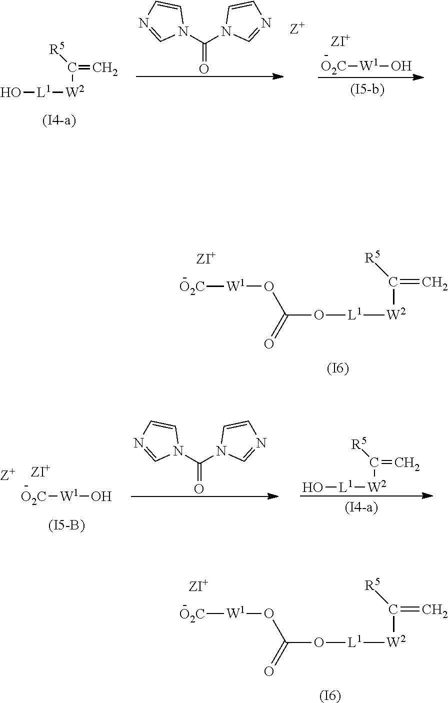

wherein all symbols are the same as defined above, respectively.

Examples of the solvent in this reaction include chloroform, acetonitrile and the like.

The reaction temperature is usually 5° C. to 80° C., and the reaction time is usually 0.5 hour to 24 hours.

It is possible to produce a carboxylate in which X²⁰ is a single bond, L² is a single bond, and X¹ is *—O— (in which * represents a bonding site to W¹) in a carboxylate (I) (carboxylate represented by formula (I7)), for example, by reacting a compound represented by formula (I4-a) with a salt represented by formula (I5-b) in the presence of a base in a solvent:

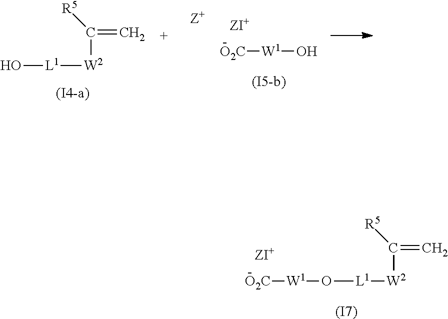

wherein all symbols are the same as defined above, respectively.

Examples of the base in this reaction include potassium hydroxide and the like.

Examples of the solvent in this reaction include chloroform, acetonitrile and the like.

The reaction temperature is usually 15° C. to 80° C., and the reaction time is usually 0.5 hour to 24 hours.

It is possible to produce a carboxylate in which X²⁰ is a single bond, L² is a single bond, L¹ is a single bond, and X¹ is *—O— (in which * represents a bonding site to W¹) in a carboxylate (I) (carboxylate represented by formula (I8)), for example, by reacting a compound represented by formula (I8-a) with a salt represented by formula (I5-b) in the presence of a base in a solvent:

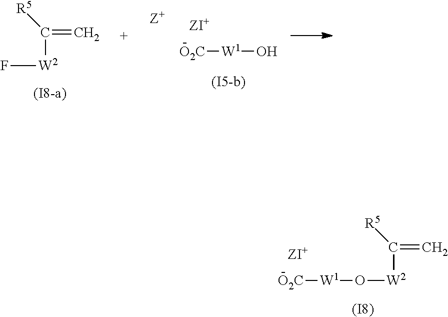

wherein all symbols are the same as defined above, respectively.

Examples of the base in this reaction include sodium hydride, potassium carbonate and the like.

Examples of the solvent in this reaction include chloroform, acetonitrile and the like.

The reaction temperature is usually 15° C. to 80° C., and the reaction time is usually 0.5 hour to 24 hours.

Examples of the compound represented by formula (I8-a) include compounds represented by the following formulas and the like, which are easily available on the market.

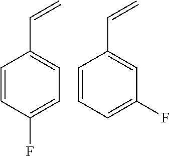

[Structural Unit Derived from Carboxylate Represented by Formula (I)]

The structural unit derived from a carboxylate represented by formula (I) of the present invention is a structural unit represented by the following formula (IP) (hereinafter sometimes referred to as “structural unit (IP)”)

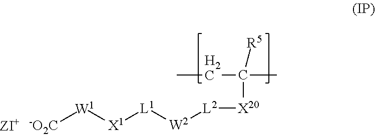

wherein, in formula (IP), all symbols are the same as defined above, respectively.

The structural unit (IP) indicates a state where a double bond of CH₂═C—R⁵ included in the carboxylate (I) is cleaved.

Such structural unit (IP) functions as a carboxylic acid generator similarly to the carboxylate (I), and also functions as a structural unit constituting a compound or a resin.

[Resin Including Structural Unit (IP) Derived from Carboxylate Represented by Formula (I)]

The resin of the present invention is a resin including a structural unit (IP) derived from a carboxylate represented by formula (I) (hereinafter sometimes referred to as “resin (Ap)”).

The resin (Ap) may be either a homopolymer including one structural unit (IP), or a copolymer including two or more structural units (IP).

The resin (Ap) may include a structural unit other than the structural unit (IP). As mentioned below, examples of the structural unit other than the structural unit (IP) include a structural unit having an acid-labile group (hereinafter sometimes referred to as “structural unit (a1)”), and a structural unit other than the structural unit (a1). Examples of the structural unit other than the structural unit (a1) include a structural unit having no acid-labile group (hereinafter sometimes referred to as “structural unit (s)”), other structural units (hereinafter sometimes referred to as “structural unit (t)”) and structural units known in the relevant field. Here, “acid-labile group” means a group having a leaving group which is eliminated by contact with an acid, thus converting a constitutional unit into a constitutional unit having a hydrophilic group (e.g., a hydroxy group or a carboxy group).

The content of the structural unit (IP) is usually 0.1 mol % or more, preferably 0.5 mol % or more, more preferably 0.8 mol % or more, and still more preferably 1 mol % or more, based on all structural units of the resin (Ap). The content is also usually 100 mol % or less, preferably 50 mol % or less, more preferably 30 mol % or less, and still more preferably 10 mol % or less. Specifically, the content is usually 0.1 to 100 mol %, preferably 0.5 to 50 mol %, more preferably 0.8 to mol %, and still more preferably 1 to 10 mol %.

Particularly, when used for the resist composition, as mentioned below, the resin (Ap) may further include, in addition to the structural unit (IP), a structural unit (a1).

When used for the resist composition, as mentioned below, the resin (Ap) may be used in combination with a resin including a structural unit (a1) (hereinafter sometimes referred to as “resin (A)”) and/or a resin or the like other than the resin (A), whether the resin includes the structural unit (a1) or not. Hereinafter the resin (Ap) and/or the resin (A) may be sometimes referred to as “resin (A) or the like”.

It is preferable that each of the resin (Ap) and the resin (A) further includes a structural unit other than the structural unit (a1).

<Structural Unit (a1)>

The structural unit (a1) is derived from a monomer having an acid-labile group (hereinafter sometimes referred to as “monomer (a1)”).

The acid-labile group contained in the resin (A) or the like is preferably a group represented by formula (1) (hereinafter also referred to as group (1)) and/or a group represented by formula (2) (hereinafter also referred to as group (2)):

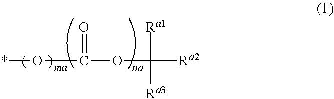

wherein, in formula (1), Rai, R^{a2} and R^{a3} each independently represent an alkyl group having 1 to 8 carbon atoms, an alkenyl group having 2 to 8 carbon atoms, an alicyclic hydrocarbon group having 3 to 20 carbon atoms, an aromatic hydrocarbon group having 6 to 18 carbon atoms, or groups obtained by combining these groups, or R^{a1} and R^{a2} are bonded to each other to form an alicyclic hydrocarbon group having 3 to 20 carbon atoms together with carbon atoms to which R^{a1} and R^{a2} are bonded, and the alkyl group, the alkenyl group, the alicyclic hydrocarbon group and the aromatic hydrocarbon group may have a halogen atom,
ma and na each independently represent 0 or 1, and at least one of ma and na represents 1, and
represents a bonding site:

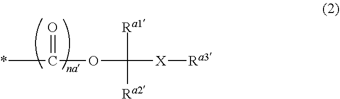

wherein, in formula (2), R^{a1′} and R^{a2′} each independently represent a hydrogen atom or a hydrocarbon group having 1 to 12 carbon atoms, R^{a3′} represents a hydrocarbon group having 1 to 20 carbon atoms, or R^{a2′} and R^{a3′} are bonded to each other to form a heterocyclic ring group having 3 to 20 carbon atoms together with carbon atoms and X to which R^{a2′} and R^{a3′} are bonded, —CH₂— included in the hydrocarbon group and the heterocyclic ring group may be replaced by —O— or —S—, and the hydrocarbon group and the heterocyclic ring group may have a halogen atom,
X represents an oxygen atom or a sulfur atom,
na′ represents 0 or 1, and
* represents a bonding site.

Examples of the alkyl group in Rai, R^{a2} and R^{a3} include a methyl group, an ethyl group, a propyl group, a butyl group, a pentyl group, a hexyl group, a heptyl group, an octyl group and the like.

Examples of the alkenyl group in Rai, R^{a2} and R^{a3} include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The alicyclic hydrocarbon group in Rai, R^{a2} and R^{a3} may be either monocyclic or polycyclic. Examples of the monocyclic alicyclic hydrocarbon group include cycloalkyl groups such as a cyclopentyl group, a cyclohexyl group, a cycloheptyl group and a cyclooctyl group. Examples of the polycyclic alicyclic hydrocarbon group include a decahydronaphthyl group, an adamantyl group, a norbornyl group and the following groups (* represents a bonding site). The number of carbon atoms of the alicyclic hydrocarbon group of Rai, R^{a2} and R^{a3} is preferably 3 to 16.

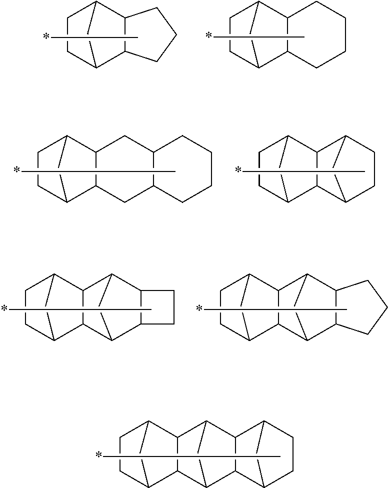

Examples of the aromatic hydrocarbon group in Rai, R^{a2} and R^{a3} include aryl groups such as a phenyl group, a naphthyl group, an anthryl group, a biphenyl group and a phenanthryl group.

Examples of the combined group include groups obtained by combining the above-mentioned alkyl group and alicyclic hydrocarbon group (e.g., alkylcycloalkyl groups or cycloalkylalkyl groups, such as a methylcyclohexyl group, a dimethylcyclohexyl group, a methylnorbornyl group, a cyclohexylmethyl group, an adamantylmethyl group, an adamantyldimethyl group and a norbornylethyl group), aralkyl groups such as a benzyl group, aromatic hydrocarbon groups having an alkyl group (a p-methylphenyl group, a p-tert-butylphenyl group, a tolyl group, a xylyl group, a cumenyl group, a mesityl group, a 2,6-diethylphenyl group, a 2-methyl-6-ethylphenyl group, etc.), aromatic hydrocarbon groups having an alicyclic hydrocarbon group (a p-cyclohexylphenyl group, a p-adamantylphenyl group, etc.), aryl-cycloalkyl groups such as a phenylcyclohexyl group, and the like.

Preferably, ma is 0 and na is 1.

When R^{a1} and R^{a2} are bonded to each other to form an alicyclic hydrocarbon group, examples of —C(R^{a1}) (R^{a2}) (R^{a3}) include the following groups. The alicyclic hydrocarbon group preferably has 3 to 12 carbon atoms. * represents a bonding site to —O—.

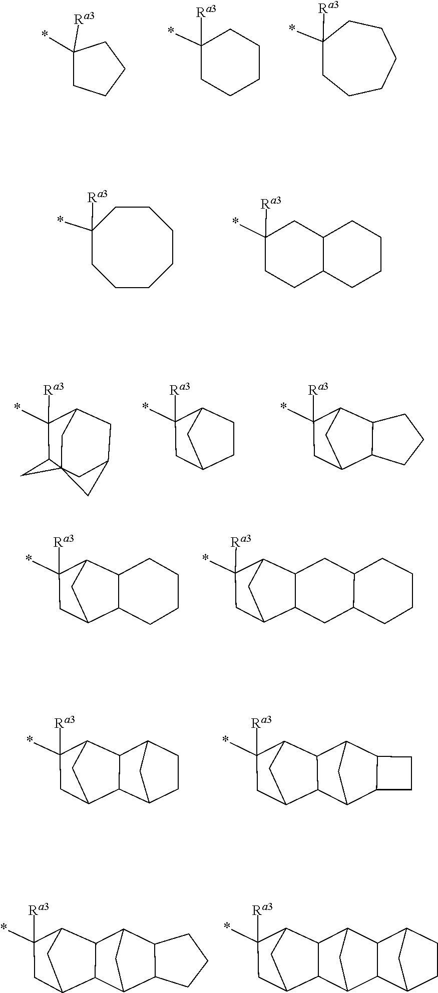

Examples of the hydrocarbon group in R^{a1′}, R^{a2′} and R^{a3′} include an alkyl group, an alicyclic hydrocarbon group, an aromatic hydrocarbon group, and groups formed by combining these groups.

Examples of the alkyl group, the alicyclic hydrocarbon group, the aromatic hydrocarbon group, and the groups obtained by combining these groups include those which are the same as mentioned as for Rai, R^{a2} and R^{a3}.

When R^{a2′} and R^{a3′} are bonded to each other to form a heterocyclic group together with carbon atoms and X to which R^{a2′} and R^{a3′} are bonded, examples of —C(R^{a1′}) (R^{a2′})—X—R^{a3′} include the following groups. * represents a bonding site.

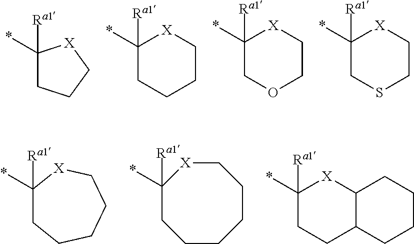

At least one of R^{a1′} and R^{a2′} is preferably a hydrogen atom.

na′ is preferably 0.

Examples of the halogen atom which may be possessed by R^{a1}, R^{a2}, R^{a3}, R^{a1′}, R^{a2′} and R^{a3′} include a fluorine atom, a chlorine atom, a bromine atom or an iodine atom.

Examples of the group (1) include the following groups.

A group wherein, in formula (1), R^{a1}, R^{a2} and R^{a3} are alkyl groups, ma=0 and na=1. The group is preferably a tert-butoxycarbonyl group.

A group wherein, in formula (1), R^{a1} and R^{a2} are bonded to each other to form an adamantyl group together with carbon atoms to which R^{a1} and R^{a2} are bonded, R^{a3} is an alkyl group, ma=0 and na=1.

A group wherein, in formula (1), R^{a1} and R^{a2} are each independently an alkyl group, R^{a3} is an adamantyl group, ma=0 and na=1.

Specific examples of the group (1) include the following groups. * represents a bonding site.

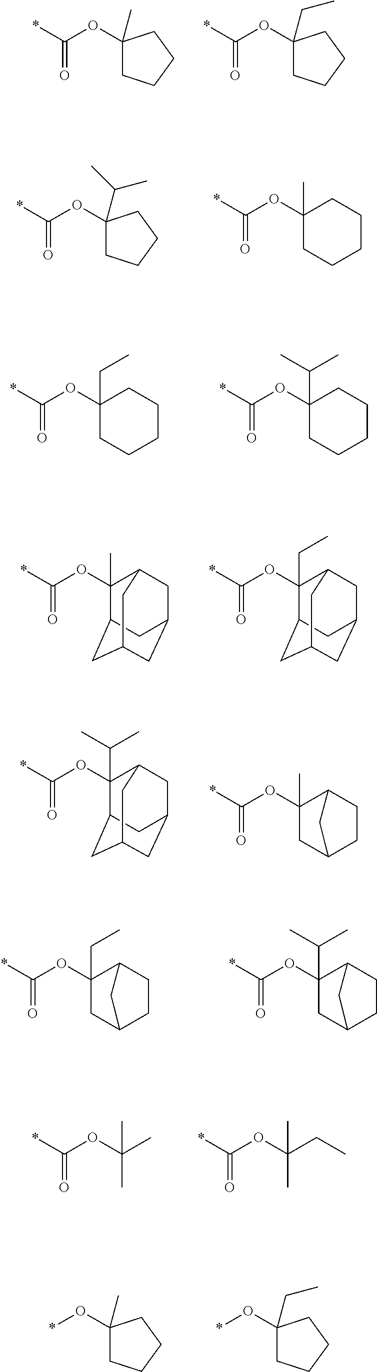

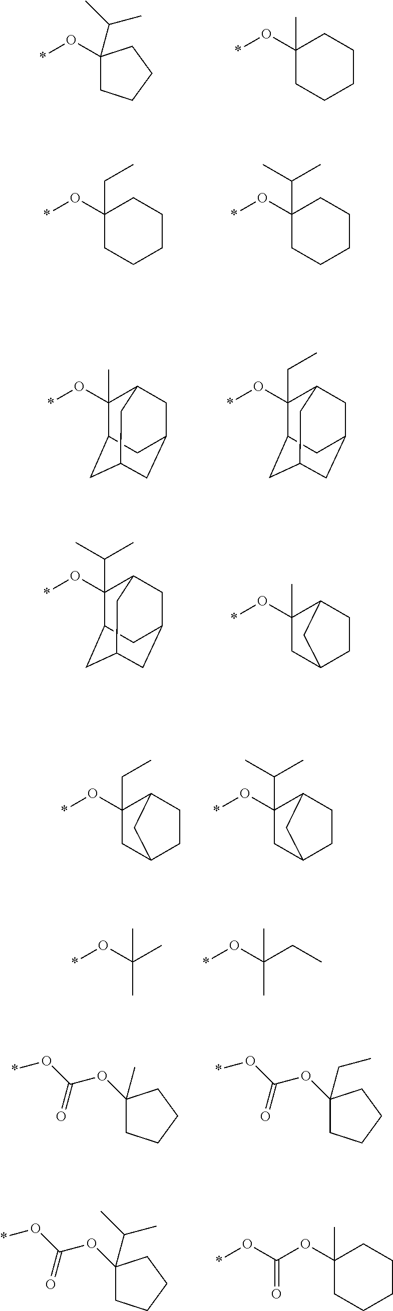

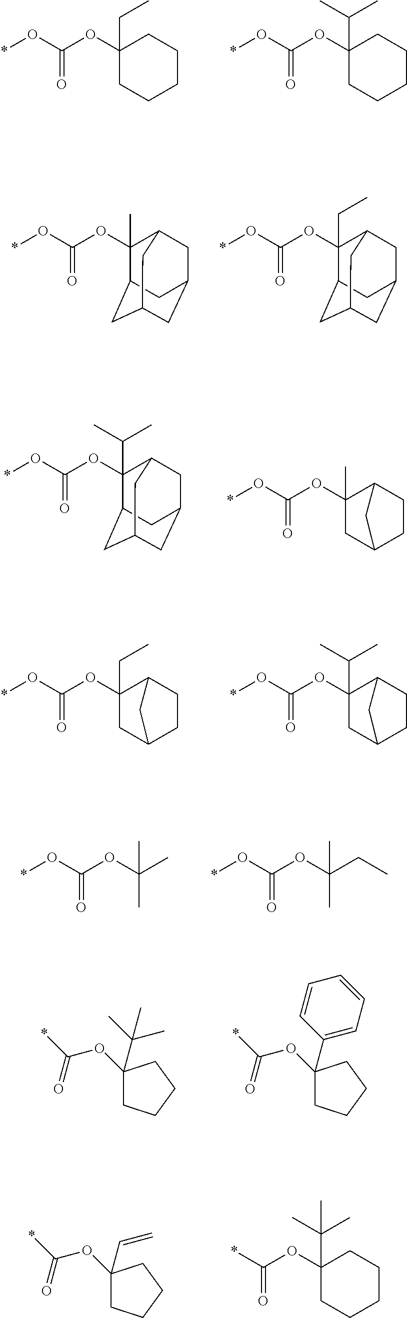

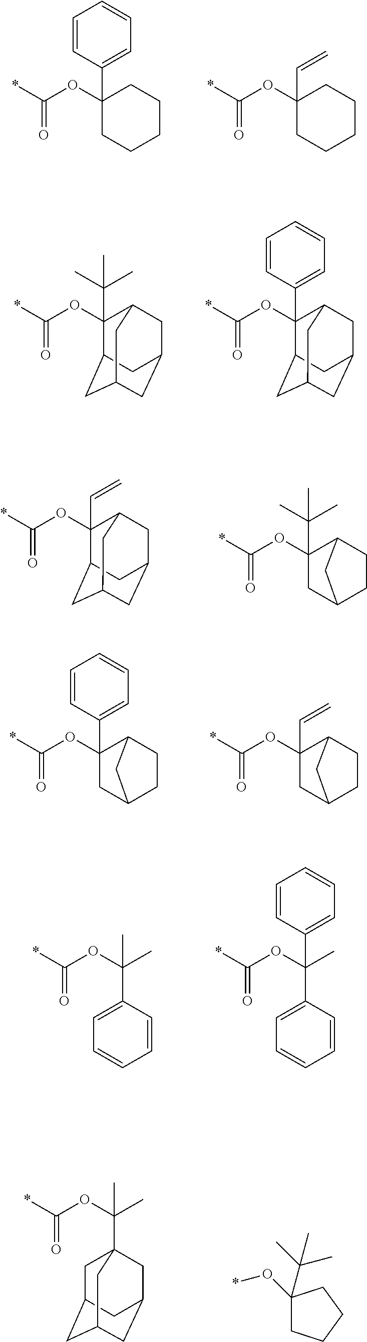

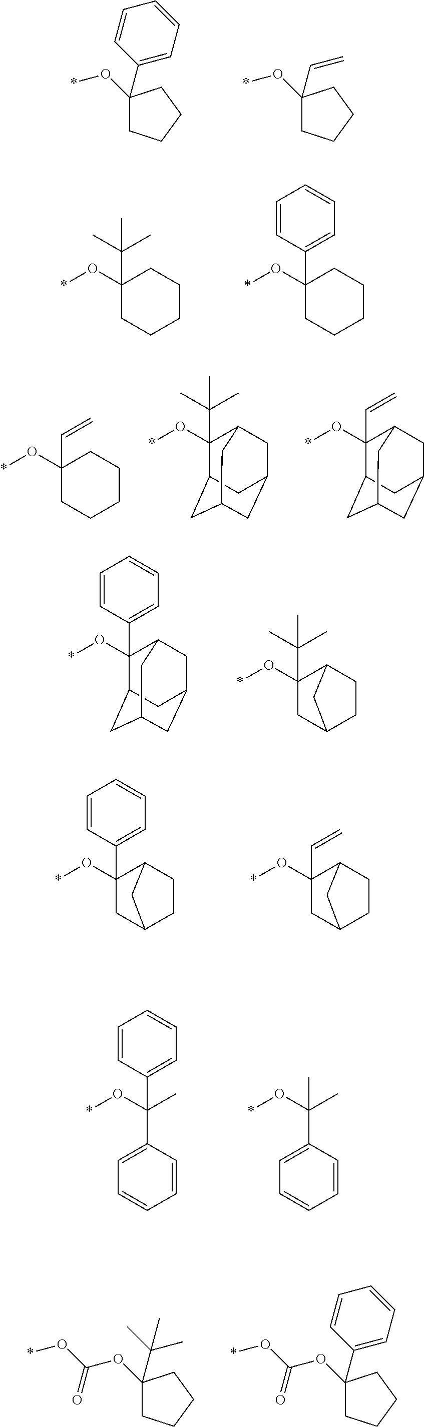

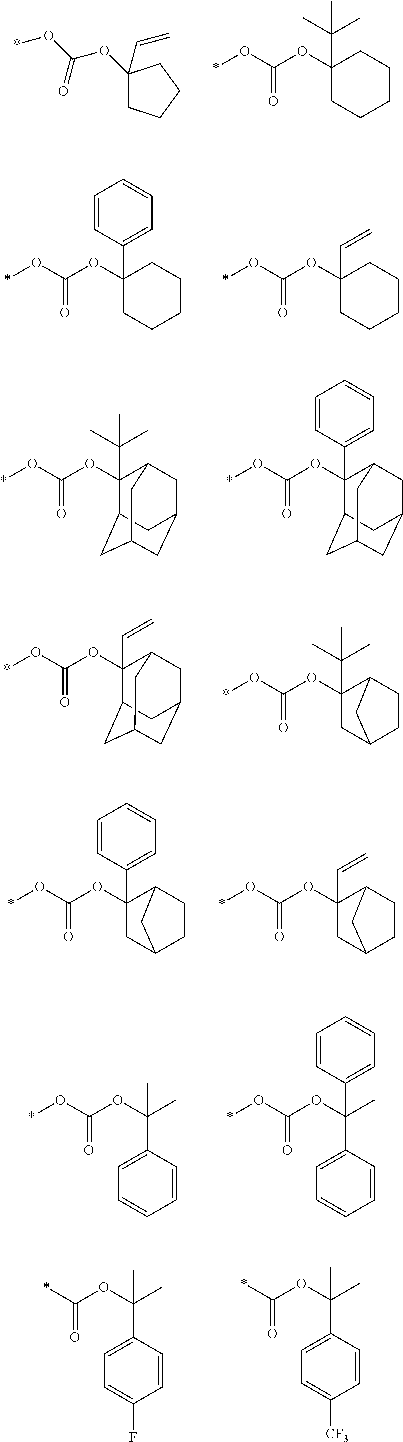

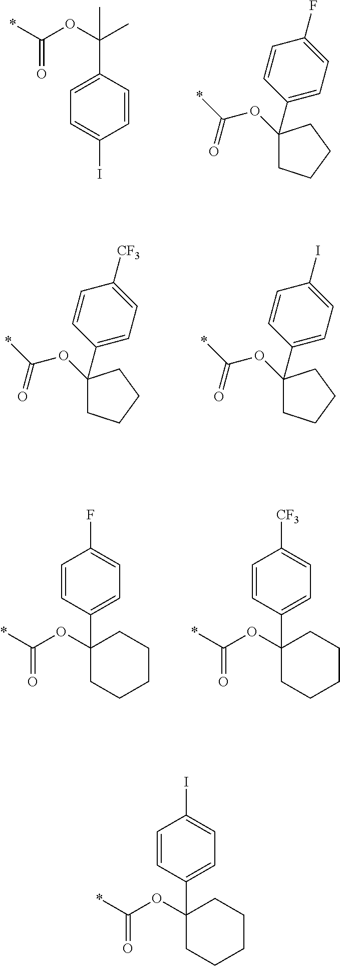

Specific examples of the group (2) include the following groups. * represents a bonding site.

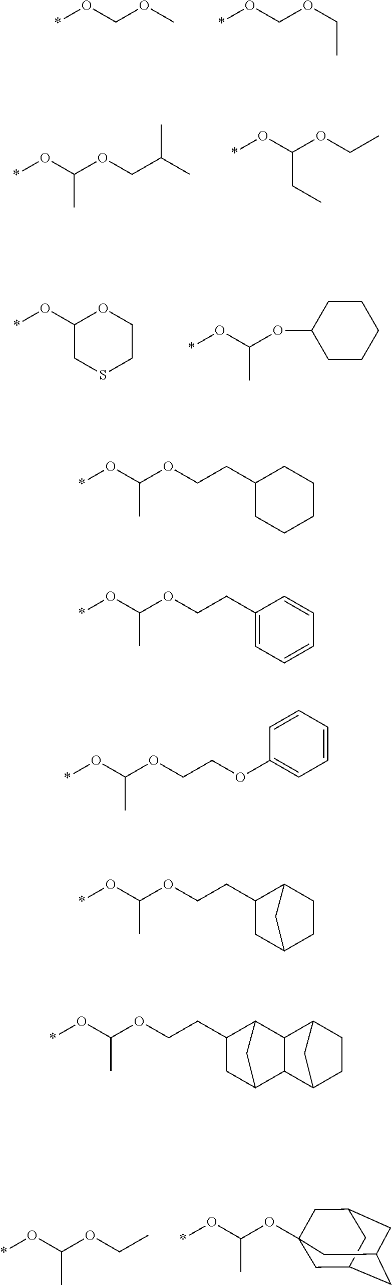

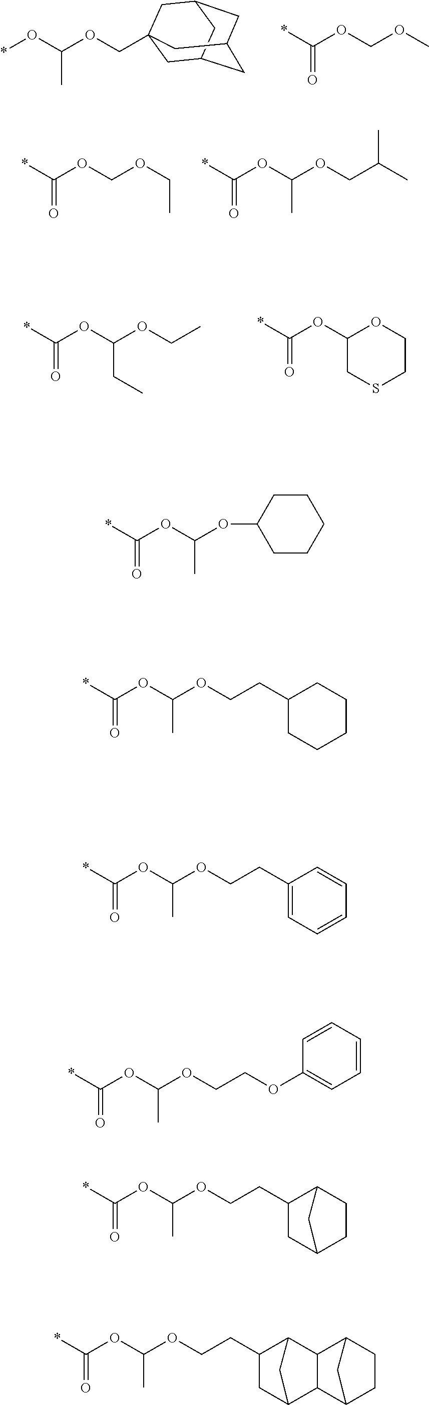

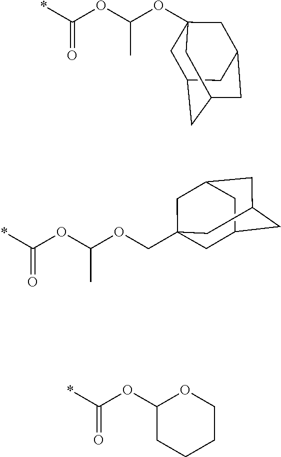 [SMILES: *C(=O)OC(C)OC12CC3CC(CC(C3)C1)C2 ; *C(=O)OC(C)OCC12CC3CC(CC(C3)C1)C2 ; *C(=O)OC1CCCCO1]

The monomer (a1) is preferably a monomer having an acid-labile group and an ethylenic unsaturated bond, and more preferably a (meth)acrylic monomer having an acid-labile group.

Of the (meth)acrylic monomers having an acid-labile group, those having an alicyclic hydrocarbon group having 5 to 20 carbon atoms are preferably exemplified. When using a resin (A) including a structural unit derived from a monomer (a1) having a bulky structure such as an alicyclic hydrocarbon group or the like in a resist composition, it is possible to improve the resolution of a resist pattern.

The structural unit derived from a (meth)acrylic monomer having a group (1) includes a structural unit represented by formula (a1-0) (hereinafter sometimes referred to as structural unit (a1-0)), a structural unit represented by formula (a1-1) (hereinafter sometimes referred to as structural unit (a1-1)) or a structural unit represented by formula (a1-2) (hereinafter sometimes referred to as structural unit (a1-2)). The structural unit is preferably at least one structural unit selected from the group consisting of a structural unit (a1-0), a structural unit (a1-1) and a structural unit (a1-2), and more preferably at least one or two structural units selected from the group consisting of a structural unit (a1-1) and a structural unit (a1-2). These structural units may be used alone, or two or more structural units may be used in combination.

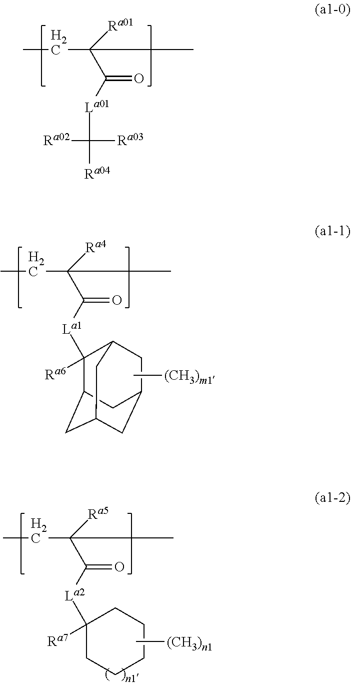

wherein, in formula (a1-0), formula (a1-1) and formula (a1-2),
L^{a01}, L^{a1} and L^{a2} each independently represent —O— or *—O—(CH₂)ₖ₁—CO—O—, k1 represents an integer of 1 to 7, and * represents a bonding site to —CO—,
R^{a01}, R^{a4} and R^{a5} each independently represent a hydrogen atom, a halogen atom or an alkyl group having 1 to 6 carbon atoms which may have a halogen atom,
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alkenyl group, the alicyclic hydrocarbon group and the aromatic hydrocarbon group may have a halogen atom.
m1′ represents an integer of 0 to 14,
n1 represents an integer of 0 to 10, and
n1′ represents an integer of 0 to 3.

Examples of the halogen atom include a fluorine atom, a chlorine atom, a bromine atom or an iodine atom.

Examples of the alkyl group as for R^{a01}, R^{a4} and R^{a5} include a methyl group, an ethyl group, a propyl group, a butyl group, a pentyl group or a hexyl group.

R^{a01}, R^{a4} and R^{a5} are preferably a hydrogen atom or a methyl group, and more preferably a methyl group.
L^{a01}, L^{a1} and L^{a2} are preferably an oxygen atom or *—O—(CH₂)ₖ₀₁—CO—O— (in which k01 is preferably an integer of 1 to 4, and more preferably 1), and more preferably an oxygen atom.

Examples of the alkyl group, the alkenyl group, the alicyclic hydrocarbon group, the aromatic hydrocarbon group, and groups obtained by combining these groups in R^{a02}, R^{a03}, R^{a04}, R^{a6} and R^{a7} include the same groups as mentioned as for R^{a1}, R^{a2} and R^{a3} of formula (1).

The alkyl group in R^{a02}, R^{a03} and R^{a04} is preferably an alkyl group having 1 to 6 carbon atoms, more preferably a methyl group or an ethyl group, and still more preferably a methyl group.

The alkyl group in R^{a6} and R^{a7} is preferably an alkyl group having 1 to 6 carbon atoms, more preferably a methyl group, an ethyl group, an isopropyl group or a t-butyl group, and still more preferably an ethyl group, an isopropyl group or a t-butyl group.

The alkenyl group in R^{a6} and R^{a7} is preferably an alkenyl group having 2 to 6 carbon atoms, and more preferably an ethenyl group, a propenyl group, an isopropenyl group or a butenyl group.

The number of carbon atoms of the alicyclic hydrocarbon group as for R^{a02}, R^{a03}, R^{a04}, R^{a6} and R^{a7} is preferably 5 to 12, and more preferably 5 to 10.

The number of carbon atoms of the aromatic hydrocarbon group of R^{a02}, R^{a03}, R^{a04}, R^{a6} and R^{a7} is preferably 6 to 12, and more preferably 6 to 10.

The total number of carbon atoms of the groups obtained by combining the alkyl group with the alicyclic hydrocarbon group is preferably 18 or less.

The total number of carbon atoms of the groups obtained by combining the alkyl group with the aromatic hydrocarbon group is preferably 18 or less.

R^{a02} and R^{a03} are preferably an alkyl group having 1 to 6 carbon atoms which may have a halogen atom or an aromatic hydrocarbon group having 6 to 12 carbon atoms which may have a halogen atom, and more preferably a methyl group, an ethyl group, a phenyl group or a naphthyl group which may have a halogen atom.

R^{a04} is preferably an alkyl group having 1 to 6 carbon atoms which may have a halogen atom or an alicyclic hydrocarbon group having 5 to 12 carbon atoms which may have a halogen atom, and more preferably a methyl group, an ethyl group, a cyclohexyl group or an adamantyl group which may have a halogen atom.

R^{a6} and R^{a7} are preferably an alkyl group having 1 to 6 carbon atoms which may have a halogen atom, an alkenyl group having 2 to 6 carbon atoms which may have a halogen atom or an aromatic hydrocarbon group having 6 to 12 carbon atoms which may have a halogen atom, more preferably a methyl group, an ethyl group, an isopropyl group, a t-butyl group, an ethenyl group, a phenyl group or a naphthyl group which may have a halogen atom, and still more preferably an ethyl group, an isopropyl group, a t-butyl group, an ethenyl group or a phenyl group which may have a halogen atom.

m1′ is preferably an integer of 0 to 3, and more preferably 0 or 1.

n1 is preferably an integer of 0 to 3, and more preferably 0 or 1.

n1′ is preferably 0 or 1.

The structural unit (a1-0) includes, for example, a structural unit represented by any one of formula (a1-0-1) to formula (a1-0-21) and a structural unit in which a methyl group corresponding to R^{a01} in the structural unit (a1-0) is substituted with a hydrogen atom, a halogen atom, a haloalkyl group (an alkyl group having a halogen atom) or other alkyl groups, and is preferably a structural unit represented by any one of formula (a1-0-1) to formula (a1-0-10), formula (a1-0-13), formula (a1-0-14) and formula (a1-0-19) to formula (a1-0-21).

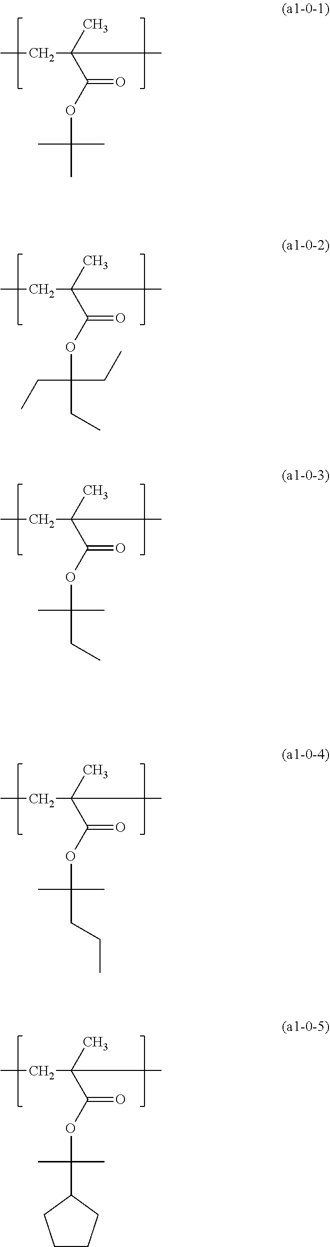

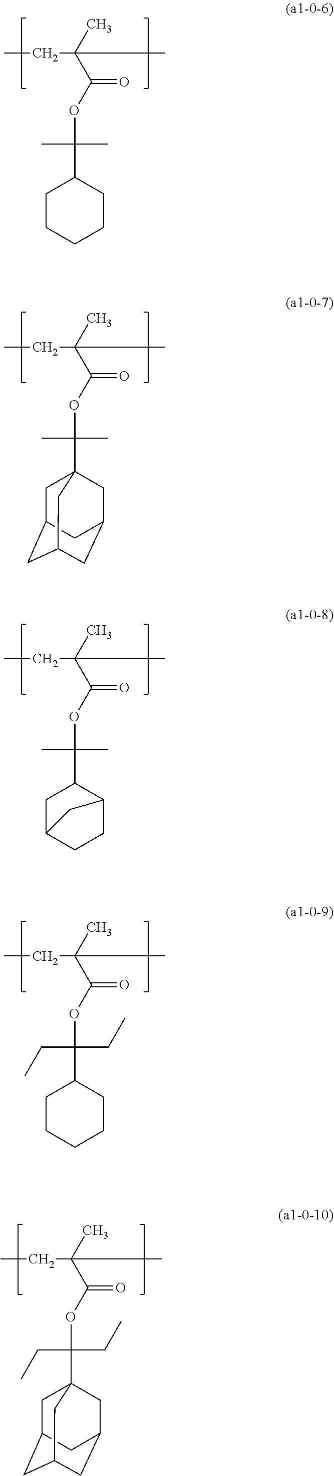

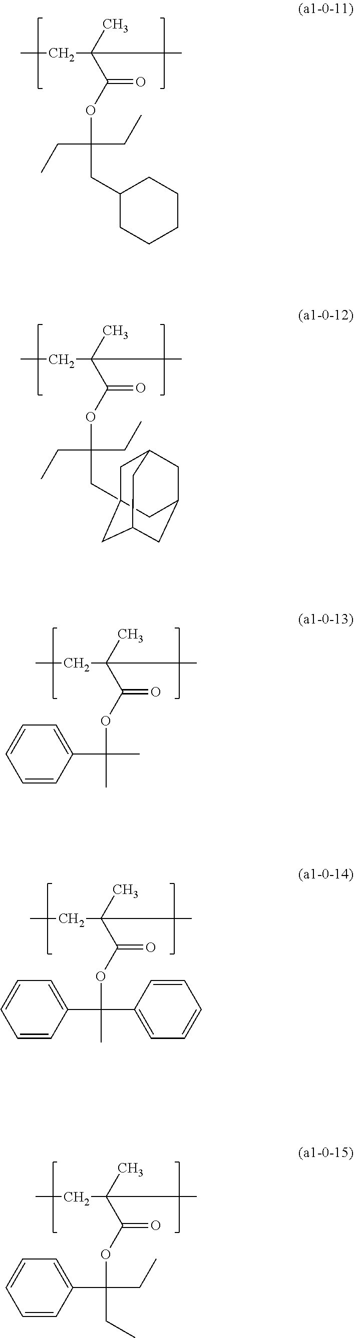

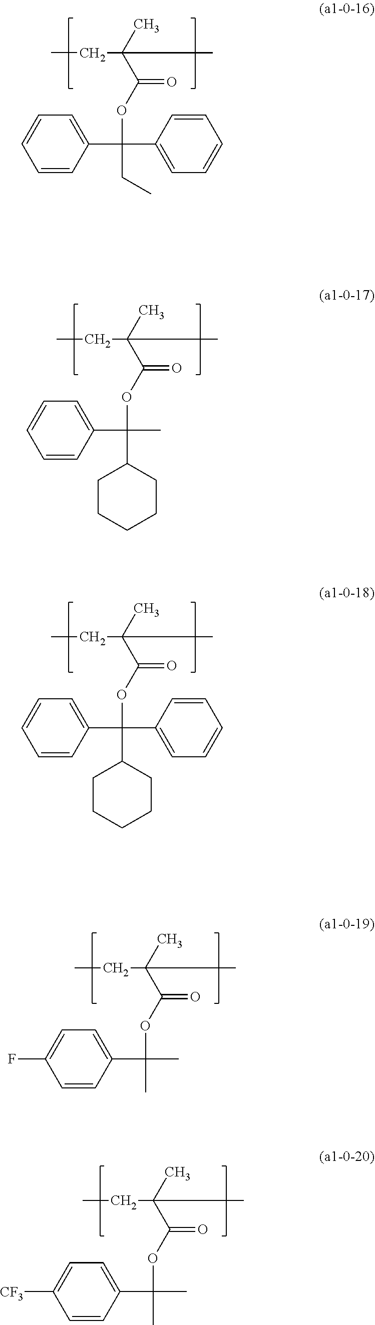

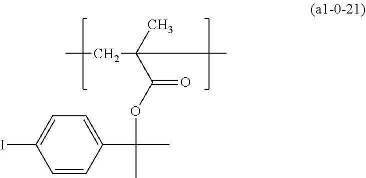

The structural unit (a1-1) includes, for example, structural units derived from the monomers mentioned in JP 2010-204646 A. Of these structural units, a structural unit represented by any one of formula (a1-1-1) to formula (a1-1-7) and a structural unit in which a methyl group corresponding to R^{a4} in the structural unit (a1-1) is substituted with a hydrogen atom, a halogen atom, a haloalkyl group or other alkyl groups are preferable, and a structural unit represented by any one of formula (a1-1-1) to formula (a1-1-4) is more preferable.

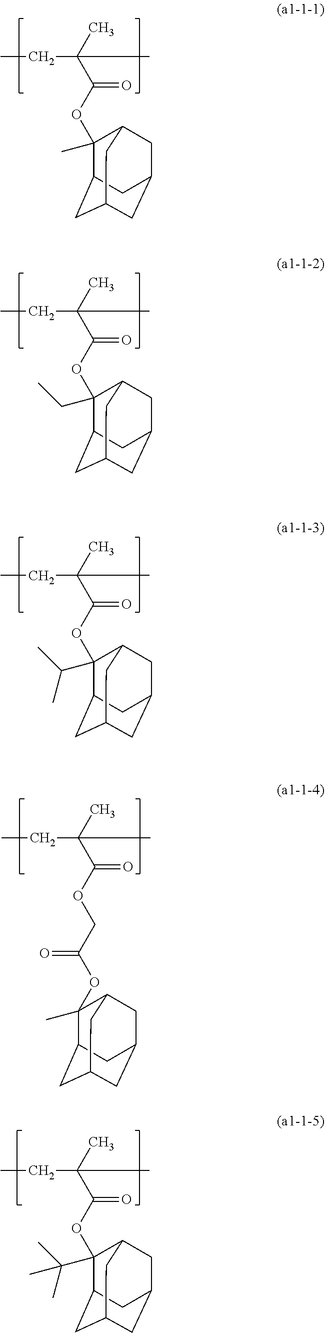

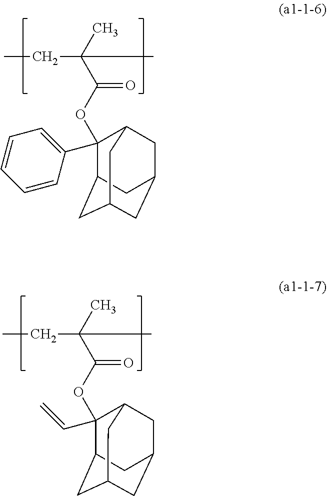

Examples of the structural unit (a1-2) include a structural unit represented by any one of formula (a1-2-1) to formula (a1-2-20), and a structural unit in which a methyl group corresponding to R^{a5} in the structural unit (a1-2) is substituted with a hydrogen atom, a halogen atom, a haloalkyl group or other alkyl groups, and a structure unit represented by any one of formula (a1-2-2), formula (a1-2-5), formula (a1-2-6) and formula (a1-2-10) to formula (a1-2-20) is preferable.

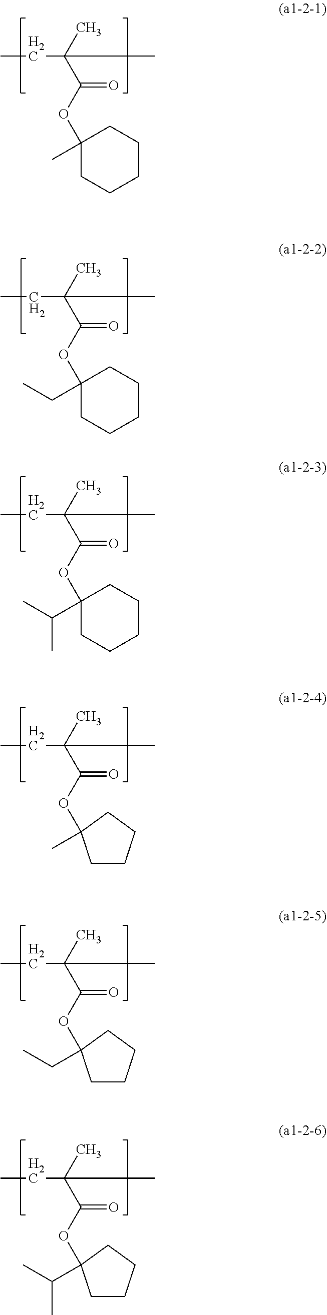

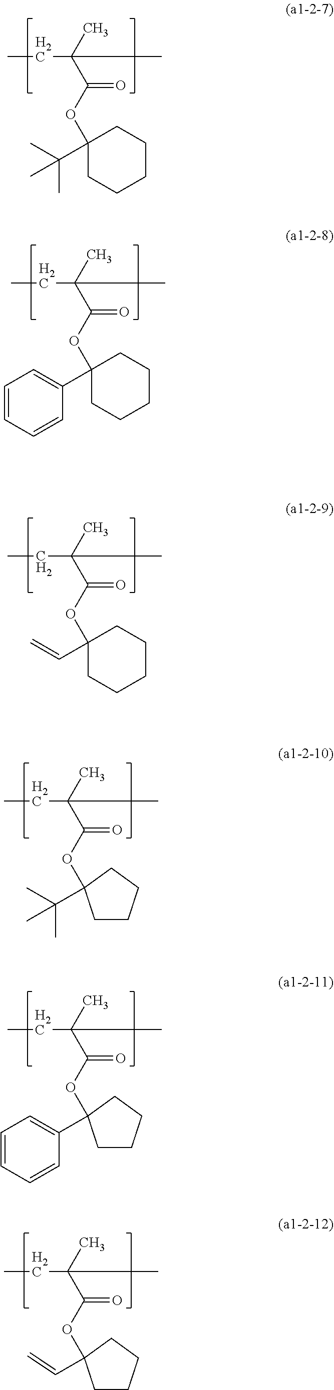

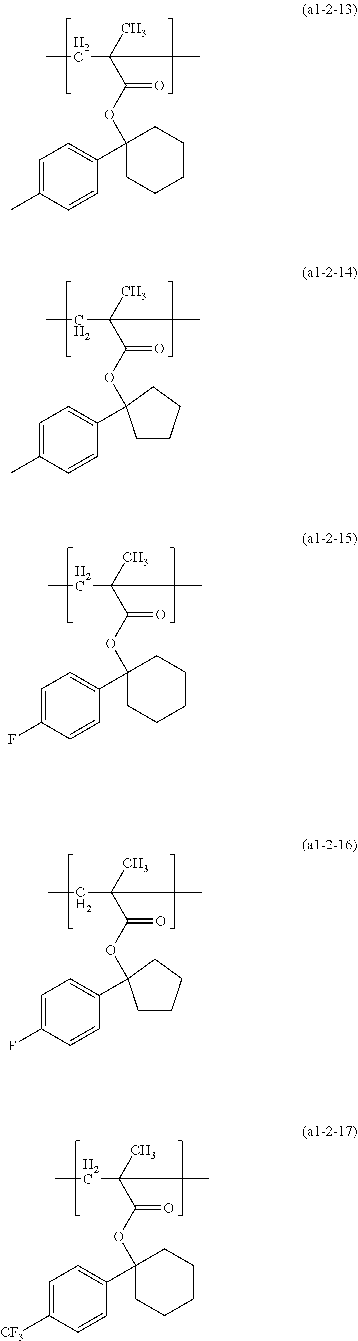

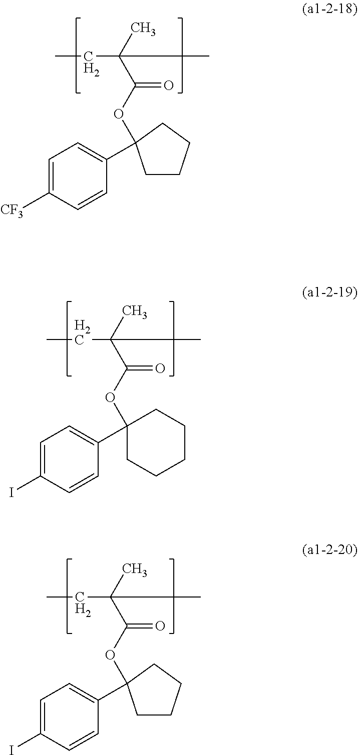

When the resin (A) or the like includes a structural unit (a1-0) and/or a structural unit (a1-1) and/or a structural unit (a1-2), the total content of them is usually mol % or more, preferably 15 mol % or more, more preferably mol % or more, still more preferably 25 mol % or more, yet more preferably 30 mol % or more, further preferably 40 mol % or more, and still further preferably 50 mol % or more, based on all structural units of the resin (A) or the like. The total content is also usually 95 mol % or less, preferably 90 mol % or less, more preferably 85 mol % or less, still more preferably 70 mol % or less, and yet more preferably 65 mol % or less, based on all structural units of the resin (A) or the like. Specifically, the total content is usually 10 to 95 mol %, preferably 15 to 90 mol %, more preferably 20 to 85 mol %, still more preferably 25 to 70 mol %, and yet more preferably 30 to 70 mol %, based on all structural units of the resin (A) or the like.

When the resin (A) or the like includes a structural unit (a1-0), the content is usually 5 mol % or more, preferably 10 mol % or more, more preferably 15 mol % or more, still more preferably 20 mol % or more, yet more preferably 25 mol % or more, further preferably 30 mol % or more, and still further preferably 35 mol % or more, based on all structural units of the resin (A) or the like. The content is also usually 80 mol % or less, preferably 75 mol % or less, more preferably 70 mol % or less, still more preferably 65 mol % or less, and yet more preferably 60 mol % or less, based on all structural units of the resin (A) or the like. Specifically, the content is usually 5 to 80 mol %, preferably 5 to 75 mol %, and more preferably 10 to 70 mol %, based on all structural units of the resin (A) or the like.

When the resin (A) or the like includes a structural unit (a1-1) and/or a structural unit (a1-2), the total content of them is usually 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. The total content is also usually 95 mol % or less, preferably 90 mol % or less, more preferably 85 mol % or less, still more preferably 80 mol % or less, yet more preferably 75 mol % or less, further preferably 70 mol % or less, and still further preferably 65 mol % or less, based on all structural units of the resin (A) or the like. Specifically, the total content is usually 10 to 90 mol %, preferably 15 to 85 mol %, more preferably 15 to 80 mol %, still more preferably 20 to 80 mol %, yet more preferably 20 to 75 mol %, and further preferably 20 to 70 mol %, based on all structural units of the resin (A) or the like.

In the structural unit (a1), examples of the structural unit having a group (2) include a structural unit represented by formula (a1-4) (hereinafter sometimes referred to as “structural unit (a1-4)”):

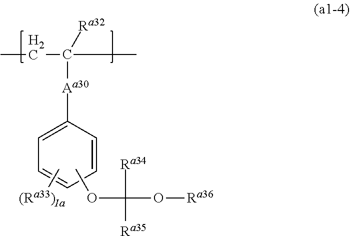

wherein, in formula (a1-4),
R^{a32} represents a hydrogen atom, a halogen atom, or an alkyl group having 1 to 6 carbon atoms which may have a halogen atom,
R^{a33} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a30} represents a single bond or *—X^{a31}-(A^{a32}-X^{a32})_{nc}- and * represents a bonding site to carbon atoms to which —R^{a32} is bonded,
A^{a32} represents an alkanediyl group having 1 to 8 carbon atoms,
X^{a31} and X^{a32} each independently represent —O—, —CO—O— or —O—CO—,
nc represents 0 or 1,
1a represents an integer of 0 to 4, and when 1a is an integer of 2 or more, a plurality of R^{a33} may be the same or different from each other, and
R^{a34} and R^{a35} each independently represent a hydrogen atom or a hydrocarbon group having 1 to 12 carbon atoms, R^{a36} represents a hydrocarbon group having 1 to 20 carbon atoms, or R^{a35} and R^{a36} may be bonded to each other to form a divalent hydrocarbon group having 2 to 20 carbon atoms together with —C—O— to which R^{a35} and R^{a36} are bonded, and —CH₂— included in the hydrocarbon group and the divalent hydrocarbon group may be replaced by —O— or —S—.

Examples of the halogen atom in R^{a32} and R^{a33} include a fluorine atom, a chlorine atom and a bromine atom.

Examples of the alkyl group having 1 to 6 carbon atoms which may have a halogen atom in R^{a32} and R^{a33} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group and a perfluorohexyl group.

R^{a32} is preferably a hydrogen atom or an alkyl group having 1 to 4 carbon atoms, more preferably a hydrogen atom, a methyl group or an ethyl group, and still more preferably a hydrogen atom or a methyl group.

Examples of the alkyl group in R^{a33} include a methyl group, an ethyl group, a propyl group, an isopropyl group, a butyl group, a sec-butyl group, a tert-butyl group, a pentyl group and a hexyl group. The alkyl group is preferably an alkyl group having 1 to 4 carbon atoms, more preferably a methyl group or an ethyl group, and still more preferably a methyl group.

Examples of the alkoxy group in R^{a33} include a methoxy group, an ethoxy group, a propoxy group, an isopropoxy group, a butoxy group, a sec-butoxy group, a tert-butoxy group, a pentyloxy group and a hexyloxy group. The alkoxy group is preferably an alkoxy group having 1 to 4 carbon atoms, more preferably a methoxy group or an ethoxy group, and still more preferably a methoxy group.

Examples of the alkoxyalkyl group in R^{a33} include a methoxymethyl group, an ethoxyethyl group, a propoxymethyl group, an isopropoxymethyl group, a butoxymethyl group, a sec-butoxymethyl group and a tert-butoxymethyl group. The alkoxyalkyl group is preferably an alkoxyalkyl group having 2 to 8 carbon atoms, more preferably a methoxymethyl group or an ethoxyethyl group, and still more preferably a methoxymethyl group.

Examples of the alkoxyalkoxy group in R^{a33} include a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The alkoxyalkoxy group is preferably an alkoxyalkoxy group having 2 to 8 carbon atoms, and more preferably a methoxyethoxy group or an ethoxyethoxy group.

Examples of the alkylcarbonyl group in R^{a33} include an acetyl group, a propionyl group and a butyryl group. The alkylcarbonyl group is preferably an alkylcarbonyl group having 2 to 3 carbon atoms, and more preferably an acetyl group.

Examples of the alkylcarbonyloxy group in R^{a33} include an acetyloxy group, a propionyloxy group and a butyryloxy group. The alkylcarbonyloxy group is preferably an alkylcarbonyloxy group having 2 to 3 carbon atoms, and more preferably an acetyloxy group.

R^{a33} is preferably a halogen atom, a hydroxy group, an alkyl group having 1 to 4 carbon atoms, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group or an ethoxyethoxy group.

Examples of the *—X^{a3}1-(A^{a32}-X^{a32})_{nc}— include *—O—, *—CO—O—, *—O—CO—, *—CO—O-A^{a32}-CO—O—, *—O—CO-A^{a32}-O—, *—O-A^{a32}-CO—O—, *—CO—O-A^{a32}-O—CO— and *—O—CO-A^{a32}-O—CO. Of these, *—CO—O—, *—CO—O-A^{a32}-CO—O— or *—O-A^{a32}-CO—O— is preferable.

Examples of the alkanediyl group in A^{a32} include a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group and a 2-methylbutane-1,4-diyl group.

A^{a32} is preferably a methylene group or an ethylene group.

A^{a30} is preferably a single bond, *—CO—O— or *—CO—O— A^{a32}-CO—O—, more preferably a single bond, *—CO—O— or *—CO—O—CH₂—CO—O—, and still more preferably a single bond or *—CO—O—.

1a is preferably 0, 1 or 2, more preferably 0 or 1, and still more preferably 0.

Examples of the hydrocarbon group in R^{a34}, R^{a35} and R^{a36} include an alkyl group, an alicyclic hydrocarbon group, an aromatic hydrocarbon group, and groups obtained by combining these groups.

Examples of the alkyl group include a methyl group, an ethyl group, a propyl group, a butyl group, a pentyl group, a hexyl group, a heptyl group, an octyl group and the like.

The alicyclic hydrocarbon group may be either monocyclic or polycyclic. Examples of the monocyclic alicyclic hydrocarbon group include cycloalkyl groups such as a cyclopentyl group, a cyclohexyl group, a cycloheptyl group and a cyclooctyl group. Examples of the polycyclic alicyclic hydrocarbon group include a decahydronaphthyl group, an adamantyl group, a norbornyl group, and the following groups (* represents a bonding site).

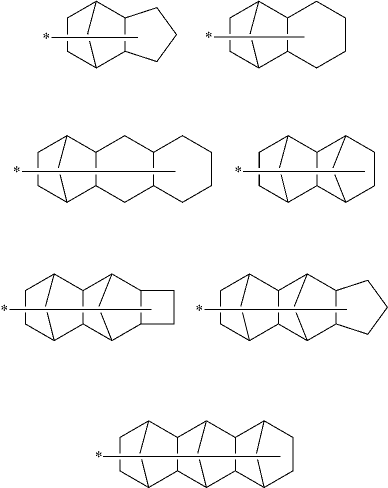

Examples of the aromatic hydrocarbon group include aryl groups such as a phenyl group, a naphthyl group, an anthryl group, a biphenyl group and a phenanthryl group.

Examples of the combined group include groups obtained by combining the above-mentioned alkyl group and alicyclic hydrocarbon group (e.g., alkylcycloalkyl groups or cycloalkylalkyl groups, such as a methylcyclohexyl group, a dimethylcyclohexyl group, a methylnorbornyl group, a cyclohexylmethyl group, an adamantylmethyl group, an adamantyldimethyl group and a norbornylethyl group), aralkyl groups such as a benzyl group, aromatic hydrocarbon groups having an alkyl group (a p-methylphenyl group, a p-tert-butylphenyl group, a tolyl group, a xylyl group, a cumenyl group, a mesityl group, a 2,6-diethylphenyl group, a 2-methyl-6-ethylphenyl group, etc.), aromatic hydrocarbon groups having an alicyclic hydrocarbon group (a p-cyclohexylphenyl group, a p-adamantylphenyl group, etc.), aryl-cycloalkyl groups such as a phenylcyclohexyl group and the like. Particularly, examples of R^{a36} include an alkyl group having 1 to 18 carbon atoms, an alicyclic hydrocarbon group having 3 to 18 carbon atoms, an aromatic hydrocarbon group having 6 to 18 carbon atoms, or groups formed by combining these groups.

When R^{a35} and R^{a36} are bonded to each other to form a divalent hydrocarbon group having 2 to 20 carbon atoms together with —C—O— to which R^{a35} and R^{a36} are bonded, examples of the group include the following groups. * represents a bonding site, and one of * is a bonding site to R^{a34}.

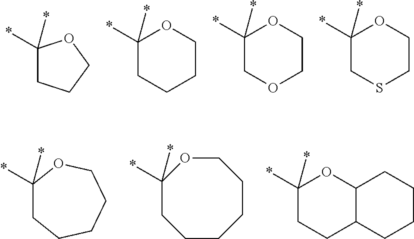

R^{a34} is preferably a hydrogen atom.

R^{a35} is preferably a hydrogen atom, an alkyl group having 1 to 12 carbon atoms or an alicyclic hydrocarbon group having 3 to 12 carbon atoms, and more preferably a methyl group or an ethyl group.

The hydrocarbon group of R^{a36} is preferably an alkyl group having 1 to 18 carbon atoms, an alicyclic hydrocarbon group having 3 to 18 carbon atoms, an aromatic hydrocarbon group having 6 to 18 carbon atoms, or groups formed by combining these groups, and more preferably an alkyl group having 1 to 18 carbon atoms, an alicyclic hydrocarbon group having 3 to 18 carbon atoms or an aralkyl group having 7 to 18 carbon atoms. The alkyl group and the alicyclic hydrocarbon group in R^{a36} are preferably unsubstituted. The aromatic hydrocarbon group in R^{a36} is preferably an aromatic ring having an aryloxy group having 6 to 10 carbon atoms.

—OC(R^{a34}) (R^{a35})O—R^{a36} in the structural unit (a1-4) is eliminated by contacting with an acid (e.g., p-toluenesulfonic acid) to form a hydroxy group or a carboxy group.

—OC(R^{a}S4) (R^{a35})—O—R^{a36} is preferably bonded at the m-position or the p-position, and more preferably at the p-position of the benzene ring.

The structural unit (a1-4) includes, for example, structural units derived from the monomers mentioned in JP 2010-204646 A. The structural unit preferably includes structural units represented by formula (a1-4-1) to formula (a1-4-32) and a structural unit in which a hydrogen atom corresponding to R^{a32} in the structural unit (a1-4) is substituted with a halogen atom, a haloalkyl group or an alkyl group, and more preferably structural units represented by formula (a1-4-1) to formula (a1-4-5), formula (a1-4-10), formula (a1-4-13), formula (a1-4-14), formula (a1-4-19) and formula (a1-4-20).

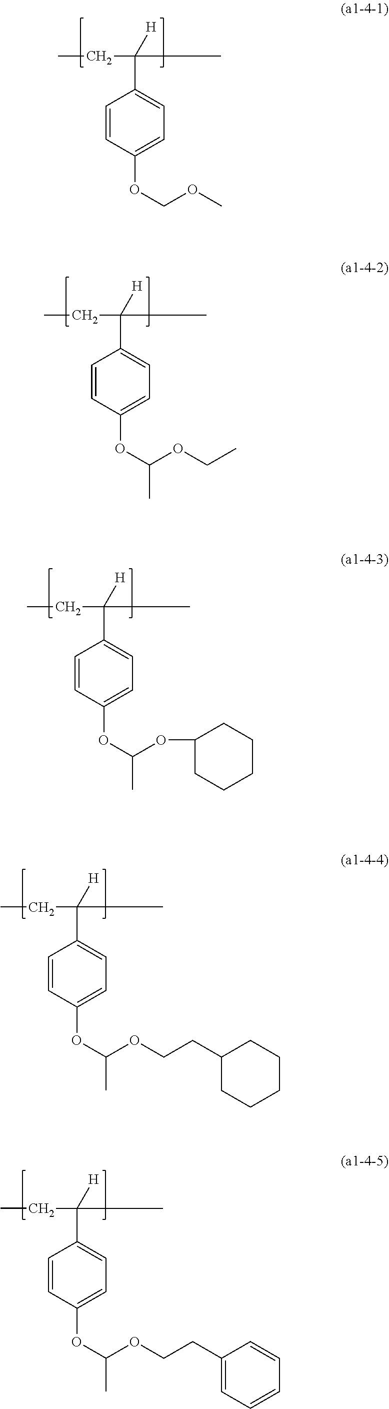

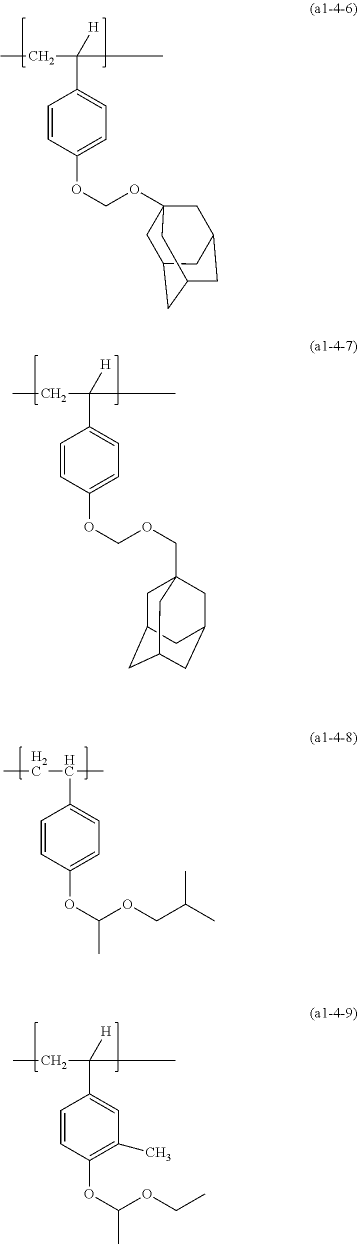

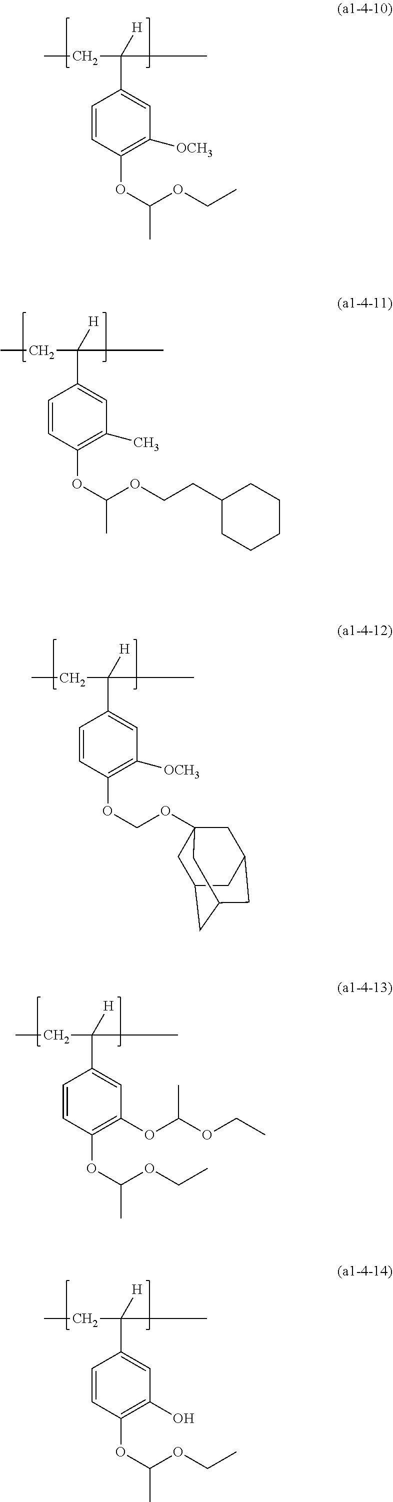

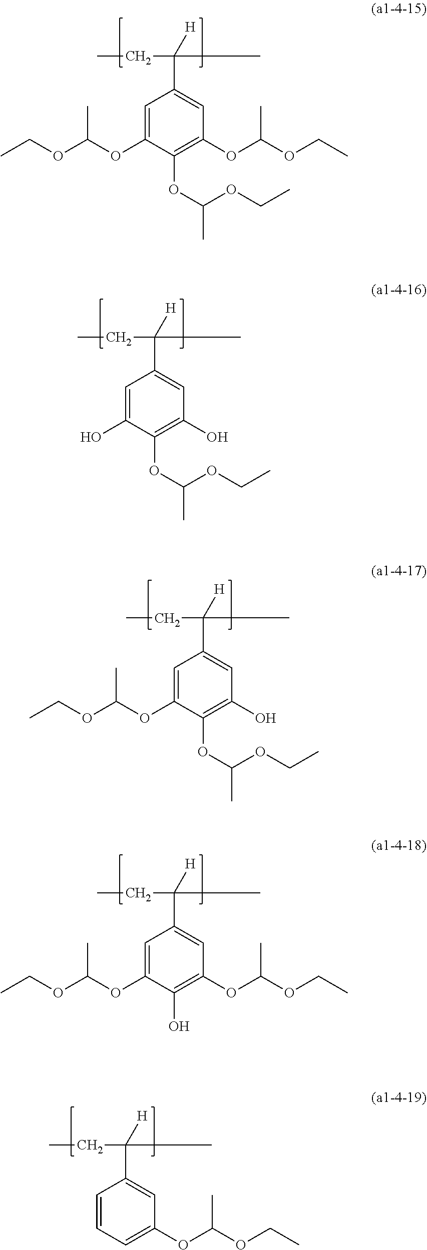

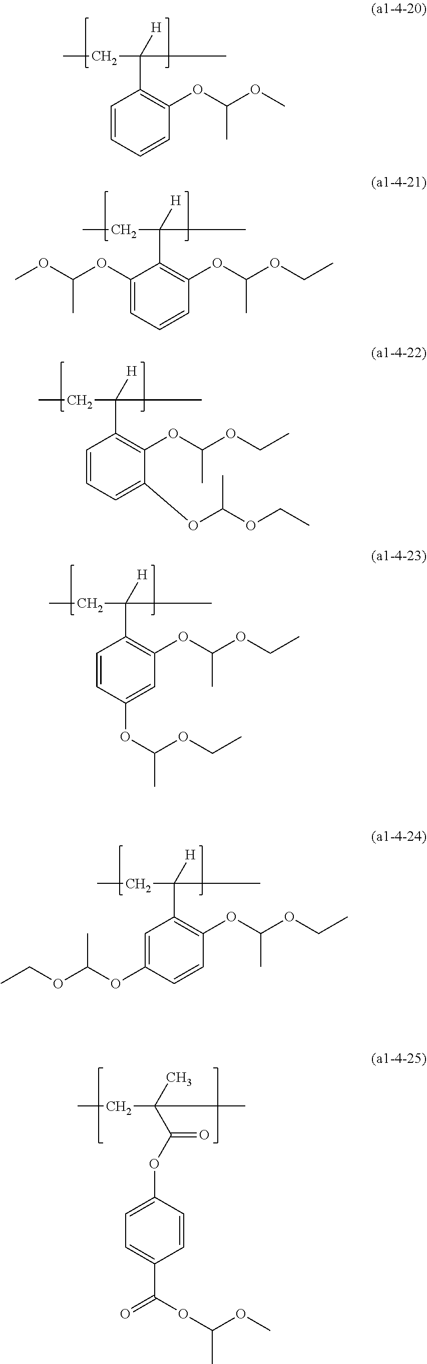

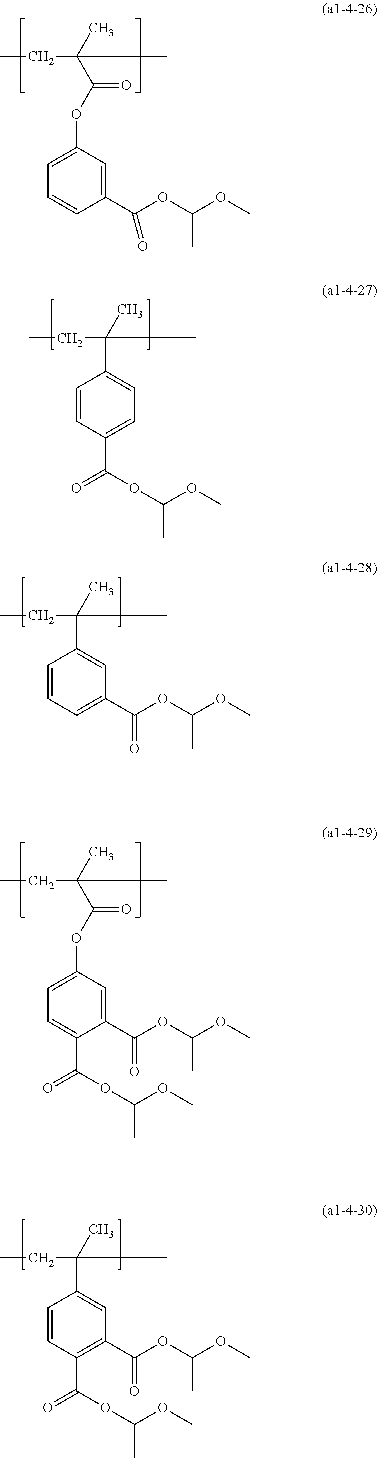

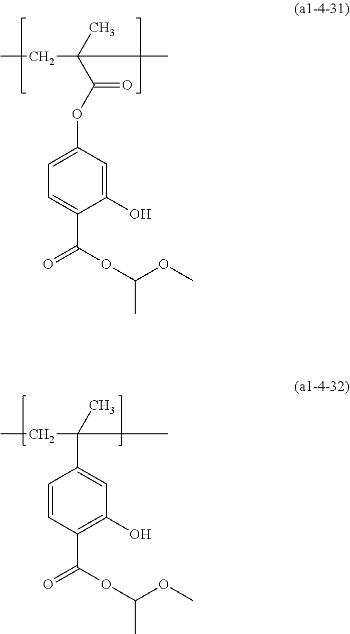

When the resin (A) or the like includes the structural unit (a1-4), the content is usually 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on the total of all structural units of the resin (A) or the like. The content is also usually 95 mol % or less, preferably 90 mol % or less, more preferably 85 mol % or less, still more preferably 80 mol % or less, yet more preferably 75 mol % or less, further preferably 70 mol % or less, and still further preferably 65 mol % or less, based on the total of all structural units of the resin (A) or the like. Specifically, the content is preferably 10 to 95 mol %, more preferably 15 to 90 mol %, still more preferably 20 to 85 mol %, yet more preferably 20 to 70 mol %, and further preferably 20 to 65 mol %, based on the total of all structural units of the resin (A) or the like.

The structural unit derived from a (meth)acrylic monomer having a group (2) also includes a structural unit represented by formula (a1-5) (hereinafter sometimes referred to as “structural unit (a1-5)”):

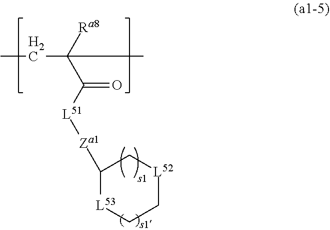

wherein, in formula (a1-5),
R^{aB} represents an alkyl group having 1 to 6 carbon atoms which may have a halogen atom, a hydrogen atom or a halogen atom,
Z^{a1} represents a single bond or *—(CH₂)ₕ₃—CO-L⁵⁴-, h3 represents an integer of 1 to 4, and * represents a bonding site to L⁵¹,
L⁵¹, L⁵², L⁵³ and L⁵⁴ each independently represent —O— or —S—,
s1 represents an integer of 1 to 3, and
s1′ represents an integer of 0 to 3.

The halogen atom includes a fluorine atom and a chlorine atom and is preferably a fluorine atom.

Examples of the alkyl group having 1 to 6 carbon atoms which may have a halogen atom include a methyl group, an ethyl group, a propyl group, a butyl group, a pentyl group, a hexyl group, a heptyl group, an octyl group, a fluoromethyl group and a trifluoromethyl group.

In formula (a1-5), R^{aB} is preferably a hydrogen atom, a methyl group or a trifluoromethyl group,
L⁵¹ is preferably an oxygen atom,
one of L⁵² and L⁵³ is preferably —O— and the other one is preferably —S—,
s1 is preferably 1,
s1′ is preferably an integer of 0 to 2, and
Z^{a1} is preferably a single bond or *—CH₂—CO—O—.

The structural unit (a1-5) includes, for example, structural units derived from the monomers mentioned in JP 2010-61117 A. Of these structural units, structural units represented by formula (a1-5-1) to formula (a1-5-4) are preferable, and structural units represented by formula (a1-5-1) or formula (a1-5-2) are more preferable.

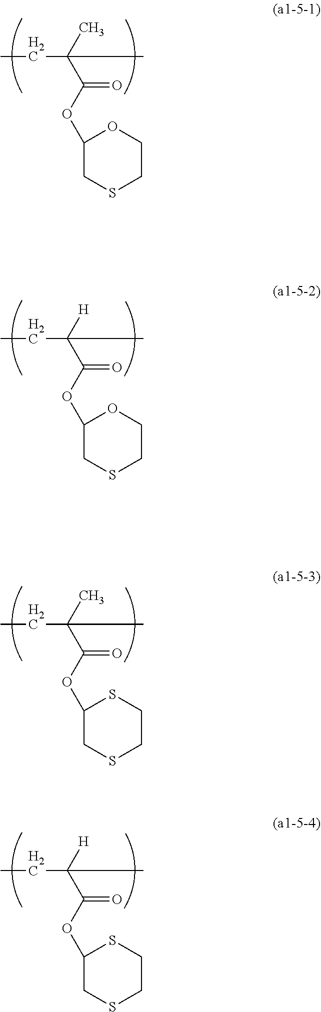

When the resin (A) or the like includes the structural unit (a1-5), the content is usually 1 mol % or more, preferably 2 mol % or more, more preferably 3 mol % or more, still more preferably 5 mol % or more, yet more preferably 10 mol % or more, further preferably 20 mol % or more, and still further preferably 25 mol % or more, based on all structural units of the resin (A) or the like. The content is also usually 80 mol % or less, preferably 70 mol % or less, more preferably 60 mol % or less, still more preferably 50 mol % or less, yet more preferably 45 mol % or less, further preferably mol % or less, and still further preferably 30 mol % or less, based on all structural units of the resin (A) or the like. Specifically, the content is preferably 1 to 50 mol %, more preferably 3 to 45 mol %, still more preferably 5 to 40 mol %, and yet more preferably 5 to 30 mol %, based on all structural units of the resin (A) or the like.

Examples of the structural unit having a group (1) in the structural unit (a1) include a structural unit represented by formula (a1-6) (hereinafter sometimes referred to as “structural unit (a1-6)”):

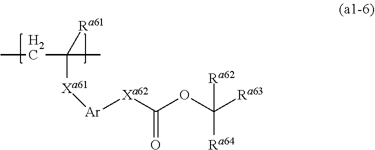

wherein, in formula (a1-6),
R^{a61} represents a hydrogen atom, a halogen atom, or an alkyl group having 1 to 6 carbon atoms which may have a halogen atom,
R^{a62}, R^{a63} and R^{a64} each independently represent an alkyl group having 1 to 6 carbon atoms or a cyclic hydrocarbon group having 3 to 18 carbon atoms which may have a substituent, or R^{a62} and R^{a63} may be bonded to each other to form a ring having 3 to 20 carbon atoms together with carbon atoms to which R^{a62} and R^{a63} are bonded,
X^{a61} represents a single bond, —CO—O—* or —CO—NR⁵—*, * represents a bonding site to —Ar, and R⁵ represents a hydrogen atom or an alkyl group having 1 to 4 carbon atoms,
X^{a62} represents a single bond, *—O-L^{a61}- or *—CO—O-L^{a62}-, * represents a bonding site to —Ar, and L^{a61} and L^{a62} each independently represent an alkanediyl group having 1 to 4 carbon atoms, and
Ar represents an aromatic hydrocarbon group having 6 to 20 carbon atoms which may have a substituent.

Examples the halogen atom in R^{a61} include a fluorine atom, an iodine atom, a chlorine atom and a bromine atom. Of these, a fluorine atom is preferable.

Examples of the alkyl group having 1 to 6 carbon atoms which may have a halogen atom in R^{a61} include a methyl group, an ethyl group, a propyl group, a butyl group, a pentyl group, a hexyl group, a fluoromethyl group, a difluoromethyl group, a trifluoromethyl group, a perfluoroethyl group, a 2,2,2-trifluoroethyl group, a 1,1,2,2-tetrafluoroethyl group, a perfluoropropyl group, a 2,2,3,3,3-pentafluoropropyl group, a perfluorobutyl group, a 1,1,2,2,3,3,4,4-octafluorobutyl group, a perfluoropentyl group, a 2,2,3,3,4,4,5,5,5-nonafluoropentyl group and a perfluorohexyl group.

R^{a61} is preferably a hydrogen atom, a methyl group or a trifluoromethyl group.

Examples of the alkyl group in R^{a62}, R^{a63} and R^{a64} include a methyl group, an ethyl group, a propyl group, a butyl group, a pentyl group, a hexyl group and the like. The alkyl group is preferably an alkyl group having 1 to 4 carbon atoms, more preferably an alkyl group having 1 to 3 carbon atoms, and still more preferably a methyl group or an ethyl group.

Examples of the cyclic hydrocarbon group in R^{a62}, R^{a63} and R^{a64} include an alicyclic hydrocarbon group and an aromatic hydrocarbon group.

The alicyclic hydrocarbon group may be either monocyclic or polycyclic. Examples of the monocyclic alicyclic hydrocarbon group include a cyclopentyl group, a cyclohexyl group, a cycloheptyl group, a cyclooctyl group and the like. Examples of the polycyclic alicyclic hydrocarbon group include a decahydronaphthyl group, an adamantyl group, a norbornyl group and the like. The number of carbon atoms of the alicyclic hydrocarbon group is preferably 3 to 16, and more preferably 3 to 12.

Examples of the aromatic hydrocarbon group include a phenylene group, a naphthylene group and the like.

Examples of the substituent which may be possessed by the cyclic hydrocarbon group include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group.

Examples of the ring formed by bonding R^{a62} and R^{a63} to each other include an adamantane ring, a cyclopentane ring or a cyclohexane ring. Specifically, when R^{a62} and R^{a63} are bonded to each other to form a ring, examples of —C(R^{a62}) (R^{a63}) (R^{a64}) include the following groups. * represents a bonding site to the oxygen atom. The number of carbon atoms of the ring is preferably 3 to 16, and more preferably 3 to 12.

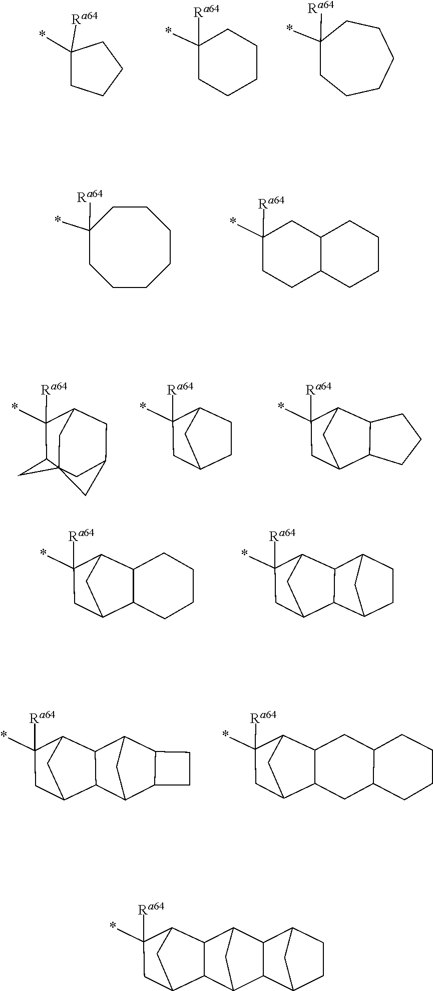

Examples of the alkanediyl group having 1 to 4 carbon atoms in L^{a61} and L^{a62} include a methylene group, an ethylene group, a propane-1,3-diyl group, a propane-1,2-diyl group, a butane-1,4-diyl group, a butane-1,3-diyl group, a 2-methylpropane-1,3-diyl group, a 2-methylpropane-1,2-diyl group and the like.

Preferably, L^{a61} and L^{a62} each independently is a methylene group or an ethylene group.

X^{a61} is preferably a single bond or —CO—O—*, and more preferably a single bond.

X^{a62} is preferably a single bond or *—O-L^{a61}-, and more preferably a single bond.

Examples of the aromatic hydrocarbon group having 6 to carbon atoms as for Ar include a phenylene group, a naphthylene group, an anthrylene group, a biphenylene group and a phenanthrylene group.

Examples of the substituent which may be possessed by the aromatic hydrocarbon group include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group.

Examples of the halogen atom include a fluorine atom, a chlorine atom and a bromine atom.

Examples of the alkyl group include a methyl group, an ethyl group, a propyl group, an isopropyl group, a butyl group, a sec-butyl group, a tert-butyl group, a pentyl group and a hexyl group. The alkyl group is preferably an alkyl group having 1 to 4 carbon atoms, more preferably a methyl group or an ethyl group, and still more preferably a methyl group.

Examples of the alkoxy group include a methoxy group, an ethoxy group, a propoxy group, an isopropoxy group, a butoxy group, a sec-butoxy group, a tert-butoxy group, a pentyloxy group and a hexyloxy group. The alkoxy group is preferably an alkoxy group having 1 to 4 carbon atoms, more preferably a methoxy group or an ethoxy group, and still more preferably a methoxy group.

Examples of the alkoxyalkyl group include a methoxymethyl group, an ethoxyethyl group, a propoxymethyl group, an isopropoxymethyl group, a butoxymethyl group, a sec-butoxymethyl group and a tert-butoxymethyl group. The alkoxyalkyl group is preferably an alkoxyalkyl group having 2 to 8 carbon atoms, more preferably a methoxymethyl group or an ethoxyethyl group, and still more preferably a methoxymethyl group.

Examples of the alkoxyalkoxy group include a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The alkoxyalkoxy group is preferably an alkoxyalkoxy group having 2 to 8 carbon atoms, and more preferably a methoxyethoxy group or an ethoxyethoxy group.

Examples of the alkylcarbonyl group include an acetyl group, a propionyl group and a butyryl group. The alkylcarbonyl group is preferably an alkylcarbonyl group having 2 to 3 carbon atoms, and more preferably an acetyl group.

Examples of the alkylcarbonyloxy group include an acetyloxy group, a propionyloxy group and a butyryloxy group. The alkylcarbonyloxy group is preferably an alkylcarbonyloxy group having 2 to 3 carbon atoms, and more preferably an acetyloxy group.

The substituent is preferably a halogen atom, a hydroxy group, an alkyl group having 1 to 4 carbon atoms, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group or an ethoxyethoxy group.

Ar is preferably a phenylene group which may have a substituent, and more preferably a phenylene group which may have a hydroxy group.

Examples of the structural unit (a1-6) include structural units represented respectively by formula (a1-6-1) to formula (a1-6-43), and structural units represented respectively by formula (a1-6-1) to formula (a1-6-9) are preferable, a structural unit represented respectively by formula (a1-6-1), formula (a1-6-2), formula (a1-6-4), formula (a1-6-5), formula (a1-6-7) or formula (a1-6-8) is more preferable, and a structural unit represented respectively by formula (a1-6-1) or formula (a1-6-2) is still more preferable.

[chemical structure]

[chemical structure]

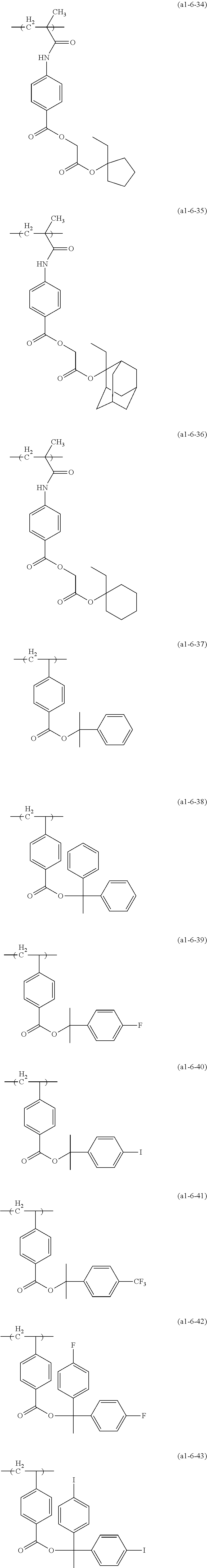

It is also possible to exemplify, as the structural unit (a1-6), structural units in which the hydrogen atom corresponding to R^{a61} is substituted with a methyl group, a halogen atom, a haloalkyl group or the like in structural units represented respectively by formula (a1-6-1) to formula (a1-6-9), formula (a1-6-16) to formula (a1-6-21), formula (a1-6-28) to formula (a1-6-30), and formula (a1-6-37) to formula (a1-6-43), and structural units in which the methyl group corresponding to R^{a61} is substituted with a hydrogen atom, a halogen atom, a haloalkyl group or the like in structural units represented respectively by formula (a1-6-10) to formula (a1-6-15), formula (a1-6-22) to formula (a1-6-27) and formula (a1-6-31) to formula (a1-6-36).

When the resin (A) includes a structural unit (a1-6), the content is usually 3 mol % or more, preferably 5 mol % or more, more preferably 7 mol % or more, still more preferably mol % or more, yet more preferably 20 mol % or more, further preferably 30 mol % or more, and still further preferably 40 mol % or more, based on all structural units of the resin (A). The content is also usually 80 mol % or less, preferably 75 mol % or less, more preferably 70 mol % or less, and still more preferably 65 mol % or less, based on all structural units of the resin (A). Specifically, the content is preferably 3 to 80 mol %, more preferably 5 to 75 mol %, still more preferably 7 to 70 mol %, further preferably 7 to 65 mol %, and still further preferably 10 to 65 mol %, based on all structural units of the resin (A).

Examples of the structural unit (a1) also include the following structural units.

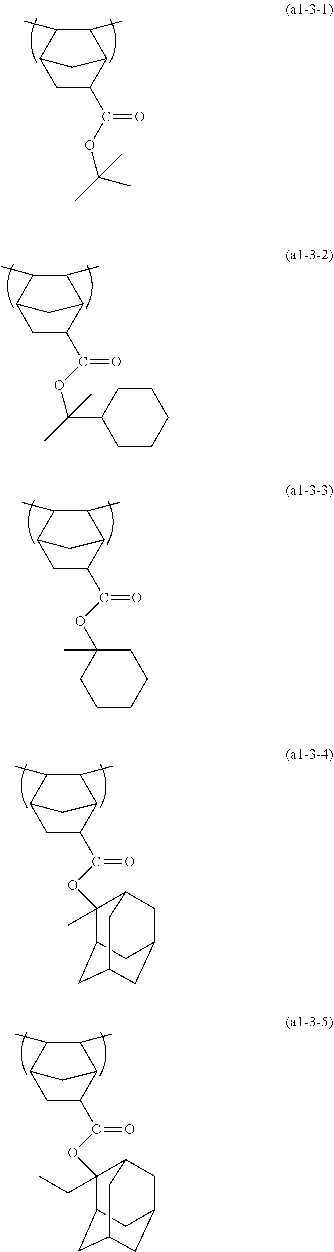

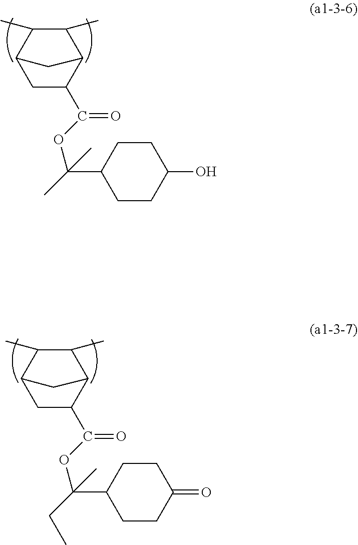

When the resin (A) or the like includes structural units represented by formulas (a1-3-1) to (a1-3-7), the content is usually 10 mol % or more, preferably 15 mol % or more, more preferably 20 mol % or more, still more preferably mol % or more, yet more preferably 30 mol % or more, further preferably 40 mol % or more, and still further preferably 50 mol % or more, based on all structural units of the resin (A) or the like. The content is also usually 95 mol % or less, preferably 90 mol % or less, more preferably 85 mol % or less, still more preferably 80 mol % or less, yet more preferably 75 mol % or less, further preferably 70 mol % or less, and still further preferably 60 mol % or less, based on all structural units of the resin (A) or the like. Specifically, the content is preferably 10 to 95 mol %, more preferably 15 to 90 mol %, still more preferably 20 to 85 mol %, yet more preferably 20 to 70 mol %, and particularly preferably 20 to 60 mol %, based on all structural units of the resin (A) or the like.

Examples of the structural unit (a1) also include the following structural units.

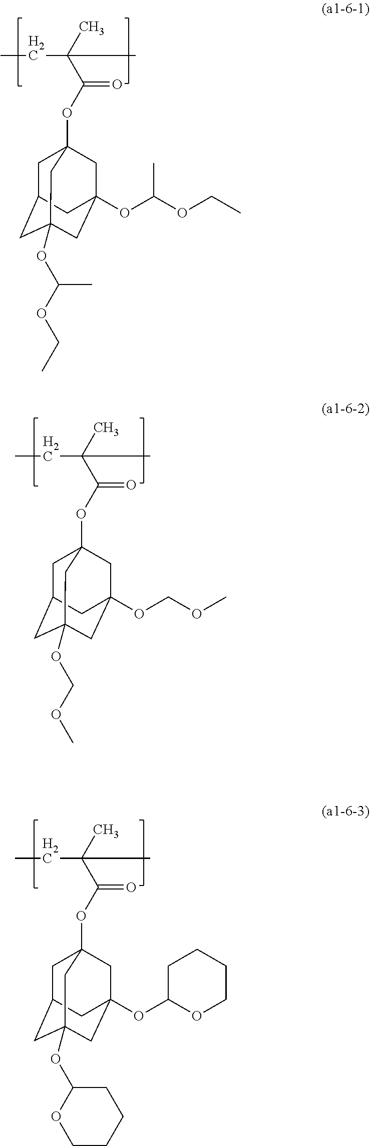

When the resin (A) or the like includes structural units represented by formulas (a1-6-1) to (a1-6-3), the content is preferably 10 to 60 mol %, more preferably 15 to 55 mol %, still more preferably 20 to 50 mol %, yet more preferably 20 to 45 mol %, and particularly preferably 20 to mol %, based on all structural units of the resin (A) or the like.

<Structural Unit (s)>

The structural unit (s) is derived from a monomer having no acid-labile group (hereinafter sometimes referred to as “monomer (s)”). It is possible to use, as the monomer from which the structural unit (s) is derived, a monomer having no acid-labile group known in the resist field.

The structural unit (s) preferably has a hydroxy group, a carboxy group or a lactone ring. When a resin including a structural unit having a hydroxy group or a carboxy group and having no acid-labile group (hereinafter sometimes referred to as “structural unit (a2)”) and/or a structural unit having a lactone ring and having no acid-labile group (hereinafter sometimes referred to as “structural unit (a3)”) is used in the resist composition of the present invention, it is possible to improve the resolution of a resist pattern and the adhesion to a substrate. Examples of the structural unit (s) include, in addition to the above-mentioned structural units, a structural unit having a halogen atom (hereinafter sometimes referred to as “structural unit (a4)”), a structural unit having a non-leaving hydrocarbon group (hereinafter sometimes referred to as “structural unit (a5)”), a structural unit having a sultone structure (hereinafter sometimes referred to as “structural unit (a6)”), a structural unit which is decomposed upon exposure to radiation to generate an acid (hereinafter sometimes referred to as “structural unit (a7)”) or other structural units known in the relevant technical field.

<Structural Unit (a2)>

The structural unit (a2) is a structural unit represented by formula (a2) and has an alcoholic hydroxy group, a phenolic hydroxy group or phenolic carboxy group:

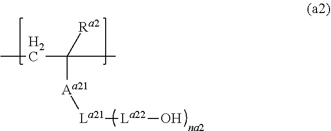

wherein, in formula (a2),
R^{a2} represents a hydrogen atom, a halogen atom or an alkyl group having 1 to 6 carbon atoms which may have a halogen atom,
A^{a21} represents a single bond or an alkanediyl group having 1 to 12 carbon atoms, and —CH₂— included in the alkanediyl group may be replaced by —O—, —CO— or —NR^{a2}—,
R^{a28} represents a hydrogen atom or an alkyl group having 1 to 4 carbon atoms,
L^{a21} represents a single bond or a hydrocarbon group having 1 to 28 carbon atoms, the hydrocarbon group may have a substituent, and —CH₂— included in the hydrocarbon group may be replaced by —O—, —CO—, —S— or —SO₂—,
L^{a22} represents a single bond or a chain hydrocarbon group having 1 to 12 carbon atoms which may have a fluorine atom, and —CH₂— included in the chain hydrocarbon group may be replaced by —O— or —CO—, and
na2 represents an integer of 1 to 5, and when na2 is 2 or more, a plurality of L^{a22} may be the same or different from each other.

Examples of the halogen atom in R^{a2} include a fluorine atom, a chlorine atom and a bromine atom.

Examples of the alkyl group having 1 to 6 carbon atoms which may have a halogen atom in R^{a2} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a tribromomethyl group, a trichloromethyl group and a triiodomethyl group. The number of carbon atoms of the alkyl group is preferably 1 to 4, more preferably 1 to 3, and still more preferably 1 or 2.

R^{a2} is preferably a hydrogen atom or an alkyl group having 1 to 4 carbon atoms, more preferably a hydrogen atom, a methyl group or an ethyl group, and still more preferably a hydrogen atom or a methyl group.

Examples of the alkanediyl group as for A^{a21} include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and branched alkanediyl groups such as a butane-1,3-diyl group, a 2-methylpropane-1,3-diyl group, a 2-methylpropane-1,2-diyl group, a pentane-1,4-diyl group, a 2-methylbutane-1,4-diyl group, a heptane-1,6-diyl group, an octane-1,7-diyl group, a nonane-1,8-diyl group and a decane-1,9-diyl group. The number of carbon atoms of the alkanediyl group is preferably 1 to 10, more preferably 1 to 8, still more preferably 1 to 6, yet more preferably 1 to 4, further preferably 1 to 3, and still further preferably 1 or 2.

A^{a21} is also preferably a methylene group or an ethylene group.

Examples of the alkyl group as for R^{a28} include a methyl group, an ethyl group, an n-propyl group, an isopropyl group, an n-butyl group and a tert-butyl group.

When —CH₂— included in the alkanediyl group as for A^{a21} is replaced by —O—, —CO— or —NR^{a28}—, the number of carbon atoms before replacement is taken as the number of carbon atoms of the alkanediyl group.

Examples of the group in which —CH₂— included in the alkanediyl group as for A^{a21} is replaced by —O—, —CO— or —NR^{a28} include a hydroxy group (a group in which —CH₂— included in the methyl group is replaced by —O—), a carboxy group (a group in which —CH₂—CH₂— included in the ethyl group is replaced by —O—CO—), a carbonyl group (a group in which —CH₂—included in the methylene group is replaced by —CO—), an oxy group (a group in which —CH₂— included in the methylene group is replaced by —O—), an amino group (a group in which —CH₂—included in the methyl group is replaced by —NR^{a28}—), an alkoxy group (a group in which —CH₂— at any position included in the alkyl group is replaced by —O—), an alkoxycarbonyl group (in which —CH₂—CH₂— at any position included in the alkyl group is replaced by —O—CO—), an alkylcarbonyl group (a group in which —CH₂— at any position included in the alkyl group is replaced by —CO—), an alkylamino group (a group in which —CH₂— at any position included in the alkyl group is replaced by —NR^{a28}—), a peptide group (a group in which —CH₂—CH₂— included in the ethylene group is replaced by —CO—NR^{a28}—), an alkanediylamino group (a group in which —CH₂— at any position included in the alkanediyl group is replaced by —NR^{a28}—) and the like. Examples of these replaced groups include those which are the same as mentioned herein.

Examples of the alkylamino group include alkylamino groups having 1 to 11 carbon atoms, for example, a methylamino group, an ethylamino group, a propylamino group, a butylamino group, a pentylamino group, a hexylamino group, an octylamino group and the like. The number of carbon atoms of the alkylamino group is preferably 1 to 8, more preferably 1 to 6, still more preferably 1 to 4, and yet more preferably 1 to 3.

Examples of the alkanediylamino group include alkanediylamino groups having 1 to 11 carbon atoms, for example a methyleneamino group, an ethyleneamino group, a propyleneamino group and the like. The number of carbon atoms of the alkanediylamino group is preferably 1 to 8, more preferably 1 to 6, still more preferably 1 to 4, and yet more preferably 1 to 3.

Of these, examples of the group in which —CH₂—included in the alkanediyl group is replaced by —O—, —CO— or —NR^{a28} as for A^{a21} include *—O—, *—CO—O—, *—O—CO—, *—CO—O-A^{a22}-CO—O—, *—O—CO-A^{a22}-O—, *—O-A^{a22}-CO—O—, *—CO—O-A^{a22}-O—CO—, *—O— CO-A^{a22}-O—CO— and *—CO—NR^{a28}—. Of these, *—CO—O—, *—CO—O-A^{a22}-CO—O—, *—O-A^{a22}-CO—O— and *—CO—NR^{a28}— are preferable. Here, A^{a22} represents an alkanediyl group having 1 to 8 carbon atoms, and * represents a bonding site to carbon atoms to which R^{a2} is bonded. Examples of the alkanediyl group as for A^{a22} include the same alkanediyl group as for A^{a21} as long as the upper limit of the number of carbon atoms permit.

A^{a21} is preferably a single bond, *—CO—O— or *—CO—O— A^{a22}-CO—O—, more preferably a single bond, *—CO—O— or *—CO—O—CH₂—CO—O—, and still more preferably a single bond or *—CO—O—.

The hydrocarbon group in L^{a21} is an (na2+1)-valent hydrocarbon group, and examples thereof include a linear or branched chain hydrocarbon group, cyclic hydrocarbon groups such as a monocyclic or polycyclic (including a spiro ring, a fused ring or a bridged ring) alicyclic hydrocarbon group and an aromatic hydrocarbon group, and the hydrocarbon group may be groups obtained by combining two or more of these groups (e.g., a hydrocarbon group formed from an alicyclic hydrocarbon group or an aromatic hydrocarbon group and a chain hydrocarbon group).

Examples of the chain hydrocarbon group as for L^{a21} include di-to hexa-valent chain hydrocarbon groups such as an alkanediyl group, an alkanetriyl group, an alkanetetrayl group, an alkanepentayl group and an alkanehexayl group.

Examples of the alkanediyl group include the same alkanediyl groups as for A^{a21}.

Examples of the alkanetriyl group include a methanetriyl group, an ethanetriyl group, a propanetriyl group, a butanetriyl group, a pentanetriyl group, a hexanetriyl group, a heptanetriyl group, an octanetriyl group, a nonanetriyl group, a decanetriyl group, an undecanetriyl group, a dodecanetriyl group, a tridecanetriyl group, a tetradecanetriyl group, a pentadecanetriyl group, a hexadecanetriyl group and a heptadecanetriyl group.

Examples of the alkanetetrayl group include a methanetetrayl group, an ethanetetrayl group, a propanetetrayl group, a butanetetrayl group, a pentanetetrayl group, a hexanetetrayl group, a heptanetetrayl group, an octanetetrayl group, a nonanetetrayl group, a decanetetrayl group, an undecanetetrayl group, a dodecanetetrayl group, a tridecanetetrayl group, a tetradecanetetrayl group, a pentadecanetetrayl group, a hexadecanetetrayl group and a heptadecanetetrayl group.

Examples thereof also include groups in which one or more hydrogen atoms of the above-mentioned groups are substituted with a bonding site.

The number of carbon atoms of the chain hydrocarbon group is preferably 1 to 18, more preferably 1 to 12, still more preferably 1 to 10, yet more preferably 1 to 9, further preferably 1 to 8, still further preferably 1 to 6, yet further preferably 1 to 5, and particularly preferably 1 to 4.

Examples of the monocyclic and polycyclic alicyclic hydrocarbon group in L^{a21} include the following alicyclic hydrocarbon groups and the like. The bonding site can be any position.

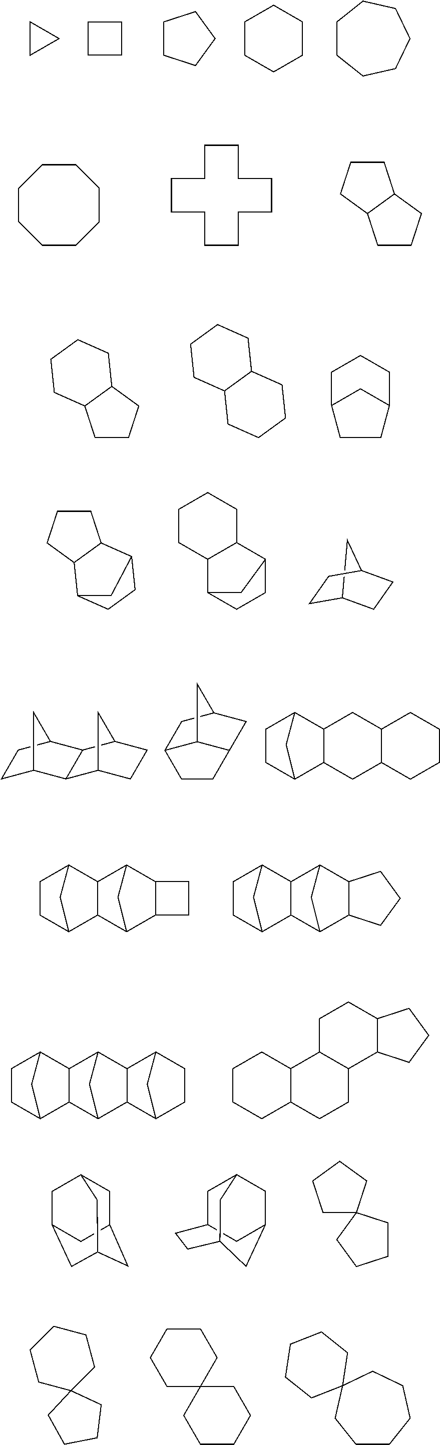

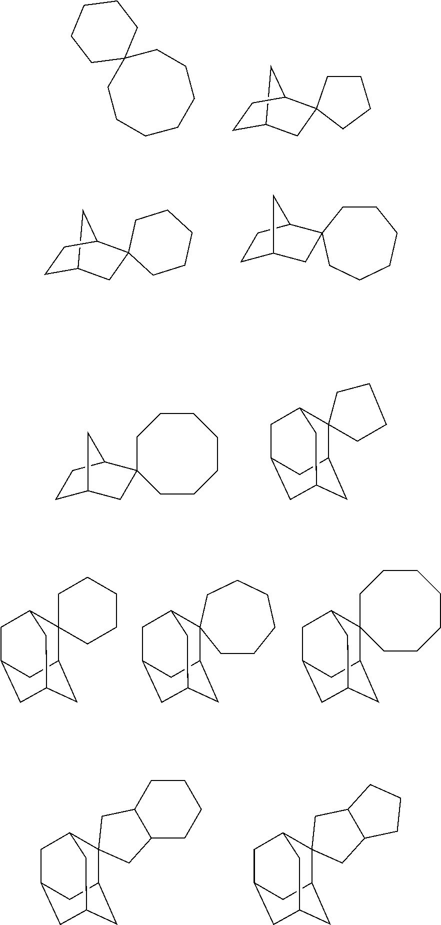

Examples thereof include di-to hexa-valent alicyclic hydrocarbon groups such as a cycloalkanediyl group, a cycloalkanetriyl group, a cycloalkanetetrayl group, a cycloalkanepentayl group and a cycloalkanehexayl group.

Specific examples thereof include monocyclic alicyclic hydrocarbon groups such as a cyclobutane-1,3-diyl group, a cyclopentane-1,3-diyl group, a cyclohexane-1,4-diyl group, a cyclooctane-1,5-diyl group, a cyclopropanetriyl group, a cyclobutanetriyl group, a cyclopentanetriyl group, a cyclohexanetriyl group, a cycloheptanetriyl group, a cyclooctanetriyl group, a cyclodecanetriyl group, a cyclopropanetetrayl group, a cyclobutanetetrayl group, a cyclopentanetetrayl group, a cyclohexanetetrayl group, a cycloheptanetetrayl group, a cyclooctanetetrayl group and a cyclodecanetetrayl group, and

polycyclic alicyclic hydrocarbon groups such as a norbornane-1,4-diyl group, a norbornane-2,5-diyl group, a adamantane-1,5-diyl group, an adamantane-2,6-diyl group, a decahydronaphthalenediyl group, a bicyclo[3.3.0]octanediyl group, a norbornanetriyl group, an adamantanetriyl group, a decahydronaphthalenetriyl group, a bicyclo[3.3.0]octanetriyl group, a norbornanetetrayl group, an adamantanetetrayl group, a decahydronaphthalenetetrayl group and a bicyclo[3.3.0]octanetetrayl group.

Examples thereof also include groups in which one or more hydrogen atoms of the above-mentioned groups are substituted with a bonding site.

The number of carbon atoms of the alicyclic hydrocarbon group is preferably 3 to 20, more preferably 3 to 18, still more preferably 3 to 16, and yet more preferably 3 to 12.

Examples of the aromatic hydrocarbon group in L^{a21} include di- to hexa-valent aromatic hydrocarbon groups such as an arylene group, an arenetriyl group, an arenetetrayl group, an arenepentayl group and an arenehexayl group.

Specific examples thereof include aromatic hydrocarbon groups such as a phenylene group, a naphthylene group, an anthrylene group, a biphenylene group, a phenanthrylene group, a benzenetriyl group, a naphthalenetriyl group, a anthracenetriyl group, a biphenylenetriyl group, a phenanthrenetriyl group, a benzenetetrayl group, a naphthalenetetrayl group, an anthracenetetrayl group, a biphenylenetetrayl group and a phenanthrenetetrayl group.

Examples thereof also include groups in which one or more hydrogen atoms of the above-mentioned groups are substituted with a bonding site.

The number of carbon atoms of the aromatic hydrocarbon group is preferably 6 to 20, more preferably 6 to 18, still more preferably 6 to 14, and yet more preferably 6 to 10.

Examples of the group obtained by combining two or more groups include a group obtained by combining an alicyclic hydrocarbon group with a chain hydrocarbon group, a group obtained by combining an aromatic hydrocarbon group with a chain hydrocarbon group, a group obtained by combining an alicyclic hydrocarbon group with an aromatic hydrocarbon group, and a group obtained by combining an alicyclic hydrocarbon group, a chain hydrocarbon group and an aromatic hydrocarbon group. In combination, two or more of alicyclic hydrocarbon groups, aromatic hydrocarbon groups and chain hydrocarbon groups may be respectively combined. Any group may be bonded to A^{a21} and L^{a22}.

Examples of the group in which —CH₂— included in the hydrocarbon group is replaced by —O—, —S—, —SO₂— or —CO— include a hydroxy group (a group in which —CH₂— included in methyl group is replaced by —O—), a carboxy group (a group in which —CH₂—CH₂— included in ethyl group is replaced by —O— CO—), a thiol group (a group in which —CH₂— included in methyl group is replaced by —S—), an alkoxy group (a group in which —CH₂— at any position included in alkyl group is replaced by —O—), an alkylthio group (a group in which —CH₂—at any position included in alkyl group is replaced by —S—), an alkoxycarbonyl group (a group in which —CH₂—CH₂— at any position included in the alkyl group is replaced by —O—CO—), an alkylsulfonyl group (a group in which —CH₂— at any position included in the alkyl group is replaced by —SO₂—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an oxy group (a group in which —CH₂— included in the methylene group is replaced by —O—), a carbonyl group (a group in which —CH₂— included in the methylene group is replaced by —CO—), a thio group (a group in which —CH₂—included in methylene group is replaced by —S—), a sulfonyl group (a group in which —CH₂— included in the methylene group is replaced by —SO₂—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), an alkanediylsulfonyl group (a group in which —CH₂—at any position included in the alkanediyl group is replaced by —SO₂—), an alkanediylthio group (a group in which —CH₂— at any position included in the alkanediyl group is replaced by —S—), a cycloalkoxy group, a cycloalkylalkoxy group, an alkoxycarbonyloxy group, an aromatic hydrocarbon group-carbonyloxy group, an aromatic hydrocarbon group-carbonyl group, an aromatic hydrocarbon group-oxy group, and groups obtained by combining two or more of these group. Examples thereof also include groups in which one or more hydrogen atoms of the above-mentioned groups are substituted with a bonding site. Examples of these replaced groups include the same groups as mentioned herein.

Examples of the group in which —CH₂— included in the alicyclic hydrocarbon group is replaced by —O—, —S—, —CO— or —SO₂— include the same groups as mentioned herein.

Examples of the group in which —CH₂— included in the combined group is replaced by —O—, —S—, —CO— or —SO₂— also include the following groups. The bonding site can be any position.

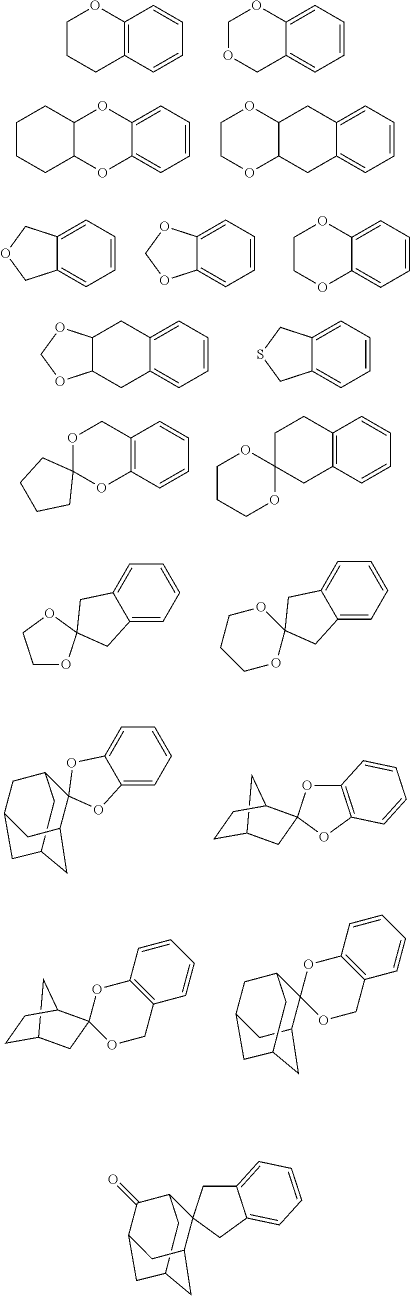

The hydrocarbon group in L^{a21} may have one or a plurality of substituents. Examples of the substituent include a halogen atom, a haloalkyl group having 1 to 12 carbon atoms, an alkyl group having 1 to 16 carbon atoms (—CH₂— included in the alkyl group may be replaced by —O— or —CO—), an acryloyloxy group or a methacryloyloxy group.

Examples of the halogen atom include a fluorine atom, a chlorine atom, a bromine atom, an iodine atom and the like.

The hydrocarbon group in L^{a21} can substantially have a substituent such as a haloalkyl group by having a halogen atom as the substituent. Examples of the haloalkyl group include an alkyl fluoride group, an alkyl chloride group, an alkyl bromide group, an alkyl iodide group, for example, a chloromethyl group, a bromomethyl group, an iodomethyl group, a fluoromethyl group, a difluoromethyl group, a trifluoromethyl group, a perfluoroethyl group, a perfluoropropyl group, a perfluorobutyl group and the like. The number of carbon atoms of the haloalkyl group is preferably 1 to 8, more preferably 1 to 6, still more preferably 1 to 4, and yet more preferably 1 to 3.

The hydrocarbon group as for L^{a21} can substantially have a substituent such as an alkyl group by including a branched structure in L^{a2}. Examples of the alkyl group include a methyl group, an ethyl group, an n-propyl group, an isopropyl group, an n-butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group. The number of carbon atoms of the alkyl group is preferably 1 to 12, more preferably 1 to 10, still more preferably 1 to 8, yet more preferably 1 to 6, further preferably 1 to 4, and still further preferably 1 to 3.

L^{a21} can substantially have a substituent such as a hydroxy group, a carboxy group, an alkoxy group, an alkoxycarbonyl group, an alkylcarbonyl group, an alkylcarbonyloxy group, an alkoxyalkyl group, an alkoxyalkoxy group, an acryloyloxy group or a methacryloyloxy group, by the group in which —CH₂— included in the hydrocarbon group is replaced by —O— or —CO— as for L^{a21}. The number of carbon atoms of the alkoxy group is preferably 1 to 12, more preferably 1 to 6, still more preferably 1 to 4, and yet more preferably 1 to 3. The number of carbon atoms of the alkoxycarbonyl group, the alkylcarbonyl group and the alkylcarbonyloxy group is preferably 2 to 12, more preferably 2 to 6, still more preferably 2 to 4, and yet more preferably 2 to 3. The number of carbon atoms of the alkoxyalkyl group and the alkoxyalkoxy group is preferably 2 to 12, more preferably 2 to 8, still more preferably 2 to 6, and yet more preferably 2 to 4.

Examples of the above group include those which are the same as mentioned herein.

The substituent which may be possessed by the hydrocarbon group in L^{a21} is preferably a halogen atom, a haloalkyl group having 1 to 8 carbon atoms or an alkyl group having 1 to 12 carbon atoms (—CH₂— included in the alkyl group may be replaced by —O— or —CO—), more preferably a halogen atom, a haloalkyl group having 1 to 6 carbon atoms or an alkyl group having 1 to 10 carbon atoms (—CH₂— included in the alkyl group may be replaced by —O— or —CO—), still more preferably a halogen atom, a haloalkyl group having 1 to 4 carbon atoms, an alkyl group having 1 to 4 carbon atoms, a hydroxy group, alkoxy group having 1 to 4 carbon atoms or an alkoxyalkoxy group having 2 to 8 carbon atoms, yet more preferably a fluorine atom, an iodine atom, a trifluoromethyl group, a methyl group, a hydroxy group, a methoxy group, an ethoxy group, an ethoxyethoxy group or an ethoxymethoxy group, and still preferably a fluorine atom, an iodine atom, a trifluoromethyl group, a methyl group, a hydroxy group, a methoxy group or an ethoxyethoxy group.

When the alkanediyl group is replaced by —O— or —CO— as for L^{a21}, for example, it is also preferably *-L^{a23}-X^{a21}— (L^{a23} represents an alkanediyl group having 1 to 8 carbon atoms, X^{a21} represents —O—, —O—CO—, —CO—O— or —O—CO—O—, and * represents a bonding site to A^{a21}).

L^{a21} is preferably a single bond, a chain hydrocarbon group having 1 to 12 carbon atoms which may have a substituent (in which —CH₂— included in the chain hydrocarbon group may be replaced by —O— or —CO—), a cyclic hydrocarbon group having 3 to 20 carbon atoms which may have a substituent (in which —CH₂— included in the cyclic hydrocarbon group may be replaced by —O—, —S—, —CO— or —SO₂—), or groups obtained by combining a chain hydrocarbon group having 1 to 8 carbon atoms which may have a substituent with a cyclic hydrocarbon group having 3 to 20 carbon atoms which may have a substituent (in which —CH₂— included in the chain hydrocarbon group may be replaced by —O— or —CO—, and —CH₂— included in the cyclic hydrocarbon group may be replaced by —O—, —S—, —SO₂— or —CO—), and more preferably a chain hydrocarbon group having 1 to 10 carbon atoms which may have a substituent (in which —CH₂— included in the chain hydrocarbon group may be replaced by —O— or —CO—), a cyclic hydrocarbon group having 3 to 18 carbon atoms which may have a substituent (in which —CH₂— included in the cyclic hydrocarbon group may be replaced by —O—, —S—, —CO— or —SO₂—), or groups obtained by combining a chain hydrocarbon group having 1 to 6 carbon atoms which may have a substituent with a cyclic hydrocarbon group having 3 to 18 carbon atoms which may have a substituent (in which —CH₂— included in the chain hydrocarbon group may be replaced by —O— or —CO—, and —CH₂— included in the cyclic hydrocarbon group may be replaced by —O—, —S—, —SO₂— or —CO—).

Examples of the chain hydrocarbon group having 1 to 12 carbon atoms as for L^{a22} include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and branched alkanediyl groups such as an ethane-1,1-diyl group, a propane-1,1-diyl group, a propane-1,2-diyl group, a propane-2,2-diyl group, a 1-methylpropane-1,3-diyl group, a 2-methylpropane-1,3-diyl group, a 2-methylpropane-1,2-diyl group, a 1-dimethylpropane-1,3-diyl group, a pentane-2,4-diyl group, a pentane-1,4-diyl group and a 2-methylbutane-1,4-diyl group. The number of carbon atoms of the chain hydrocarbon group is preferably 1 to 10, more preferably 1 to 8, still more preferably 1 to 6, and yet more preferably 1 to 4.

Examples of the group in which —CH₂— included in the chain hydrocarbon group is replaced by —O— or —CO— include the same groups as mentioned herein.

The number of fluorine atoms possessed by L^{a22} may be either 1, or 2 or more.

na2 is preferably an integer of 1 to 4, and more preferably an integer of 1 to 3.

Examples of the structural unit (a2) when L^{a21} is a single bond or a chain hydrocarbon group include the following structural units. Of the following structural units, the structural unit in which the methyl group corresponding to R^{a2} is substituted with a hydrogen atom or the like is preferable structural unit of the structural unit (a2), similarly to the following structural units.

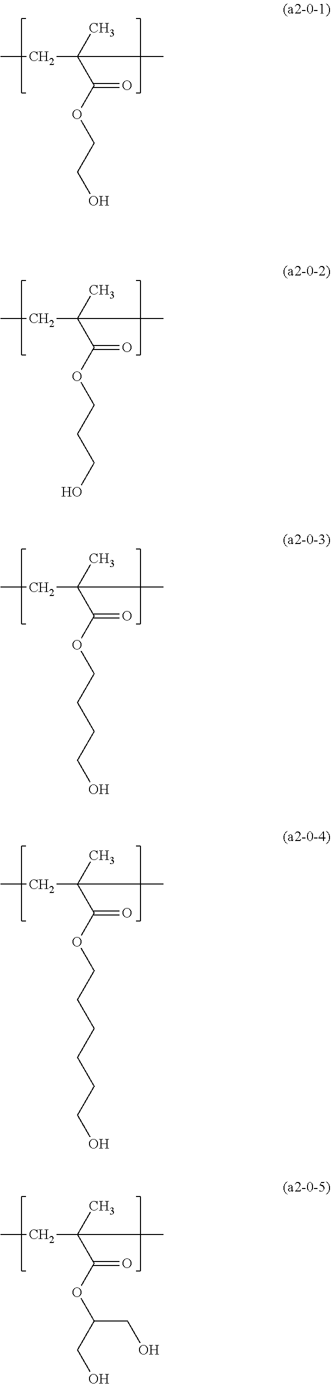

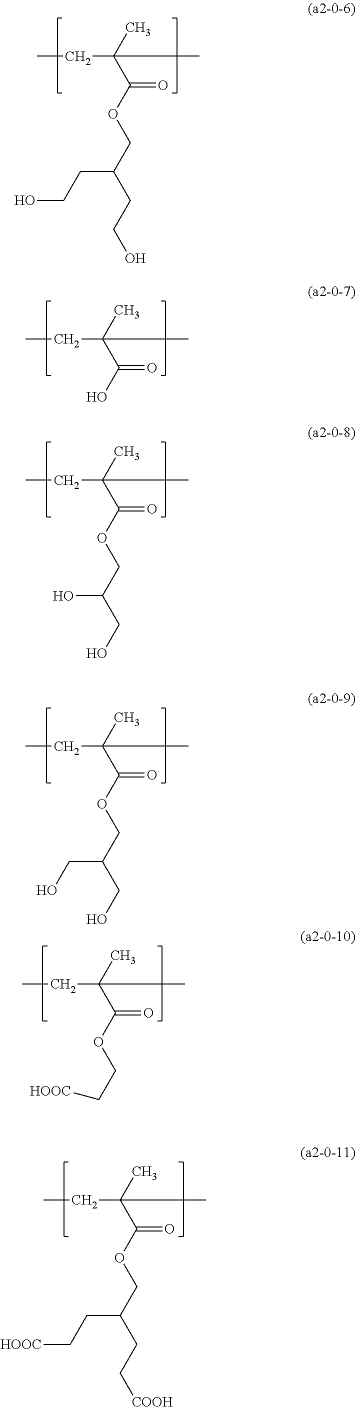

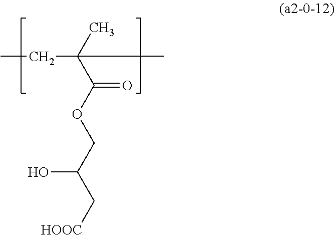

When a resist pattern is produced from the resist composition of the present invention, in the case of using, as an exposure source, high energy rays such as KrF excimer laser (248 nm), electron beam or extreme ultraviolet light (EUV), a structural unit (a2) having a phenolic hydroxy group is preferably used, and the below-mentioned structural unit (a2-A) is more preferably used, as the structural unit (a2). When using ArF excimer laser (193 nm) or the like, a structural unit (a2) having an alcoholic hydroxy group is preferably used, and the below-mentioned structural unit (a2-1) is more preferably used, as the structural unit (a2). The structural unit (a2) may be included alone, or two or more structural units may be included.

In the structural unit (a2), when L^{a21} is a cyclic hydrocarbon group, examples of the structural unit having a phenolic hydroxy group include a structural unit represented by formula (a2-A) (hereinafter sometimes referred to as “structural unit (a2-A)”):

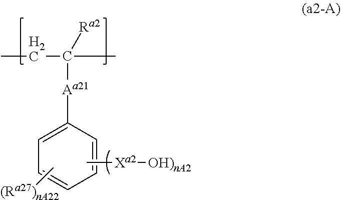

wherein, in formula (a2-A),
R^{a2} represents a hydrogen atom, a halogen atom, or an alkyl group having 1 to 6 carbon atoms which may have a halogen atom,
A^{a21} represents a single bond or an alkanediyl group having 1 to 12 carbon atoms, and —CH₂— included in the alkanediyl group may be replaced by —O—, —CO— or —NR^{a28}—,
R^{a28} represents a hydrogen atom or an alkyl group having 1 to 4 carbon atoms,
X^{a2} represents a single bond or —CO—,
R^{a27} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
nA2 represents an integer of 1 to 5, and when nA2 is 2 or more, a plurality of X^{a2} may be the same or different from each other, and
nA22 represents an integer of 0 to 4, and when nA22 is 2 or more, a plurality of R^{a27} may be the same or different from each other.

Examples of R^{a2}, A^{a21} and R^{a28} include the same groups as mentioned in formula (a2), respectively.

Examples of the halogen atom and the alkyl group which may have a halogen atom as for R^{a27} include the same groups as mentioned as for R^{a2} or the substituent of L^{a21} in formula (a2), respectively.

Examples of the alkoxy group as for R^{a27} include a methoxy group, an ethoxy group, a propoxy group, an isopropoxy group, a butoxy group, a sec-butoxy group and a tert-butoxy group. The number of carbon atoms of the alkoxy group is preferably 1 to 4, and more preferably 1 to 3. The alkoxy group is preferably a methoxy group or an ethoxy group, and more preferably a methoxy group.

Examples of the alkoxyalkyl group as for R^{a27} include a methoxymethyl group, an ethoxyethyl group, a propoxymethyl group, an isopropoxymethyl group, a butoxymethyl group, a sec-butoxymethyl group and a tert-butoxymethyl group. The number of carbon atoms of the alkoxyalkyl group is preferably 2 to 8, and more preferably 2 to 4. The alkoxyalkyl group is preferably a methoxymethyl group or an ethoxyethyl group, and more preferably a methoxymethyl group.

Examples of the alkoxyalkoxy group as for R^{a27} include a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The number of carbon atoms of the alkoxyalkoxy group is preferably 2 to 8, and more preferably 2 to 4. The alkoxyalkoxy group is preferably a methoxyethoxy group or an ethoxyethoxy group.

Examples of the alkylcarbonyl group as for R^{a27} include an acetyl group, a propionyl group and a butyryl group. The number of carbon atoms of the alkylcarbonyl group is preferably 2 to 4, and more preferably 2 to 3. The alkylcarbonyl group is preferably an acetyl group.

Examples of the alkylcarbonyloxy group as for R^{a27} include an acetyloxy group, a propionyloxy group and a butyryloxy group. The number of carbon atoms of the alkylcarbonyloxy group is preferably 2 to 4, and more preferably 2 to 3. The alkylcarbonyloxy group is preferably an acetyloxy group.

R^{a27} is preferably a halogen atom, a hydroxy group, a carboxy group, an alkyl group having 1 to 4 carbon atoms which may have a halogen atom, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group or an ethoxyethoxy group.

X^{a2} is preferably a single bond.

nA22 is preferably 0, 1, 2 or 3, more preferably 0, 1 or 2, and still more preferably 0 or 1.

nA2 is preferably 1, 2, 3 or 4, more preferably 1, 2 or 3, and still more preferably 1 or 2.

At least one hydroxy group is preferably bonded at the meta-position or the para-position of the benzene ring. When two or more hydroxy groups are bonded to the benzene ring, two hydroxy groups are preferably bonded at the meta-position and the para-position.

Examples of the structural unit (a2-A) include structural units derived from the monomers mentioned in JP 2010-204634 A and JP 2012-12577 A.

Examples of the structural unit (a2-A) include structural units represented by formula (a2-2-1) to formula (a2-2-32) and a structural unit in which a methyl group corresponding to R^{a2} in the structural unit (a2-A) is substituted with a hydrogen atom, a halogen atom, a haloalkyl group or other alkyl groups in structural units represented by formula (a2-2-1) to formula (a2-2-32). The structural unit (a2-A) is preferably structural units represented by formula (a2-2-1) to formula (a2-2-4), a structural unit represented by formula (a2-2-6), a structural unit represented by formula (a2-2-8), and structural units represented by formula (a2-2-12) to formula (a2-2-18), and a structural unit in which a methyl group corresponding to R^{a2} in the structural unit (a2-A) is substituted with a hydrogen atom in structural units represented by formula (a2-2-1) to formula (a2-2-4), a structural unit represented by formula (a2-2-6), a structural unit represented by formula (a2-2-8) and structural units represented by formula (a2-2-12) to formula (a2-2-18), more preferably a structural unit represented by formula (a2-2-3), a structural unit represented by formula (a2-2-4), a structural unit represented by formula (a2-2-8), structural units represented by formula (a2-2-12) to formula (a2-2-14) and a structural unit represented by formula (a2-2-18), and a structural unit in which a methyl group corresponding to R^{a2} in the structural unit (a2-A) is substituted with a hydrogen atom in a structural unit represented by formula (a2-2-3), a structural unit represented by formula (a2-2-4), a structural unit represented by formula (a2-2-8), structural units represented by formula (a2-2-12) to formula (a2-2-14) and a structural unit represented by formula (a2-2-18), and still more preferably a structural unit represented by formula (a2-2-3), a structural unit represented by formula (a2-2-4) and a structural unit represented by formula (a2-2-8), and a structural unit in which a methyl group corresponding to R^{a2} in the structural unit (a2-A) is substituted with a hydrogen atom in a structural unit represented by formula (a2-2-3), a structural unit represented by formula (a2-2-4) and a structural unit represented by formula (a2-2-8).

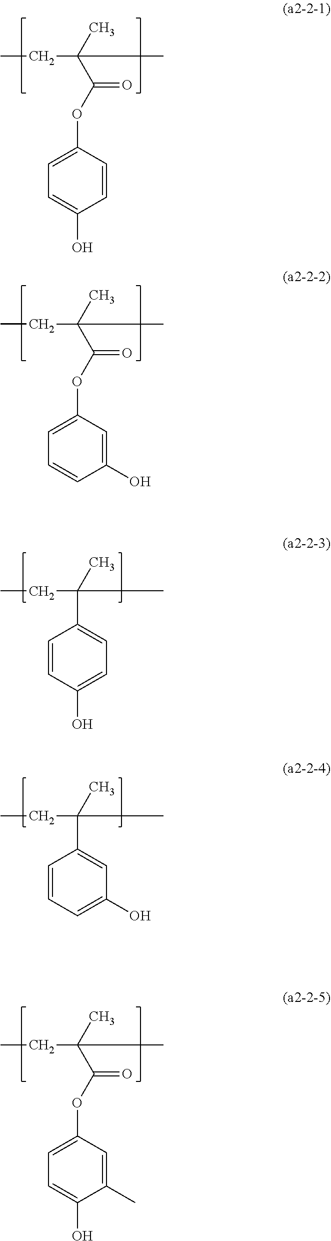

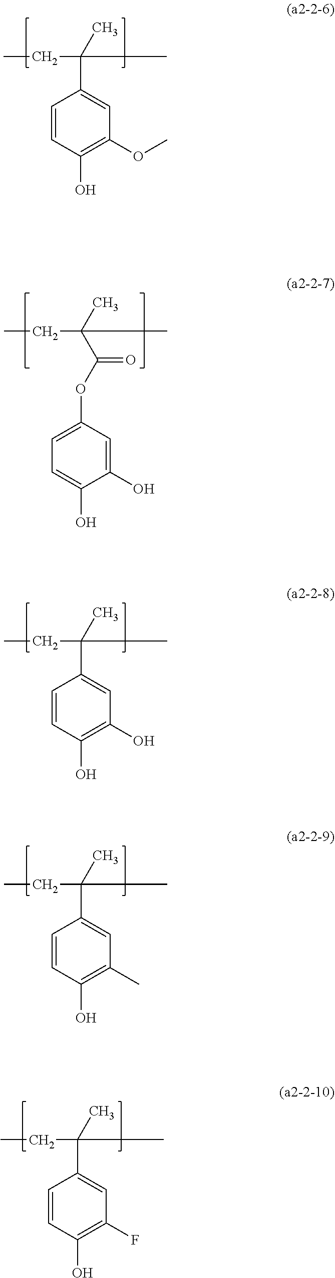

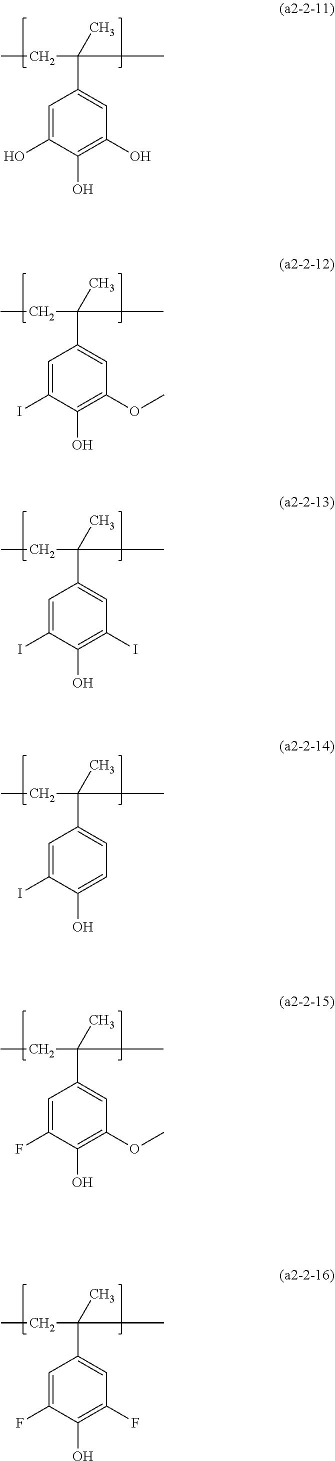

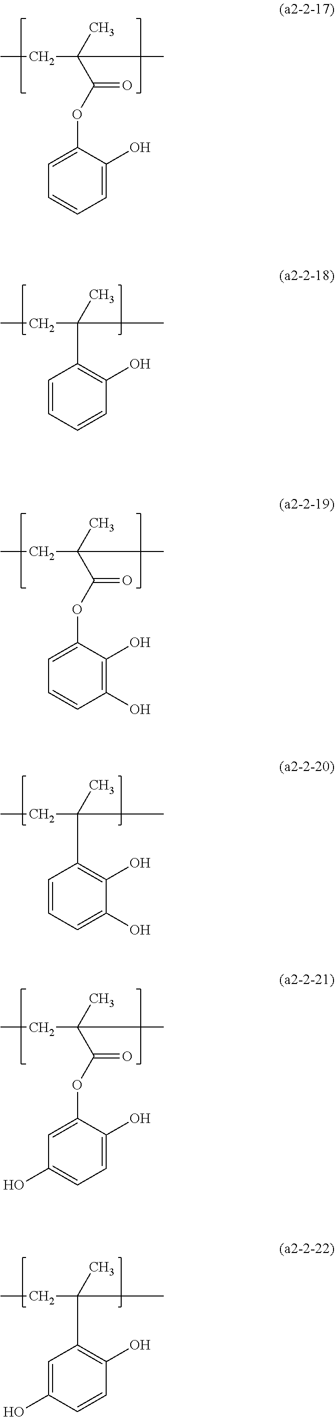

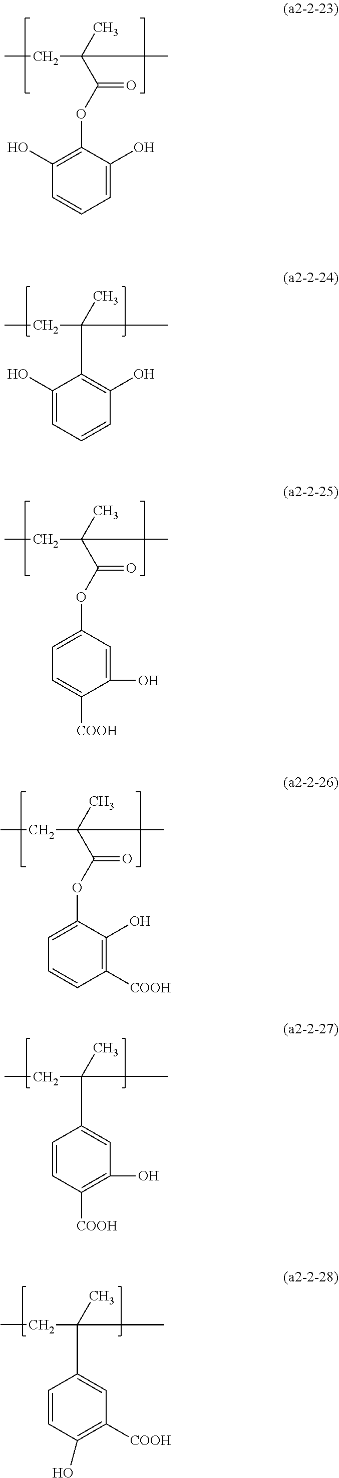

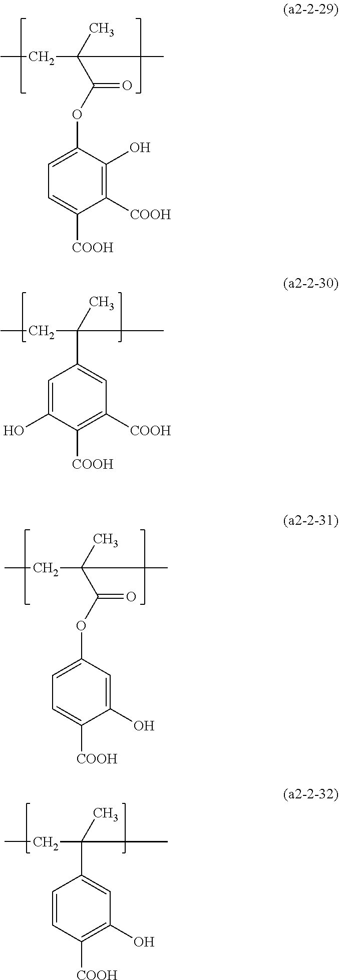

When the structural unit (a2-A) is included in the resin (A) or the like, the content of the structural unit (a2-A) is preferably 5 mol % or more, more preferably 10 mol % or more, still more preferably 15 mol % or more, and yet more preferably 20 mol % or more, based on all structural units. The content is also preferably 80 mol % or less, more preferably 70 mol % or less, still more preferably 65 mol % or less, yet more preferably 60 mol % or less, further preferably 50 mol % or less, still further preferably 45 mol % or less, and yet further preferably 40 mol % or less, based on all structural units. Specifically, the content is preferably 5 to 80 mol %, more preferably 10 to 70 mol %, still more preferably 15 to 65 mol %, and yet more preferably 20 to 65 mol %, based on all structural units.

The structural unit (a2-A) can be included in the resin (A) or the like by polymerizing, for example, with a structural unit (a1-4) and treating with an acid such as p-toluenesulfonic acid. The structural unit (a2-A) can also be included in the resin (A) or the like by polymerizing with acetoxystyrene and treating with an alkali such as tetramethylammonium hydroxide.

In the structural unit (a2), when L^{a21} is a cyclic hydrocarbon group, examples of the structural unit having an alcoholic hydroxy group or the like include a structural unit represented by formula (a2-B) (hereinafter sometimes referred to as “structural unit (a2-B)”) and a structural unit represented by formula (a2-C) (hereinafter sometimes referred to as “structural unit (a2-C)”):

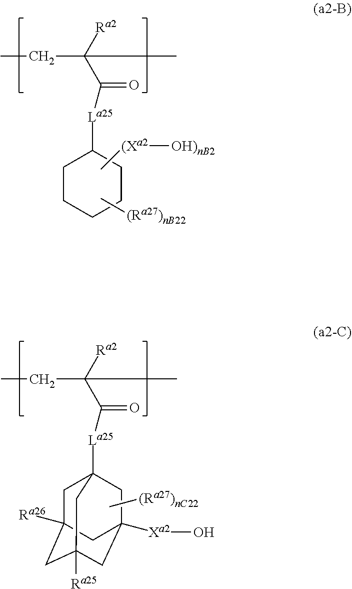

wherein, in formula (a2-B) and formula (a2-C),
R^{a2} is the same as defined in formula (a2),
R^{a27} is the same as defined in formula (a2-A),
L^{a25} represents —O— or *—O—(CH₂)ₖ₂—CO—O—,
k2 represents an integer of 1 to 7, and * represents a bonding site to —CO—,
X^{a2} represents a single bond or —CO—,
R^{a25} and R^{a26} each independently represent a hydrogen atom, a methyl group or a hydroxy group,
nB2 represents an integer of 1 to 5, and when nB2 is 2 or more, a plurality of X^{a2} may be the same or different from each other,
nB22 represents an integer of 0 to 8, and when nB22 is 2 or more, a plurality of R^{a27} may be the same or different from each other, and
nC22 represents an integer of 0 to 10, and when nC22 is 2 or more, a plurality of R^{a27} may be the same or different from each other.

L^{a25} is preferably —O—, —O—(CH₂)_{f1}—CO—O— (f1 represents an integer of 1 to 4), and more preferably —O—.

R^{a2} is preferably a methyl group.

X^{a2} is preferably a single bond.

R^{a25} is preferably a hydrogen atom.

R^{a26} is preferably a hydrogen atom or a hydroxy group.

R^{a27} is preferably a halogen atom, a hydroxy group, a carboxy group, an alkyl group having 1 to 4 carbon atoms which may have a halogen atom, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, still more preferably a fluorine atom, an iodine atom, a hydroxy group, a methyl group, a methoxy group or an ethoxyethoxy group.

nB2 is preferably 1, 2, 3 or 4, more preferably 1, 2 or 3, and still more preferably 1 or 2.

nB22 is preferably an integer of 0 to 3, more preferably 0, 1 or 2, and still more preferably 0 or 1.

nC22 is preferably an integer of 0 to 6, more preferably an integer of 0 to 3, and still more preferably 0 or 1.

Examples of the structural unit (a2-B) and the structural unit (a2-C) include a structural unit derived from the monomer mentioned in JP 2010-204646 A, the following structural units, and structural units in which the methyl group or the hydrogen atom corresponding to R^{a2} in the following structural units is substituted with a hydrogen atom, a halogen atom, a haloalkyl group, or other alkyl groups. Of these, a structural unit represented by any one of formula (a2-B-1) to formula (a2-B-5) and formula (a2-C-1) to formula (a2-C-9) is preferable.

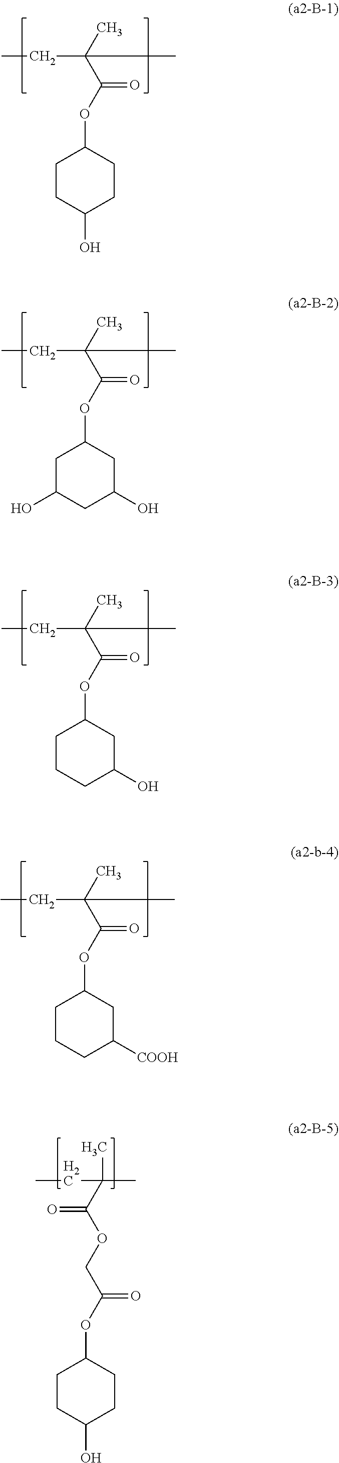

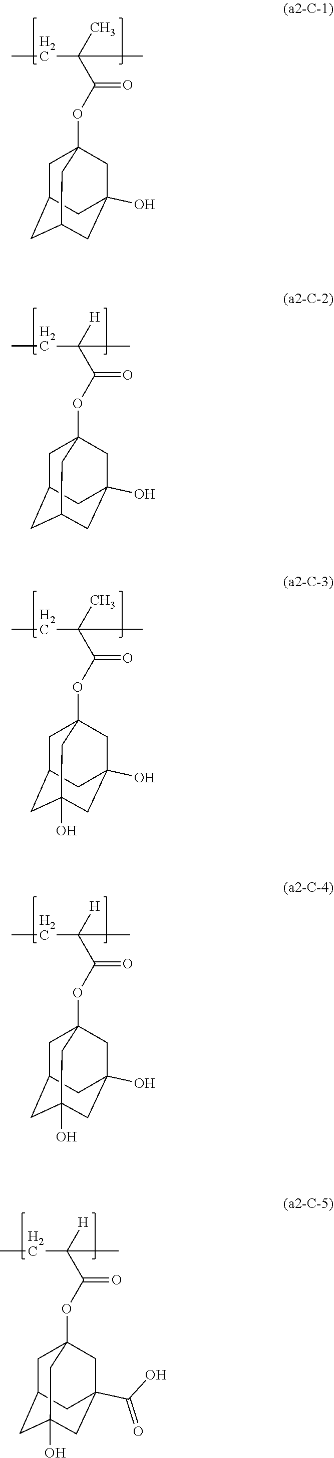

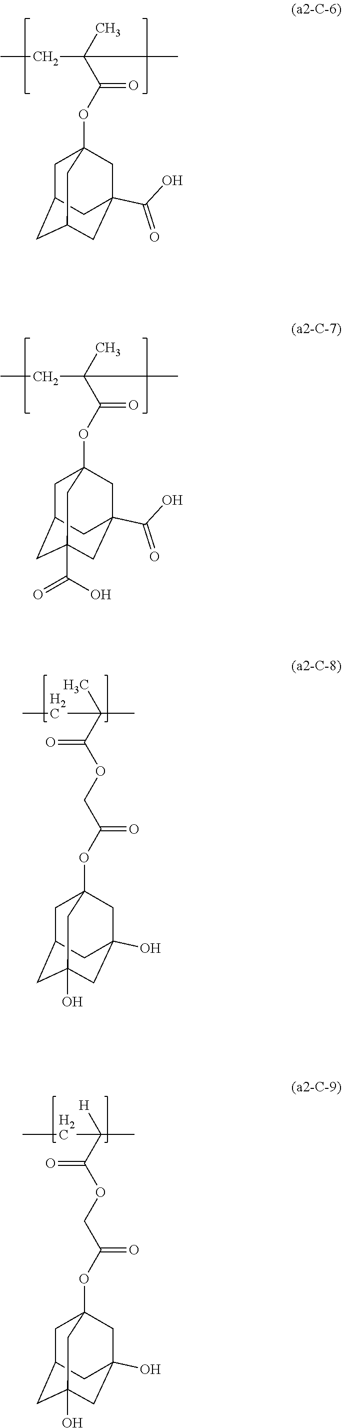

When the resin (A) or the like includes a structural unit (a2-B) or a structural unit (a2-C), the content is usually 1 mol % or more, and preferably 2 mol % or more, based on all structural units of the resin (A) or the like. The content is also usually 45 mol % or less, preferably 40 mol % or less, more preferably 35 mol % or less, still more preferably 20 mol % or less, and yet more preferably 10 mol % or less, based on all structural units of the resin (A) or the like. Specifically, the content is usually 1 to 45 mol %, preferably 1 to 40 mol %, more preferably 1 to 35 mol %, still more preferably 1 to 20 mol %, and yet more preferably 1 to 10 mol %, based on all structural units of the resin (A) or the like.

In the structural unit (a2), when L^{a21} is a cyclic hydrocarbon group, examples of the structural unit having an alcoholic hydroxy group or the like include a structural unit represented by formula (a2-D) (hereinafter sometimes referred to as “structural unit (a2-D)”):

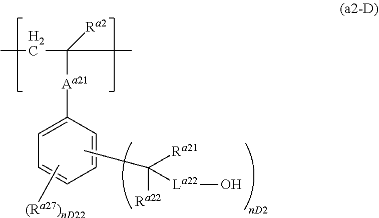

wherein, in formula (a2-D),
R^{a2} and A^{a21} are respectively the same as defined in formula (a2),
R^{a27} is the same as defined in formula (a2-A),
R^{a21} and R^{a22} each independently represent an alkyl fluoride group having 1 to 4 carbon atoms,
L^{a24} represents a single bond or an alkanediyl group having 1 to 3 carbon atoms, and the alkanediyl group may be substituted with a fluorine atom,
nD2 represents an integer of 1 to 5, and when nD2 is 2 or more, a plurality of groups in parentheses may be the same or different from each other, and
nD22 represents an integer of 0 to 4, and when nD22 is 2 or more, a plurality of R^{a27} may be the same or different from each other,
in which 1≤nD2+≤nD22≤5.

Examples of the alkyl fluoride group as for R^{a21} and R^{a22} each independently include a trifluoromethyl group, a 2,2,2-trifluoroethyl group, a 3,3,3-trifluoropropyl group, a 4,4,4-trifluorobutyl group and the like. R^{a21} and R^{a22} are preferably a trifluoromethyl group.

Examples of the alkanediyl group as for L^{a24} include a methylene group, an ethane-1,1-diyl group, a propane-1,1-diyl group, a propane-2,2-diyl group and the like. L^{a24} is preferably a single bond or a methylene group.

R^{a27} is preferably a halogen atom, a hydroxy group, a carboxy group, an alkyl group having 1 to 4 carbon atoms which may have a halogen atom, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably, a fluorine atom, an iodine atom, a hydroxy group, a methyl group, a methoxy group or an ethoxyethoxy group.

nD2 is preferably 1, 2, 3 or 4, more preferably 1, 2 or 3, still more preferably 1 or 2, and yet more preferably 1. Preferably, nD2 is 1, and a group in parentheses is bonded at the para-position.

nD22 is preferably an integer of 0 to 3, more preferably an integer of 0 to 2, still more preferably 0 or 1, and yet more preferably 0.

The structural unit (a2-D) is more preferably a structural unit represented by the following formula (a2-D1) (hereinafter sometimes referred to as “structural unit (a2-D1)”):

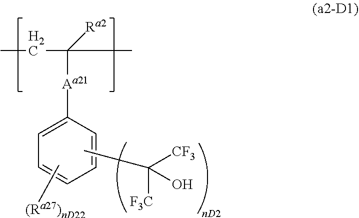

wherein, in formula (a2-D1),

R^{a2}, A^{a21}, R^{a27}, nD2 and nD22 are respectively the same as defined in formula (a2-D).

In formula (a2-D1), R^{a2} is preferably a hydrogen atom.

A^{a21} is preferably a single bond.

Examples of the structural unit (a2-D) include structural units mentioned below.

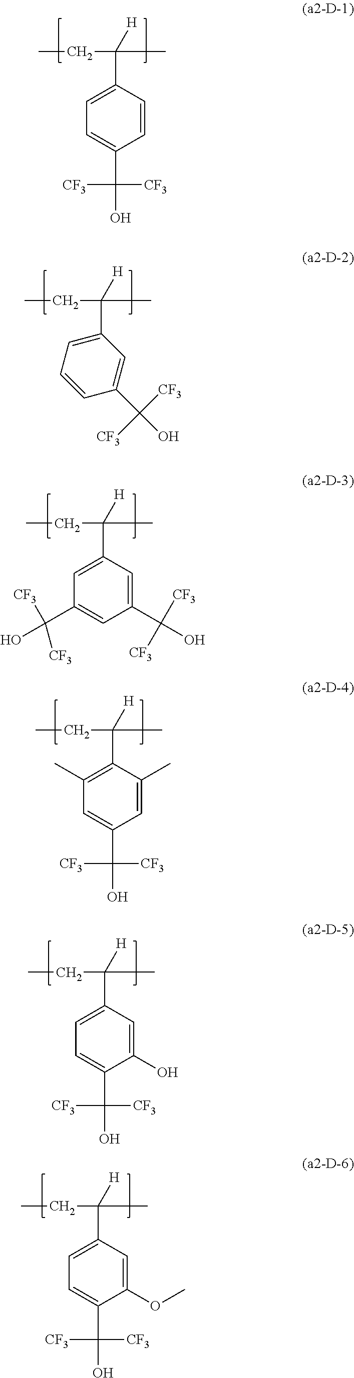

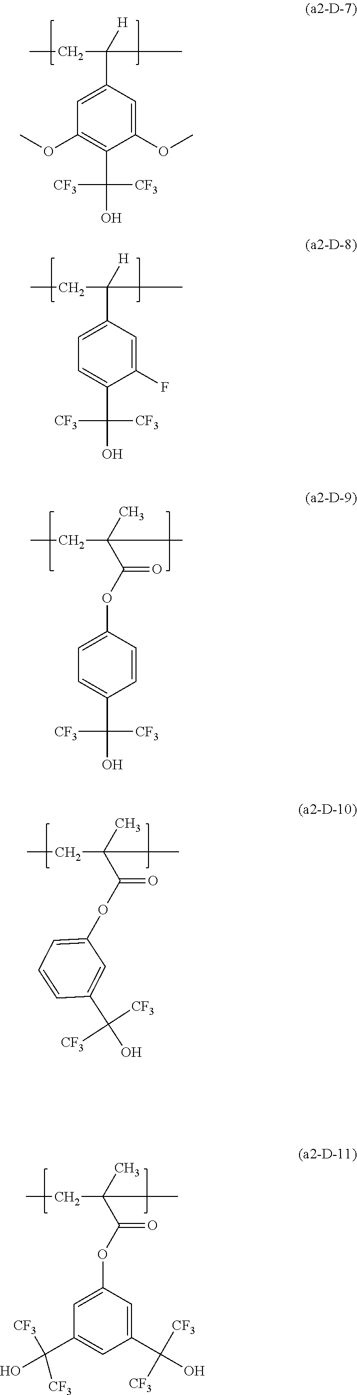

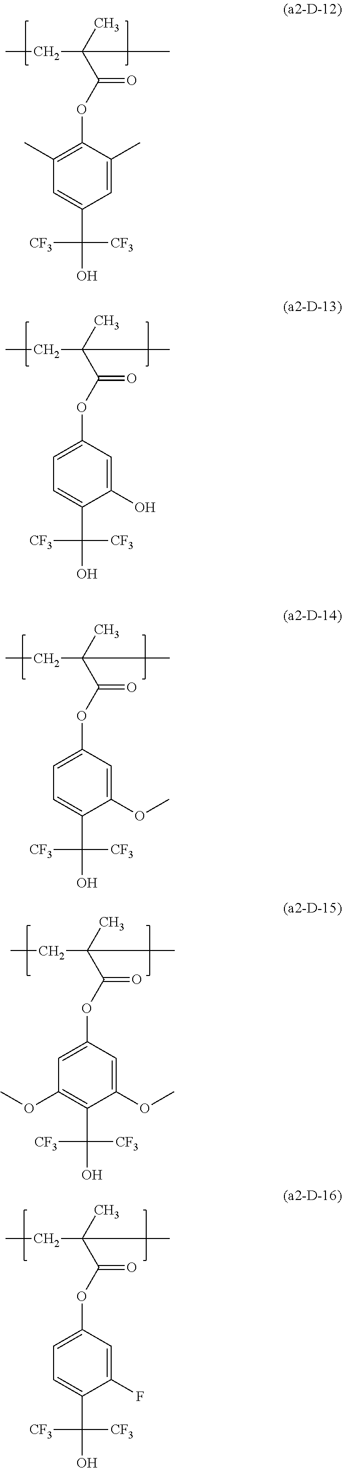

It is possible to exemplify structural units in which the hydrogen atom corresponding to R^{a2} is substituted with a methyl group or the like in structural units represented by formula (a2-D-1) to formula (a2-D-8), and structural units in which the methyl group corresponding to R^{a2} is substituted with a hydrogen atom or the like in structural units represented by formula (a2-D-9) to formula (a2-D-16) as specific examples of the structural unit (a2-D). Of these, structural units represented by formula (a2-D-1) to formula (a2-D-8) are preferable, structural units represented by formula (a2-D-1) to formula (a2-D-4) are more preferable, and a structural unit represented by formula (a2-D-1) is still more preferable.

When the resin (A) or the like includes the structural unit (a2-D), the content is preferably 3 mol % or more, more preferably 5 mol % or more, and still more preferably 10 mol % or more, based on all structural units of the resin (A) or the like. The content is also preferably 80 mol % or less, more preferably 75 mol % or less, still more preferably 70 mol % or less, and yet more preferably 65 mol % or less, based on all structural units of the resin (A) or the like. Specifically, the content is preferably 3 to 80 mol %, more preferably 5 to 75 mol %, still more preferably 10 to 70 mol %, and yet more preferably 10 to 65 mol %, based on all structural units of the resin (A) or the like.

<Structural Unit (a3)>

The lactone ring possessed by the structural unit (a3) may be a monocyclic ring such as a β-propiolactone ring, a γ-butyrolactone ring or a δ-valerolactone ring, or a fused ring of a monocyclic lactone ring and the other ring. Preferably, a γ-butyrolactone ring, an adamantanelactone ring or a bridged ring including a γ-butyrolactone ring structure (e.g., a structural unit represented by the following formula (a3-2)) is exemplified.

The structural unit (a3) is preferably a structural unit represented by formula (a3-1), formula (a3-2), formula (a3-3) or formula (a3-4). These structural units may be included alone, or two or more structural units may be included:

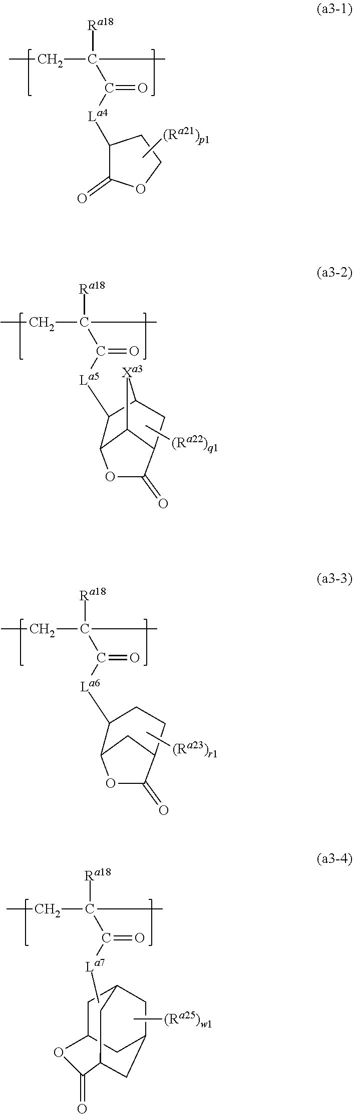

wherein, in formula (a3-1), formula (a3-2), formula (a3-3) and formula (a3-4),
L^{a4}, L^{a5} and L^{a6} each independently represent —O— or a group represented by *—O—(CH₂)ₖ₃—CO—O— (k3 represents an integer of 1 to 7),
L^{a7} represents —O—, *—O-L^{a8}-O—, *—O-L^{a8}-CO—O—, *—O— L^{a8}-CO—O-L^{a9}-CO—O— or *—O-L^{a8}-O—CO-L^{a9}-O—,
L^{a8} and L^{a9} each independently represent an alkanediyl group having 1 to 6 carbon atoms,
* represents a bonding site to a carbonyl group,
R^{a18}, R^{a19}, R^{a20} and R^{a24} each independently represent a hydrogen atom, a halogen atom, or an alkyl group having 1 to 6 carbon atoms which may have a halogen atom,
X^{a3} represents —CH₂— or an oxygen atom,
R^{a21} represents an aliphatic hydrocarbon group having 1 to 4 carbon atoms,
R^{a22}, R^{a23} and R^{a25} each independently represent a carboxy group, a cyano group, or an aliphatic hydrocarbon group having 1 to 4 carbon atoms,
p1 represents an integer of 0 to 5,
q1 represents an integer of 0 to 3,
r1 represents an integer of 0 to 3,
w1 represents an integer of 0 to 8, and
when p1, q1, r1 and/or w1 are 2 or more, a plurality of R^{a2}, R^{a22}, R^{a23} and/or R^{a25} may be the same or different from each other.

Examples of the aliphatic hydrocarbon group in R^{a21}, R^{a22}, R^{a23} and R^{a25} include alkyl groups such as a methyl group, an ethyl group, a propyl group, an isopropyl group, a butyl group, a sec-butyl group and a tert-butyl group.

Examples of the halogen atom in R^{a18}, R^{a19}, R^{a20} and R^{a24} include a fluorine atom, a chlorine atom, a bromine atom and an iodine atom.

Examples of the alkyl group in R^{a18}, R^{a19}, R^{a20} and R^{a24} include a methyl group, an ethyl group, a propyl group, an isopropyl group, a butyl group, a sec-butyl group, a tert-butyl group, a pentyl group and a hexyl group, and the alkyl group is preferably an alkyl group having 1 to 4 carbon atoms, and more preferably a methyl group or an ethyl group.

Examples of the alkyl group having a halogen atom in R^{a18}, R^{a19}, R^{a20} and R^{a24} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a trichloromethyl group, a tribromomethyl group, a triiodomethyl group and the like.

Examples of the alkanediyl group in L^{as} and L^{a9} include a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group and a 2-methylbutane-1,4-diyl group.

In formula (a3-1) to formula (a3-3), preferably, L^{a4} to L^{a6} are each independently —O— or a group in which k3 is an integer of 1 to 4 in *—O—(CH₂)ₖ₃—CO—O—, more preferably —O— and *—O—CH₂—CO—O—, and still more preferably an oxygen atom,
R^{a18} to R^{a21} are preferably a methyl group,
preferably, R^{a22} and R^{a23} are each independently a carboxy group, a cyano group or a methyl group, and
preferably, p1, q1 and r1 are each independently an integer of 0 to 2, and more preferably 0 or 1.

In formula (a3-4), R^{a24} is preferably a hydrogen atom or an alkyl group having 1 to 4 carbon atoms, more preferably a hydrogen atom, a methyl group or an ethyl group, and still more preferably a hydrogen atom or a methyl group,
R^{a25} is preferably a carboxy group, a cyano group or a methyl group,
L^{a7} is preferably —O— or *—O-L^{a8}-CO—O—, and more preferably —O—, —O—CH₂—CO—O— or —O—C₂H₄—CO—O—, and
w1 is preferably an integer of 0 to 2, and more preferably 0 or 1.

Particularly, formula (a3-4) is preferably formula (a3-4)′:

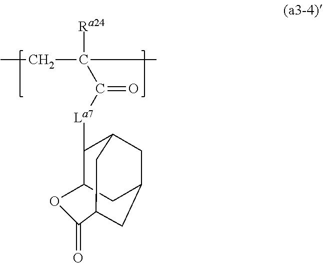

wherein R^{a24} and L^{a7} are the same as defined above.

Examples of the structural unit (a3) include structural units derived from the monomers mentioned in JP 2010-204646 A, the monomers mentioned in JP 2000-122294 A and the monomers mentioned in JP 2012-41274 A. The structural unit (a3) is preferably a structural unit represented by any one of formula (a3-1-1), formula (a3-1-2), formula (a3-2-1), formula (a3-2-2), formula (a3-3-1), formula (a3-3-2) and formula (a3-4-1) to formula (a3-4-12), and structural units in which methyl groups corresponding to R^{a18}, R^{a19}, R^{a20} and R^{a24} in formula (a3-1) to formula (a3-4) are substituted with hydrogen atoms in the above structural units.

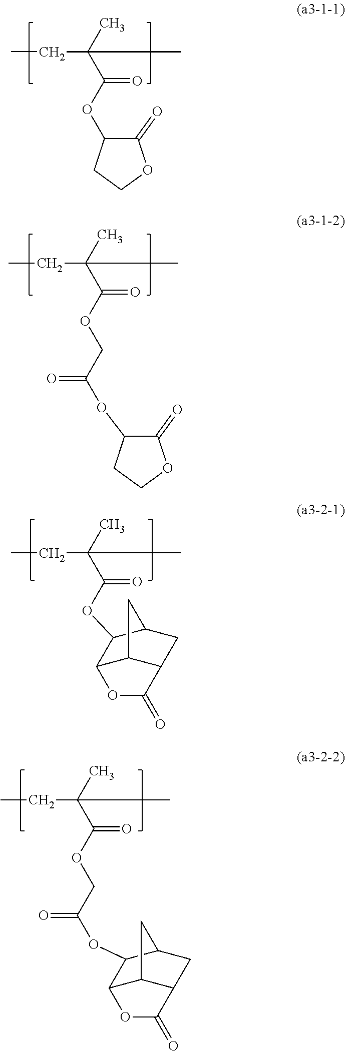

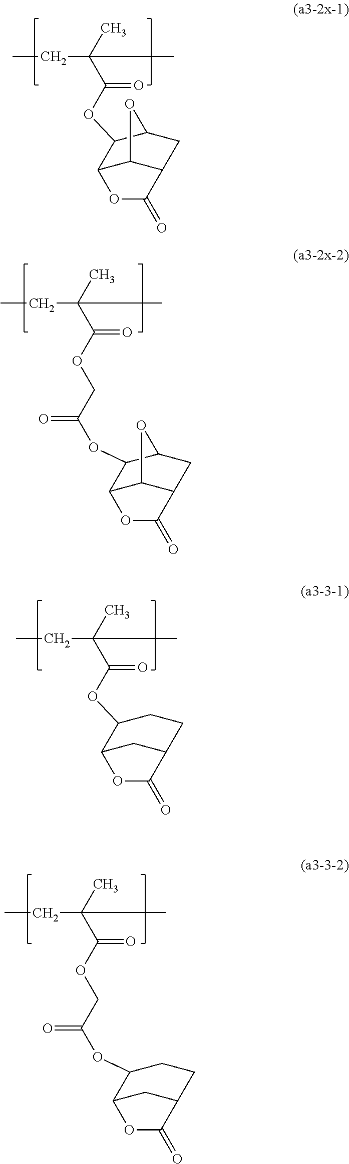

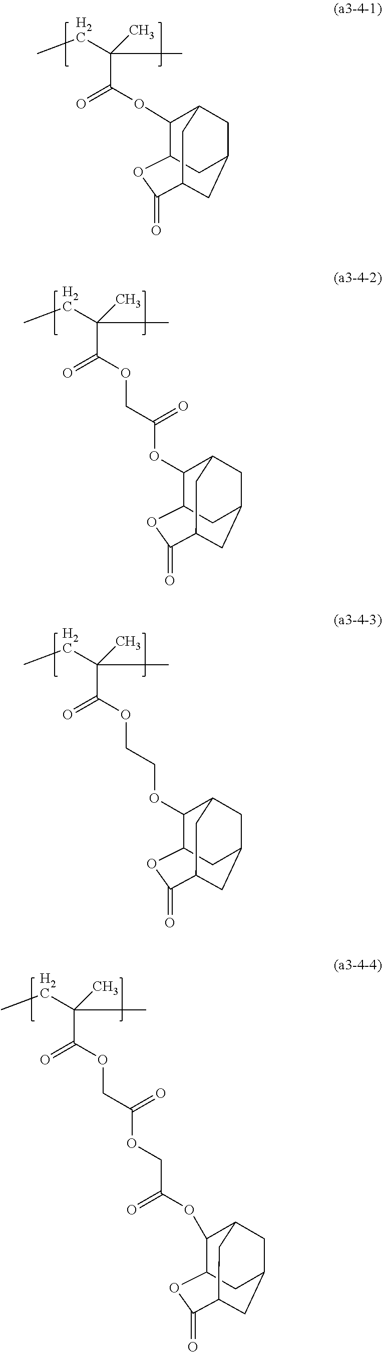

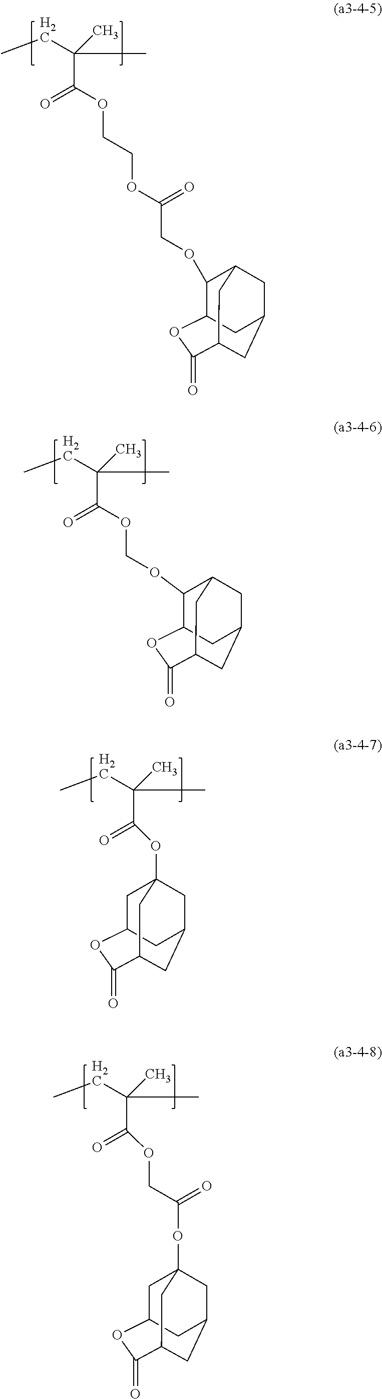

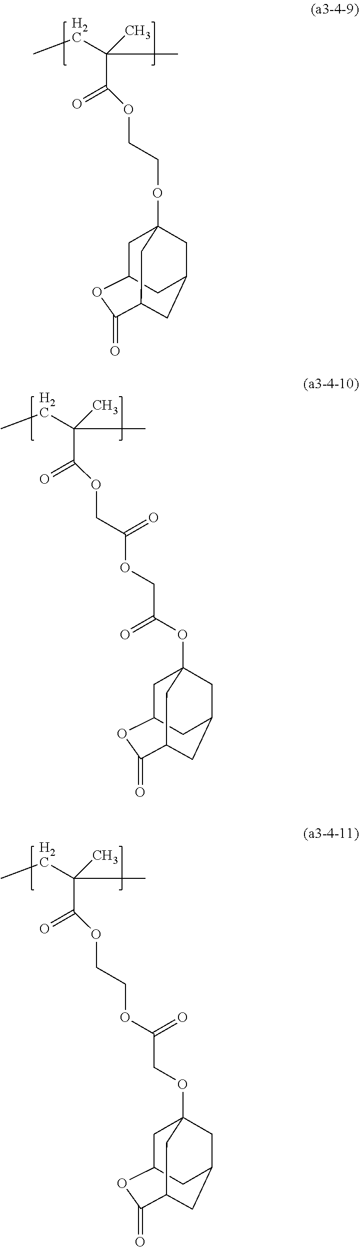

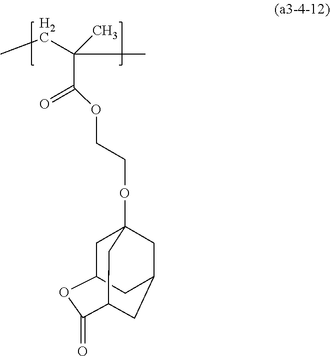

When the resin (A) or the like includes the structural unit (a3), the total content is usually 1 mol % or more, preferably 3 mol % or more, more preferably 5 mol % or more, and still more preferably 10 mol or more, based on all structural units of the resin (A) or the like. The total content is also usually 70 mol or less, preferably 65 mol or less, more preferably 60 mol or less, still more preferably 50 mol or less, yet more preferably 40 mol or less, further preferably 30 mol or less, still further preferably 25 mol % or less, and yet further preferably 20 mol % or less, based on all structural units of the resin (A) or the like. Specifically, the total content is usually 1 to 70 mol %, preferably 3 to 70 mol %, more preferably 3 to 65 mol %, still more preferably 5 to 65 mol %, yet more preferably to 60 mol %, and further preferably 10 to 60 mol %, based on all structural units of the resin (A) or the like.

Each content of the structural unit (a3-1), the structural unit (a3-2), the structural unit (a3-3) or the structural unit (a3-4) is preferably 1 mol % or more, more preferably 3 mol % or more, still more preferably 5 mol % or more, and yet more preferably 10 mol % or more, based on all structural units of the resin (A) or the like. The content is also preferably 60 mol % or less, more preferably 55 mol % or less, still more preferably 50 mol % or less, further preferably 40 mol % or less, still further preferably 30 mol % or less, yet further preferably 25 mol % or less, and yet still further preferably 20 mol % or less, based on all structural units of the resin (A) or the like. Specifically, the content is preferably 1 to 60 mol %, more preferably 3 to 60 mol %, still more preferably 3 to 55 mol %, yet more preferably 5 to 55 mol %, further preferably 5 to 50 mol %, and still further preferably 10 to 50 mol %, based on all structural units of the resin (A) or the like.

<Structural Unit (a4)>

Examples of the structural unit (a4) include the following structural unit:

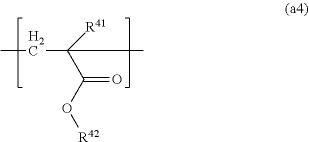

wherein, in formula (a4),
R⁴¹ represents a hydrogen atom or a methyl group, and
R⁴² represents a saturated hydrocarbon group having 1 to 24 carbon atoms which has a halogen atom, and —CH₂— included in the saturated hydrocarbon group may be replaced by —O— or —CO—.

Examples of the saturated hydrocarbon group represented by R⁴² include a linear or branched chain saturated hydrocarbon group and a monocyclic or polycyclic alicyclic saturated hydrocarbon group, and groups formed by combining these groups.

Examples of the chain saturated hydrocarbon group include linear alkyl groups such as a methyl group, an ethyl group, a propyl group, a butyl group, a pentyl group, a hexyl group, a heptyl group, an octyl group, a decyl group, a dodecyl group, a pentadecyl group, a hexadecyl group, a heptadecyl group and an octadecyl group; and branched alkyl groups such as an isopropyl group and an isobutyl group.

Examples of the monocyclic or polycyclic saturated alicyclic hydrocarbon group include monocyclic alicyclic saturated hydrocarbon groups which are monocyclic cycloalkyl groups such as a cyclopentyl group, a cyclohexyl group, a cycloheptyl group and a cyclooctyl group; and polycyclic alicyclic saturated hydrocarbon groups which are polycyclic cycloalkyl groups such as a decahydronaphthyl group, an adamantyl group, a norbornyl group and the following groups (* represents a bonding site).

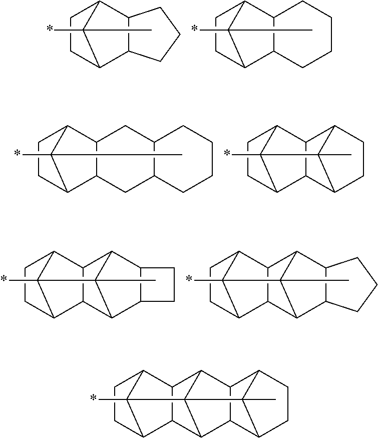

Examples of the groups formed by combination include groups formed by combining one or more alkyl groups or one or more alkanediyl groups with one or more alicyclic saturated hydrocarbon groups, for example, an -alkanediyl group-alicyclic saturated hydrocarbon group, an -alicyclic saturated hydrocarbon group-alkyl group, an -alkanediyl group-alicyclic saturated hydrocarbon group-alkyl group and the like.

Examples of the alkanediyl group include linear alkanediyl groups such as a methylene group, an ethylene group, a propane-1,3-diyl group, a butane-1,4-diyl group, a pentane-1,5-diyl group and a hexane-1,6-diyl group; and branched alkanediyl groups such as a propane-1,2-diyl group, a butane-1,3-diyl group, a 2-methylpropane-1,2-diyl group, a 1-methylbutane-1,4-diyl group and a 2-methylbutane-1,4-diyl group. The end of the branched alkanediyl group may be a methyl group.

Examples of the halogen atom possessed by the saturated hydrocarbon group included in R⁴² include a fluorine atom, a chlorine atom, a bromine atom or an iodine atom.

Examples of the group in which —CH₂— of the saturated hydrocarbon group included in R⁴² is replaced by —O— or —CO— include a hydroxy group (a group in which —CH₂— included in the methyl group is replaced by —O—), a carboxy group (a group in which —CH₂—CH₂— included in the ethyl group is replaced by —O—CO—), a carbonyl group (a group in which —CH₂—included in the methylene group is replaced by —CO—), an oxy group (a group in which —CH₂— included in the methylene group is replaced by —O—), an alkoxy group (a group in which —CH₂—at any position included in the alkyl group is replaced by —O—), an alkoxycarbonyl group (a group in which —CH₂—CH₂— at any position included in the alkyl group is replaced by —O— CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), a cycloalkoxy group, a cycloalkylalkoxy group, groups obtained by combining two or more of these groups and the like. Examples of these replaced groups include the same groups as mentioned herein as long as the upper limit of the number of carbon atoms permits.

The structural unit (a4) is preferably a structural unit in which R⁴² is a saturated hydrocarbon group having a fluorine atom. An example of the structural unit (a4) in which R⁴² is a saturated hydrocarbon group having a fluorine atom include a structural unit represented by formula (a4-1) (hereinafter sometimes referred to as “structural unit (a4-1)”), a structural unit represented by formula (a4-2) (hereinafter sometimes referred to as “structural unit (a4-2)”) and a structural unit represented by formula (a4-3) (hereinafter sometimes referred to as “structural unit (a4-3)”).

The structural unit represented by formula (a4-1) is a structural unit represented by the following:

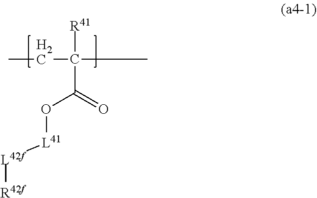

wherein, in formula (a4-1),
R⁴¹ represents a hydrogen atom or a methyl group,
L⁴¹ represents a single bond or an alkanediyl group having 1 to 4 carbon atoms,
L^{42f} represents an alkanediyl group having 1 to 8 carbon atoms which has a fluorine atom, or a cycloalkanediyl group having 3 to 12 carbon atoms which has a fluorine atom, and
R^{42f} represents a fluorine atom or a hydrogen atom.

Examples of the alkanediyl group in L⁴¹ include linear alkanediyl groups such as a methylene group, an ethylene group, a propane-1,3-diyl group and a butane-1,4-diyl group; and branched alkanediyl groups such as an ethane-1,1-diyl group, a propane-1,2-diyl group, a butane-1,3-diyl group, a 2-methylpropane-1,3-diyl group and a 2-methylpropane-1,2-diyl group. The end of the branched alkanediyl group may be a methyl group.

Examples of the alkanediyl group having a fluorine atom in L^{42f} include linear alkanediyl groups having a fluorine atom, such as a methylene group having a fluorine atom, an ethylene group having a fluorine atom, a propane-1,3-diyl group having a fluorine atom, a butane-1,4-diyl group having a fluorine atom, a pentane-1,5-diyl group having a fluorine atom, a hexane-1,6-diyl group having a fluorine atom, a heptane-1,7-diyl group having a fluorine atom and an octane-1,8-diyl group having a fluorine atom; and

branched alkanediyl group having a fluorine atom, such as an ethane-1,1-diyl group having a fluorine atom, a propane-1,1-diyl group having a fluorine atom, a propane-1,2-diyl group having a fluorine atom, a propane-2,2-diyl group having a fluorine atom, a pentane-2,4-diyl group having a fluorine atom, a 2-methylpropane-1,3-diyl group having a fluorine atom, a 2-methylpropane-1,2-diyl group having a fluorine atom, a pentane-1,4-diyl group having a fluorine atom and a 2-methylbutane-1,4-diyl group having a fluorine atom. The end of the branched alkanediyl group may be a methyl group which may have a fluorine atom.

Examples of cycloalkanediyl group having a fluorine atom in L^{42f} include monocyclic cycloalkanediyl groups having a fluorine atom, such as a cyclobutane-1,3-diyl group having a fluorine atom, a cyclopentane-1,3-diyl group having a fluorine atom, a cyclohexane-1,4-diyl group having a fluorine atom, a cyclohexene-3,6-diyl group having a fluorine atom, a cycloheptane-1,4-diyl group having a fluorine atom and a cyclooctane-1,5-diyl group having a fluorine atom, and

polycyclic cycloalkanediyl groups having a fluorine atom, such as a norbornane-1,4-diyl group having a fluorine atom, a norbornane-2,5-diyl group having a fluorine atom, a 5-norbornene-2,3-diyl group having a fluorine atom, an adamantane-1,5-diyl group having a fluorine atom, and an adamantane-2,6-diyl group having a fluorine atom.

The number of fluorine atoms of the alkanediyl group and the cycloalkanediyl group each having a fluorine atom in L^{42f} may be 1 or more, and preferably 2 or more.

The alkanediyl group and the cycloalkanediyl group each having a fluorine atom in L^{42f} are preferably a perfluoroalkanediyl group and a perfluorocycloalkanediyl group, respectively.

Examples of the perfluoroalkanediyl group in L^{42f} include a difluoromethylene group, a perfluoroethylene group, a perfluoroethylfluoromethylene group, a perfluoropropane-1,3-diyl group, a perfluoropropane-1,2-diyl group, a perfluoropropane-2,2-diyl group, a perfluorobutane-1,4-diyl group, a perfluorobutane-2,2-diyl group, a perfluorobutane-1,2-diyl group, a perfluoropentane-1,5-diyl group, a perfluoropentane-2,2-diyl group, a perfluoropentane-3,3-diyl group, a perfluorohexane-1,6-diyl group, a perfluorohexane-2,2-diyl group, a perfluorohexane-3,3-diyl group, a perfluoroheptane-1,7-diyl group, a perfluoroheptane-2,2-diyl group, a perfluoroheptane-3,4-diyl group, a perfluoroheptane-4,4-diyl group, a perfluorooctane-1,8-diyl group, a perfluorooctane-2,2-diyl group, a perfluorooctane-3,3-diyl group, a perfluorooctane-4,4-diyl group and the like.

Examples of the perfluorocycloalkanediyl group in L^{42f} include a perfluorocyclohexanediyl group, a perfluorocyclopentanediyl group, a perfluorocycloheptanediyl group, a perfluoroadamantanediyl group and the like.

L⁴¹ is preferably a single bond or an alkanediyl group having 1 to 3 carbon atoms, more preferably a single bond, a methylene group or an ethylene group, and still more preferably a single bond or a methylene group.

L^{42f} is preferably a perfluoroalkanediyl group having 1 to 8 carbon atoms or a perfluorocycloalkanediyl group having 3 to 12 carbon atoms, more preferably a perfluoroalkanediyl group having 1 to 6 carbon atoms, and still more preferably a perfluoroalkanediyl group having 1 to 3 carbon atoms.

Examples of the structural unit (a4-1) include the following structural units, and structural units in which a methyl group corresponding to R⁴¹ in the structural unit (a4-1) in the following structural units is substituted with a hydrogen atom.

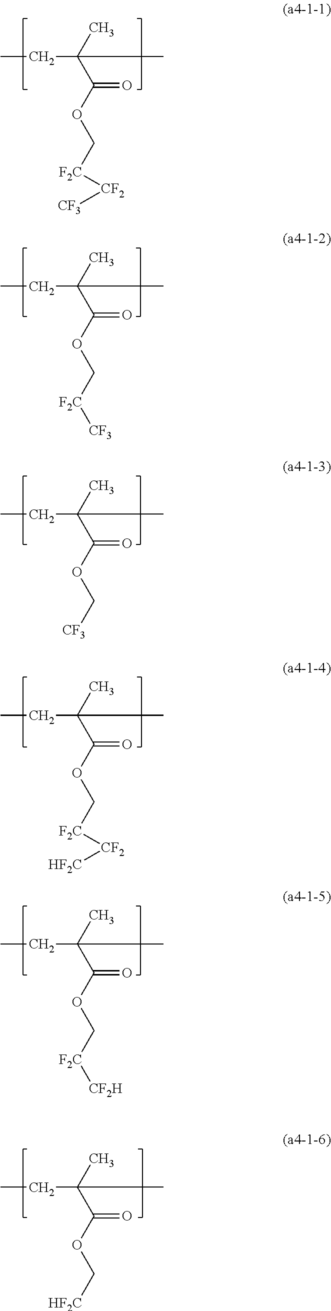

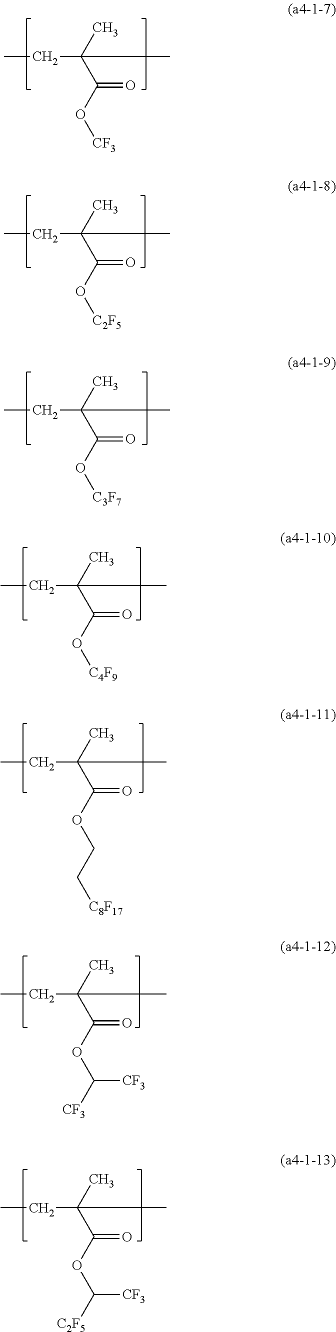

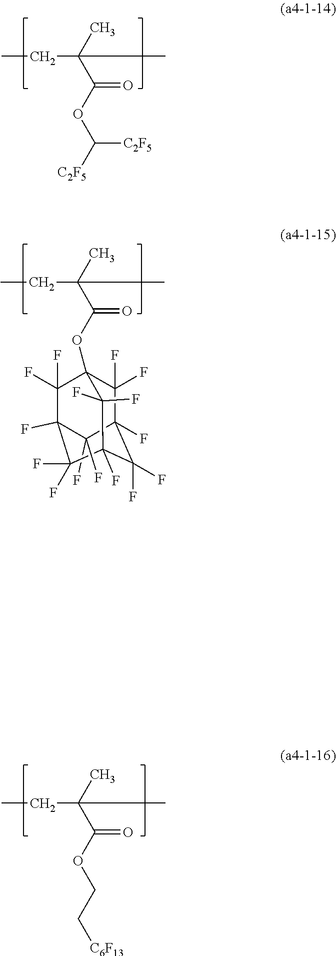

Examples of the structural unit represented by formula (a4-2) include the following structural unit:

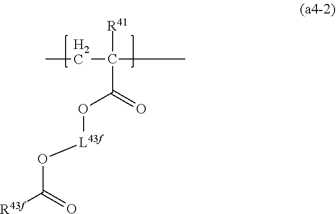

wherein, in formula (a4-2),
R⁴¹ represents a hydrogen atom or a methyl group,
L^{43f} represents a saturated hydrocarbon group having 1 to 20 carbon atoms which may have a fluorine atom, and —CH₂— included in the saturated hydrocarbon group may be replaced by —O— or —CO—, and
R^{43f} represents a saturated hydrocarbon group having 1 to 20 carbon atoms which may have a fluorine atom, and —CH₂— included in the saturated hydrocarbon group may be replaced by —O— or —CO—,
in which at least one of L^{43f} and R^{43f} has a fluorine atom, and the upper limit of the total number of carbon atoms of L^{43f} and R^{43f} is 21.

Examples of the saturated hydrocarbon group in L^{43f} and R^{43f} include a linear or branched chain saturated hydrocarbon group and a monocyclic or polycyclic alicyclic saturated hydrocarbon group, and groups formed by combining these groups. Examples of the saturated hydrocarbon group in L^{43f} and R^{43f} include groups which are the same as the saturated hydrocarbon groups mentioned as the saturated hydrocarbon group as for R⁴² in formula (a4) as long as the upper limit of the total number of carbon atoms of L^{43f} and R^{43f} permit.

The saturated hydrocarbon group as for L^{43f} is preferably an alkanediyl group having 1 to 6 carbon atoms or a group represented by formula (L^{43f}-1):

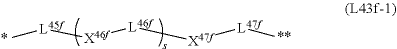

wherein, in formula (L^{43f}-1),
s represents 0 or 1,
L^{45f} and L^{46f} each independently represent a divalent saturated hydrocarbon group having 1 to 5 carbon atoms which may have a fluorine atom,
L^{47f} represents a single bond or a divalent saturated hydrocarbon group having 1 to 5 carbon atoms which may have a fluorine atom,
X^{46f} and X^{47f} each independently represent —O—, —CO—, —CO—O— or —O—CO—, in which the total number of carbon atoms of L^{45f},
L^{46f}, L^{47f}, X^{46f} and X^{47f} is 7 or less, and
* and ** are bonding sites, and ** is a bonding site to —O—CO—R^{43f}.

Examples of the divalent saturated hydrocarbon group represented by L^{45f}, L^{46f} and L^{47f} in the group represented by formula (L43f-1) include a linear or branched alkanediyl group and a monocyclic or polycyclic divalent alicyclic saturated hydrocarbon group, and a divalent saturated hydrocarbon group formed by combining an alkanediyl group and a divalent alicyclic saturated hydrocarbon group. Specific examples thereof include a methylene group, an ethylene group, a propane-1,3-diyl group, a propane-1,2-diyl group, a butane-1,4-diyl group, a 1-methylpropane-1,3-diyl group, a 2-methylpropane-1,3-diyl group, a 2-methylpropane-1,2-diyl group and the like.

S is preferably 0.

Examples of the group represented by formula (L^{43f}-1) include the following groups. In the following, * and ** each represent a bonding site, and ** is a bonding site to —O—CO—R^{43f}.

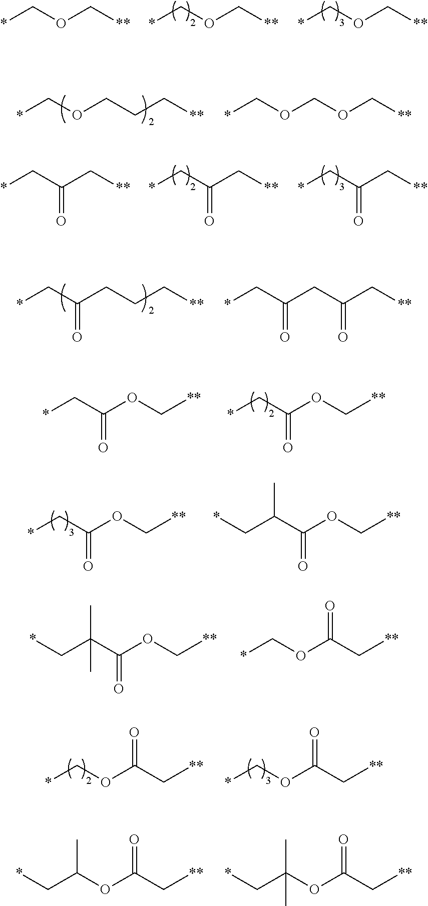

R^{43f} is preferably a saturated hydrocarbon group having a fluorine atom.

In this case, examples of the structural unit represented by formula (a4-2) include a structural unit represented by formula (a4-2A) or formula (a4-2B):

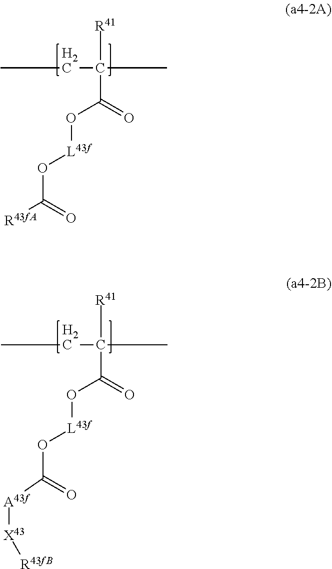

wherein, in formula (a4-2A) and formula (a4-2B),
R⁴¹ and L^{43f} are the same as defined in formula (a4-2),
R^{43f} A represents a saturated hydrocarbon group having 1 to 20 carbon atoms which has a fluorine atom,
A^{43f} represents a divalent saturated hydrocarbon group having 1 to 17 carbon atoms which may have a fluorine atom, and —CH₂— included in the saturated hydrocarbon group may be replaced by —O— or —CO—,
X⁴³ represents **—O—CO— or **—CO—O—, and ** represents a bonding site to A^{43f}, and
R^{43fB} represents a saturated hydrocarbon group having 1 to 17 carbon atoms which may have a fluorine atom,
in which, in formula (a4-2A), the total number of carbon atoms of L^{43f} and R^{43f} A is 21 or less, and in formula (a4-2B), the total number of carbon atoms of L^{43f}, A^{43f}, X⁴³ and R^{43fB} is 21 or less, and at least one of A^{43f} and R^{43f} B has at least one fluorine atom.

Examples of the saturated hydrocarbon group as for R^{43f} A include the same saturated hydrocarbon groups as mentioned as for R⁴² as long as the upper limit of the number of carbon atoms permit.

R^{43fA} is preferably an alkyl group having 1 to 13 carbon atoms which has a fluorine atom, a cycloalkyl group having 3 to 12 carbon atoms which has a fluorine atom, or group obtained by combining these groups, and more preferably *—(CF₂)_{n43f}—R^{42′} (* represents a bonding site to a carbonyl group, n43f represents an integer of 1 to 6, and R^{42′} represents a hydrogen atom or a fluorine atom) or a perfluoro cycloalkyl group having 3 to 12 carbon atoms.

Examples of the saturated hydrocarbon group as for A^{43f} and R^{43fB} include the same saturated hydrocarbon groups as mentioned as for R⁴² in formula (a4) as long as the upper limit of the number of carbon atoms permit.

A^{43f} is preferably a divalent chain saturated hydrocarbon group which may have a fluorine atom, a divalent alicyclic saturated hydrocarbon group, or groups obtained by combining these groups, more preferably a divalent chain saturated hydrocarbon group having a fluorine atom, and still more preferably an alkanediyl fluoride group having 1 to 6 carbon atoms.

The saturated hydrocarbon group which may have a fluorine atom as for R^{43fB} is preferably a chain saturated hydrocarbon group, an alicyclic saturated hydrocarbon group, or groups obtained by combining these groups which may have a fluorine atom, more preferably an alkyl group having 1 to 12 carbon atoms, a cycloalkyl group having 3 to 12 carbon atoms, or groups obtained by combining these groups which may have a fluorine atom, and still more preferably alkyl fluoride groups such as a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a heptyl group, a perfluoro heptyl group, an octyl group and a perfluorooctyl group; a cyclopropylmethyl group, a cyclopropyl group, a cyclobutylmethyl group, a cyclopentyl group, a cyclohexyl group, a perfluoro cyclohexyl group, an adamantyl group, an adamantylmethyl group, an adamantyldimethyl group, a norbornyl group, a norbornylmethyl group, a perfluoroadamantyl group, a perfluoroadamantylmethyl group and the like.

In formula (a4-2B), an example of the structure that the group represented by *-A^{43f}-X⁴³—R^{43fB} can take includes the following structures (* is a bonding site to a carbonyl group).

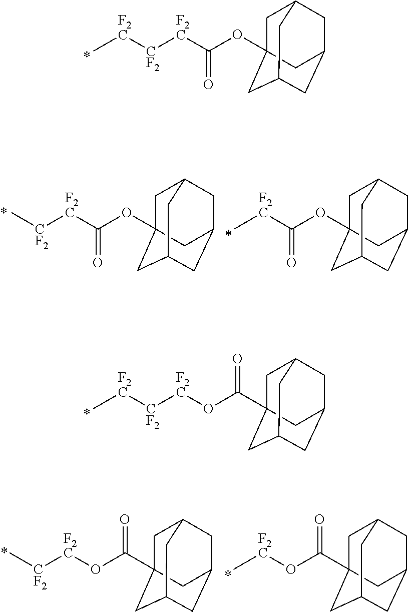

Examples of the structural unit represented by formula (a4-2A) include the following structural units, and structural units in which the methyl group corresponding to R⁴¹ in the structural unit represented by formula (a4-2A) in the following structural units is substituted with a hydrogen atom.

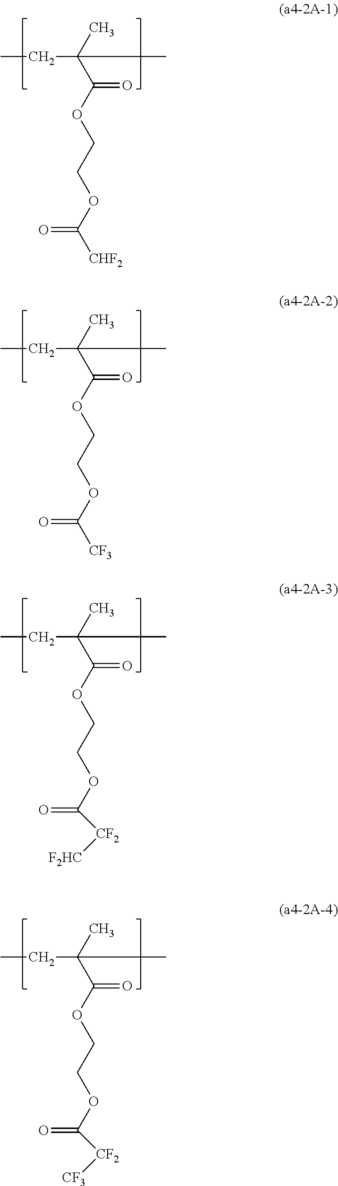

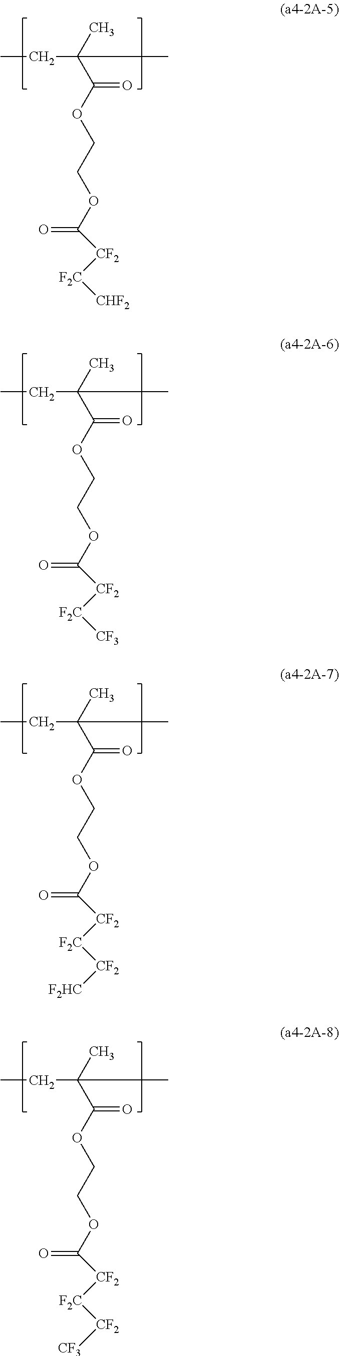

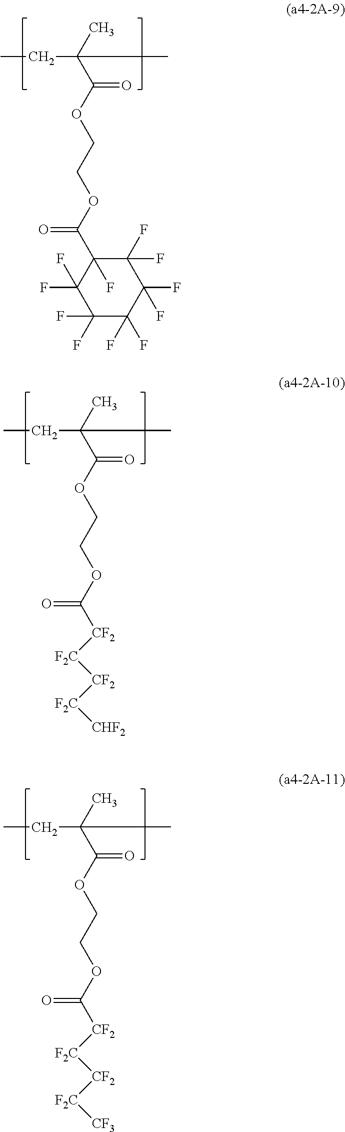

Examples of the structural unit represented by formula (a4-2B) include the following structural units, and structural units in which the methyl group corresponding to R⁴¹ in the structural unit represented by formula (a4-2B) in the following structural units is substituted with a hydrogen atom.

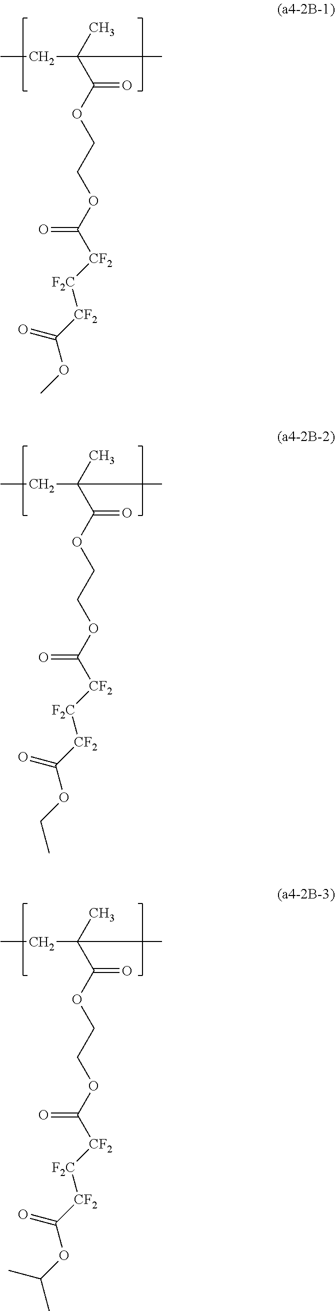

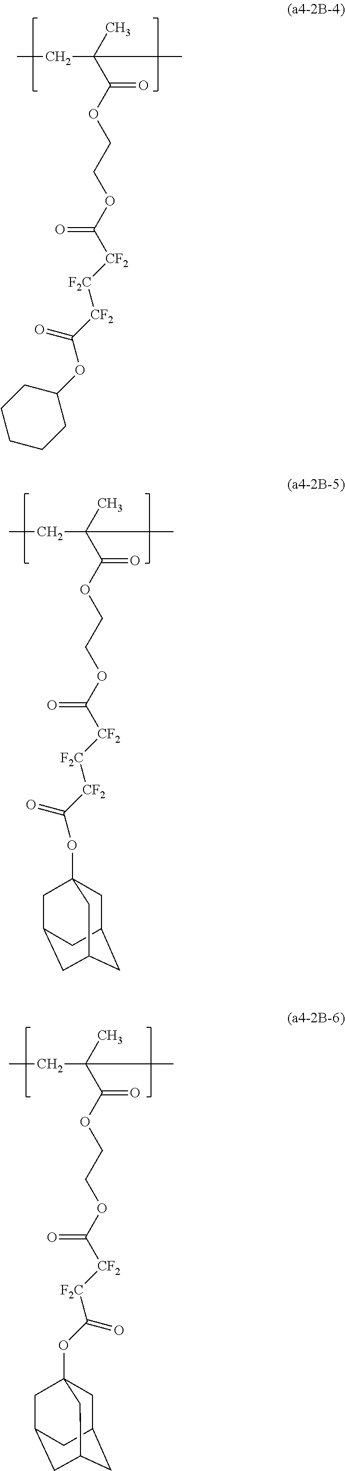

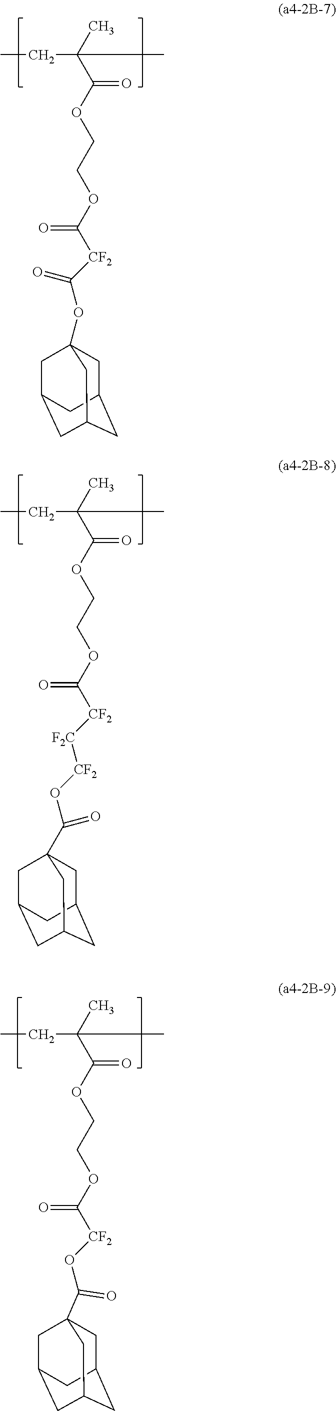

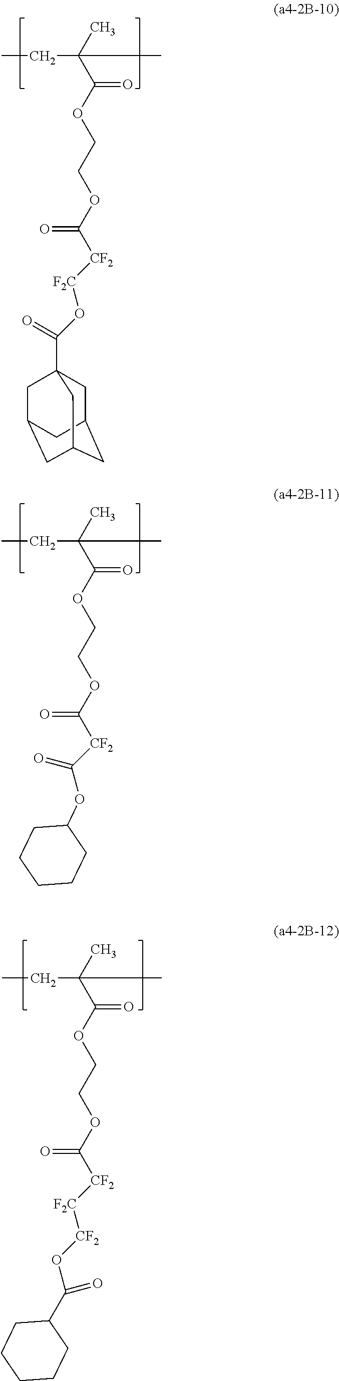

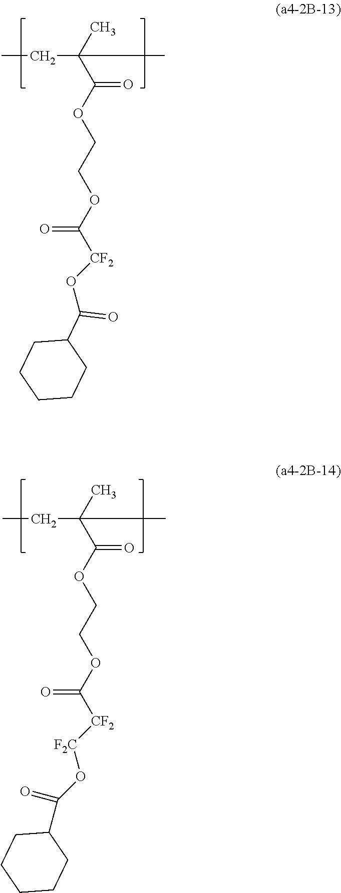

The structural unit (a4-3) is a structural unit represented by the following formula (a4-3)

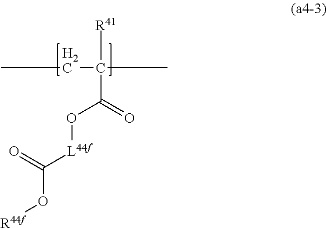

wherein, in formula (a4-3),
R⁴¹ represents a hydrogen atom or a methyl group,
L⁴⁴ represents a saturated hydrocarbon group having 1 to 20 carbon atoms which may have a fluorine atom, and —CH₂— included in the saturated hydrocarbon group may be replaced by —O— or —CO—, and
R^{44f} represents a saturated hydrocarbon group having 1 to 20 carbon atoms which may have a fluorine atom, and —CH₂— included in the saturated hydrocarbon group may be replaced by —O— or —CO—,
in which at least one of L^{44f} and R^{44f} has a fluorine atom, and the upper limit of the total number of carbon atoms of L^{44f} and R^{44f} is 21.

Examples of the saturated hydrocarbon group in L⁴⁴f and R^{44f} include a linear or branched chain saturated hydrocarbon group and a monocyclic or polycyclic alicyclic saturated hydrocarbon group, and groups formed by combining these groups. Examples of the saturated hydrocarbon group in L^{44f} and R^{44f} include groups which are the same as saturated hydrocarbon groups as mentioned as for R⁴² in formula (a4) as long as the upper limit of the number of carbon atoms of L⁴⁴f and R^{44f} permit.

L^{44f} is preferably an alkanediyl group having 1 to 14 carbon atoms (—CH₂— included in the alkanediyl group may be replaced by —O— or —CO—), and more preferably a group represented by —(CH₂)ⱼ₁—, —(CH₂)ⱼ₂—O—(CH₂)ⱼ₃— or —(CH₂)ⱼ₄—CO— O—(CH₂)ⱼ₅— (j1 to j5 each independently represent an integer of 1 to 6 represents). It is also preferably an alkanediyl group having 1 to 4 carbon atoms (one —CH₂— included in the alkanediyl group may be replaced by —O—, and one —CH₂—CH₂—included in the alkanediyl group may be replaced by —CO—O— or —O—CO—).

R^{44f} is preferably a saturated hydrocarbon group having 1 to 10 carbon atoms which has a fluorine atom, more preferably an alkyl group having 1 to 10 carbon atoms which has a fluorine atom, an alicyclic saturated hydrocarbon group having 3 to 10 carbon atoms which has a fluorine atom, or groups obtained by combining these groups, still more preferably an alkyl group having 1 to 10 carbon atoms which has a fluorine atom, and yet more preferably an alkyl group having 1 to 6 carbon atoms which has a fluorine atom.

Examples of the structural unit represented by formula (a4-3) include the following structural units, and structural units in which the methyl group corresponding to R⁴¹ in the structural unit (a4-3) is substituted with a hydrogen atom in the structural units represented by the following formulas.

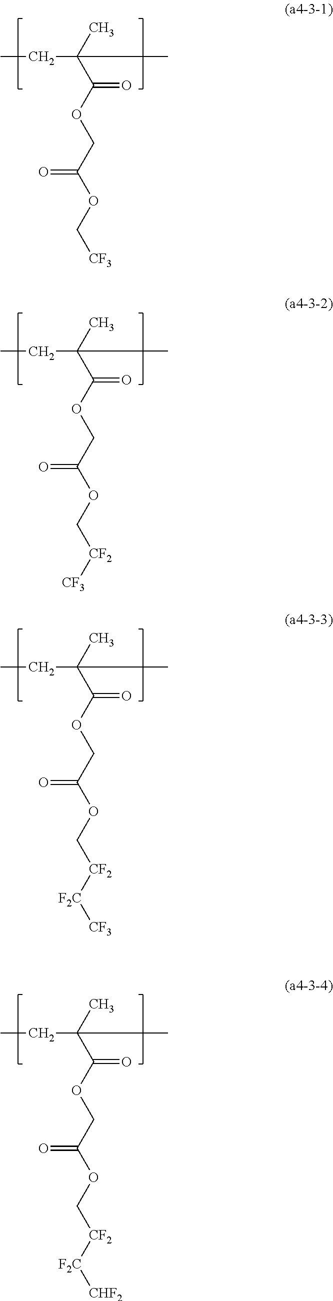

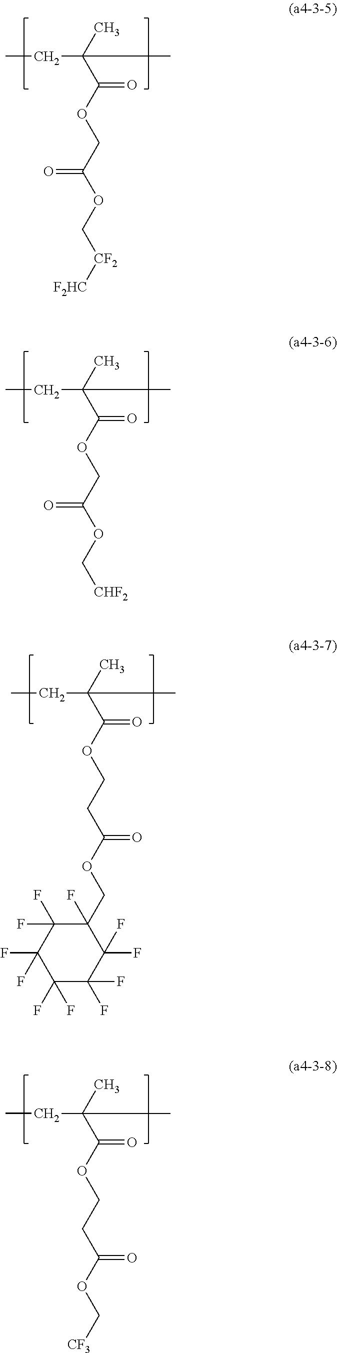

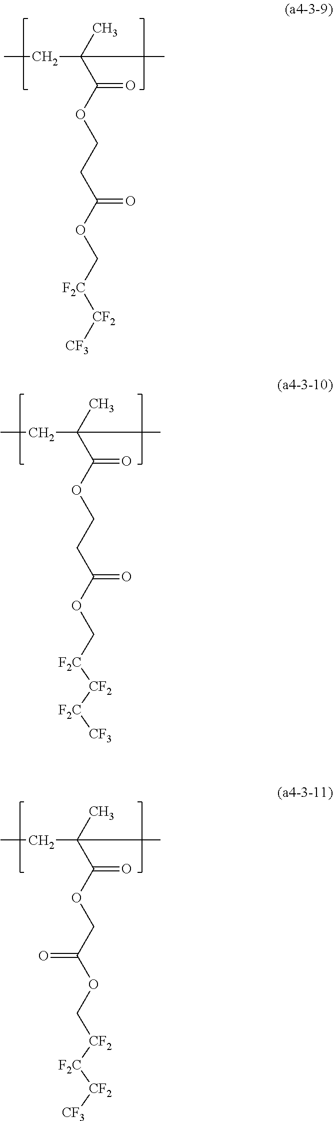

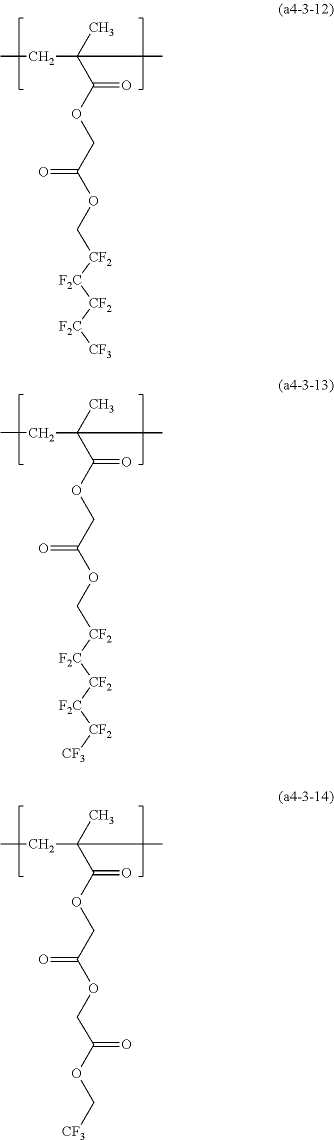

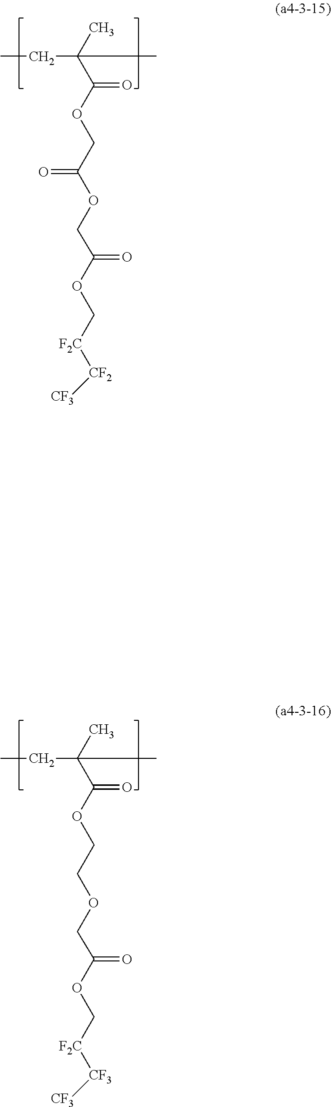

When the resin (A) or the like includes the structural unit (a4), the content is preferably 1 to 20 mol %, more preferably 2 to 15 mol %, and still more preferably 3 to 10 mol %, based on all structural units of the resin (A) or the like.

<Structural Unit (a5)>

Examples of a non-leaving hydrocarbon group possessed by the structural unit (a5) include groups having a linear, branched or cyclic hydrocarbon group. Of these, the structural unit (a5) is preferably a group having an alicyclic hydrocarbon group.

The structural unit (a5) includes, for example, a structural unit represented by formula (a5-1):

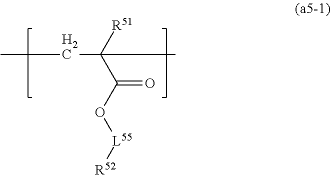

wherein, in formula (a5-1),
R⁵¹ represents a hydrogen atom or a methyl group,
R⁵² represents an alicyclic hydrocarbon group having 3 to 18 carbon atoms, and a hydrogen atom included in the alicyclic hydrocarbon group may be substituted with an aliphatic hydrocarbon group having 1 to 8 carbon atoms, and
L⁵⁵ represents a single bond or a divalent saturated hydrocarbon group having 1 to 18 carbon atoms, and —CH₂— included in the saturated hydrocarbon group may be replaced by —O— or —CO—.

The alicyclic hydrocarbon group in R⁵² may be either monocyclic or polycyclic. The monocyclic alicyclic hydrocarbon group includes, for example, a cyclopropyl group, a cyclobutyl group, a cyclopentyl group and a cyclohexyl group. The polycyclic alicyclic hydrocarbon group includes, for example, an adamantyl group and a norbornyl group.

The aliphatic hydrocarbon group having 1 to 8 carbon atoms includes, for example, alkyl groups such as a methyl group, an ethyl group, a propyl group, an isopropyl group, a butyl group, a sec-butyl group, a tert-butyl group, a pentyl group, a hexyl group, an octyl group and a 2-ethylhexyl group.

Examples of the alicyclic hydrocarbon group having a substituent includes a 3-methyladamantyl group and the like.

R⁵² is preferably an unsubstituted alicyclic hydrocarbon group having 3 to 18 carbon atoms, and more preferably an adamantyl group, a norbornyl group or a cyclohexyl group.

Examples of the divalent saturated hydrocarbon group in L⁵⁵ include a divalent chain saturated hydrocarbon group and a divalent alicyclic saturated hydrocarbon group, and a divalent chain saturated hydrocarbon group is preferable.

The divalent chain saturated hydrocarbon group includes, for example, alkanediyl groups such as a methylene group, an ethylene group, a propanediyl group, a butanediyl group and a pentanediyl group.

The divalent alicyclic saturated hydrocarbon group may be either monocyclic or polycyclic. Examples of the monocyclic alicyclic saturated hydrocarbon group include cycloalkanediyl groups such as a cyclopentanediyl group and a cyclohexanediyl group. Examples of the polycyclic divalent alicyclic saturated hydrocarbon group include an adamantanediyl group and a norbornanediyl group.

The group in which —CH₂— included in the divalent saturated hydrocarbon group represented by L⁵⁵ is replaced by —O— or —CO— includes, for example, groups represented by formula (L1-1) to formula (L1-4). In the following formulas, * and ** each represent a bonding site, and * represents a bonding site to an oxygen atom:

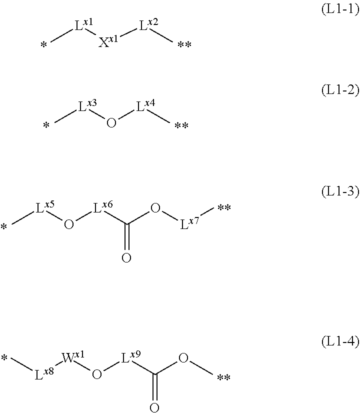

wherein, in formula (L1-1),
X^{x1} represents *—O—CO— or *—CO—O— (* represents a bonding site to L^{x1}),
L^{x1} represents a divalent aliphatic saturated hydrocarbon group having 1 to 16 carbon atoms,
L^{x2} represents a single bond or a divalent aliphatic saturated hydrocarbon group having 1 to 15 carbon atoms, and
the total number of carbon atoms of L^{x1} and L^{x2} is 16 or less:
   wherein, in formula (L1-2),
L^{x3} represents a divalent aliphatic saturated hydrocarbon group having 1 to 17 carbon atoms,
L^{x4} represents a single bond or a divalent aliphatic saturated hydrocarbon group having 1 to 16 carbon atoms, and
the total number of carbon atoms of L^{x3} and L^{x4} is 17 or less:
   wherein, in formula (L1-3),
L^{x5} represents a divalent aliphatic saturated hydrocarbon group having 1 to 15 carbon atoms,
L^{x6} and L^{x7} each independently represent a single bond or a divalent aliphatic saturated hydrocarbon group having 1 to 14 carbon atoms, and
the total number of carbon atoms of L^{x5}, L^{x6} and L^{x7} is 15 or less:
wherein, in formula (L¹-4),
L^{x8} and L^{x9} represent a single bond or a divalent aliphatic saturated hydrocarbon group having 1 to 12 carbon atoms,
W^{x1} represents a divalent alicyclic saturated hydrocarbon group having 3 to 15 carbon atoms, and
the total number of carbon atoms of L^{x8}, L^{x9} and W^{x1} is 15 or less.

L^{x1} is preferably a divalent aliphatic saturated hydrocarbon group having 1 to 8 carbon atoms, and more preferably a methylene group or an ethylene group.

L^{x2} is preferably a single bond or a divalent aliphatic saturated hydrocarbon group having 1 to 8 carbon atoms, and more preferably a single bond.

L^{x3} is preferably a divalent aliphatic saturated hydrocarbon group having 1 to 8 carbon atoms.

L^{x4} is preferably a single bond or a divalent aliphatic saturated hydrocarbon group having 1 to 8 carbon atoms.

L^{x5} is preferably a divalent aliphatic saturated hydrocarbon group having 1 to 8 carbon atoms, and more preferably a methylene group or an ethylene group.

L^{x6} is preferably a single bond or a divalent aliphatic saturated hydrocarbon group having 1 to 8 carbon atoms, and more preferably a methylene group or an ethylene group.

L^{x7} is preferably a single bond or a divalent aliphatic saturated hydrocarbon group having 1 to 8 carbon atoms.

L^{x8} is preferably a single bond or a divalent aliphatic saturated hydrocarbon group having 1 to 8 carbon atoms, and more preferably a single bond or a methylene group.

L^{x9} is preferably a single bond or a divalent aliphatic saturated hydrocarbon group having 1 to 8 carbon atoms, and more preferably a single bond or a methylene group.

W^{x1} is preferably a divalent alicyclic saturated hydrocarbon group having 3 to 10 carbon atoms, and more preferably a cyclohexanediyl group or an adamantanediyl group.

The group represented by formula (L1-1) includes, for example, the following divalent groups.

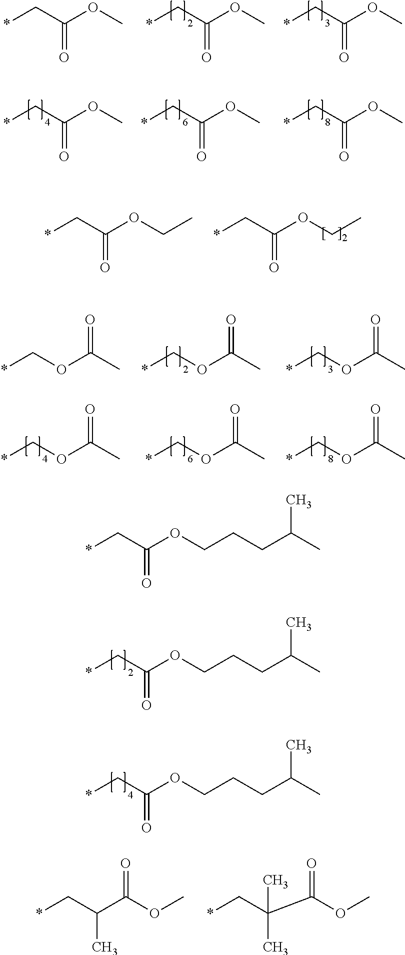

The group represented by formula (L1-2) includes, for example, the following divalent groups.

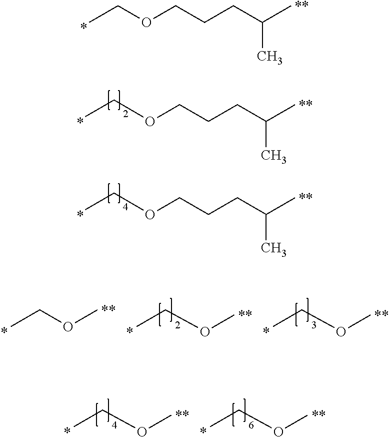

The group represented by formula (L1-3) includes, for example, the following divalent groups.

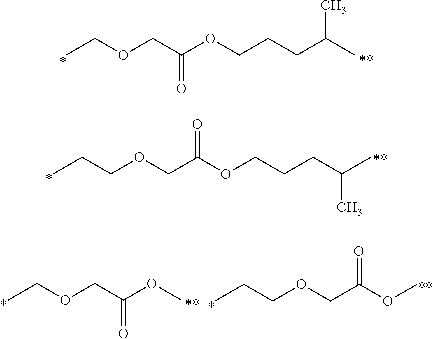

The group represented by formula (L1-4) includes, for example, the following divalent groups.

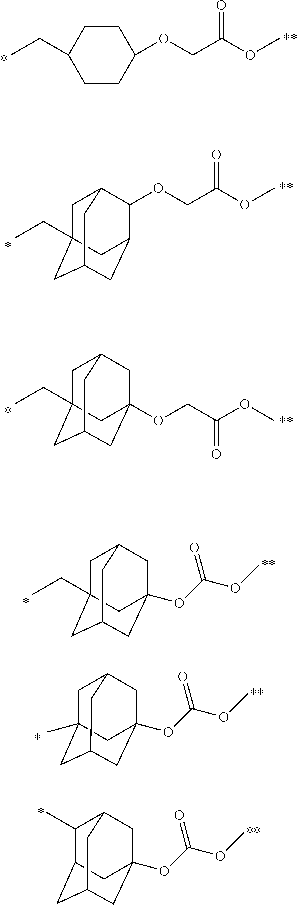

L⁵⁵ is preferably a single bond or a group represented by formula (L1-1).

Examples of the structural unit (a5-1) include the following structural units and structural units in which a methyl group corresponding to R⁵¹ in the structural unit (a5-1) in the following structural units is substituted with a hydrogen atom.

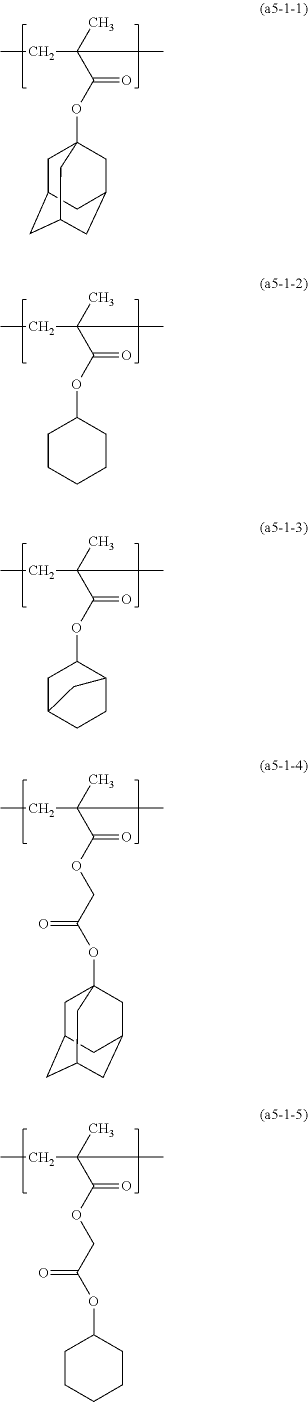

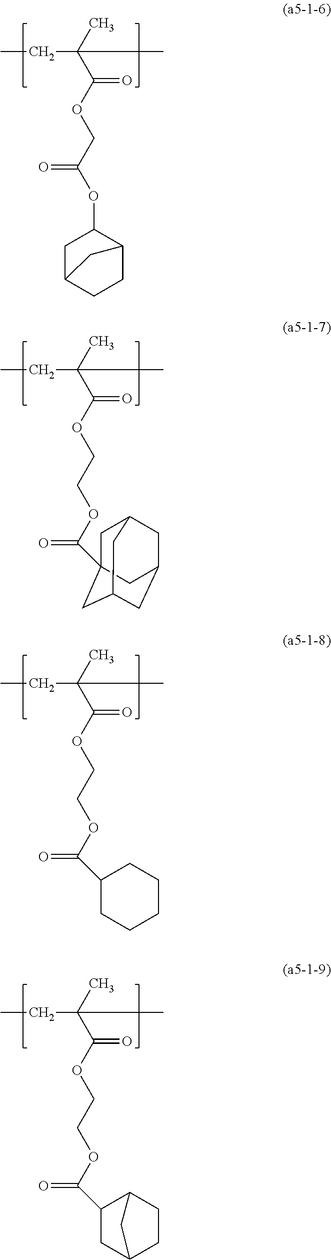

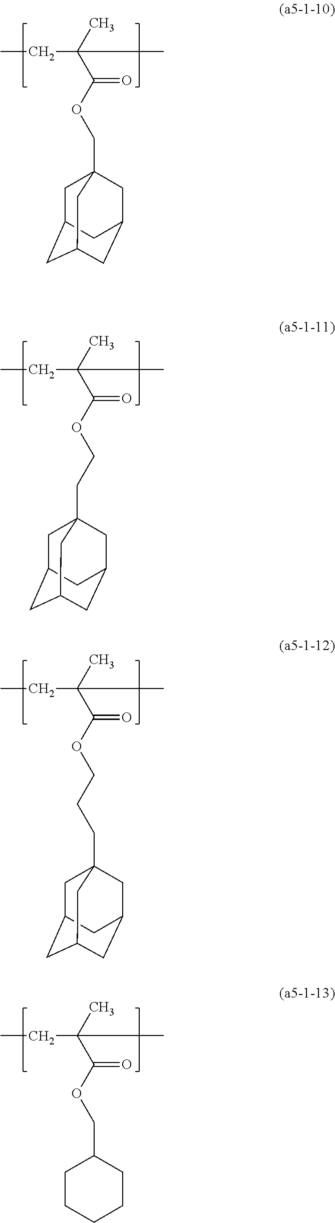

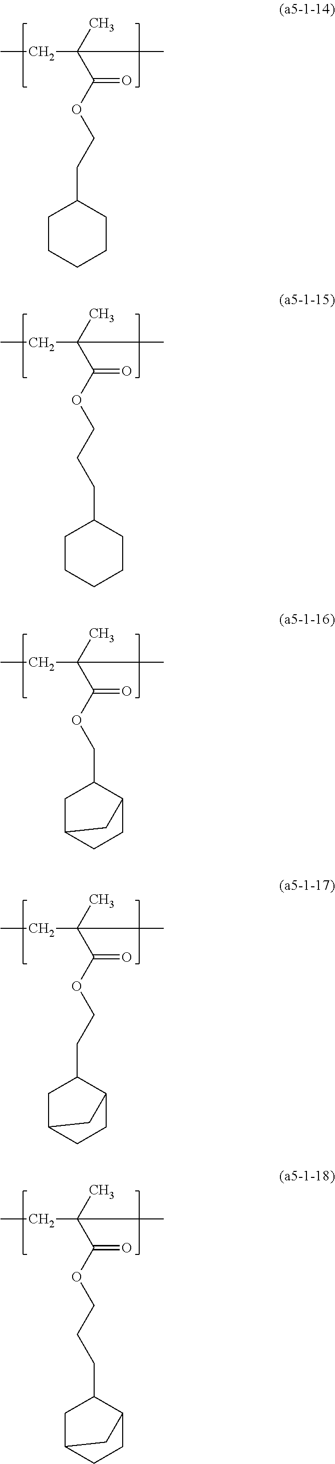

When the resin (A) or the like includes the structural unit (a5), the content is preferably 1 to 30 mol %, more preferably 2 to 20 mol %, and still more preferably 3 to 15 mol %, based on all structural units of the resin (A) or the like.

<Structural Unit (a6)>

The structural unit (a6) is a structural unit having an —SO₂— group, and it is preferable to have an —SO₂— group in a side chain.

The structural unit having an —SO₂— group may have a linear structure having an —SO₂— group, a branched structure having an —SO₂— group, or a cyclic structure (monocyclic and polycyclic structure) having an —SO₂— group. The structural unit is preferably a structural unit which has a cyclic structure having an —SO₂— group, and more preferably a structural unit which has a cyclic structure (sultone ring) having —SO₂—O—.

Examples of the sultone ring include rings represented by the following formula (T1-1), formula (T1-2), formula (T1-3) and formula (T1-4). The bonding site can be any position. The sultone ring may be monocyclic, and is preferably polycyclic. The polycyclic sultone ring means a bridged ring which has —SO₂—O— as an atomic group constituting the ring, and examples thereof include rings represented by formula (T1-1) and formula (T1-2). The sultone ring may have, as the atomic group constituting the ring, a heteroatom, in addition to —SO₂—O—, like the ring represented by formula (T1-2). Examples of the heteroatom include an oxygen atom, a sulfur atom or a nitrogen atom, and an oxygen atom is preferable.

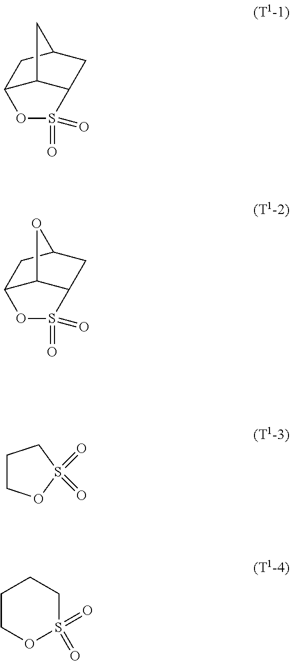 [SMILES: O=S1(=O)OC2CC3CC2C1C3 ; O=S1(=O)OC2CC3CC1C2O3 ; O=S1(=O)CCCO1 ; O=S1(=O)CCCCO1]

The sultone ring may have a substituent, and examples of the substituent include an alkyl group having 1 to 12 carbon atoms which may have a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms and an alkylcarbonyl group having 2 to 4 carbon atoms.

Examples of the halogen atom include a fluorine atom, a chlorine atom, a bromine atom and an iodine atom.

Examples of the alkyl group include a methyl group, an ethyl group, a propyl group, a butyl group, a pentyl group, a hexyl group, an octyl group and a decyl group, and the alkyl group is preferably an alkyl group having 1 to 6 carbon atoms, and more preferably a methyl group.

Examples of the alkyl group having a halogen atom include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a trichloromethyl group, a tribromomethyl group and a triiodomethyl group, and a trifluoromethyl group is preferable.

Examples of the alkyl group having a hydroxy group include hydroxyalkyl groups such as a hydroxymethyl group and a 2-hydroxyethyl group.

Examples of the alkoxy group include a methoxy group, an ethoxy group, a propoxy group, a butoxy group, a pentyloxy group, a hexyloxy group, a heptyloxy group, an octyloxy group, a decyloxy group and a dodecyloxy group.

Examples of the aryl group include a phenyl group, a naphthyl group, an anthryl group, a p-methylphenyl group, a p-tert-butylphenyl group, a p-adamantylphenyl group, a tolyl group, a xylyl group, a cumyl group, a mesityl group, a biphenyl group, a phenanthryl group, a 2,6-diethylphenyl group and a 2-methyl-6-ethylphenyl group.

Examples of the aralkyl group include a benzyl group, a phenethyl group, a phenylpropyl group, a naphthylmethyl group and a naphthylethyl group.

Examples of the alkoxycarbonyl group include groups in which an alkoxy group is bonded with a carbonyl group, such as a methoxycarbonyl group or an ethoxycarbonyl group, and preferably include an alkoxycarbonyl group having 6 or less carbon atoms and more preferably include a methoxycarbonyl group.

Examples of the alkylcarbonyl group include an acetyl group, a propionyl group and a butyryl group.

From the viewpoint that it is easy to produce a monomer from which the structural unit (a6) is derived, a sultone ring having no substituent is preferable.

The sultone ring is preferably a ring represented by the following formula (T1′):

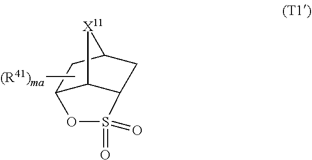

wherein, in formula (T1′),
X¹¹ represents an oxygen atom, a sulfur atom or a methylene group,
R⁴¹ represents an alkyl group having 1 to 12 carbon atoms which may have a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms,
ma represents an integer of 0 to 9, and when ma is 2 or more, a plurality of R⁴¹ may be the same or different, and
the bonding site may be any position.

X¹¹ is preferably an oxygen atom or a methylene group, and more preferably a methylene group.

Examples of R⁴¹ include those which are the same as the substituent of the sultone ring mentioned above, and an alkyl group having 1 to 12 carbon atoms which may have a halogen atom or a hydroxy group is preferable.

ma is preferably 0 or 1, and more preferably 0.

Examples of the ring represented by formula (T1′) include the following rings. The bonding site may be any position. Particularly, the bonding site is preferably the 1-position or the 3-position.

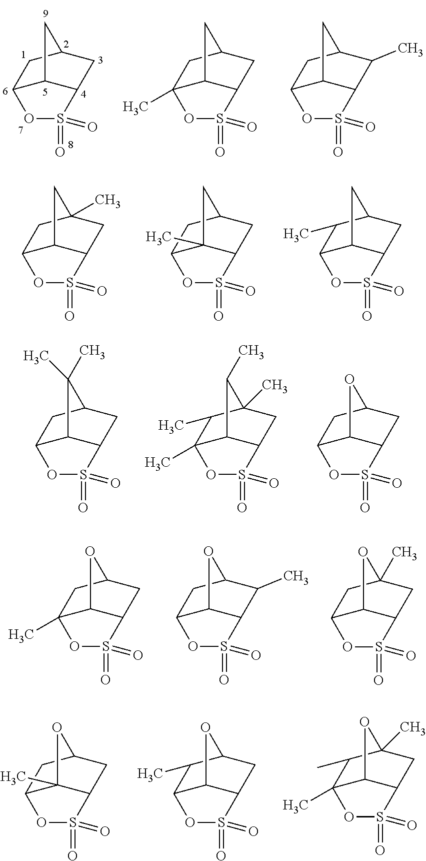

It is preferable that the structural unit having an —SO₂— group further has a group derived from a polymerizable group. Examples of the polymerizable group include a vinyl group, an acryloyl group, a methacryloyl group, an acryloyloxy group, a methacryloyloxy group, an acryloylamino group, a methacryloylamino group, an acryloylthio group, a methacryloylthio group and the like.

Particularly, the monomer from which the structural unit (a6) is derived is preferably a monomer having an ethylenically unsaturated bond, and more preferably a (meth) acrylic monomer.

The structural unit (a6) is preferably a structural unit represented by formula (a6-0):

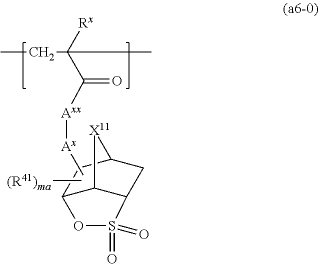

wherein, in formula (a6-0),
R^{x} represents an alkyl group having 1 to 6 carbon atoms which may have a halogen atom, a hydrogen atom or a halogen atom,
A^{xx} represents an oxygen atom, —N(R^{c})—or a sulfur atom,
A^{x} represents a single bond or a divalent saturated hydrocarbon group having 1 to 18 carbon atoms, and —CH₂— included in the saturated hydrocarbon group may be replaced by —O—, —CO— or —N(R^{d})—,
X¹¹, R⁴¹ and ma are the same as defined above, and
R^{c} and R^{d} each independently represent a hydrogen atom or an alkyl group having 1 to 6 carbon atoms.

Examples of the halogen atom as for R^{x} include a fluorine atom, a chlorine atom, a bromine atom and an iodine atom.

Examples of the alkyl group as for R^{x} include a methyl group, an ethyl group, an n-propyl group, an isopropyl group, an n-butyl group, a sec-butyl group, a tert-butyl group, an n-pentyl group and an n-hexyl group, and an alkyl group having 1 to 4 carbon atoms is preferable, and a methyl group or an ethyl group is more preferable.

Examples of the alkyl group having a halogen atom as for R^{x} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a trichloromethyl group, a tribromomethyl group and a triiodomethyl group.

R^{x} is preferably a hydrogen atom or an alkyl group having 1 to 4 carbon atoms, more preferably a hydrogen atom, a methyl group or an ethyl group, and still more preferably a hydrogen atom or a methyl group.

Examples of the divalent saturated hydrocarbon group as for A^{x} include a linear alkanediyl group, a branched alkanediyl group and a monocyclic or polycyclic divalent alicyclic saturated hydrocarbon group, and the divalent saturated hydrocarbon group may be those obtained by combining two or more of these groups.

Specific examples thereof include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, a heptadecane-1,17-diyl group, an ethane-1,1-diyl group, a propane-1,1-diyl group and a propane-2,2-diyl group;
branched alkanediyl groups such as a butane-1,3-diyl group, a 2-methylpropane-1,3-diyl group, a 2-methylpropane-1,2-diyl group, a pentane-1,4-diyl group and a 2-methylbutane-1,4-diyl group;
monocyclic divalent alicyclic saturated hydrocarbon groups which are cycloalkanediyl groups such as a cyclobutane-1,3-diyl group, a cyclopentane-1,3-diyl group, a cyclohexane-1,4-diyl group and a cyclooctane-1,5-diyl group; and
polycyclic divalent alicyclic saturated hydrocarbon groups such as a norbornane-1,4-diyl group, a norbornane-2,5-diyl group, an adamantane-1,5-diyl group and an adamantane-2,6-diyl group.

The bonding site to the sultone ring as for A^{x} can be any position and is preferably the 1-position.

Examples of the structural unit (a6-0) include the following structural units.

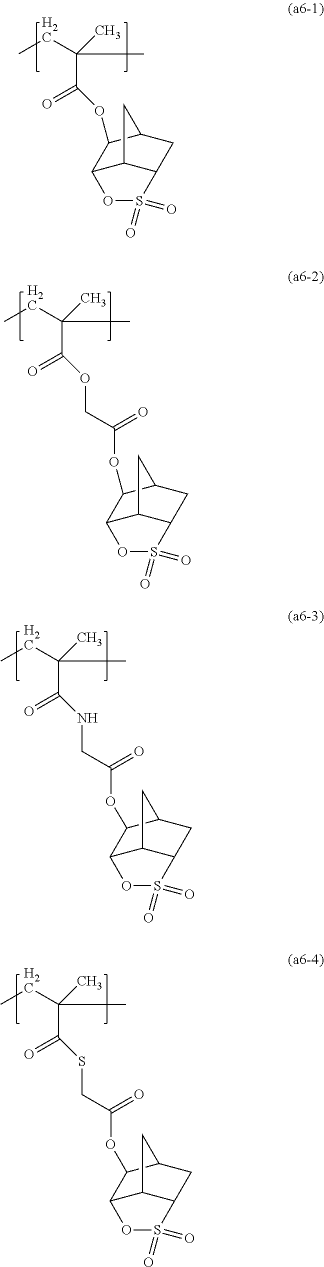

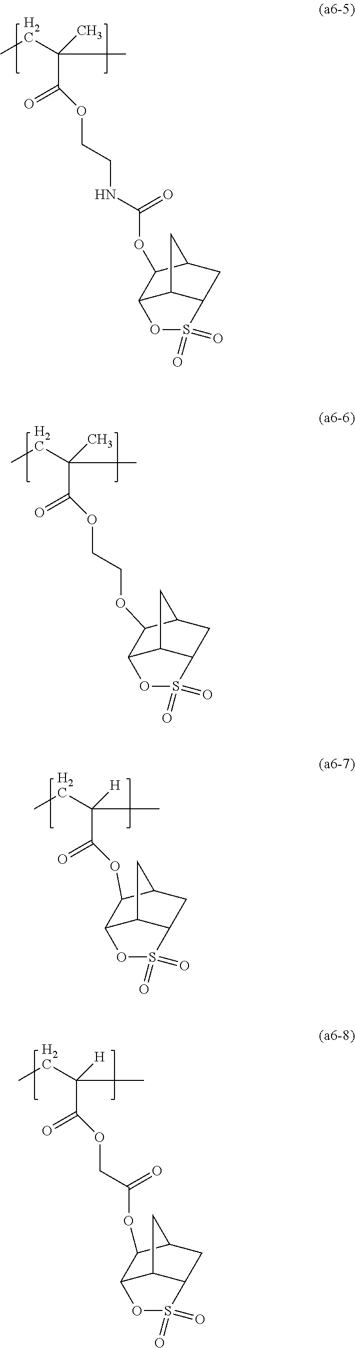

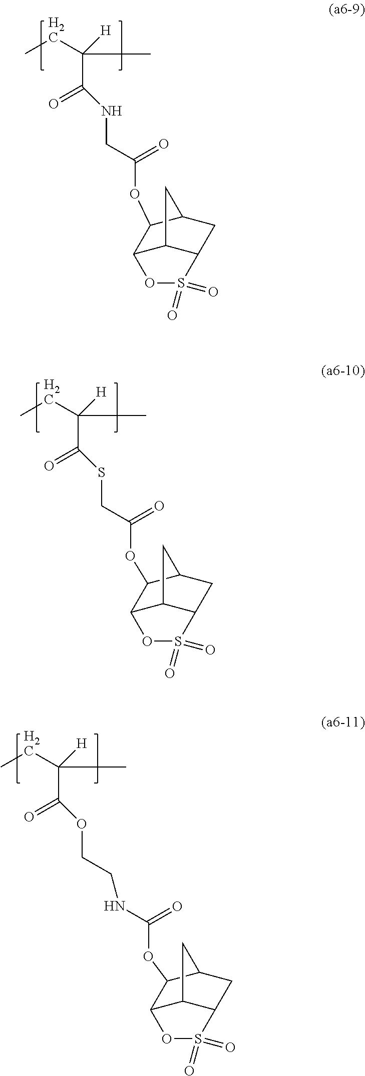

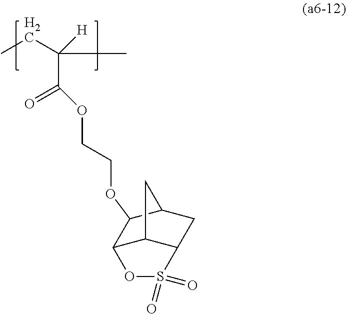

Of these, structural units represented by formula (a6-1), formula (a6-2), formula (a6-6), formula (a6-7), formula (a6-8) and formula (a6-12) are preferable, and structural units represented by formula (a6-1), formula (a6-2), formula (a6-7) and (a6-8) are more preferable.

When the resin (A) or the like includes the structural unit (a6), the content is preferably 1 to 50 mol %, more preferably 2 to 40 mol %, and still more preferably 3 to 30 mol %, based on all structural units of the resin (A) or the like.

<Structural Unit (a7)>

The resin (A) or the like may further include a structural unit which is decomposed upon exposure to radiation to generate an acid (hereinafter sometimes referred to as “structural unit (a7)”, in which a structural unit represented by formula (IP) is excluded). Specific examples of the structural unit (a7) include the structural units mentioned in JP 2016-79235 A, and a structural unit having a sulfonate group or a carboxylate group and an organic cation in a side chain or a structural unit having a sulfonio group and an organic anion in a side chain are preferable.

The structural unit having a sulfonate group or a carboxylate group and an organic cation in a side chain is preferably a structural unit represented by formula (a7-A):

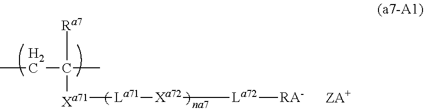

wherein, in formula (a7-A),
R^{a7} represents a hydrogen atom, a halogen atom, or an alkyl group having 1 to 6 carbon atoms which may have a halogen atom,
X^{a71} represents a single bond, *—O—**, *—CO—O—**, *—O—CO—O—**, *—CO—NR^{a71}—**, *—NR^{a71}—CO—O—**, *—O—CO—NR^{a71}—** or *-Ax-Ph-Ay-**,
R^{a71} represents a hydrogen atom or an alkyl group having 1 to 6 carbon atoms,
Ph represents a phenylene group which may have a substituent,
Ax and Ay each independently represent one bond species selected from the group consisting of a single bond, an ether bond, a thioether bond, an ester bond, an amide bond and a carbonic acid ester bond,
* and ** represent a bonding site, and * represent a bonding site to carbon atoms to which R^{a7} is bonded,
L^{a71} and L^{a72} each independently represent a hydrocarbon group having 1 to 24 carbon atoms which may have a substituent, and —CH₂— included in the hydrocarbon group may be replaced by —O—, —S—, —CO— or —SO₂—,
X^{a72} represents ***—CO—O—, ***—O—CO—, ***—O—CO—O—, *** —O—, and *** represents a bonding site to L^{a71},
na7 represents an integer of 0 to 2, and when na7 is 2, a plurality of groups in parentheses may be the same or different from each other,
RA⁻ represents a sulfonate anion or a carboxyl anion, and
ZA⁺ represents an organic cation.

Examples of the alkyl group having 1 to 6 carbon atoms in R^{a7} and R^{a71} include a methyl group, an ethyl group, an n-propyl group, an isopropyl group, an n-butyl group, a sec-butyl group, a tert-butyl group, a pentyl group and a hexyl group.

Examples of the halogen atom in R^{a7} include a fluorine atom, a chlorine atom, a bromine atom and an iodine atom.

Examples of the alkyl group having 1 to 6 carbon atoms which has a halogen atom in R^{a7} include a trifluoromethyl group, a difluoromethyl group, a perfluoroethyl group, a 2,2,2-trifluoroethyl group, a 1,1,2,2-tetrafluoroethyl group, a perfluoropropyl group, a 2,2,3,3,3-pentafluoropropyl group, a perfluorobutyl group, a 1,1,2,2,3,3,4,4-octafluorobutyl group, a perfluoropentyl group, a 2,2,3,3,4,4,5,5,5-nonafluoropentyl group, a perfluorohexyl group, a trichloromethyl group, a dichloromethyl group, a triiodomethyl group, a diiodo methyl group, a tribromomethyl group, a dibromo methyl group and the like.

R^{a7} is preferably a hydrogen atom or an alkyl group having 1 to 4 carbon atoms, more preferably a hydrogen atom or an alkyl group having 1 to 3 carbon atoms, still more preferably a hydrogen atom, a methyl group or an ethyl group, and yet more preferably a hydrogen atom or a methyl group.

When X^{a71} is a group represented by *-Ax-Ph-Ay-**, preferred is a linking group represented by the following formula (X¹⁰):

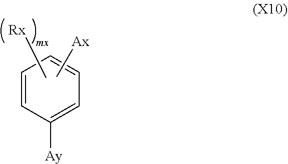

wherein, in formula (X¹⁰),
Ax represents bond species bonded to carbon atoms to which R^{a7} is bonded, and represents one bond species selected from the group consisting of a single bond, an ether bond, a thioether bond, an ester bond, a carbonic acid ester bond and an amide bond,
Ay represents bond species to which L^{a71} is bonded, and represents one bond species selected from the group consisting of a single bond, an ether bond, a thioether bond, an ester bond, a carbonic acid ester bond and an amide bond,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms, and
mx represents an integer of 0 to 4, and when mx is an integer of 2 or more, a plurality of Rx may be the same or different from each other.

When one of Ax and Ay is a single bond, the other is preferably one selected from the group consisting of an ether bond, a thioether bond, an ester bond, a carbonic acid ester bond and an amide bond.

When either Ax or Ay is an amide bond, a bond represented by —CO—NR^{a71}—is preferable.

The bonding site of Ay in the phenylene group is preferably the m-position or the p-position, and more preferably the p-position, with respect to the bonding site of Ax.

Particularly, Rx is preferably a fluorine atom, an iodine atom, a trifluoromethyl group, a methyl group or an ethyl group,

mx is preferably 0, 1 or 2.

Examples of X^{a71} include a single bond and groups represented by the following formula (X⁰-1) to formula (X⁰-10). * represents a bonding site to carbon atoms to which —R^{a7} is bonded. ** represents a bonding site to L^{a71}. X²⁰ represents —O— or —NR^{a71}—

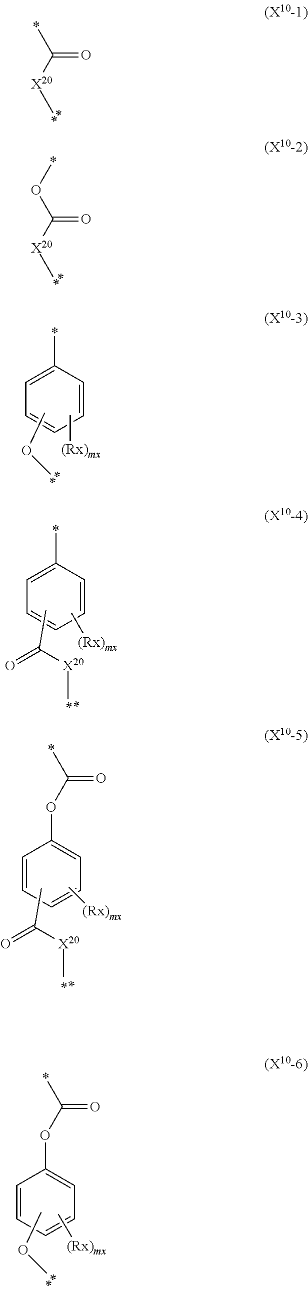

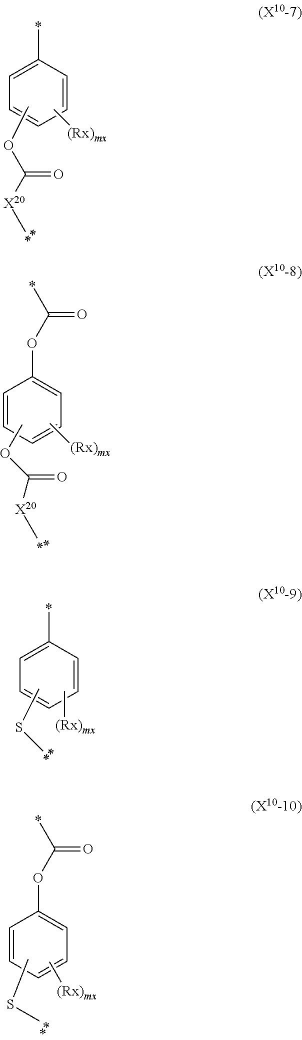

Specific examples of the groups represented by formula (X¹⁰-1) to formula (X¹⁰-10) include the following groups.

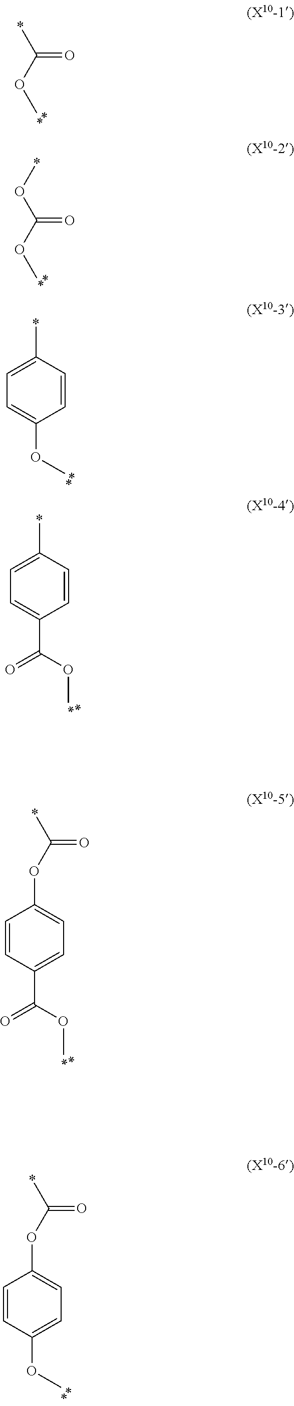

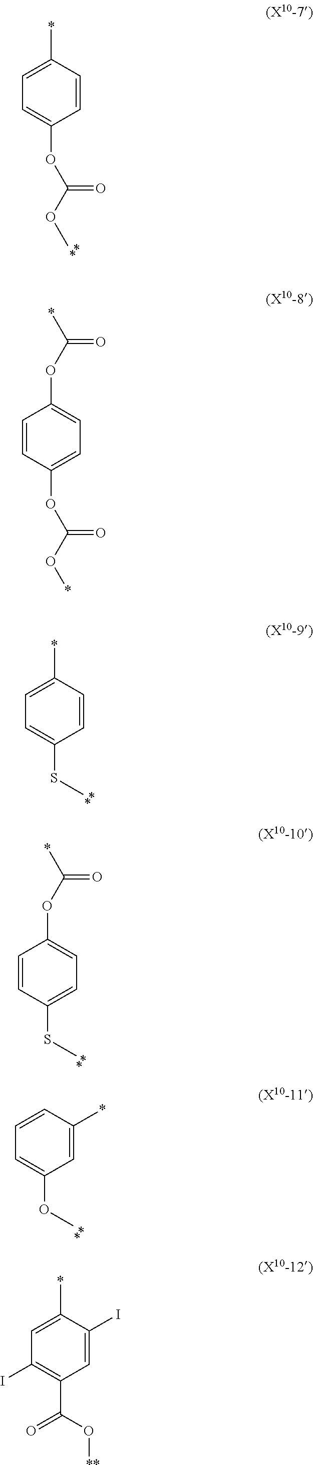

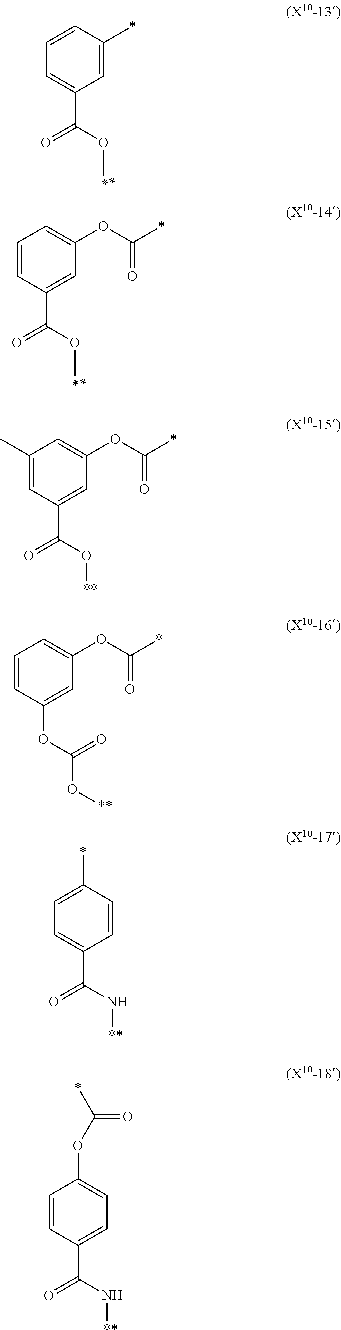

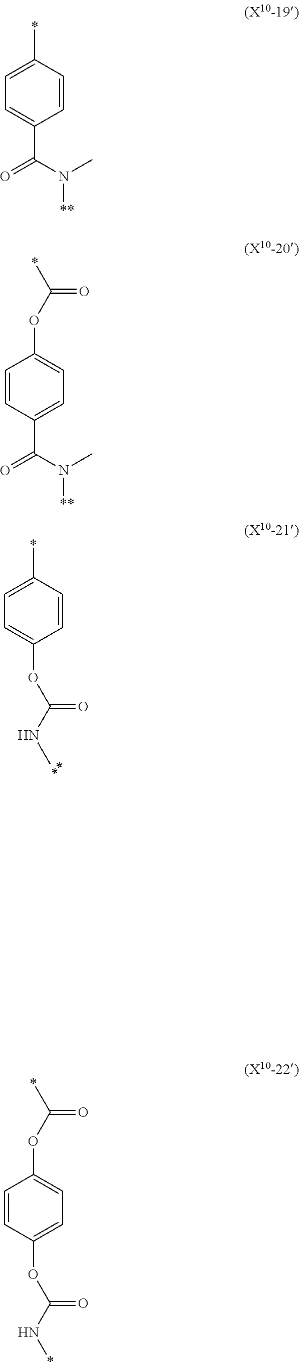

Particularly, X^{a7′} is preferably a single bond and a group represented by any one of formula (X¹⁰-1′) and formula (X¹⁰-3′) to formula (X¹⁰-9′), more preferably a single bond or a group represented by any one of formula (X¹⁰-1′), formula (X¹⁰-4′), formula (X¹⁰-5′), formula (X¹⁰-6′) and formula (X¹⁰-9′), and still more preferably a single bond, a group represented by formula (X¹⁰-1′), a group represented by formula (X¹⁰-5′) or a group represented by formula (X¹⁰-6′) or formula (X¹⁰-9′).

Examples of the hydrocarbon group as for L^{a7} include groups which are obtained by removing one hydrogen atom from a chain hydrocarbon group, an alicyclic hydrocarbon group, an aromatic hydrocarbon group, and a group formed by combining two or more of these groups, and bonding to X^{a71} and X^{a72}. Examples of the hydrocarbon group as for L^{a72} include groups which are obtained by removing one hydrogen atom from a chain hydrocarbon group, an alicyclic hydrocarbon group, an aromatic hydrocarbon group, and a group formed by combining two or more of these groups, and bonding X^{a71} and RA⁻.

Examples of the chain hydrocarbon group as for L^{a71} and L^{a72} include groups obtained by removing one hydrogen atom of an alkyl group or an alkenyl group. The alkyl group may be either linear or branched, and specific examples thereof include a methyl group, an ethyl group, an n-propyl group, an iso-propyl group, an n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentanedecyl group, a heptadecyl group and the like. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group, a nonenyl group and the like.

The number of carbon atoms of the chain hydrocarbon group is preferably 1 to 20, more preferably 1 to 18, still more preferably 1 to 10 carbon atoms.

Examples of the alicyclic hydrocarbon group as for L^{a71} and L^{a72} include groups obtained by removing one hydrogen atom of a monocyclic or polycyclic cycloalkyl group. Examples of the monocyclic cycloalkyl group include a cyclobutyl group, a cycloheptyl group, a cyclohexyl group, a cyclopentyl group, a cyclooctyl group and the like.

Examples of the polycyclic cycloalkyl group as for L^{a71} and L^{a72} include a cycloalkyl group having a crosslinked structure, a cycloalkyl group in which two or more rings are fused, or a cycloalkyl group in which two rings are bonded by spiro bonding. Examples of the cycloalkyl group having a crosslinked structure include a norbornyl group, an adamantyl group and the like. Examples of the cycloalkyl group in which two or more rings are fused include a bicyclo[4,4,0]decane group, a steroid group (steroid skeleton) and the like. Examples of the cycloalkyl group in which two rings are bonded by spiro bonding include a spirocyclic cycloalkyl group in which one cycloalkyl group selected from the group consisting of a cyclopentyl group, a cyclohexyl group, a norbornyl group and an adamantyl group, and a cycloalkyl group having 5 to 8 carbon atoms are bonded by spiro bonding, and the like. A double bond may be formed between two carbon atoms included in the alicyclic hydrocarbon group. More specifically, alicyclic hydrocarbon groups represented by the following formulas are exemplified.

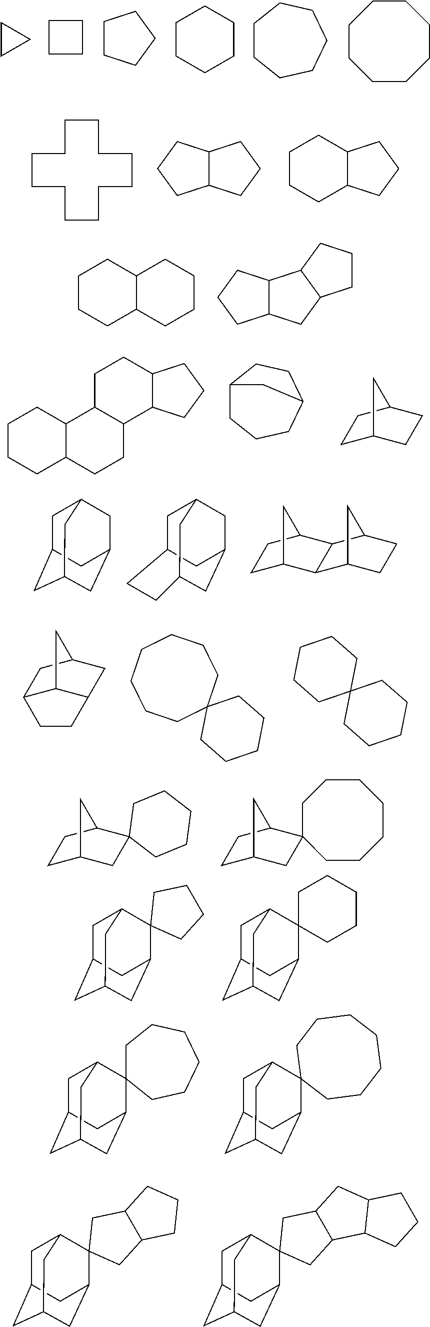

When the alicyclic hydrocarbon group is a monocyclic cycloalkyl group, the number of carbon atoms of the alicyclic hydrocarbon group is preferably 3 to 18, more preferably 3 to 12, and still more preferably 3 to 8. When the alicyclic hydrocarbon group is a polycyclic cycloalkyl group, the number of carbon atoms of the alicyclic hydrocarbon group is preferably 6 to 18, and more preferably 7 to 12.

Examples of the aromatic hydrocarbon group L^{a71} and L^{a72} include groups obtained by removing one hydrogen atom of an aryl group. Examples of the aryl group include a phenyl group, a naphthyl group, an anthryl group, a biphenyl group, a fluorenyl group and the like. The number of carbon atoms of the aromatic hydrocarbon group is preferably 5 to 14, more preferably 6 to 14, and still more preferably 6 to 10.

When —CH₂— included in the hydrocarbon group as for L^{a71} and L^{a72} is replaced by —O—, —CO—, —S— or —SO₂—, the number of carbon atoms before replacement is taken as the total number of the hydrocarbon group.

Of the hydrocarbon groups as for L^{a71} and L^{a72}, examples of the group in which —CH₂— included in the chain hydrocarbon group is replaced by —O—, —CO—, —S— or —SO₂— include a hydroxy group (a group in which —CH₂— included in the methyl group is replaced by —O—), a carboxy group (a group in which —CH₂—CH₂— included in the ethyl group is replaced by —O—CO—), a thiol group (a group in which —CH₂— included in the methyl group is replaced by —S—), a carbonyl group (a group in which —CH₂— included in the methylene group is replaced by —CO—), an oxy group (a group in which —CH₂— included in the methylene group is replaced by —O—), a thio group (a group in which —CH₂— included in the methylene group is replaced by —S—), a sulfonyl group (a group in which —CH₂— included in the methylene group is replaced by —SO₂—), an alkoxy group (a group in which —CH₂— at any position included in the alkyl group is replaced by —O—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—) and the like. Examples of these replaced groups include the same groups as mentioned herein as long as the upper limit of the number of carbon atoms permits.

Of the hydrocarbon groups as for L^{a71} and L^{a72}, examples of the group in which —CH₂— included in the alicyclic hydrocarbon group is replaced by —O—, —CO—, —S— or —SO₂— include groups having a structure such as cyclic ether, cyclic ketone, cyclic ester (lactone), cyclic thioether, cyclic acetal or cyclic sulfonic acid ester (sultone). Specific examples thereof include alicyclic hydrocarbon groups represented by the following formulas. The bonding site of the alicyclic hydrocarbon group represented by the following formulas can be any position.

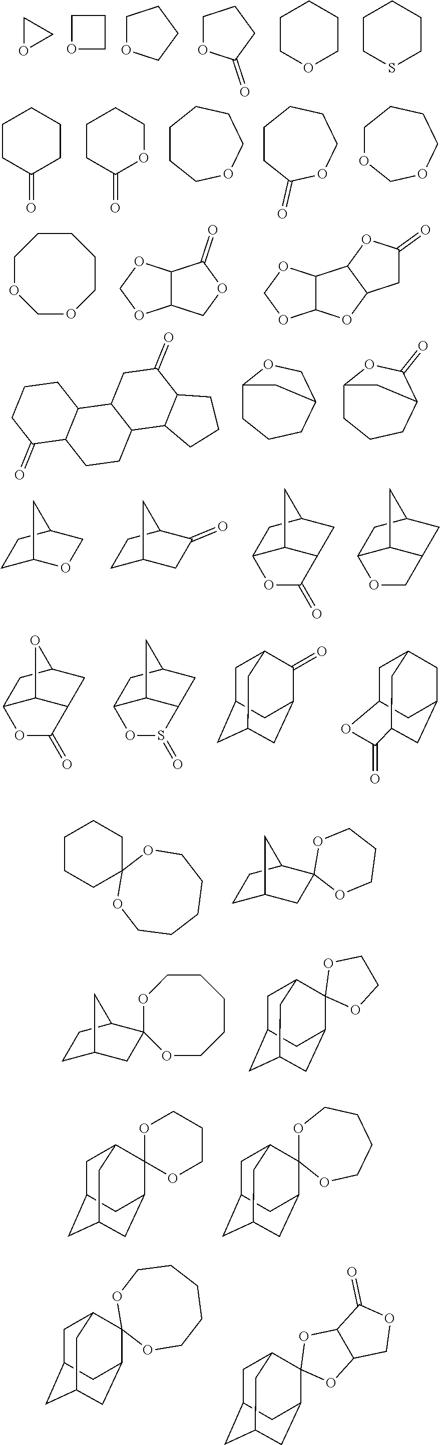

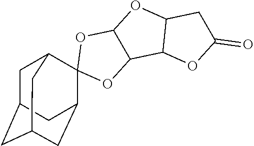 [SMILES: O=C1CC2OC3OC4(OC3C2O1)C1CC2CC(C1)CC4C2]

Of the hydrocarbon groups as for L^{a71} and L^{a72}, —CH₂— included in the aromatic hydrocarbon group may be replaced by —O— or —S—, and examples of the group in which —CH₂— is replaced by —O— or —S— include groups derived from a furan ring or a thiophene ring, respectively.

Examples of the groups obtained by combining two or more groups of the chain hydrocarbon group, the alicyclic hydrocarbon group and the aromatic hydrocarbon group as for L^{a71} and L^{a72} include a group obtained by combining the chain hydrocarbon group with the alicyclic hydrocarbon group, a group obtained by combining the chain hydrocarbon group with the aromatic hydrocarbon group, a group obtained by combining the alicyclic hydrocarbon group with the aromatic hydrocarbon group, a group obtained by combining the chain hydrocarbon group, the alicyclic hydrocarbon group and the aromatic hydrocarbon group. The group obtained by combining the alicyclic hydrocarbon group with the aromatic hydrocarbon group may also be a fused ring.

Examples of the substituent which may be possessed by the hydrocarbon group as for L^{a71} and L^{a72} include a halogen atom, a cyano group and a nitro group. Examples of the halogen atom include a fluorine atom, a chlorine atom, a bromine atom, an iodine atom and the like.

When L^{a71} and L^{a72} are groups obtained by combining an alicyclic hydrocarbon group or an aromatic hydrocarbon group with a chain hydrocarbon group, the chain hydrocarbon group may be substantially regarded as a substituent which is possessed by the alicyclic hydrocarbon group or the aromatic hydrocarbon group. By replacing —CH₂— of the chain hydrocarbon group included in the hydrocarbon group by —O—, —CO—, —S— or —SO₂—as for L^{a71} and L^{a72}, the hydrocarbon group as for L^{a71} and L^{a72} can substantially have a substituent such as a hydroxy group, a carboxy group, an alkoxy group, an alkylcarbonyl group, an alkoxycarbonyl group, an alkylcarbonyloxy group, a thiol group or a sulfonyl group.

na7 is preferably 0 or 1.

Examples of ZA⁺ in formula (a7-A) include the same cations as in the salt represented by formula (B1) or the like.

In formula (a7-A), when the hydrocarbon group as for L^{a71} and L^{a72} is a saturated hydrocarbon group, it is possible to include the same groups as mentioned as divalent linking group as for A^{b7} in formula (a7-B) mentioned below.

Examples of the structural unit represented by formula (a7-A) also include a structural unit or the like represented by formula (a7-A1)

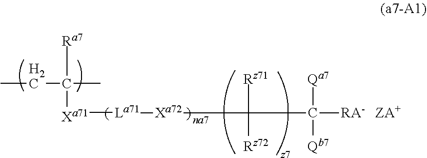

wherein, in formula (a7-A1),
R^{a7}, X^{a71}, L^{a71}, X^{a72}, na7, RA⁻ and ZA⁺ are the same as defined above,
z7 represents an integer of 0 to 6, and
Q^{a7}, Q^{b7}, R^{z71} and R^{z72} each independently represent a hydrogen atom, a fluorine atom, a perfluoroalkyl group having 1 to 6 carbon atoms or an alkyl group having 1 to 6 carbon atoms, and when z7 is 2 or more, a plurality of R^{z71} and R^{z72} may be the same or different from each other.

Examples of the perfluoroalkyl group having 1 to 6 carbon atoms or alkyl group having 1 to 6 carbon atoms in Q^{a7}, Q^{b7}, R^{z71} and R^{z72} include the same groups as mentioned as for R^{a7}.

Examples of the structural unit represented by formula (a7-A) include the following structural units, structural units in which the group corresponding to the methyl group as for R^{a7} is substituted with a hydrogen atom, a halogen atom (e.g., a fluorine atom) or an alkyl group having 1 to 6 carbon atoms which may have a halogen atom (e.g., a trifluoromethyl group, etc.), and structural units mentioned in W0 2012/050015 A. ZA⁺ represents an organic cation.

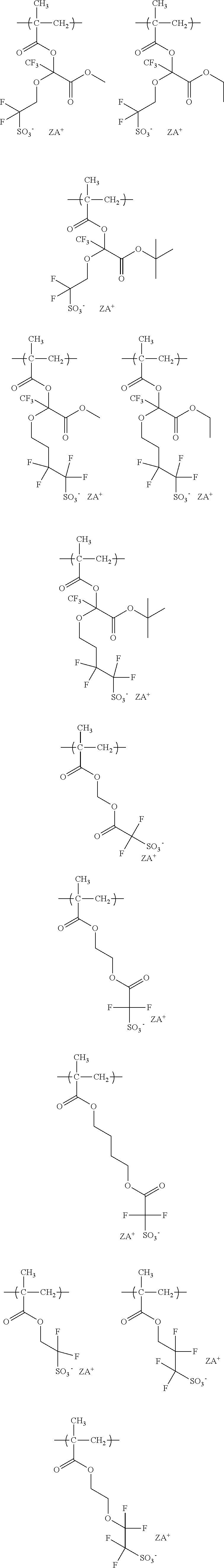

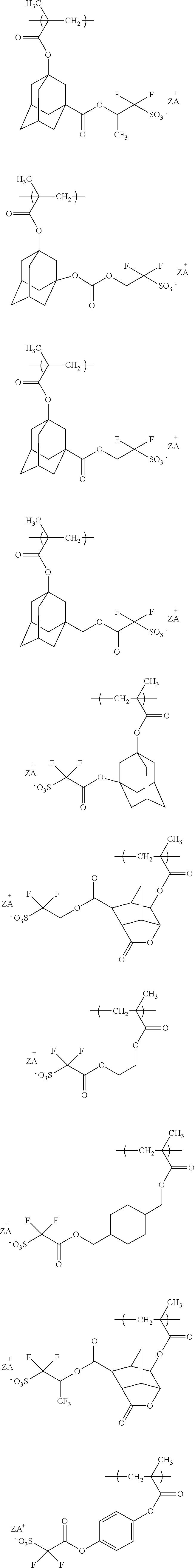

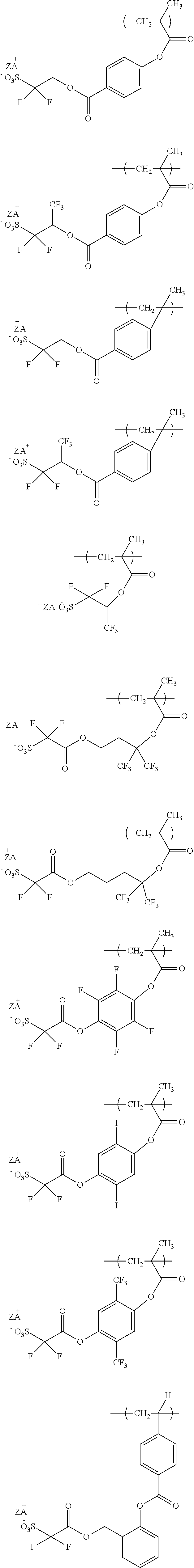

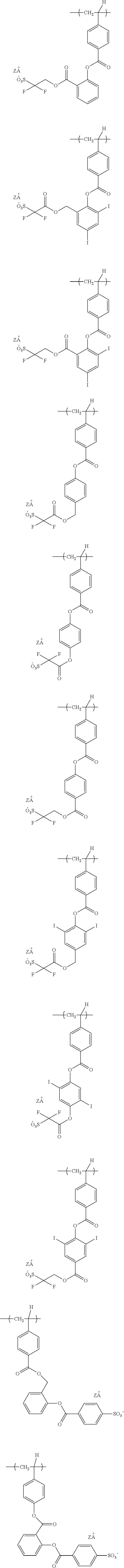

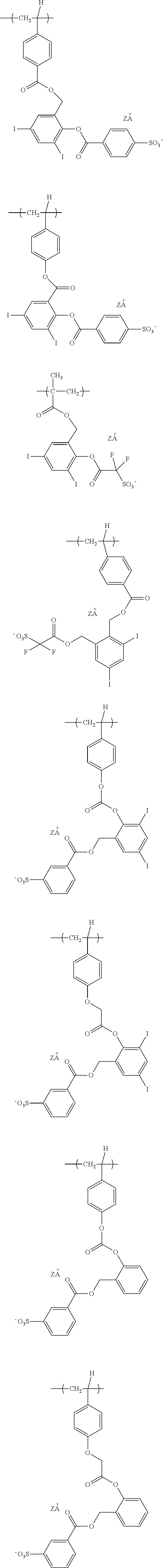

The structural unit having a sulfonio group and an organic anion in the side chain is preferably a structural unit represented by formula (a7-B):

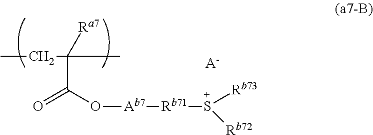

wherein, in formula (a7-B),
R^{a7} represents a hydrogen atom, a halogen atom, or an alkyl group having 1 to 6 carbon atoms which may have a halogen atom,
A^{b7} represents a single bond or a divalent linking group,
R^{b71} represents a divalent aromatic hydrocarbon group having 6 to 18 carbon atoms which may have a substituent,
R^{b72} and R^{b73} each independently represent a hydrocarbon group having 1 to 18 carbon atoms which may have a substituent, R^{b72} and R^{b73} may be bonded to each other to form a ring together with sulfur atoms to which R^{b72} and R^{b73} are bonded, and
A⁻ represents an organic anion.

Examples of the halogen atom and the alkyl group which may have a halogen atom as for R^{a7} include the same halogen atoms and the alkyl groups which may have a halogen atom of formula (a7-A).

Examples of the divalent aromatic hydrocarbon group having 6 to 18 carbon atoms represented by R^{b71} include a phenylene group and a naphthylene group.

Examples of the hydrocarbon group represented by R^{b72} and R^{b73} include an alkyl group, an alicyclic hydrocarbon group, an aromatic hydrocarbon group, and groups formed by combining these groups.

Examples of the alkyl group, the alicyclic hydrocarbon group, the aromatic hydrocarbon group, and the groups formed by combining these groups include the same groups as mentioned above.

Examples of the divalent linking group represented by A^{b7} include a divalent saturated hydrocarbon group having 1 to 18 carbon atoms, and —CH₂— included in the divalent saturated hydrocarbon group may be replaced by —O—, —S— or —CO—.

Examples of the divalent saturated hydrocarbon group include divalent chain saturated hydrocarbon groups such as a linear or branched alkanediyl group, a monocyclic or polycyclic divalent alicyclic saturated hydrocarbon group, or a combination thereof.

Specific examples thereof include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; branched alkanediyl groups such as a butane-1,3-diyl group, a 2-methylpropane-1,3-diyl group, a 2-methylpropane-1,2-diyl group, a pentane-1,4-diyl group and a 2-methylbutane-1,4-diyl group; divalent monocyclic alicyclic saturated hydrocarbon groups which are monocyclic cycloalkanediyl groups, such as a cyclobutane-1,3-diyl group, a cyclopentane-1,3-diyl group, a cyclohexane-1,4-diyl group and a cyclooctane-1,5-diyl group; and divalent polycyclic alicyclic saturated hydrocarbon groups which are polycyclic cycloalkanediyl groups, such as a norbornane-1,4-diyl group, a norbornane-2,5-diyl group, an adamantane-1,5-diyl group and an adamantane-2,6-diyl group.

Those in which —CH₂— included in the saturated hydrocarbon group are replaced by —O—, —S— or —CO— include, for example, the following divalent groups. Before replacing —CH₂— included in the saturated hydrocarbon group by —O—, —S— or —CO—, the number of carbon atoms is 17 or less. In the following formulas, * and ** represent a bonding site, and * represents a bonding site to R^{b71}.

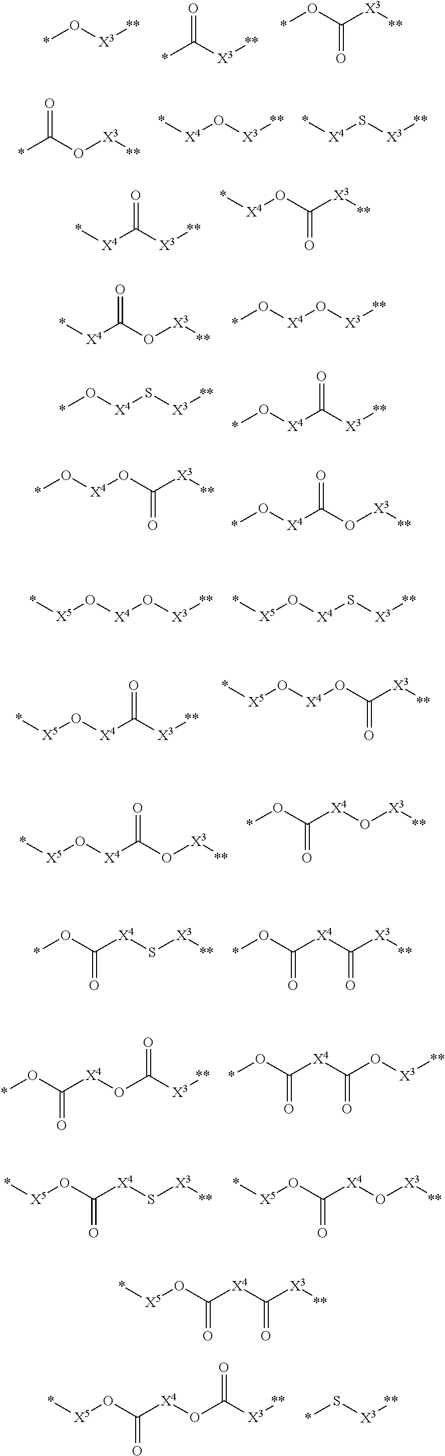

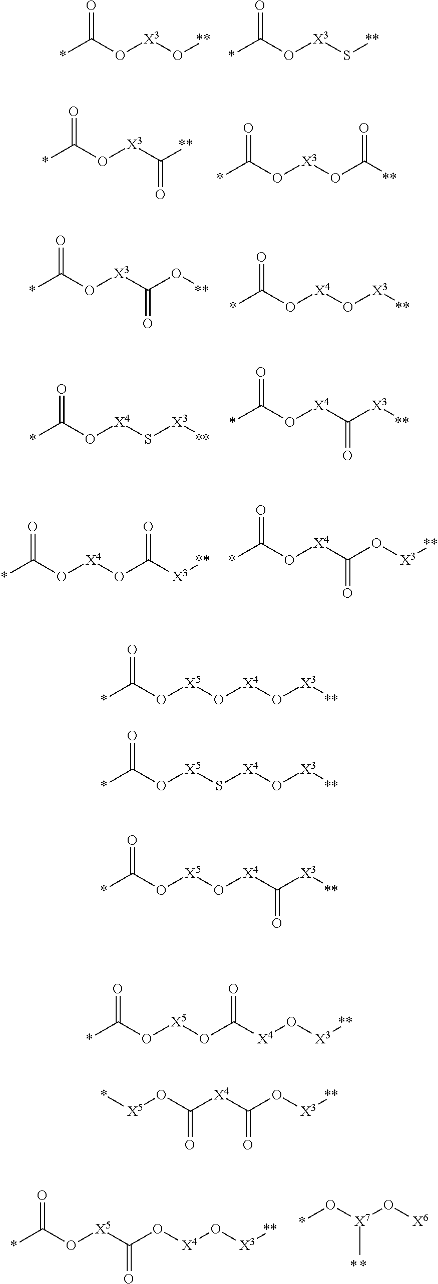

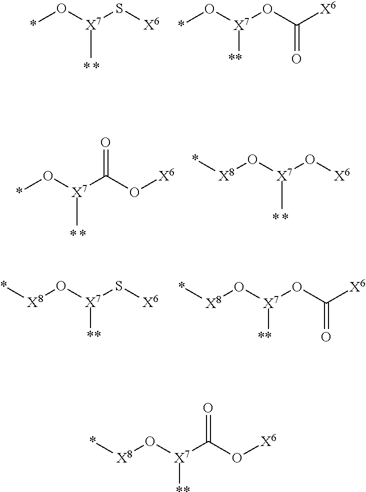

X³ represents a divalent saturated hydrocarbon group having 1 to 16 carbon atoms.

X⁴ represents a divalent saturated hydrocarbon group having 1 to 15 carbon atoms.

X⁵ represents a divalent saturated hydrocarbon group having 1 to 13 carbon atoms.

X⁶ represents a divalent saturated hydrocarbon group having 1 to 14 carbon atoms.

X⁷ represents a trivalent saturated hydrocarbon group having 1 to 14 carbon atoms.

X⁸ represents a divalent saturated hydrocarbon group having 1 to 13 carbon atoms.

Examples of the structural unit including a cation in formula (a7-B) include the following structural units and structural units in which a group corresponding to a methyl group of R^{a7} is substituted with a hydrogen atom, a halogen atom (e.g., a fluorine atom, etc.) or an alkyl group having 1 to 6 carbon atoms which may have a halogen atom (e.g., a trifluoromethyl group, etc.) and the like.

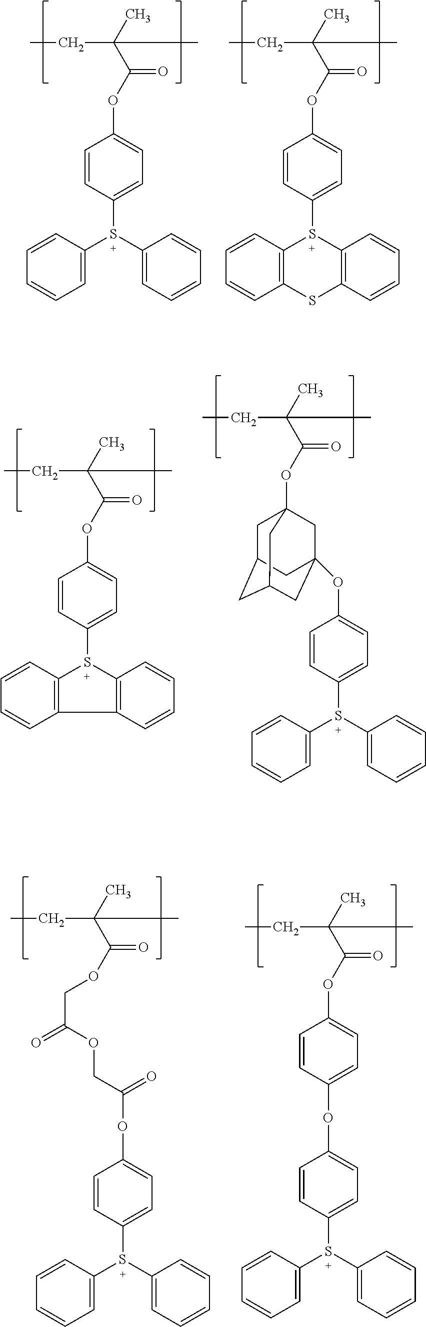

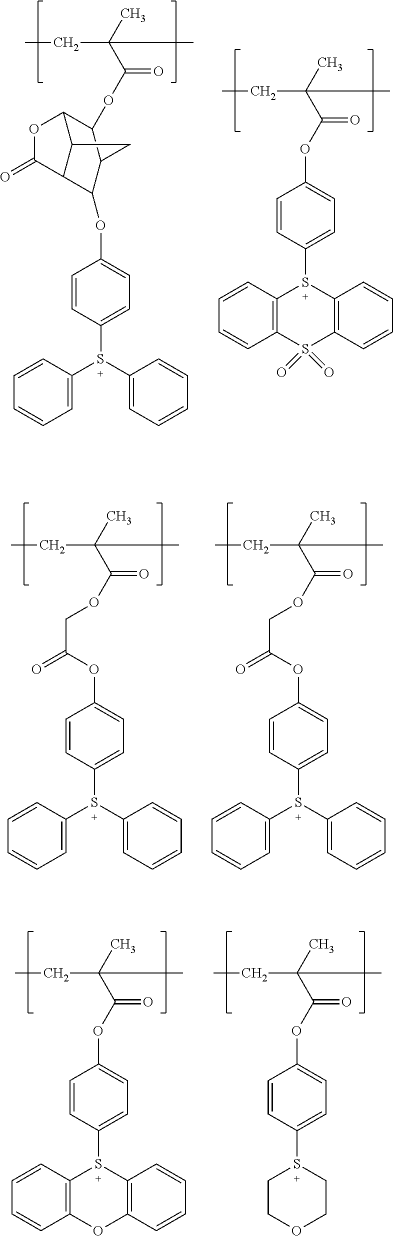

Examples of the organic anion represented by A⁻ include a sulfonic acid anion, a sulfonylimide anion, a sulfonylmethide anion and a carboxylic acid anion. The organic anion represented by A⁻ is preferably a sulfonic acid anion. The sulfonic acid anion is more preferably those which are the same as anions to be mentioned in the acid generator (B). Examples of the sulfonic acid anion, the sulfonylimide anion, the sulfonylmethide anion and the carboxylic acid anion include those which are the same as anions to be mentioned in the acid generator (B).

Examples of the structural unit represented by formula (a7-B) include the followings.

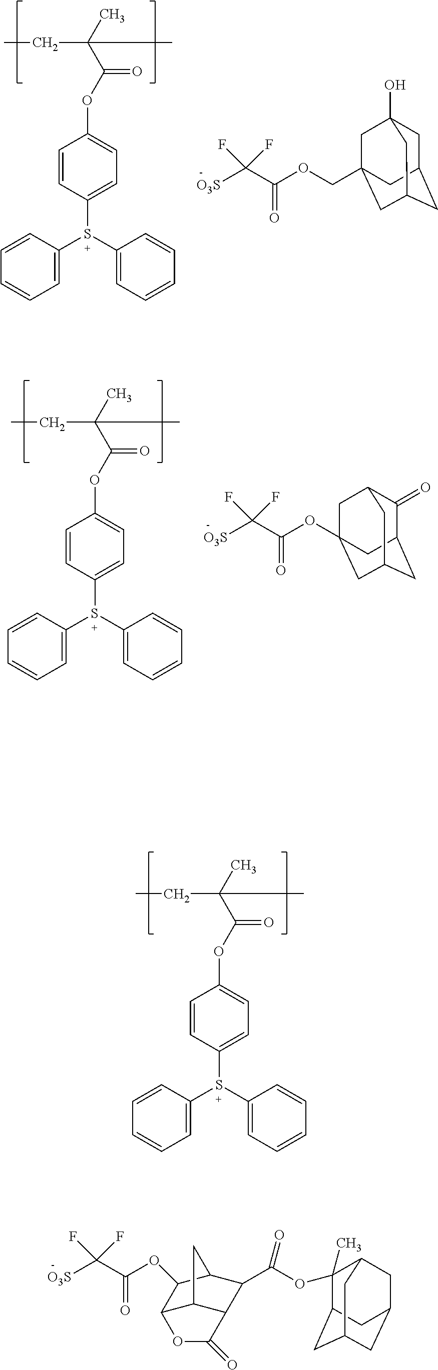

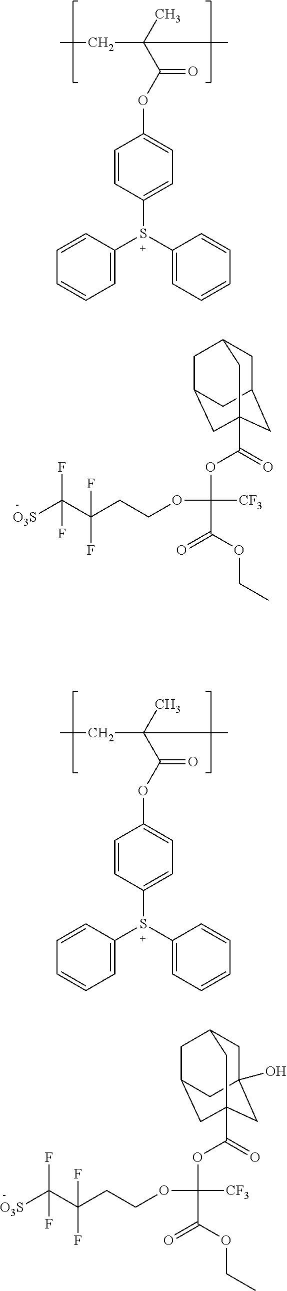

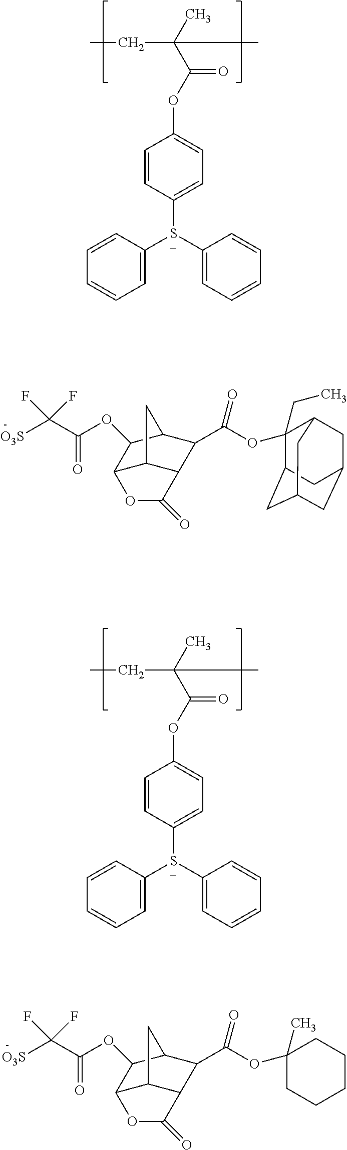

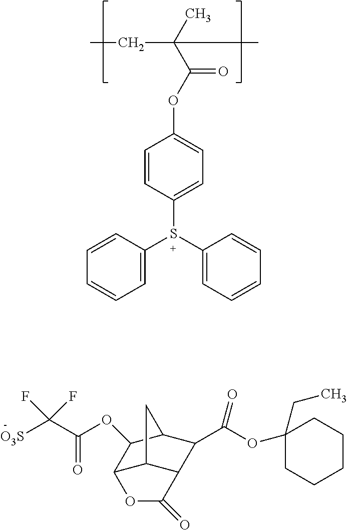

When the resin (A) or the like includes a structural unit (a7), the structural unit (a7) may be included alone, or two or more structural units may be included. The total content of the structural unit (a7) is preferably 1 to 30 mol %, more preferably 1 to 25 mol %, still more preferably 2 to 20 mol %, yet more preferably still more preferably 3 to 15 mol %, and further preferably 3 to 10 mol %, based on all structural units of the resin (A) or the like.

The resin (A) or the like may include a structural unit other than the above-mentioned structural units, and examples of such structural unit include structural units known in the relevant technical field.

The resin (A) or the like is preferably a resin including a structural unit (a1). Particularly, the resin (Ap) is more preferably a resin composed of a structural unit (IP), a structural unit (a1) and a structural unit (s), that is, a copolymer of a carboxylate (I), a monomer (a1) and a monomer (s). The resin (A) including no structural unit (IP) is preferably a resin composed of a structural unit (a1) and a structural unit (s), that is, a copolymer of a monomer (a1) and a monomer (s).

The structural unit (a1) is preferably at least one selected from the group consisting of a structural unit (a1-0), a structural unit (a1-1), a structural unit (a1-2) (preferably the structural unit having a cyclohexyl group or a cyclopentyl group), a structural unit (a1-4), a structural unit (a1-5) and a structural unit (a1-6), more preferably at least two, and still more preferably at least two selected from the group consisting of a structural unit (a1-1) and a structural unit (a1-2).

The structural unit (s) is preferably at least one selected from the group consisting of a structural unit (a2) and a structural unit (a3). The structural unit (a2) is preferably a structural unit (a2-C) or a structural unit (a2-A). The structural unit (a3) is preferably at least one selected from the group consisting of a structural unit represented by formula (a3-1), a structural unit represented by formula (a3-2) and a structural unit represented by formula (a3-4).

The respective structural units constituting the resin (A) or the like may be used alone, or two or more structural units may be used in combination. Using a monomer from which these structural units are derived, it is possible to produce by a known polymerization method (e.g., radical polymerization method). The content of the respective structural units included in the resin (A) or the like can be adjusted according to the amount of the monomer used in the polymerization.

The weight-average molecular weight of the resin (A) and the resin (Ap) is preferably 2,000 or more (more preferably 2,500 or more, and still more preferably 3,000 or more), and 50,000 or less (more preferably 30,000 or less, and still more preferably 15,000 or less), and an oligomer having a weight-average molecular weight of less than the above value may be used. As used herein, the weight-average molecular weight is a value determined by gel permeation chromatography under the analysis conditions mentioned in Examples. The structural unit (IP) may constitute a dimer, a trimer, and a compound having a weight-average molecular weight of less than 2,000, or an oligomer.

### <Carboxylic Acid Generator>

The carboxylic acid generator of the present invention is a carboxylic acid generator including a carboxylate (I) or a structural unit (IP) of the present invention. The structural unit (IP) can be included as a compound or a resin obtained by polymerizing a plurality thereof. The carboxylic acid generator may include the carboxylate (I) alone, or two or more thereof. The compound or resin including the structural unit (IP) may be used alone or in combination of two or more thereof. The carboxylic acid generator of the present invention may include both the carboxylate (I) and the structural unit (IP).

The carboxylic acid generator of the present invention may further include an acid generator known in the resist field other than the carboxylate (I) (hereinafter sometimes referred to as “acid generator (B)” or “compound (B)”) and/or a carboxylic acid generator known in the resist field other than the carboxylate (I).

When the carboxylic acid generator includes the acid generator (B), a ratio of the content of the carboxylate (I) to that of the acid generator (B) (mass ratio; carboxylate (I):acid generator (B)) is usually 1:99 to 100:0, preferably 1:99 to 99:1, more preferably 2:98 to 98:2, still more preferably 5:95 to 95:5, yet more preferably 10:90 to 90:10, further preferably 15:85 to 85:15, and particularly preferably 40:60 to 60:40.

In the resist composition of the present invention, the content of the carboxylate (I) of the present invention is preferably about 0.001 to 15% by mass, more preferably about 0.001 to 10% by mass, still more preferably about 0.001 to 8% by mass, and yet more preferably about 0.005 to 7% by mass, based on the amount of the solid component of the resist composition.

In the resist composition of the present invention, the content of the resin (Ap) of the present invention is preferably 80% by mass or more and 99% by mass or less, and more preferably 90% by mass or more and 99% by mass or less, relative to the solid component of the resist composition.

### [Resist Composition]

The resist composition of the present invention includes the carboxylic acid generator of the present invention. The carboxylic acid generator here may be either a carboxylate (I) or a resin (Ap) including a structural unit (IP). The resist composition of the present invention may include, in addition to the carboxylic acid generator of the present invention, a resin. The resin may be either a resin including a structural unit (a1) having an acid-labile group, or a resin including no structural unit (a1). However, the resist composition of the present invention includes at least one of the carboxylate (I) and the structural unit (IP), and may include both of them. That is, the resist composition of the present invention may include a carboxylic acid generator including the structural unit (IP) of the present invention or the carboxylate (I) of the present invention. The structural unit (IP) may be in a form of either compound or resin. In other words, the resist composition of the present invention may include, as the carboxylic acid generator, a resin (Ap) and/or a resin (A), and a carboxylate (I). The resist composition of the present invention preferably include a resin including a structural unit (a1) having an acid-labile group. That is, the resist composition preferably includes at least:
(a) a carboxylate (I) and a resin (A) including a structural unit (a1) having an acid-labile group,
(b) a resin (Ap) including a structural unit (IP) and a structural unit (a1) having an acid-labile group, or
(c) a resin (Ap) including a structural unit (IP) and a resin (A) including a structural unit (a1) having an acid-labile group.

Of these, the resist composition (b) is preferable. Two or more resins (A) and/or resins (Ap) may be included.

It is preferable that the resist composition of the present invention further includes an acid generator other than the carboxylic acid generator (hereinafter sometimes referred to as “acid generator (B)” or “compound (B)”), a quencher (hereinafter sometimes referred to as “quencher (C)”) and/or a solvent (hereinafter sometimes referred to as “solvent (E)”). The resist composition of the present invention may further include a resin other than the resin (A) or the like mentioned above.

<Resin Other than Resin (A) or the Like>

The resist composition of the present invention may include a resin other than the resin (Ap) and the resin (A). Examples of the resin other than the resin (Ap) and the resin (A) include a resin (AX) including the same structural unit as that of the resin (A), except that no structural unit (a1) is included in the above-mentioned resin (A), a resin including a structural unit (a4) and/or a structural unit (a5) (including neither structural unit (IP) nor structural unit (a1), hereinafter sometimes referred to as “resin (X)”) and the like.

Examples of the resin (AX) include a resin including a structural unit (a2), and a resin including a structural unit (a2-A) is preferable. In the resin (AX), the content of the structural unit (a2-A) is preferably 5 mol % or more, more preferably 10 mol % or more, and still more preferably 15 mol % or more, and is preferably 80 mol % or less, and more preferably 70 mol % or less, based on the total of all structural units of the resin (AX).

Examples of the structural unit, which may be further included in the resin (X), include a structural unit (a2), a structural unit (a3) and structural units derived from other known monomers. Particularly, the resin (X) is preferably a resin composed only of a structural unit (a4) and/or a structural unit (a5), and more preferably a resin composed only of a structural unit (a4).

When the resin (X) includes a structural unit (a4), the content is usually 20 mol % or more, preferably 30 mol % or more, more preferably 40 mol % or more, and still more preferably 45 mol % or more, based on all structural units of the resin (X). The content is usually 100 mol % or less, preferably 80 mol % or less, more preferably 70 mol % or less, still more preferably 60 mol % or less, and yet more preferably 55 mol % or less, based on all structural units of the resin (X). Specifically, the content is usually 20 to 100 mol %, preferably 20 to 80 mol %, more preferably 30 to 70 mol %, still more preferably 40 to 60 mol %, and yet more preferably 45 to 55 mol %, based on all structural units of the resin (X). When the resin (X) includes a structural unit (a5), the content is usually 20 mol % or more, preferably 30 mol % or more, more preferably 40 mol % or more, and still more preferably 45 mol % or more, based on all structural units of the resin (X). The content is usually 100 mol % or less, preferably 80 mol % or less, more preferably 70 mol % or less, still more preferably 60 mol % or less, and yet more preferably 55 mol % or less, based on all structural units of the resin (X). Specifically, the content is usually 20 to 100 mol %, preferably 20 to 80 mol %, more preferably 30 to 70 mol %, still more preferably 40 to 60 mol %, and yet more preferably 45 to 55 mol %, based on all structural units of the resin (X). When the resin (X) includes a structural unit (a4) and a structural unit (a5), the total content is usually mol % or more, preferably 60 mol % or more, more preferably 70 mol % or more, and still more preferably 80 mol % or more, based on all structural units of the resin (X). The total content is usually 100 mol % or less, based on all structural units of the resin (X). Specifically, the total content is usually 40 to 100 mol %, preferably 60 to 100 mol %, more preferably 70 to 100 mol %, and still more preferably 80 to 100 mol %, based on all structural units of the resin (X).

The respective structural unit constituting the resin (AX) and the resin (X) may be used alone, or two or more structural units may be used in combination. Using a monomer from which these structural units are derived, it is possible to produce by a known polymerization method (e.g. radical polymerization method). The content of the respective structural units included in the resin (AX) and the resin (X) can be adjusted according to the amount of the monomer used in the polymerization.

The weight-average molecular weight of the resin (AX) and the resin (X) is preferably 6,000 or more (more preferably 7,000 or more) and 80,000 or less (more preferably 60,000 or less), and an oligomer having a weight-average molecular weight of less than the above range may be used. The measurement means of the weight-average molecular weight of the resin (AX) and the resin (X) is the same as in the case of the resin (A) or the like.

When the resist composition of the present invention includes the resin (X), the content is preferably 1 to 60 parts by mass, more preferably 1 to 50 parts by mass, still more preferably 1 to 40 parts by mass, yet more preferably 1 to 30 parts by mass, and further preferably 1 to 8 parts by mass, based on 100 parts by mass of the total of the resin (A) or the like.

The content of the resin (A) or the like in the resist composition is preferably 80% by mass or more and 99% by mass or less, and more preferably 90% by mass or more and 99% by mass or less, based on the solid content of the resist composition. The content of the resin (Ap) is preferably 80% by mass or more and 99% by mass or less, and more preferably 90% by mass or more and 99% by mass or less, based on the solid content of the resist composition. When including resins other than the resin (A) or the like, the total content of the resin (A) or the like and resins other than the resin (A) or the like is preferably 80% by mass or more and 99% by mass or less, and more preferably 90% by mass or more and 99% by mass or less, based on the solid content of the resist composition. The solid content of the resist composition and the content of the resin thereto can be measured by a known analysis means such as liquid chromatography or gas chromatography.

### <Acid Generator>

Either nonionic or ionic acid generator may be used as the acid generator (B). Examples of the nonionic acid generator include sulfonate esters (e.g., 2-nitrobenzyl ester, aromatic sulfonate, oxime sulfonate, N-sulfonyloxyimide, sulfonyloxyketone, diazonaphthoquinone 4-sulfonate), sulfones (e.g., disulfone, ketosulfone, sulfonyldiazomethane) and the like. Typical examples of the ionic acid generator include onium salts containing an onium cation (e.g., diazonium salt, phosphonium salt, sulfonium salt, iodonium salt). Examples of the anion of the onium salt include sulfonic acid anion, sulfonylimide anion, sulfonylmethide anion, carboxylic acid anion (in which an anion in the carboxylate (I) is excluded) and the like.

Specific examples of the acid generator (B) include compounds generating an acid upon exposure to radiation mentioned in JP 63-26653 A, JP 55-164824 A, JP 62-69263 A, JP 63-146038 A, JP 63-163452 A, JP 62-153853 A, JP 63-146029 A, U.S. Pat. Nos. 3,779,778, 3,849,137, DE Patent No. 3914407 and EP Patent No. 126,712. Compounds produced by a known method may also be used. Two or more acid generators (B) may also be used in combination.

When the acid generator (B) is a sulfonic acid anion, preferred is a salt represented by formula (B1) (hereinafter sometimes referred to as “salt (B1)” or “acid generator (B1)”)

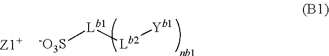

wherein, in formula (B1),
L^{b1} represents a single bond or an (nb1⁺1)-valent hydrocarbon group which may have a substituent, and —CH₂— included in the hydrocarbon group may be replaced by —O—, —S—, —CO— or —SO₂—,
L^{b2} represents a single bond or a divalent hydrocarbon group having 1 to 24 carbon atoms which may have a substituent, and —CH₂— included in the hydrocarbon group may be replaced by —O—, —S—, —CO— or —SO₂—,
Y^{b1} represents a methyl group which may have a substituent or a cyclic hydrocarbon group having 3 to 24 carbon atoms which may have a substituent, and —CH₂— included in the cyclic hydrocarbon group may be replaced by —O—, —S—, —CO— or —SO₂—,
nb1 represents an integer of 1 to 6, and when nb1 is 2 or more, a plurality of groups in parentheses may be the same or different from each other, and
Z1⁺ represents an organic cation.

Examples of the (nb1⁺1)-valent hydrocarbon group as for L^{b1} include groups which are obtained by removing nb1 hydrogen atoms from a monovalent chain hydrocarbon group, a monovalent alicyclic hydrocarbon group, a monovalent aromatic hydrocarbon group, and a monovalent group formed by combining these two or more groups, and bonding to one or more L^{b2}.

The number of carbon atoms of the hydrocarbon group is preferably 1 to 48, more preferably 1 to 42, still more preferably 1 to 36, yet more preferably 1 to 30, and further preferably 1 to 24.

Examples of the chain hydrocarbon group include groups obtained by removing nb1 hydrogen atoms of the alkyl group or alkenyl group. The alkyl group may be either linear or branched, and specific examples thereof include a methyl group, an ethyl group, an n-propyl group, an iso-propyl group, an n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentanedecyl group, a heptadecyl group and the like. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The number of carbon atoms of the chain hydrocarbon group is preferably 1 to 36, more preferably 1 to 24, still more preferably 1 to 20, yet more preferably 1 to 18, further preferably 1 to 12, and still further preferably 1 to 10.

Examples of the alicyclic hydrocarbon group include groups obtained by removing nb1 hydrogen atoms of the monocyclic or polycyclic cycloalkyl group. Examples of the monocyclic cycloalkyl group include a cyclobutyl group, a cyclopentyl group, a cyclohexyl group, a cycloheptyl group, a cyclooctyl group and the like.

Examples of the polycyclic cycloalkyl group include a cycloalkyl group having a crosslinked structure, a cycloalkyl group in which two or more rings are fused, or a cycloalkyl group in which two rings are bonded by spiro bonding. Examples of the cycloalkyl group having a crosslinked structure include a norbornyl group, an adamantyl group and the like. Examples of the cycloalkyl group in which two or more rings are fused include a bicyclo[4,4,0]decane group, a steroid group (steroid skeleton) and the like. Examples of the cycloalkyl group in which two rings are bonded by spiro bonding include a spirocyclic cycloalkyl group in which one cycloalkyl group selected from the group consisting of a cyclopentyl group, a cyclohexyl group, a norbornyl group and an adamantyl group, and a cycloalkyl group having 5 to 8 carbon atoms are bonded by spiro bonding, and the like. A double bond may be formed between two carbon atoms included in the alicyclic hydrocarbon group.

More specifically, alicyclic hydrocarbon groups represented by the following formulas are exemplified.

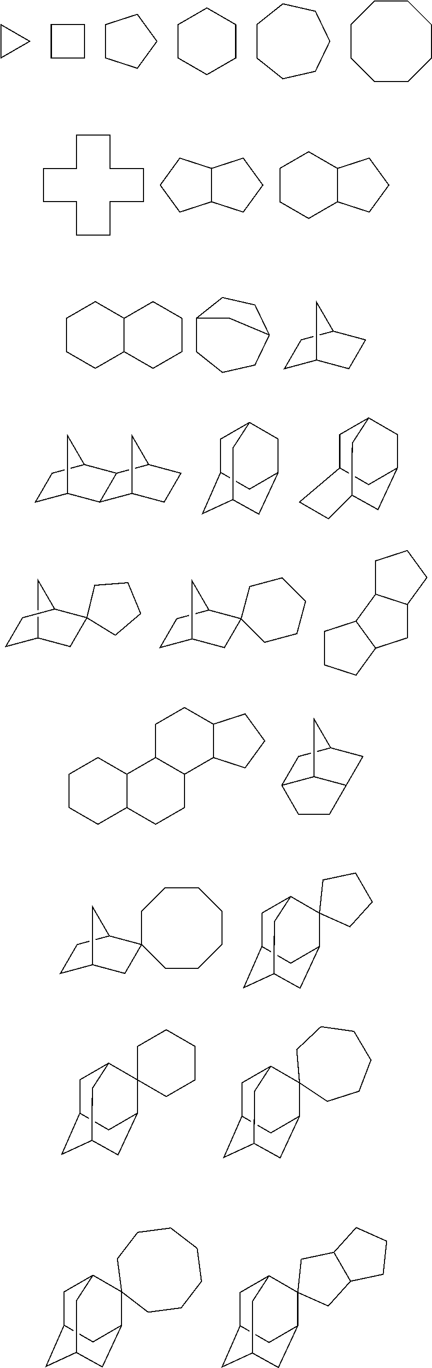

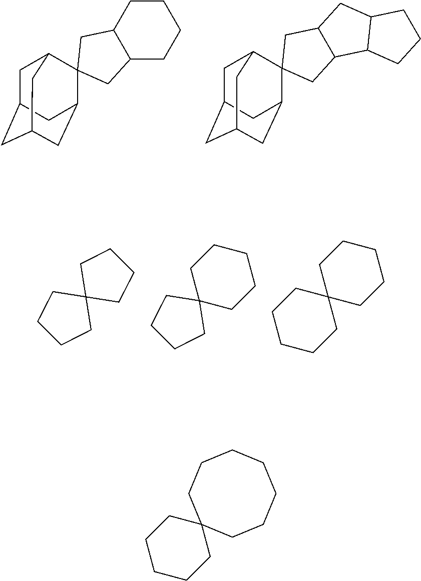

When the alicyclic hydrocarbon group is a monocyclic cycloalkyl group, the number of carbon atoms of the alicyclic hydrocarbon group is preferably 3 to 24, more preferably 3 to 20, still more preferably 3 to 18, yet more preferably 3 to 12, further preferably 3 to 10, and still further preferably 3 to 8. When the alicyclic hydrocarbon group is a polycyclic cycloalkyl group, the number of carbon atoms of the alicyclic hydrocarbon group is preferably 6 to 24, more preferably 6 to 20, still more preferably 6 to 18, yet more preferably 6 to 12, and further preferably 7 to 12.

Examples of the aromatic hydrocarbon group include groups obtained by removing nb1 hydrogen atoms of the aryl group. Examples of the aryl group include a phenyl group, a naphthyl group, an anthryl group, a biphenyl group, a fluorenyl group and the like.

More specifically, aromatic hydrocarbon groups represented by the following formulas are exemplified.

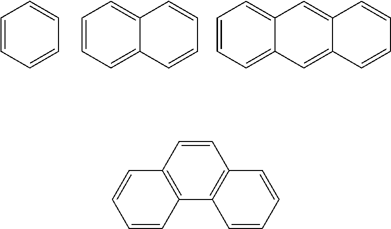

The number of carbon atoms of the aromatic hydrocarbon group is preferably 4 to 24, more preferably 4 to 20, still more preferably 4 to 18, yet more preferably 5 to 14, further preferably 5 to 10, and still further preferably 6 to 10.

When —CH₂— included in the hydrocarbon group as for L^{b1} is replaced by —O—, —CO—, —S— or —SO₂—, the number of carbon atoms before replacement is taken as the number of carbon atoms of the hydrocarbon group.

Of the hydrocarbon group as for L^{b1}, examples of the group in which —CH₂— included in the chain hydrocarbon group is replaced by —O—, —CO—, —S— or —SO₂— include a hydroxy group (a group in which —CH₂— included in the methyl group is replaced by —O—), a carboxy group (a group in which —CH₂—CH₂—included in the ethyl group is replaced by —O—CO—), a carbonyl group (a group in which —CH₂— included in the methylene group is replaced by —CO—), an oxy group (a group in which —CH₂— included in the methylene group is replaced by —O—), an alkoxy group (a group in which —CH₂— at any position included in the alkyl group is replaced by —O—), an alkoxycarbonyl group (a group in which —CH₂—CH₂— at any position included in alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—) and the like. Examples of these replaced groups include the same groups as mentioned herein as long as the upper limit of the number of carbon atoms permits.

Of the hydrocarbon group as for L^{b1}, examples of the group in which —CH₂— included in the alicyclic hydrocarbon group is replaced by —O—, —CO—, —S— or —SO₂— include groups having a structure such as cyclic ether, cyclic ketone, cyclic ester (lactone), cyclic thioether, cyclic acetal or cyclic sulfonic acid ester (sultone). Specific examples thereof include alicyclic hydrocarbon groups represented by the following formulas. The bonding site of the alicyclic hydrocarbon group represented by the following formulas can be any position.

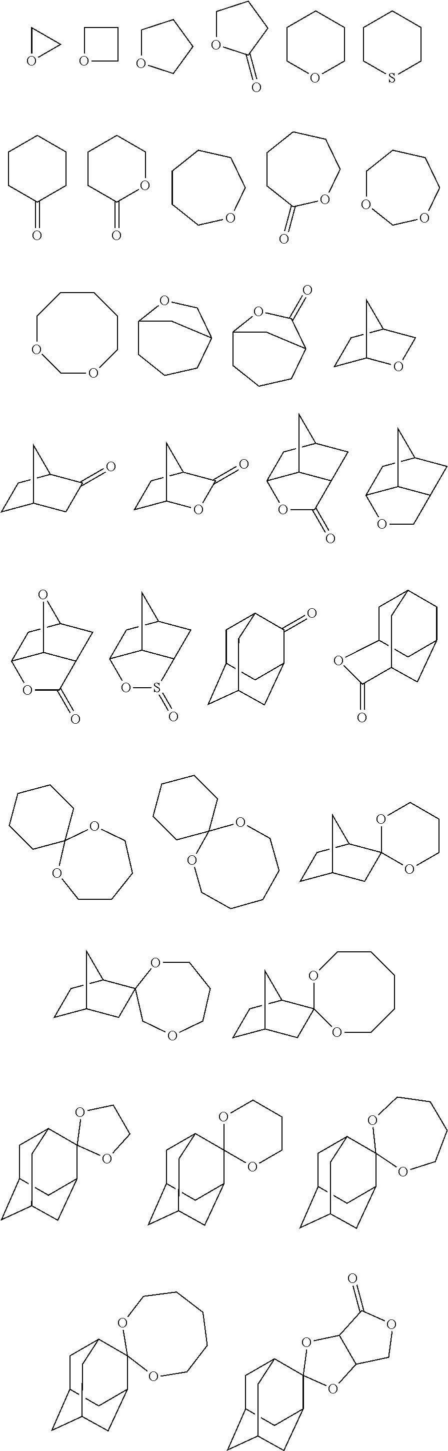

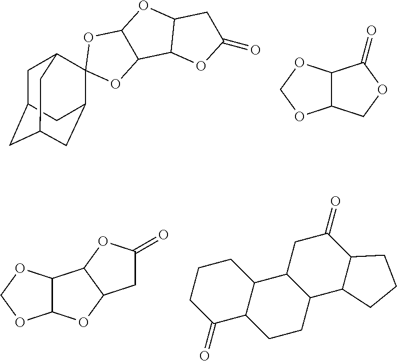

Of the hydrocarbon group as for L^{b1}, —CH₂— included in the aromatic hydrocarbon group may be replaced by —O— or —S—, and examples of the group in which —CH₂— is replaced by —O— or —S— include groups derived from a furan ring or a thiophene ring, respectively. Specifically, aromatic hydrocarbon groups represented by the following formulas are exemplified.

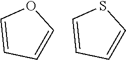

Examples of the group obtained by combining two or more groups of the chain hydrocarbon group, the alicyclic hydrocarbon group and the aromatic hydrocarbon group include a group obtained by combining the chain hydrocarbon group with the alicyclic hydrocarbon group, a group obtained by combining the chain hydrocarbon group with the aromatic hydrocarbon group, a group obtained by combining the alicyclic hydrocarbon group with the aromatic hydrocarbon group, and a group obtained by combining the chain hydrocarbon group, the alicyclic hydrocarbon group and the aromatic hydrocarbon group. The group obtained by combining the alicyclic hydrocarbon group with the aromatic hydrocarbon group may also be a fused ring.

Examples of the divalent hydrocarbon group as for L^{b2} include a divalent chain hydrocarbon group, a divalent alicyclic hydrocarbon group, a divalent aromatic hydrocarbon group, and groups obtained by combining these two or more groups, and groups which are obtained by removing one hydrogen atom from a monovalent hydrocarbon group, and bonding to Y^{b1}.

Examples of the divalent chain hydrocarbon group, the divalent alicyclic hydrocarbon group, the divalent aromatic hydrocarbon group, and the divalent group obtained by combining these two or more groups as for L^{b2} include groups obtained by removing one hydrogen atom from the monovalent chain hydrocarbon group, the monovalent alicyclic hydrocarbon group, the monovalent aromatic hydrocarbon group, and the monovalent group formed by combining these two or more groups mentioned as for L^{b1}, respectively, as long as the upper limit of the number of carbon atoms permits.

Examples of the substituent which may be possessed by the hydrocarbon group as for L^{b1} and L^{b2} include a halogen atom, a cyano group, a nitro group and the like.

Examples of the halogen atom include a fluorine atom, a chlorine atom, a bromine atom, an iodine atom and the like.

When L^{b1} and L^{b2} are groups obtained by combining an alicyclic hydrocarbon group or an aromatic hydrocarbon group with a chain hydrocarbon group, the chain hydrocarbon group may be substantially regarded as a substituent which is possessed by the alicyclic hydrocarbon group or the aromatic hydrocarbon group. By replacing —CH₂— of the chain hydrocarbon group included in the hydrocarbon group as for L^{b1} and L^{b2} by —O—, —CO—, —S— or —SO₂—, the hydrocarbon group as for L^{b1} and L^{b2} can substantially have a substituent such as a hydroxy group, a carboxy group, a carbonyl group, an oxycarbonyl group, a carbonyloxy group, an alkoxy group, an alkylcarbonyl group, an alkoxycarbonyl group, an alkylcarbonyloxy group, a thiol group or a sulfonyl group.

The number of substituents which may be possessed by L^{b1} and L^{b2} is not particularly limited, and they may have a plurality of substituents.

Examples of the cyclic hydrocarbon group as for Y^{b1} include an alicyclic hydrocarbon group, an aromatic hydrocarbon group, and groups obtained by combining an alicyclic hydrocarbon group with an aromatic hydrocarbon group.

Examples of the alicyclic hydrocarbon group and the aromatic hydrocarbon group as for Y^{b1} include same alicyclic hydrocarbon groups and aromatic hydrocarbon groups as mentioned as for L^{b1}, respectively, and when having no substituent, they may be a monovalent alicyclic hydrocarbon group and a monovalent aromatic hydrocarbon group.

Examples of the substituent which may be possessed by the methyl group as for Y^{b1} include a halogen atom, a cyano group, a hydroxy group, a nitro group and the like.

Examples of the substituent which may be possessed by the cyclic hydrocarbon group as for Y^{b1} include a halogen atom, a cyano group, a nitro group, or a hydrocarbon group having 1 to 18 carbon atoms which may have a halogen atom, a cyano group or a nitro group (—CH₂— included in the hydrocarbon group may be replaced by —O—, —S—, —CO— or —SO₂—).

The number of carbon atoms of the hydrocarbon group which may be possessed by the cyclic hydrocarbon group as for Y^{b1} is not included in the number of carbon atoms of the cyclic hydrocarbon group as for Y^{b1}.

Examples of the halogen atom include the same halogen atoms as those mentioned as the substituent as for L^{b1} and L^{b2}.

Examples of the hydrocarbon group having 1 to 18 carbon atoms which may be possessed, as the substituent, by the cyclic hydrocarbon group as for Y^{b1} include a chain hydrocarbon group, an alicyclic hydrocarbon group, an aromatic hydrocarbon group, and groups obtained by combining these groups. Examples of the chain hydrocarbon group, the alicyclic hydrocarbon group, the aromatic hydrocarbon group, and groups obtained by combining these two or more groups include the same groups as mentioned in the chain hydrocarbon group, the alicyclic hydrocarbon group, the aromatic hydrocarbon group, and groups obtained by combining these two or more groups as for L^{b1} as long as the upper limit of the number of carbon atoms permit. Examples of the group in which —CH₂— included in the hydrocarbon group having 1 to 18 carbon atoms is replaced by —O—, —S—, —CO— or —SO₂—which may be possessed, as the substituent, by the cyclic hydrocarbon group as for Y^{b1} include the same groups as mentioned in the group in which —CH₂— included in the hydrocarbon group is replaced by —O—, —S—, —CO— or —SO₂—as for L^{b1} as long as the upper limit of the number of carbon atoms permit. The hydrocarbon group having 1 to 18 carbon atoms which may be possessed, as the substituent, by the cyclic hydrocarbon group as for Y^{b1} may constitute a protecting group or a leaving group (an acid-labile group or a base-labile group) which is generally used in the relevant field.

Examples of the anion of the salt represented by formula (B1) include an anion represented by the following formula (B1-A1) (hereinafter sometimes referred to as anion (B1-A1)″) or an anion represented by formula (B1-A2) (hereinafter sometimes referred to as “anion (B1-A2)”):

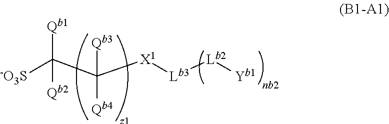

wherein, in formula (B1-A1),
L^{b2} and Y^{b1} are the same as defined in formula (B1),
Q^{b1}, Q^{b2}, Q^{b3} and Q^{b4} each independently represent a hydrogen atom, a fluorine atom, a perfluoroalkyl group having 1 to 6 carbon atoms or an alkyl group having 1 to 6 carbon atoms,
z1 represents an integer of 0 to 6, and when z1 is 2 or more, a plurality of groups in parentheses may be the same or different from each other,
X¹ represents —O—CO—, —CO—O—, —O—CO—O— or —O—,
L^{b3} represents single bond or a (nb2+1)-valent hydrocarbon group having 1 to 24 carbon atoms which may have a substituent, and —CH₂— included in the hydrocarbon group may be replaced by —O—, —S—, —CO— or —SO₂—, and
nb2 represents an integer of 1 to 3, and when nb2 is 2 or more, a plurality of groups in parentheses may be the same or different from each other.

Examples of the perfluoroalkyl group as for Q^{b1}, Q^{b2}, Q^{b3} and Q^{b4} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group and a perfluorohexyl group.

Examples of the alkyl group as for Q^{b1}, Q^{b2}, Q^{b3} and Q^{b4} include a methyl group, an ethyl group, a propyl group, an isopropyl group, a butyl group, a sec-butyl group, a tert-butyl group, a pentyl group and a hexyl group.

Q^{b1} and Q^{b2} preferably includes a fluorine atom or a perfluoroalkyl group in at least one of Q^{b1} and Q^{b2}, more preferably a fluorine atom or a perfluoroalkyl group, still more preferably a fluorine atom or a trifluoromethyl group, and yet more preferably both are fluorine atoms.

Preferably, Q^{b3} and Q^{b4} are each independently a hydrogen atom, a fluorine atom or a perfluoroalkyl group having 1 to 3 carbon atoms, Q^{b3} is preferably a hydrogen atom, a fluorine atom or a perfluoroalkyl group having 1 to 3 carbon atoms, and Q^{b4} is preferably a hydrogen atom or a fluorine atom.

z1 is preferably an integer of 0 to 3, and more preferably 0, 1 or 2.

X¹ is preferably —O—CO— or —CO—O—.

Examples of the hydrocarbon group in L^{b3} include the same hydrocarbon groups as mentioned as for L^{b1} of formula (B1) as long as the upper limit of the number of carbon atoms permit.

L^{b3} is preferably a single bond, a chain hydrocarbon group having 1 to 12 carbon atoms which may have a substituent (—CH₂— included in the chain hydrocarbon group may be replaced by —O— or —CO—), an alicyclic hydrocarbon group having 3 to 18 carbon atoms which may have a substituent (—CH₂— included in the alicyclic hydrocarbon group may be replaced by —O—, —S—, —CO— or —SO₂—), an aromatic hydrocarbon group having 6 to 10 carbon atoms which may have a substituent (—CH₂— included in the aromatic hydrocarbon group may be replaced by —O— or —S—) or groups obtained by combining these two or more groups, and more preferably a single bond, a chain hydrocarbon group having 1 to 6 carbon atoms, or a group represented by the following formula (Lb3-1). When —CH₂— included in the chain hydrocarbon group is replaced by —O— or —CO—, the number thereof is preferably 1 to 4, and one —CH₂—CH₂— included in the chain hydrocarbon group is preferably replaced by —O—CO— or —CO—O—, or one —CH₂—CH₂—CH₂— included in the chain hydrocarbon group is preferably replaced by —O—CO—O—:

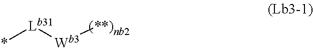

wherein, in formula (L^{b3}-1),
nb2 is the same as defined in formula (B1-A1),
L^{b31} represents a single bond or a chain hydrocarbon group having 1 to 12 carbon atoms, —CH₂— included in the chain hydrocarbon group may be replaced by —O— or —CO—, and the chain hydrocarbon group may have a substituent,
W^{b3} represents an alicyclic hydrocarbon group having 3 to 18 carbon atoms or an aromatic hydrocarbon group having 6 to 10 carbon atoms, —CH₂— included in the alicyclic hydrocarbon group may be replaced by —O—, —S—, —CO— or —SO₂—, —CH₂— included in the aromatic hydrocarbon group may be replaced by —O— or —S—, and the alicyclic hydrocarbon group and the aromatic hydrocarbon group may have a substituent, and
* and ** represent a bonding site, and * represents a bonding site to X¹.

In formula (Lb3-1), examples of the chain hydrocarbon group as for L^{b31} include the same chain hydrocarbon groups as mentioned as for L^{b1} as long as the upper limit of the number of carbon atoms permit.

In formula (Lb3-1), examples of the alicyclic hydrocarbon group and the aromatic hydrocarbon group as for W^{b3} include the same alicyclic hydrocarbon groups and aromatic hydrocarbon groups as mentioned as for L^{b1} as long as the upper limit of the number of carbon atoms permit.

In formula (Lb3-1), examples of the substituent which may be possessed by the chain hydrocarbon group as for L^{b31} and the substituent which may be possessed by the alicyclic hydrocarbon group and the aromatic hydrocarbon group as for W^{b1} include the same substituents as mentioned as for the substituent which may be possessed by the hydrocarbon group as for L^{b1}.

L^{b31} is preferably a single bond or an alkanediyl group having 1 to 6 carbon atoms (—CH₂— included in the alkanediyl group may be replaced by —O— or —CO—).

Particularly, the alicyclic hydrocarbon group and the aromatic hydrocarbon group as for W^{b3} are preferably an alicyclic hydrocarbon group and an aromatic hydrocarbon group mentioned below. In the alicyclic hydrocarbon group and the aromatic hydrocarbon group mentioned below, * and ** represent a bonding site, * represents a bonding site to X¹ or L^{b31}, ** represents a bonding site to a hydrogen atom, a substituent or L^{b2}, and at least one ** represents a bonding site to L^{b2}. In the alicyclic hydrocarbon group mentioned below, —CH₂— included in the alicyclic hydrocarbon group may be replaced by —O—, —S—, —CO— or —SO₂—. When —CH₂— included in the alicyclic hydrocarbon group is replaced by —O—, —S—, —CO— or —SO₂—, it is preferable to form an ether ring, an ester ring (lactone), a carbonic acid ester ring, a sulfonic acid ester ring (sultone) or an acetal ring.

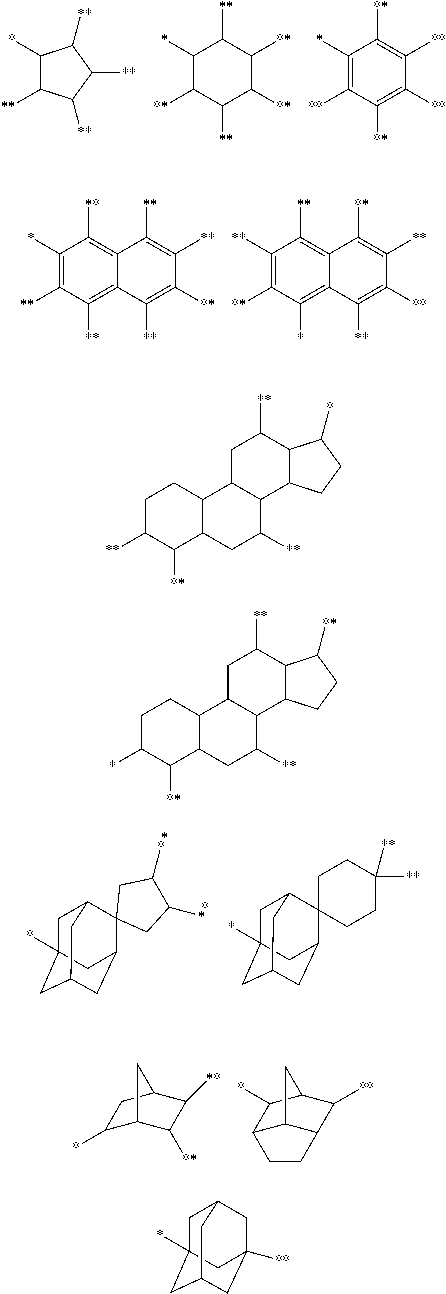

In formula (B1-A1), L^{b2} is preferably a single bond or a chain hydrocarbon group having 1 to 12 carbon atoms (—CH₂—included in the chain hydrocarbon group may be replaced by —O— or —CO—), and more preferably a single bond, —O—, —O—CO—, —CO—O—, —O—CO—O— or *-L^{b21}-X²-L^{b22}-** (one of L^{b21} and L^{b22} represents a chain hydrocarbon group having 1 to 6 carbon atoms, and the other one represents a single bond or a chain hydrocarbon group having 1 to 6 carbon atoms, X² represents —O—, —CO—O—, —O—CO— or —O—CO—O—, * and ** represent a bonding site, and ** represents a bonding site to Y^{b1}, in which the total number of carbon atoms of L^{b21}, X² and L^{b22} is 12 or less).

In formula (B1-A1), Y^{b1} is preferably a cyclic hydrocarbon group having 3 to 20 carbon atoms which may have a substituent (—CH₂— included in the cyclic hydrocarbon group may be replaced by —O—, —CO—, —S— or —SO₂—), more preferably, an alicyclic hydrocarbon group having 3 to 18 carbon atoms which may have a substituent (—CH₂— included in the alicyclic hydrocarbon group may be replaced by —O—, —CO—, —S— or —SO₂—) or an aromatic hydrocarbon group having 6 to 18 carbon atoms which may have a substituent, and still more preferably an alicyclic hydrocarbon group having 3 to 16 carbon atoms which may have a substituent (—CH₂— included in the alicyclic hydrocarbon group may be replaced by —O—, —CO—, —S— or —SO₂—) or an aromatic hydrocarbon group having 6 to 10 carbon atoms which may have a substituent. Specifically, groups represented by the following formula (Y1) to formula (Y36) are preferable. In formula (Y1) to formula (Y36), R^{Yb} represents a hydrogen atom or an alkyl group having 1 to 4 carbon atoms of substituents which may be possessed by the cyclic hydrocarbon group as for Y^{b1}, R^{Yc} represents a hydrogen atom or a substituent which may be possessed by the cyclic hydrocarbon group as for Y^{b1}, and * represents a bonding site to L^{b2}. The alicyclic hydrocarbon group and the aromatic hydrocarbon group represented by the following formula (Y1) to formula (Y36) are not particularly shown in the following formulas, but may have any other substituents which may be possessed by the cyclic hydrocarbon as for Y^{b1}.

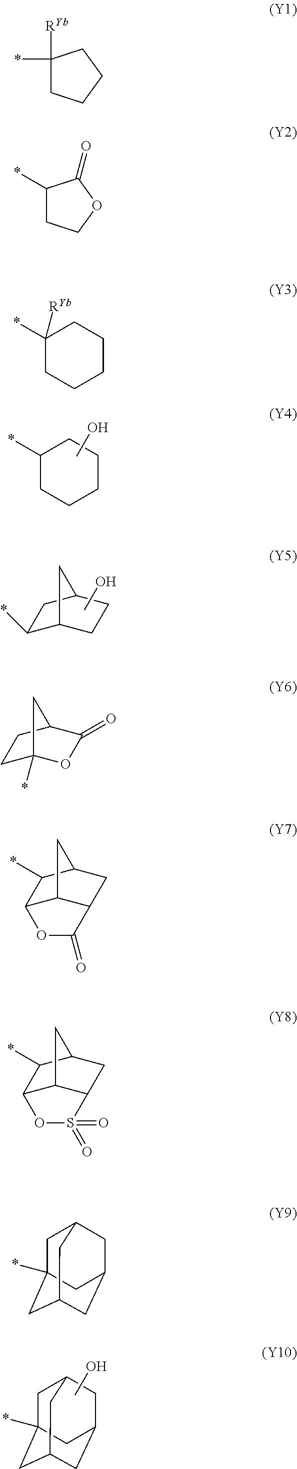

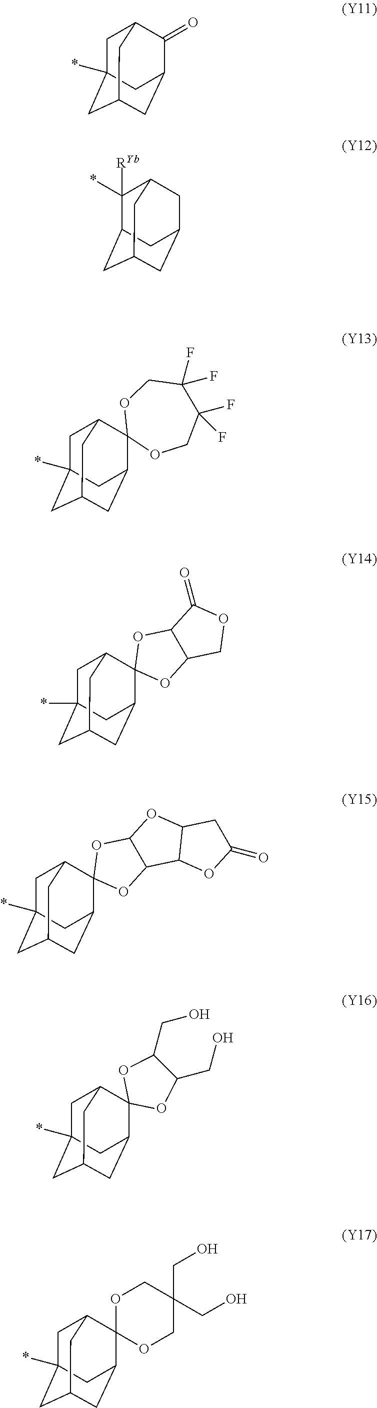

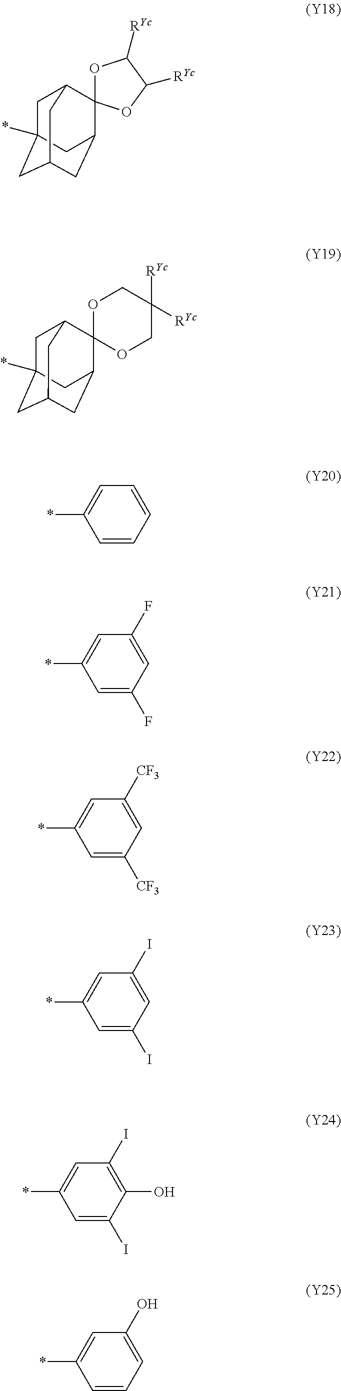

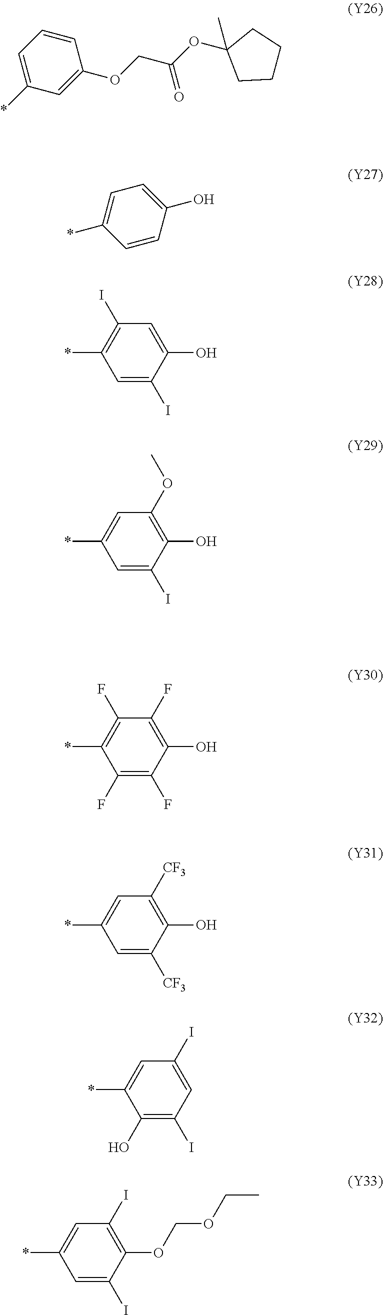

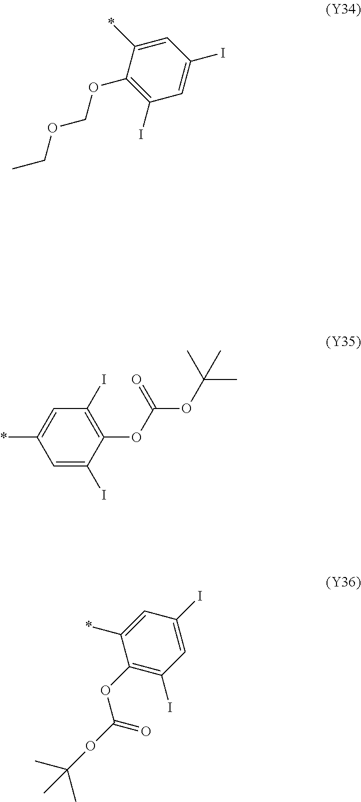

The anion represented by formula (B1-A1) is preferably an anion represented by formula (B1-A1-1) to formula (B1-A1-85) [hereinafter sometimes referred to as “anion (B1-A1-1)” or the like according to the number of formula number], and more preferably an anion represented by any one of formula (B1-A1-1) to formula (B1-A1-4), formula (B1-A1-9), formula (B1-A1-10), formula (B1-A1-24) to formula (B1-A1-33), formula (B1-A1-36) to formula (B1-A1-40) and formula (B1-A1-47) to formula (B1-A1-85).

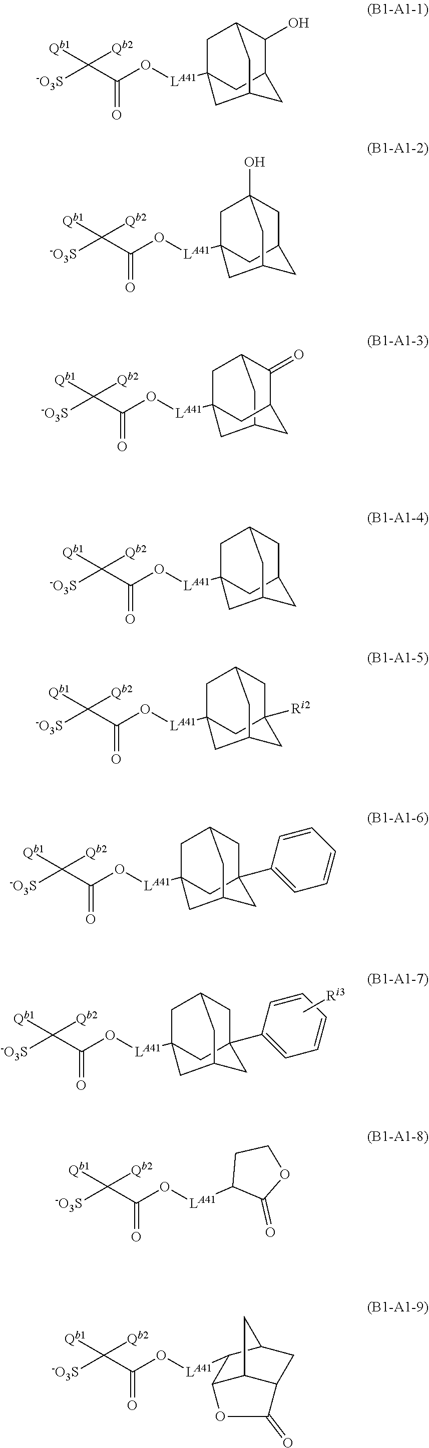

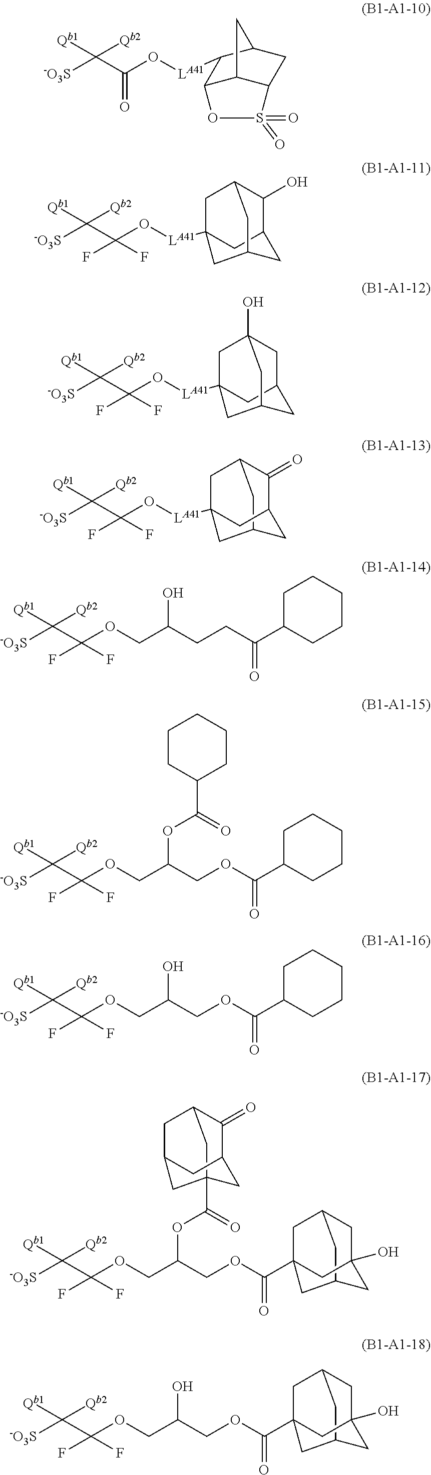

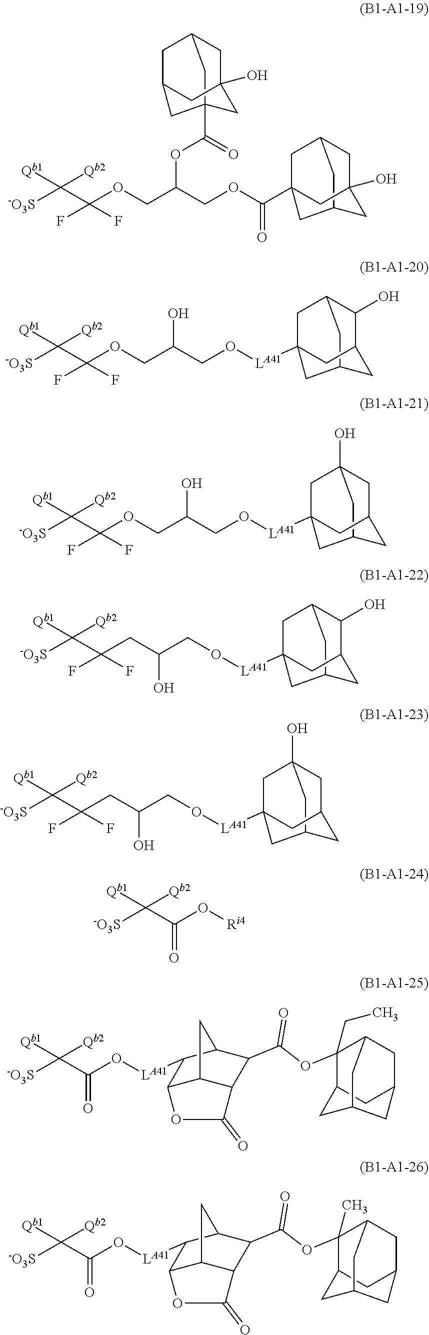

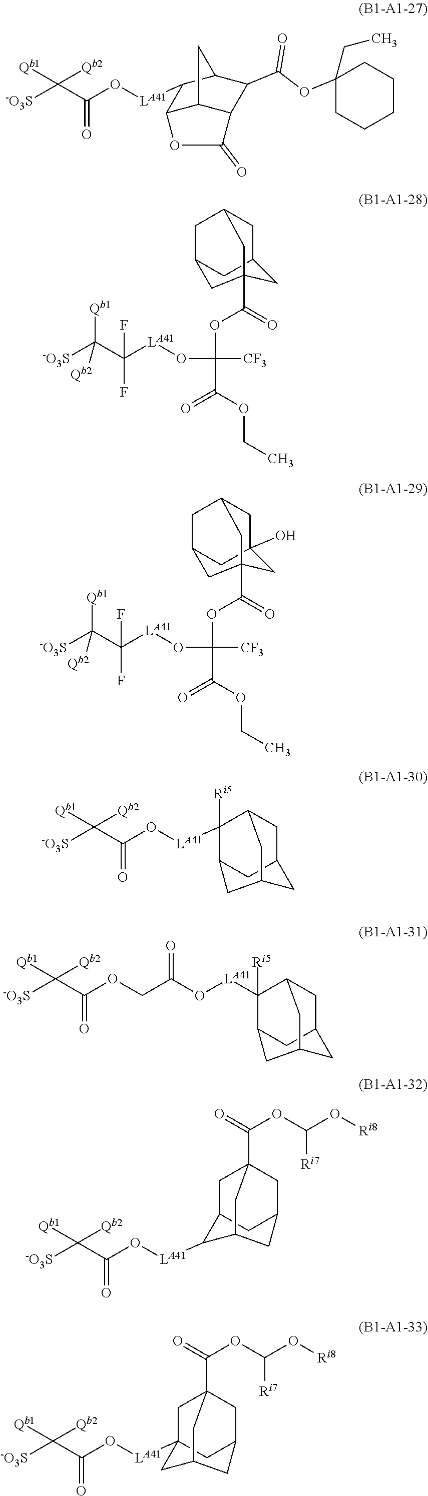

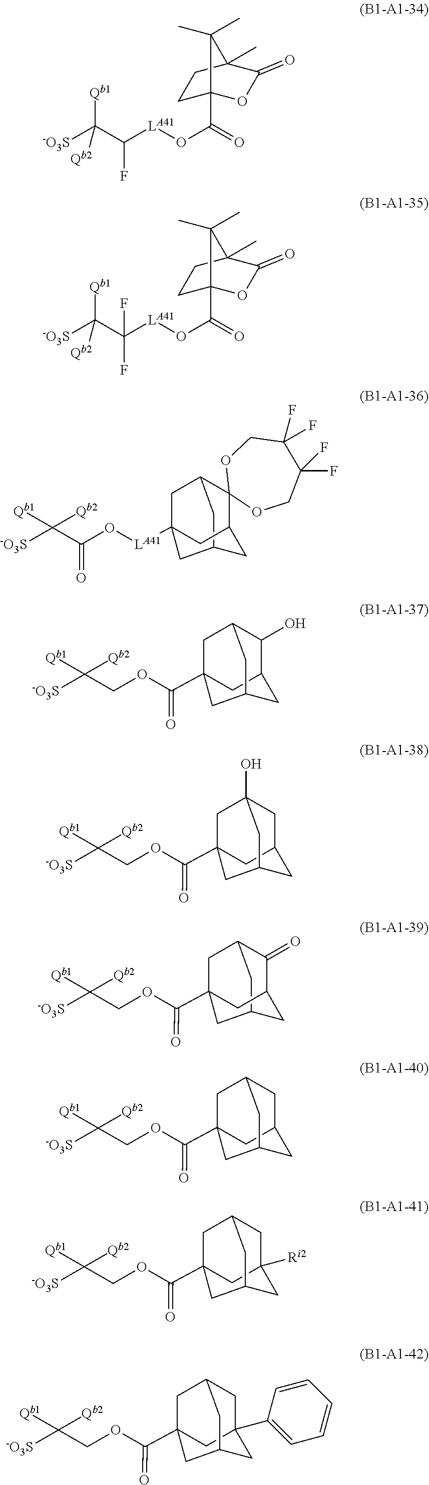

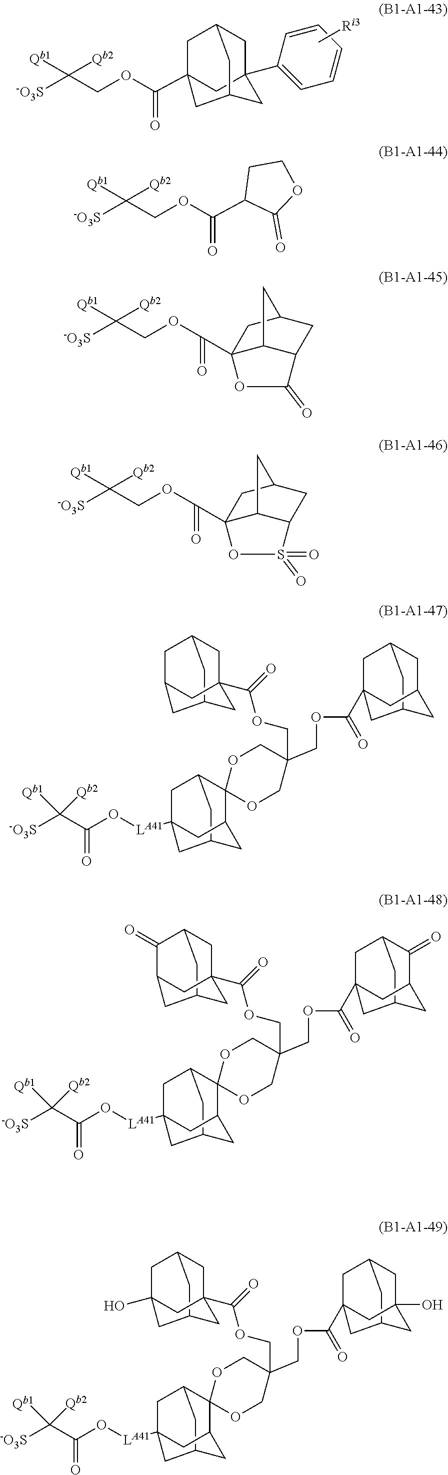

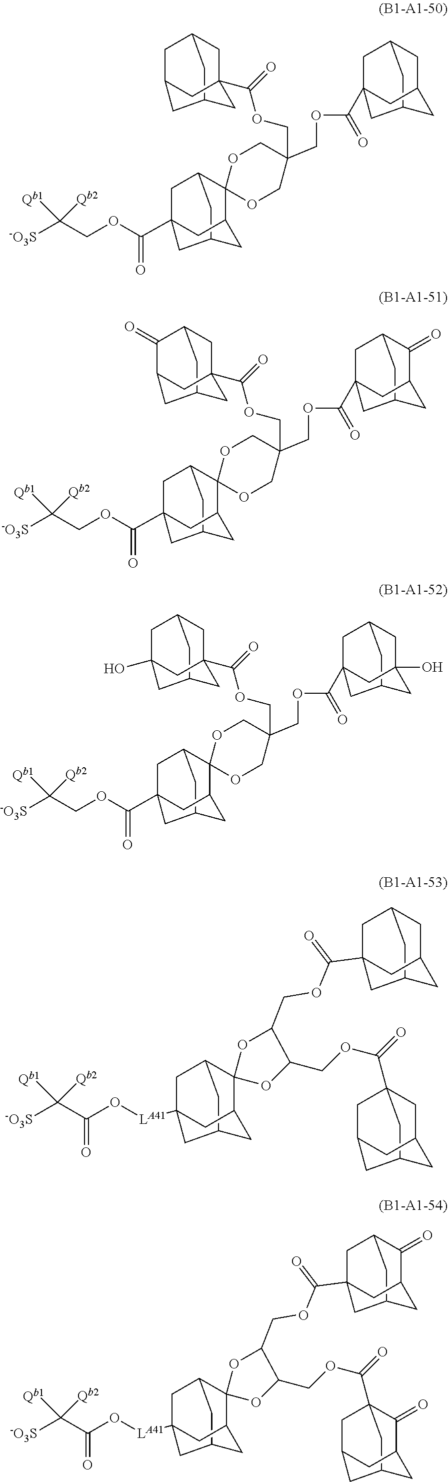

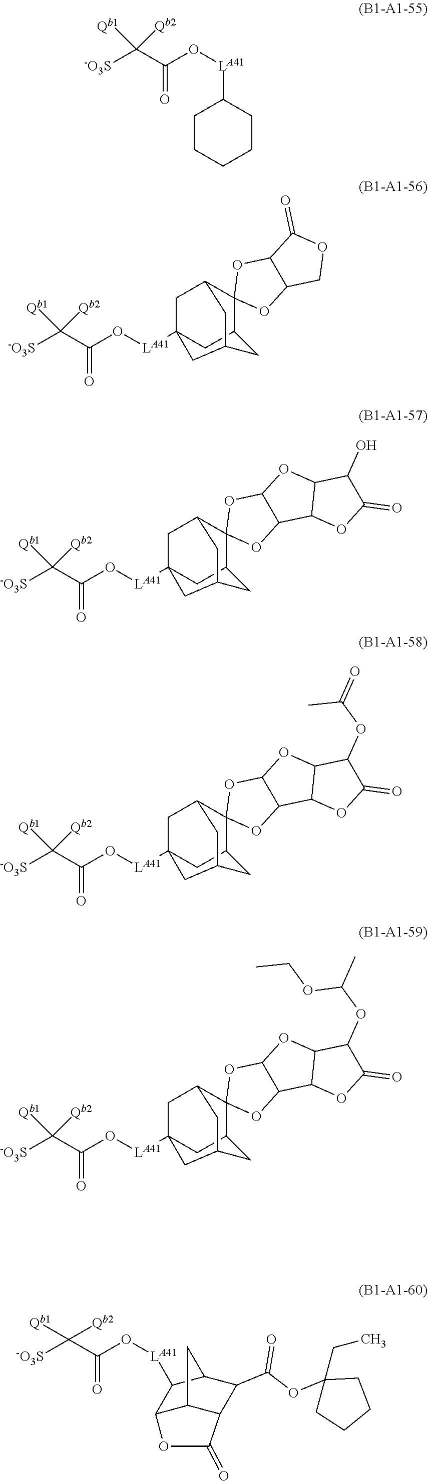

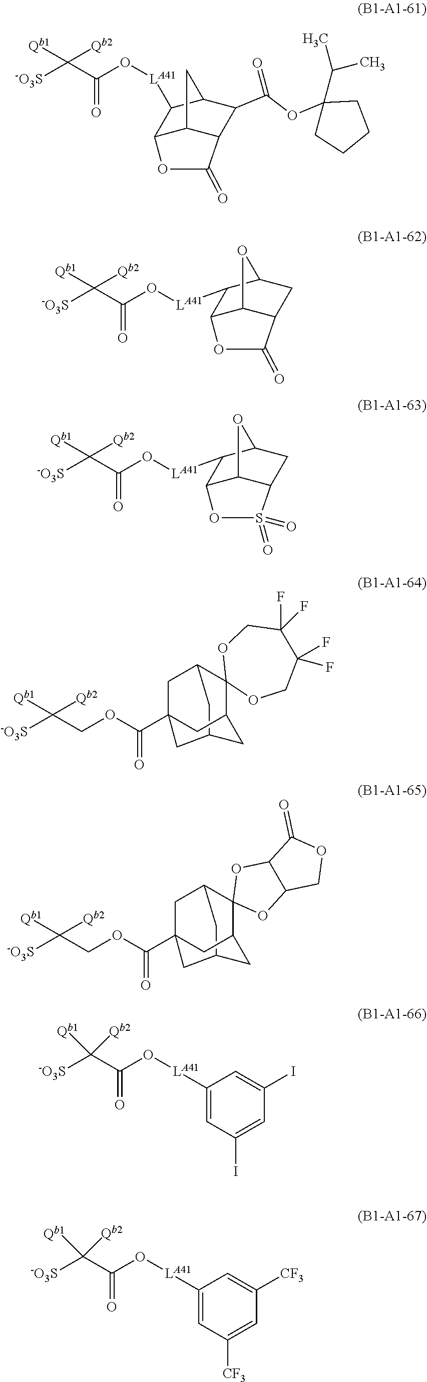

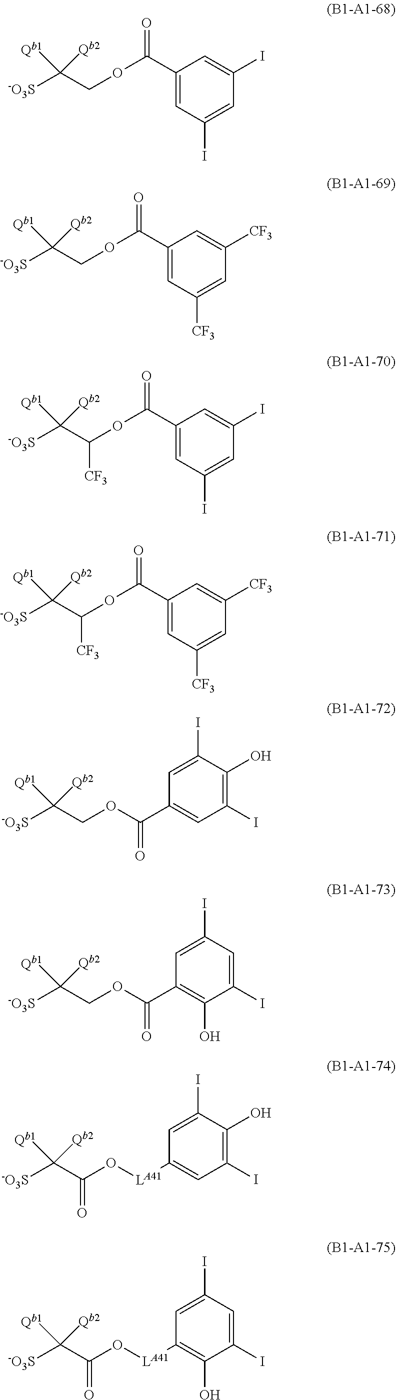

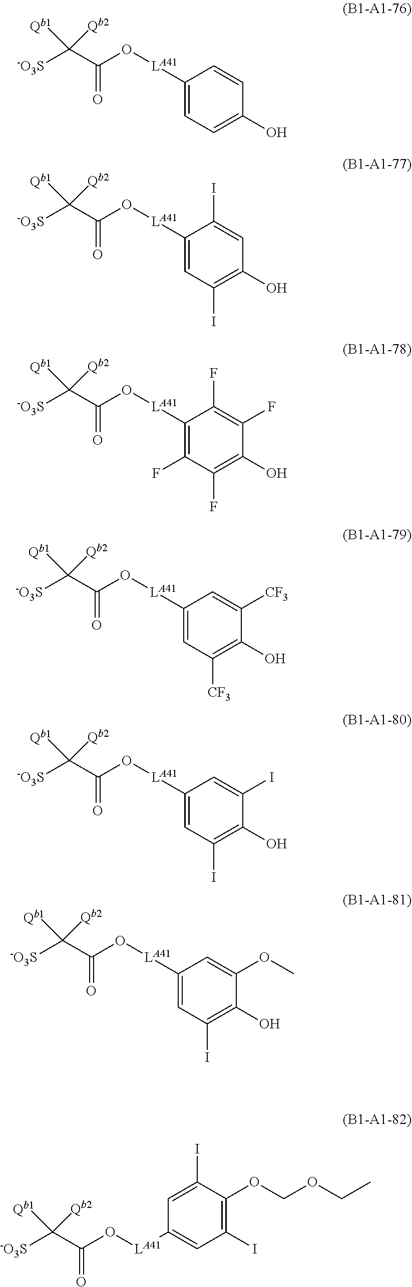

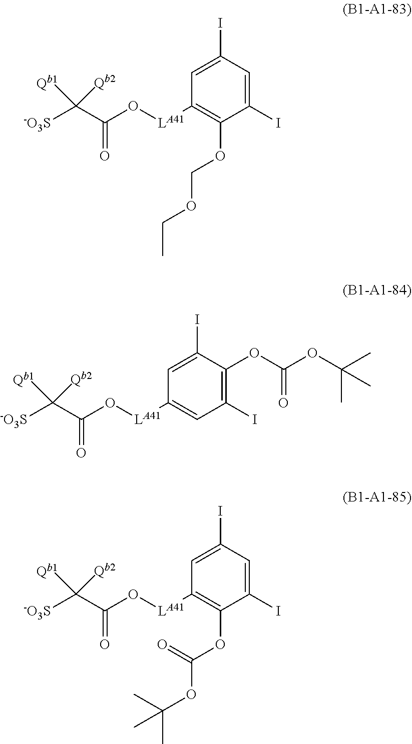

In formula (B1-A1-1) to formula (B1-A1-85), Rⁱ² to Rⁱ⁷ each independently represent, for example, an alkyl group having 1 to 4 carbon atoms, and preferably a methyl group or an ethyl group. Rⁱ⁸ is, for example, a chain hydrocarbon group having 1 to 12 carbon atoms, preferably an alkyl group having 1 to 4 carbon atoms, an alicyclic hydrocarbon group having 5 to 12 carbon atoms, or a group formed by combining these groups, and more preferably a methyl group, an ethyl group, a cyclohexyl group or an adamantyl group. L^{A41} is a single bond or an alkanediyl group having 1 to 4 carbon atoms. Q^{b1} and Q^{b2} are the same as defined above.

Specific examples of the anion represented by formula (B1-A1) include anions mentioned in JP 2010-204646 A.

The anion represented by formula (B1-A1) preferably includes anions represented by formula (B1a-1) to formula (B1a-70). Of these, an anion represented by any one of formula (B1a-1) to formula (B1a-4), formula (B1a-7) to formula (B1a-11), formula (B1a-14) to formula (B1a-30) and formula (B1a-35) to formula (B1a-70) is preferable.

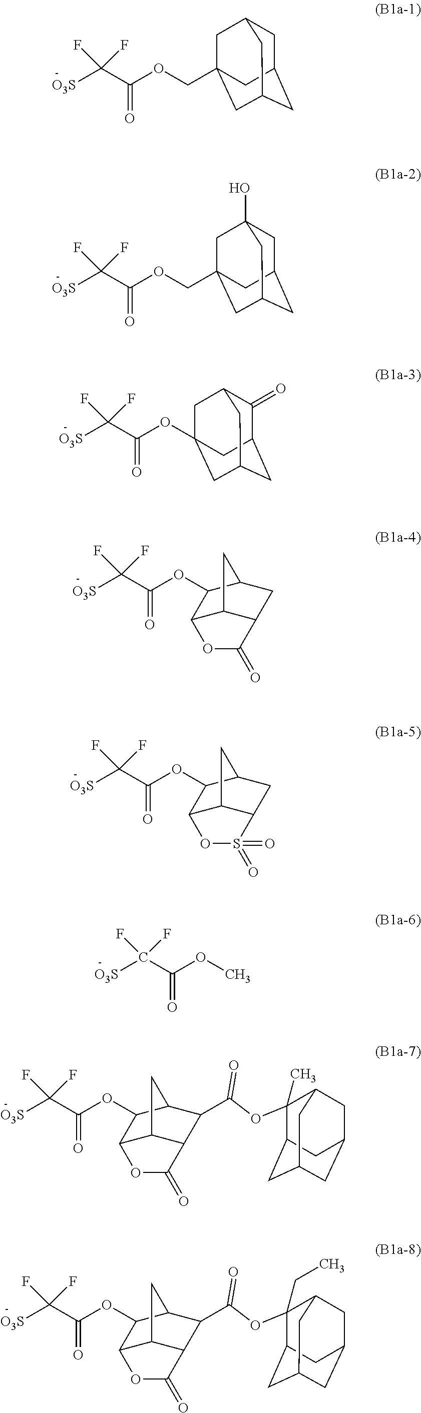

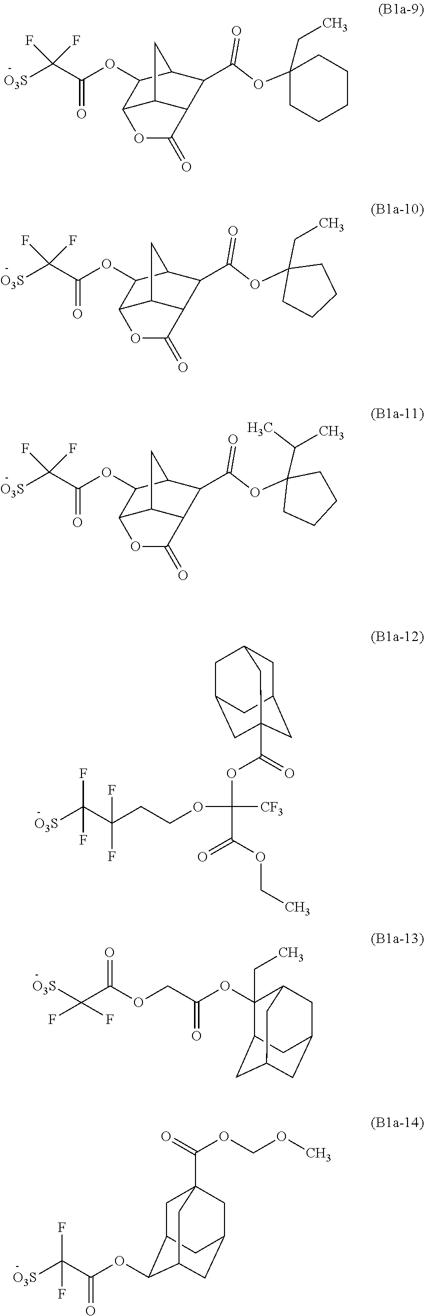

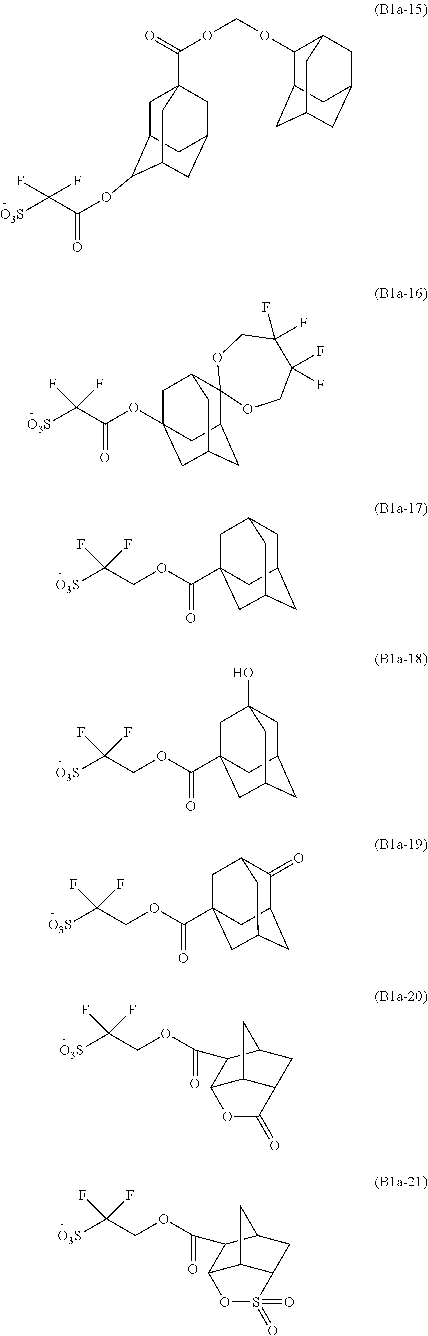

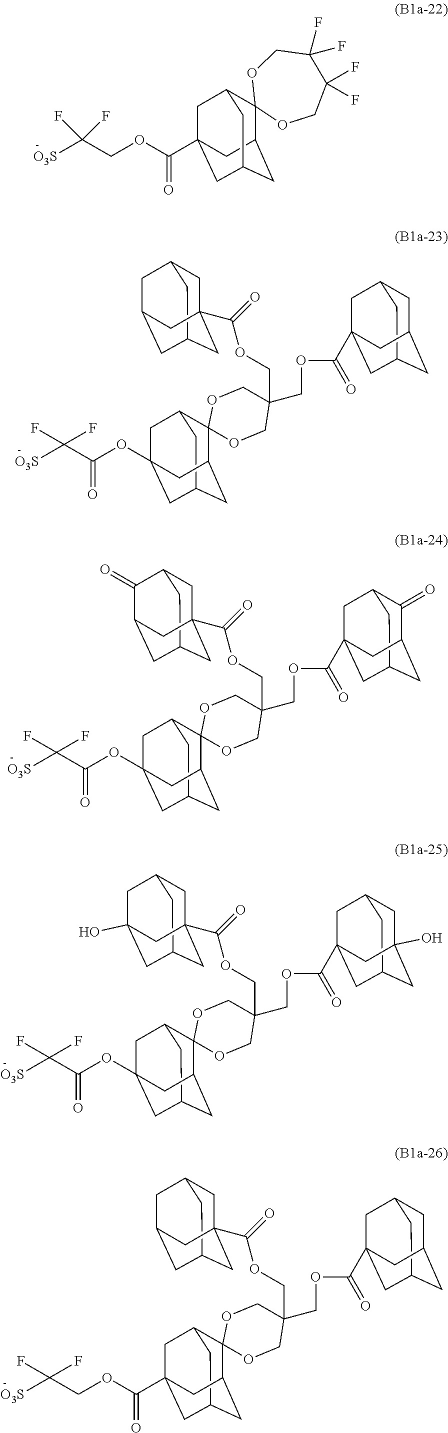

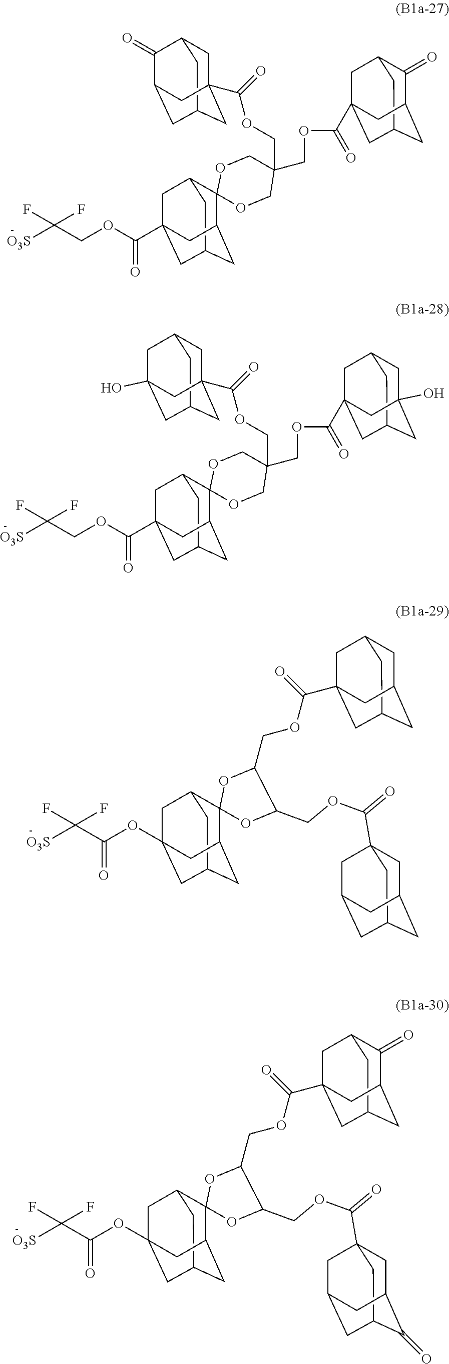

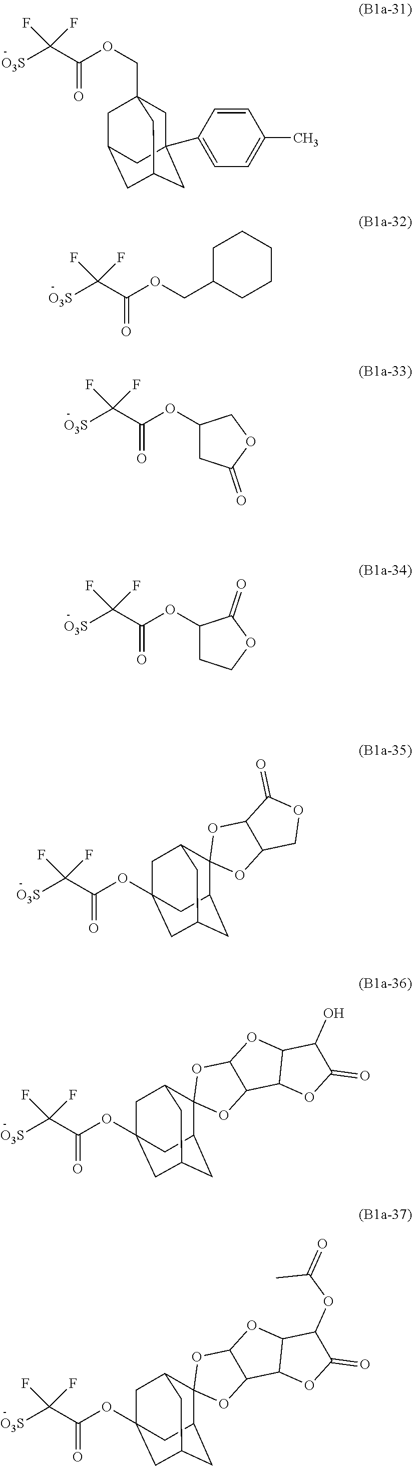

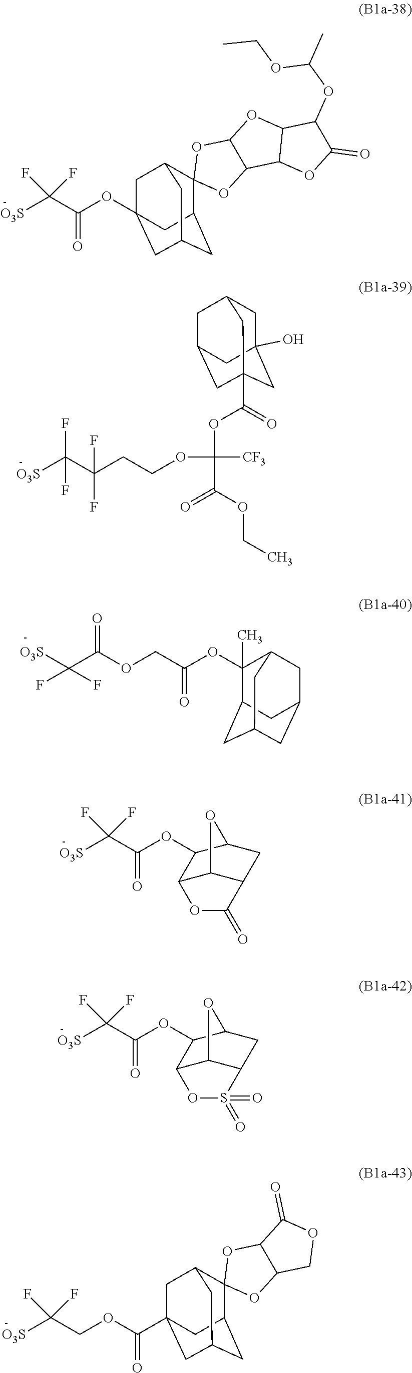

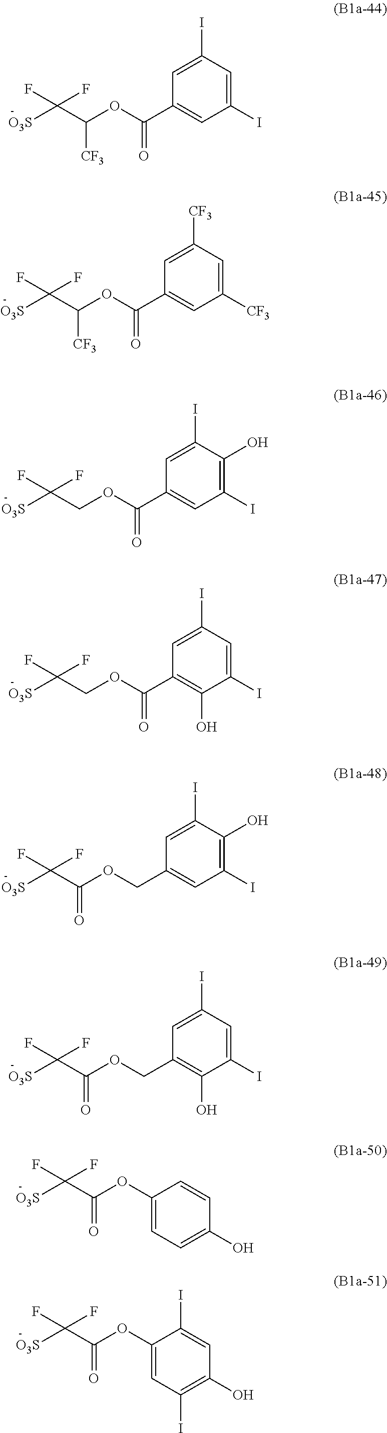

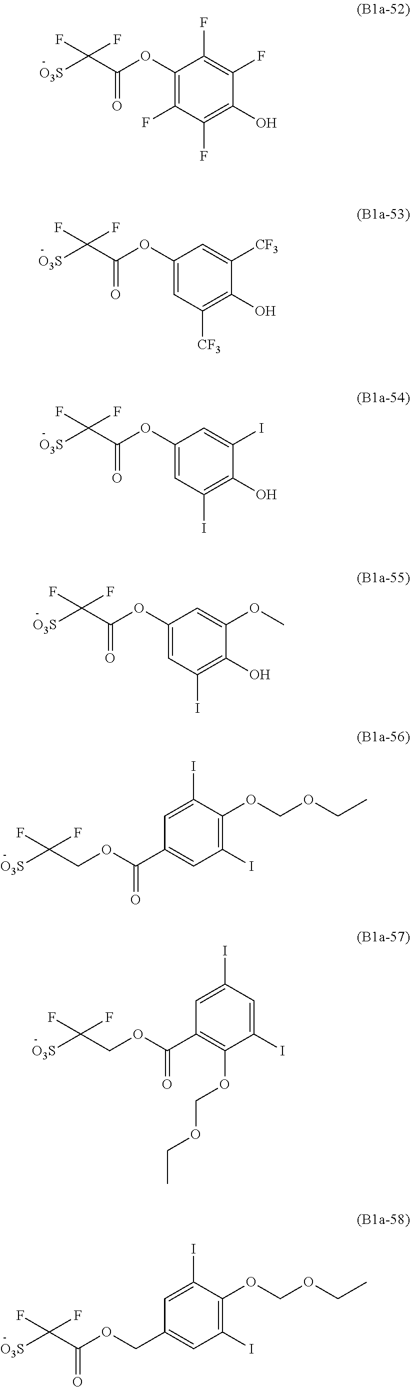

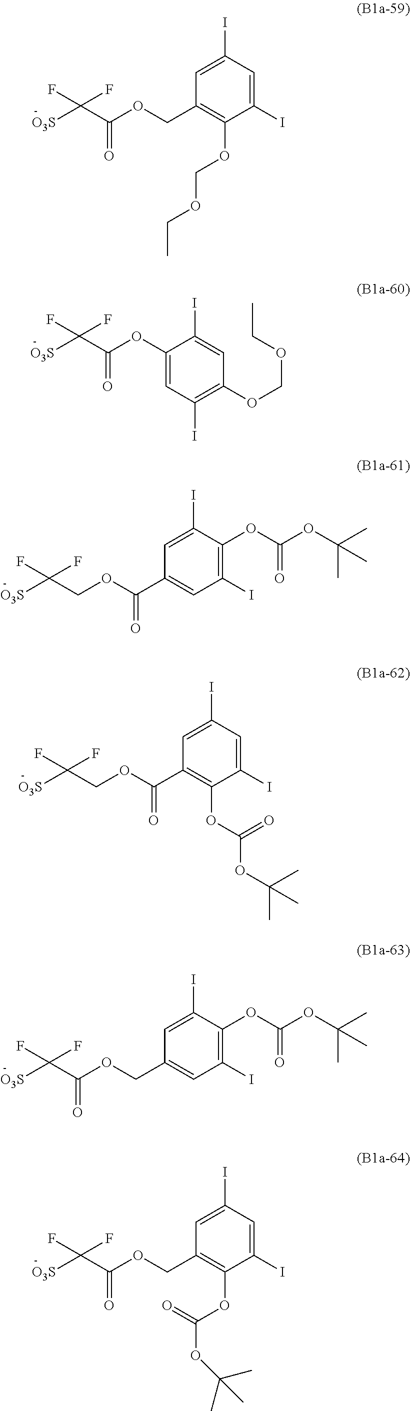

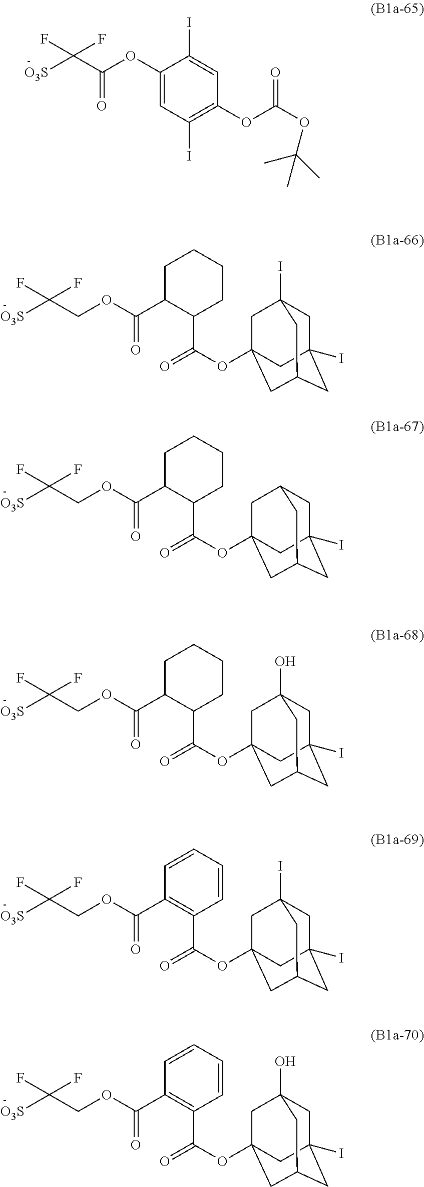

The anion represented by formula (B1-A2) is represented by the following formula:

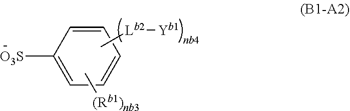

wherein, in formula (B1-A2),
L^{b2} and Y^{b1} are the same as defined in formula (B1),
R^{b1} represents a halogen atom or an alkyl group having 1 to 6 carbon atoms, and —CH₂— included in the alkyl group may be replaced by —O— or —CO—,
nb4 represents an integer of 1 to 5, and when nb4 is 2 or more, a plurality of groups in parentheses may be the same or different from each other, and
nb3 represents an integer of 0 to 4, and when nb3 is 2 or more, a plurality of R^{b1} may be the same or different from each other,
in which nb4 and nb3 satisfy: 1≤nb4+nb3≤5.

In formula (B1-A2), examples of the hydrocarbon group as for L^{b2} and the cyclic hydrocarbon group as for Y^{b1} include the same hydrocarbon groups as for L^{b2} and the same cyclic hydrocarbon groups as for Y^{b1} in formula (B1) as long as the upper limit of the number of carbon atoms permit. Examples of the substituent which may be possessed by the hydrocarbon group as for L^{b2}, and the methyl group and the cyclic hydrocarbon group as for Y^{b1} also include the same substituents which may be possessed by the hydrocarbon groups as for L^{b2}, and the methyl group and the cyclic hydrocarbon group as for Y^{b1} of formula (B1). These hydrocarbon group, methyl group and cyclic hydrocarbon group may have one substituent or a plurality of substituents.

L^{b2} is preferably a chain hydrocarbon group having 1 to 12 carbon atoms (—CH₂— included in the chain hydrocarbon group may be replaced by —O— or —CO—), more preferably *—CO— O-L^{b41}-(L^{b4}1 is a single bond or a chain hydrocarbon group having 1 to 6 carbon atoms, —CH₂— included in the chain hydrocarbon group may be replaced by —O— or —CO—, * represents a bonding site to the benzene ring to which SO₃ is bonded, and L^{b4}1 is preferably a single bond or a chain hydrocarbon group having 1 to 3 carbon atoms), and still more preferably *—CO—O—.

Y^{b1} is preferably a cyclic hydrocarbon group having 3 to 20 carbon atoms which may have a substituent (—CH₂—included in the cyclic hydrocarbon group may be replaced by —O—, —CO—, —S— or —SO₂—), more preferably an alicyclic hydrocarbon group having 3 to 18 carbon atoms which may have a substituent (—CH₂— included in the alicyclic hydrocarbon group may be replaced by —O—, —CO—, —S— or —SO₂—) or an aromatic hydrocarbon group having 6 to 18 carbon atoms which may have a substituent, and still more preferably an alicyclic hydrocarbon group or an aromatic hydrocarbon group exemplified as for Y^{b1} or L^{b1} of formula (B1-A1). Specifically, groups represented by formula (Y1) to formula (Y36) mentioned above are preferable, and groups represented by formula (Y1) to formula (Y19) mentioned above are more preferable.

In formula (B1-A2), nb4 is preferably an integer of 1 to 4, more preferably an integer of 1 to 3, still more preferably 1 or 2, and yet more preferably 2. When nb4 is 1 or 2, the bonding site of -L^{b2}-Y^{b1} is preferably the m-position of the benzene ring, with respect to the bonding site of SO₃⁻, as shown in the following structures:

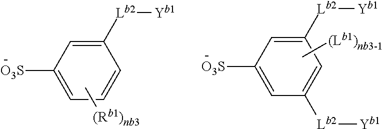

wherein, in the above formulas, L^{b2}, Y^{b1}, R^{b1} and nb3 are the same as defined in formula (B1-A2).

When nb4 is 2 or more, a plurality of L^{b2} and Y^{b1} are preferably the same groups as each other.

Examples of the alkyl group having 1 to 6 carbon atoms as for R^{b1} include alkyl groups such as a methyl group, an ethyl group, a propyl group, an isopropyl group, a butyl group, an isobutyl group, a tert-butyl group, a pentyl group and a hexyl group. The number of carbon atoms of the alkyl group is preferably 1 to 4, and more preferably 1 to 3.

Examples of the halogen atom as for R^{b1} include a fluorine atom, a chlorine atom, a bromine atom and an iodine atom.

Examples of the group in which —CH₂— included in the alkyl group is replaced by —O— or —CO— include a hydroxy group, a carboxy group, an alkoxy group, an alkoxycarbonyl group, an alkylcarbonyl group, an alkylcarbonyloxy group, an oxy group, a carbonyl group and the like. These specific examples are the same as those mentioned above.

Preferably, R^{b1} is each independently a halogen atom or an alkyl group having 1 to 4 carbon atoms (—CH₂— included in the alkyl group may be replaced by —O— or —CO—), more preferably a fluorine atom, an iodine atom or an alkyl group having 1 to 3 carbon atoms (—CH₂— included in the alkyl group may be replaced by —O— or —CO—), and still more preferably a fluorine atom, an iodine atom, a hydroxy group, a methoxy group or a methyl group.

nb3 is preferably an integer of 0 to 3, and more preferably an integer of 0 to 2. In one embodiment, nb3 is preferably 0. In another embodiment, nb3 is preferably 1 or 2. When nb3 is 1, R^{b1} is preferably a halogen atom, and more preferably a fluorine atom or an iodine atom. When nb3 is 2, it is preferable that one of two R^{b1} is a halogen atom, and the other one is a halogen atom or an alkyl group having 1 to 4 carbon atoms, and it is more preferable that one of R^{b1} is a fluorine atom or an iodine atom, and the other one is a fluorine atom, an iodine atom or an alkyl group having 1 to 3 carbon atoms.

Examples of the anion represented by formula (B1-A2) include the following anions. Of these, anions represented by formula (B2a-1) to formula (B2a-20) are preferable, and anions represented by formula (B2a-1) to formula (B2a-11) and formula (B2a-16) to formula (B2a-20) are more preferable. The anions of the following formula (B2a-1) to formula (B2a-20) may have a substituent not shown, with some R^{b1} being omitted.

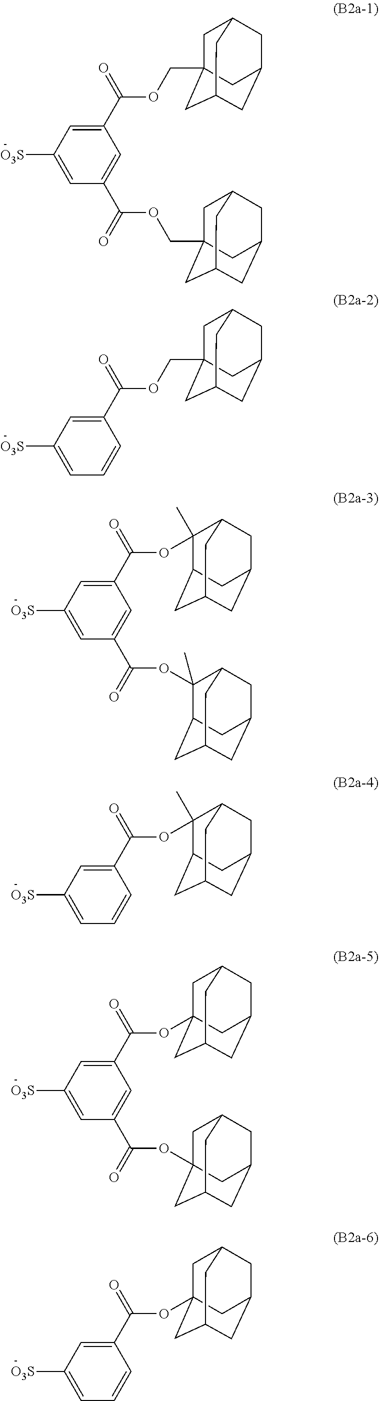

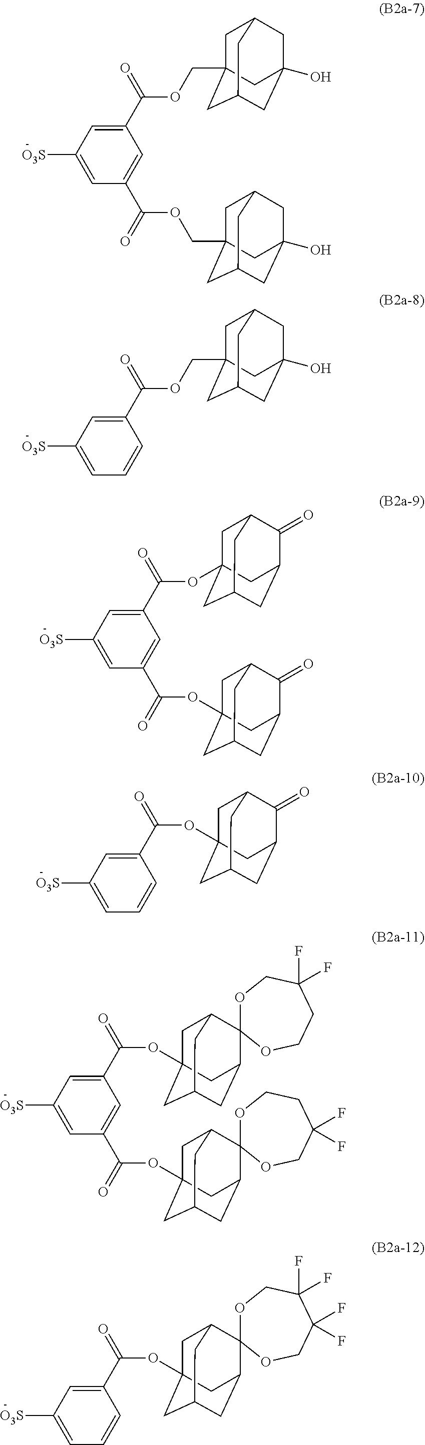

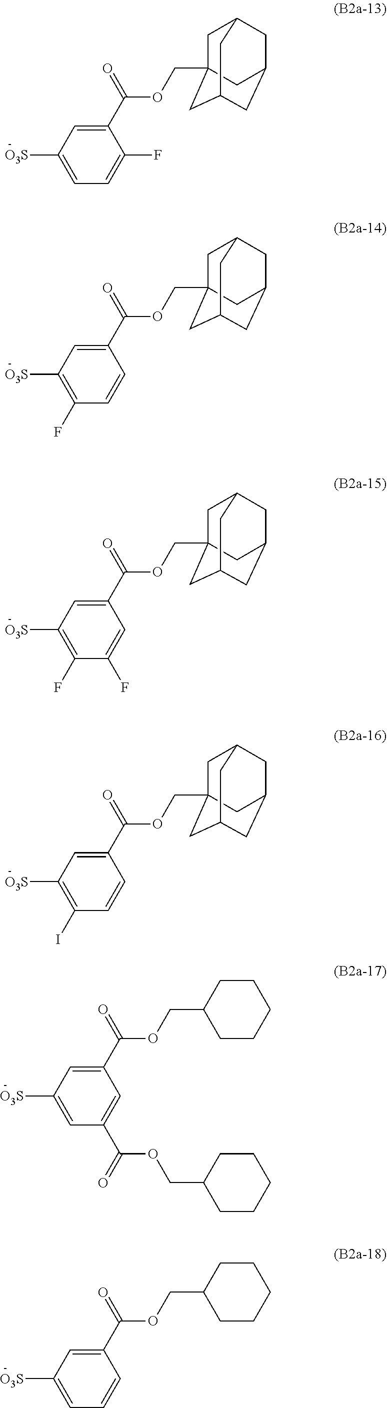

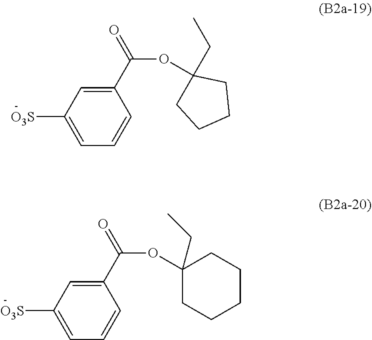

In another embodiment of the acid generator (B), it is also possible to preferably use, as the acid generator (B), a salt in which the sulfonic acid anion in the salt represented by formula (B1) is replaced by a sulfonylimide anion, a sulfonylmethide anion or a carboxylic acid anion.

Examples of the sulfonylimide anion or sulfonylmethide anion include an anion represented by the following formula (B1-A3) (hereinafter sometimes referred to as “anion (B1-A3)”):

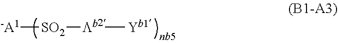

wherein, in formula (B1-A3),
A¹ represents a nitrogen atom or a carbon atom,
L^{b2′} represents a single bond or a divalent hydrocarbon group having 1 to 24 carbon atoms which may have a substituent, and —CH₂— included in the hydrocarbon group may be replaced by —O—, —S—, —CO— or —SO₂—,
Y^{b1′} represents a methyl group which may have a substituent or a cyclic hydrocarbon group having 3 to 24 carbon atoms which may have a substituent, and —CH₂— included in the cyclic hydrocarbon group may be replaced by —O—, —S—, —SO₂— or —CO—, and two —SO₂-L^{b2′}-Y^{b1′} may be bonded to each other to form a ring containing A¹, and
nb5 represents 2 or 3, and when nb5 is 2, A¹ represents a nitrogen atom, and when nb5 is 3, A¹ represents a carbon atom, and a plurality of groups in parentheses may be the same or different from each other.

In formula (B1-A3), examples of the hydrocarbon group, the cyclic hydrocarbon group and the substituent as for L^{b2}, and Y^{b1′} include the same groups as L^{b2} and Y^{b1} of formula (B1-A1).

In formula (B1-A3), L^{b2′} is preferably a single bond, *-L^{b23}-, *-Lb²³-X²—or *-Lb²³-X²—W²—X³— (L^{b23} represents a chain hydrocarbon group having 1 to 6 carbon atoms which may have a fluorine atom, X² and X³ each independently represent —O—, —CO—O—, —O—CO—, —O—CO—O— or —O—, W² represents an alicyclic hydrocarbon group having 3 to 12 carbon atoms, —CH₂— included in the alicyclic hydrocarbon group may be replaced by —O— or —CO—, and * represents a bonding site to SO₂).

In formula (B1-A3), Y^{b1′} is preferably a methyl group having a fluorine atom or a cyclic hydrocarbon group having 3 to 20 carbon atoms which may have a substituent (—CH₂—included in the cyclic hydrocarbon group may be replaced by —O—, —CO—, —S— or —SO₂—), and more preferably a trifluoromethyl group, an alicyclic hydrocarbon group having 3 to 18 carbon atoms which may have a substituent (—CH₂—included in the alicyclic hydrocarbon group may be replaced by —O—, —S—, —CO— or —SO₂—) or an aromatic hydrocarbon group having 6 to 18 carbon atoms which may have a substituent. Specifically, a trifluoromethyl group or groups represented by formula (Y1) to formula (Y36) exemplified in formula (B1-A1) is/are preferable.

In formula (B1-A3), when two —SO₂-L^{b2′}-Y^{b1′} combine to form a ring containing A¹, examples include an anion represented by formula (B1-A³′):

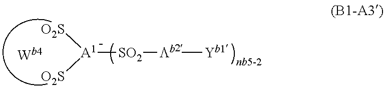

wherein, in formula (B1-A³′),
A¹, Y^{b1′}, L^{b2′} and nb5 are the same as defined in formula (B1-A3), and
W^{b4} represents a disulfonylimide ring or disulfonylmethide ring having 2 to 12 carbon atoms which may have a fluorine atom.

The disulfonylimide ring or disulfonylmethide ring as for W^{b4} preferably has 3 to 12 carbon atoms, and more preferably 3 to 6 carbon atoms, and the hydrogen atom of the methylene group included in the ring is preferably substituted with a fluorine atom.

Examples of the anion represented by formula (B1-A3) include the following. Of these, an anion represented by formula (B3a-1), formula (B3a-2) is preferable.

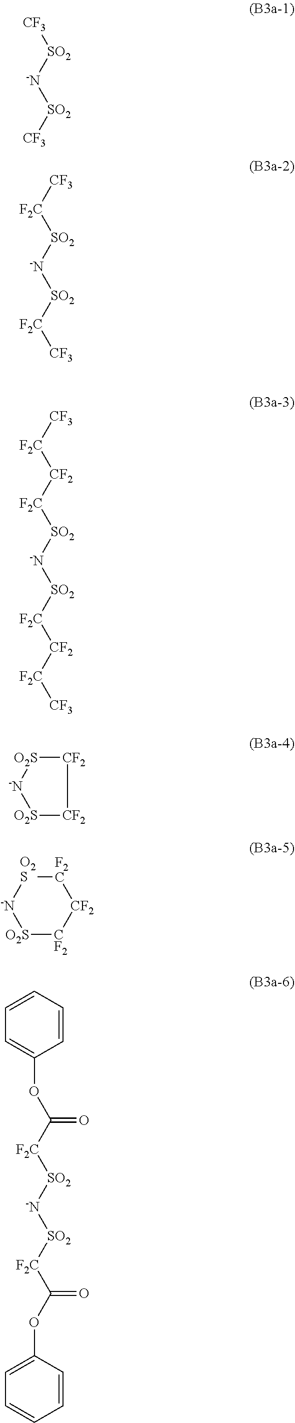

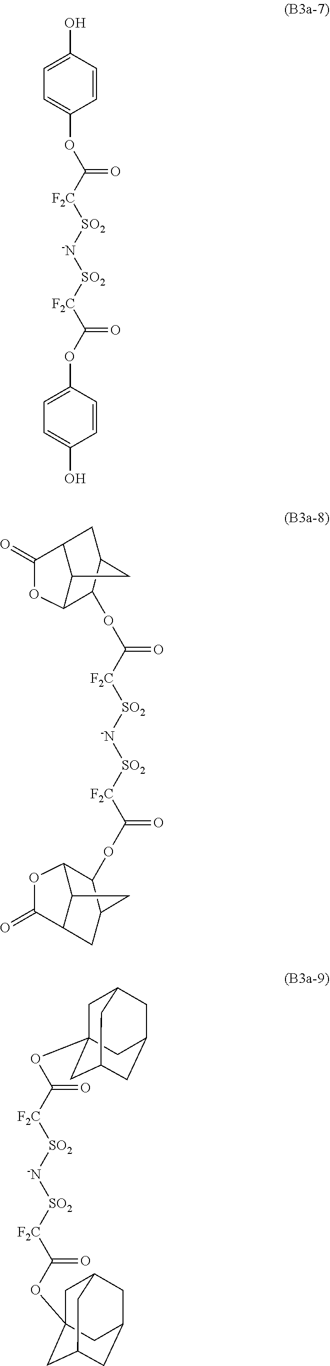

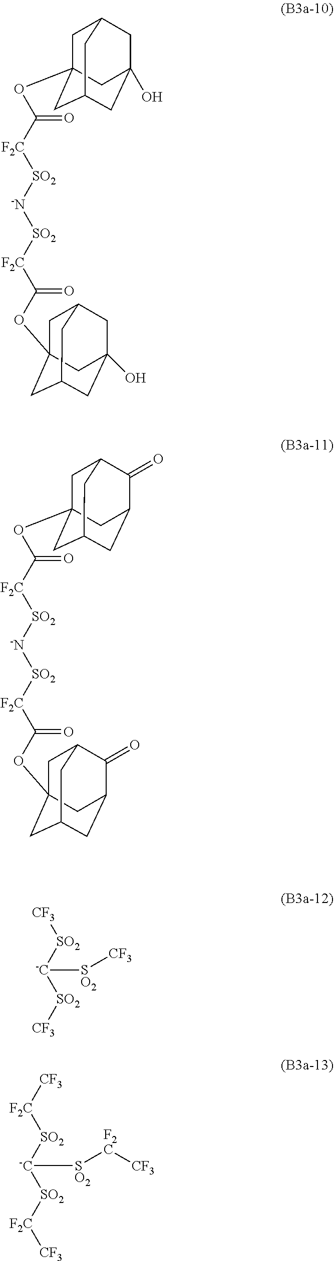

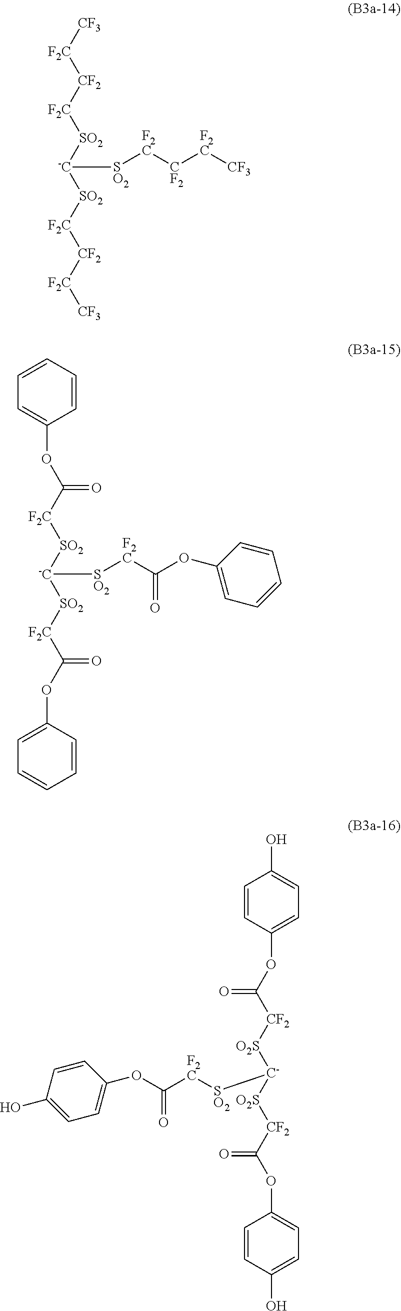

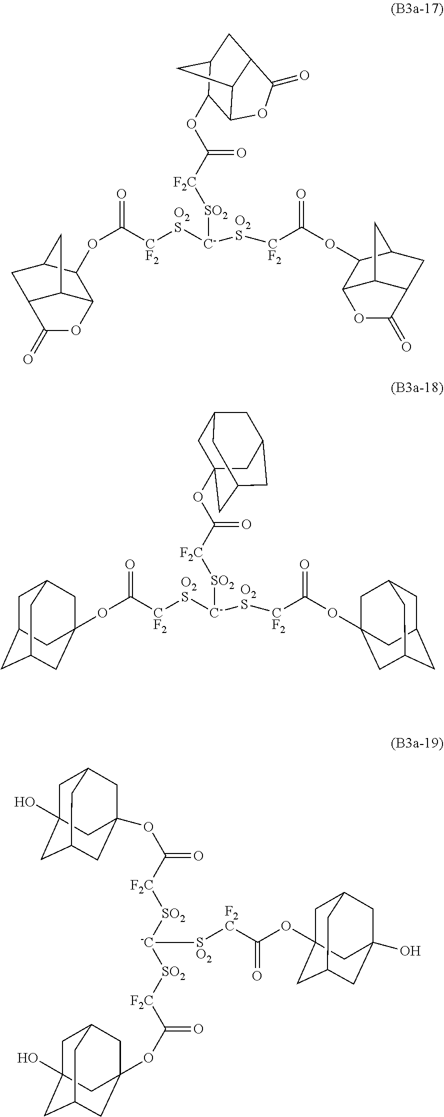

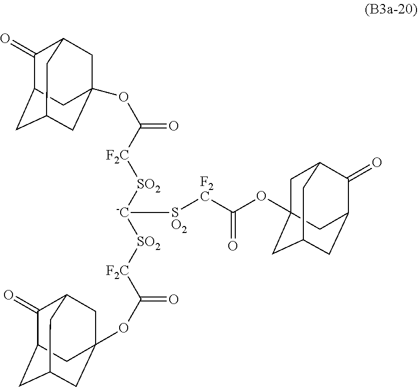

Examples of the carboxylic acid anion include the following.

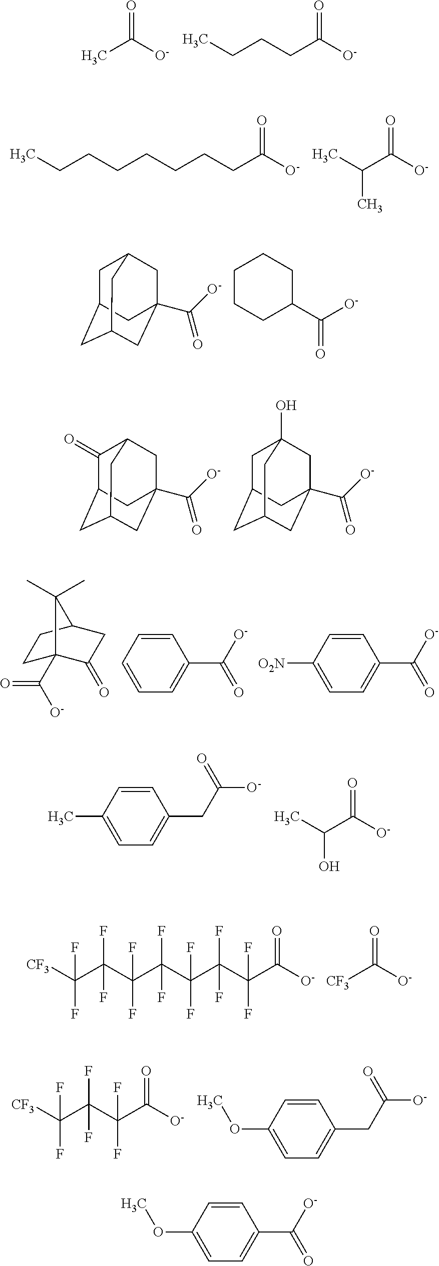

Examples of the organic cation as for Z1⁺ include an organic onium cation, an organic sulfonium cation, an organic iodonium cation, an organic ammonium cation, a benzothiazolium cation and an organic phosphonium cation. Of these, an organic sulfonium cation and an organic iodonium cation are preferable, and an arylsulfonium cation is more preferable. Specific examples thereof include those which are the same as the organic cation ZI⁺ of formula (I).

The acid generator (B) is a combination of the anion mentioned above and the organic cation mentioned above, and these can be optionally combined. The acid generator (B) preferably includes a combination of an anion represented by any one of formula (B1a-1) to formula (B1a-4), formula (B1a-7) to formula (B1a-11), formula (B1a-14) to formula (B1a-30), formula (B1a-35) to formula (B1a-70), formula (B2a-1) to formula (B2a-20) and formula (B3a-1) to formula (B3a-20) with a cation (b2-1), a cation (b2-2), a cation (b2-3), a cation (b2-4) or a cation (b2-5).

The acid generator (B) preferably includes those represented by formula (B1-1) to formula (B1-105), formula (B2-1) to formula (B2-20) and formula (B3-1) to formula (B3-28). Of these, those containing an arylsulfonium cation are preferable and those represented by formula (B1-1) to formula (B1-3), formula (B1-5) to formula (B1-7), formula (B1-11) to formula (B1-14), formula (B1-20) to formula (B1-26), formula (B1-29), formula (B1-31) to formula (B1-105), formula (B2-1) to formula (B2-20) and formula (B3-1) to formula (B3-28) are particularly preferable.

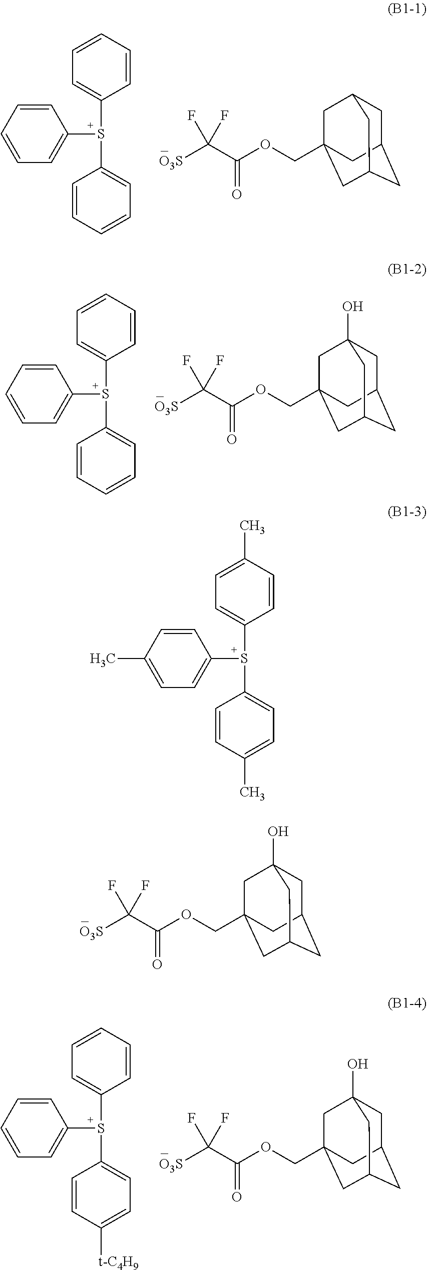

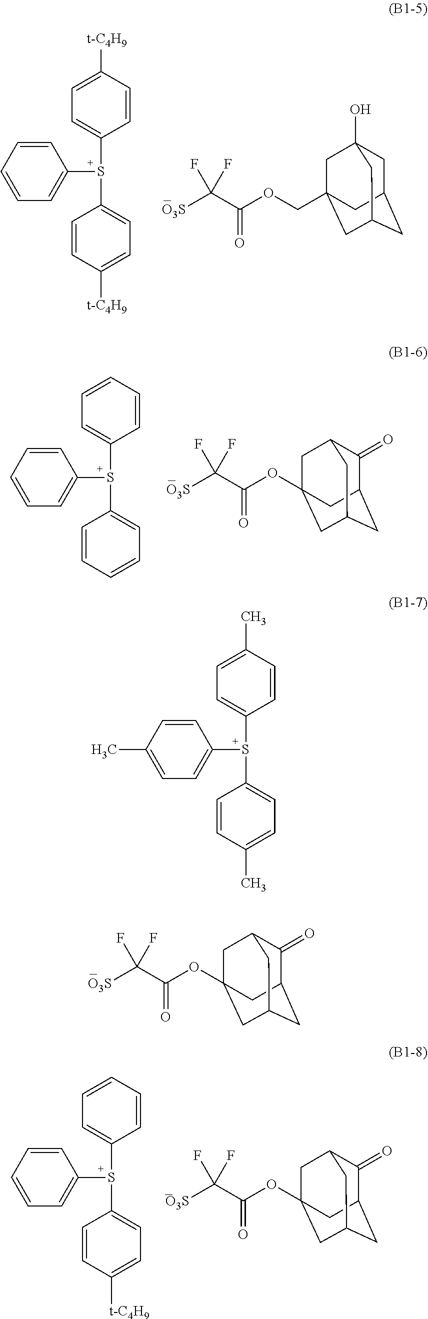

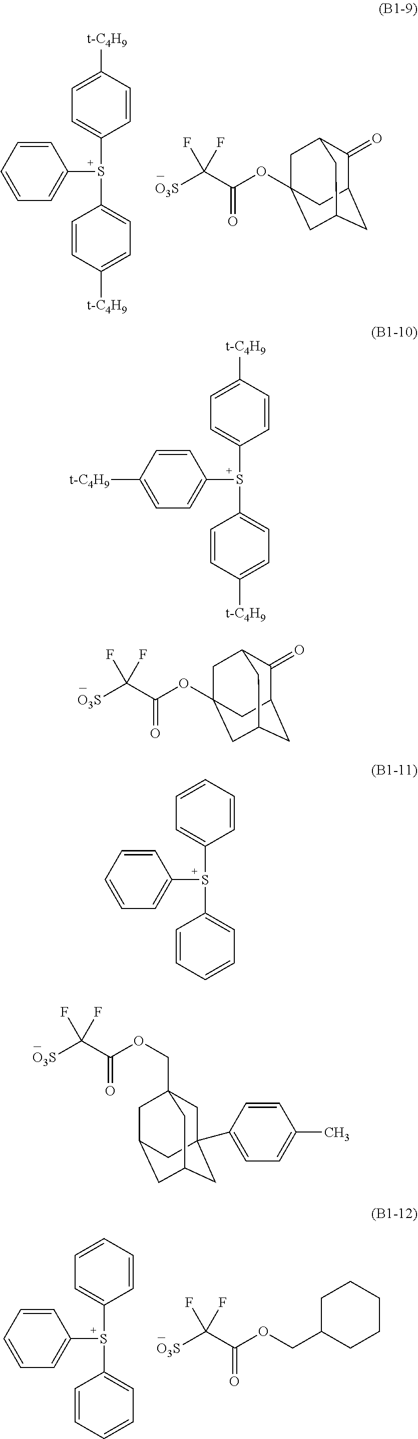

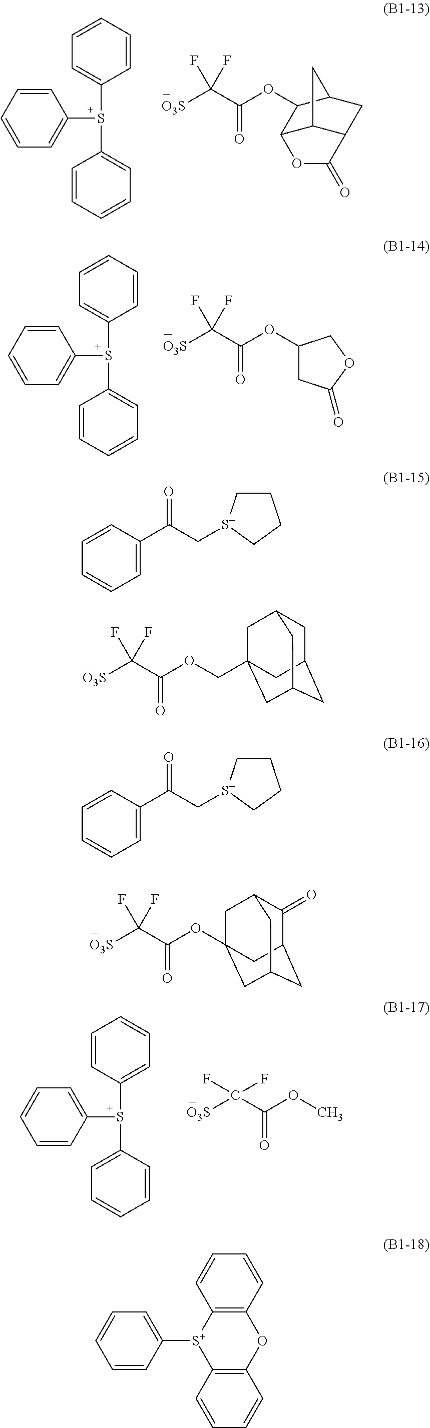

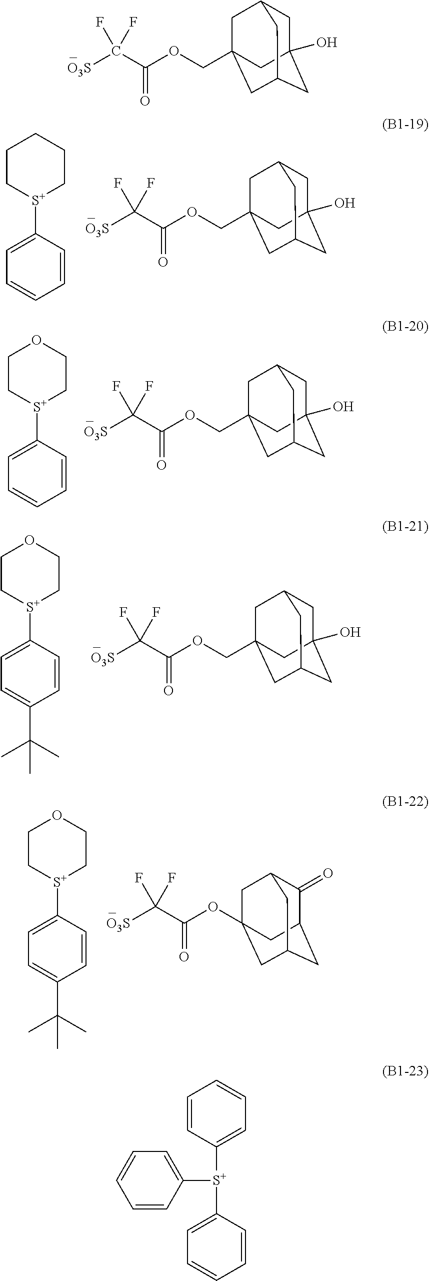

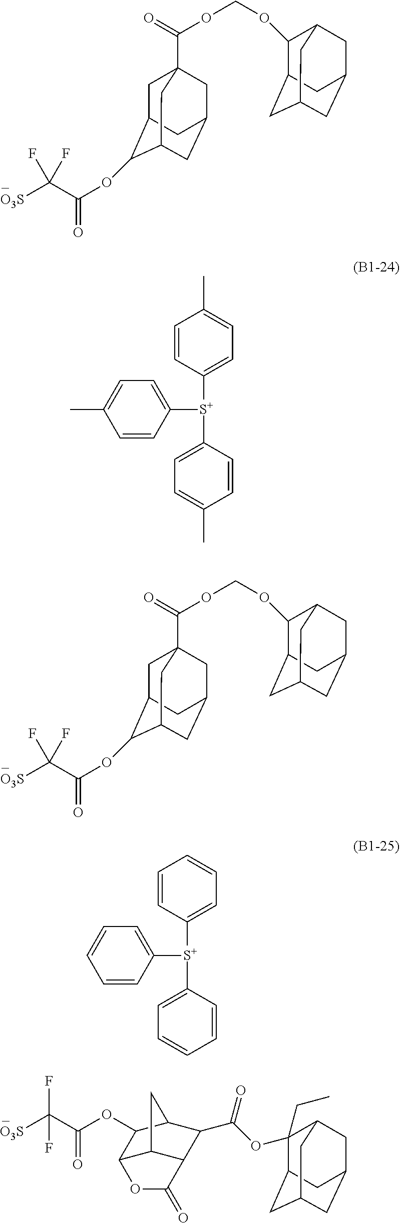

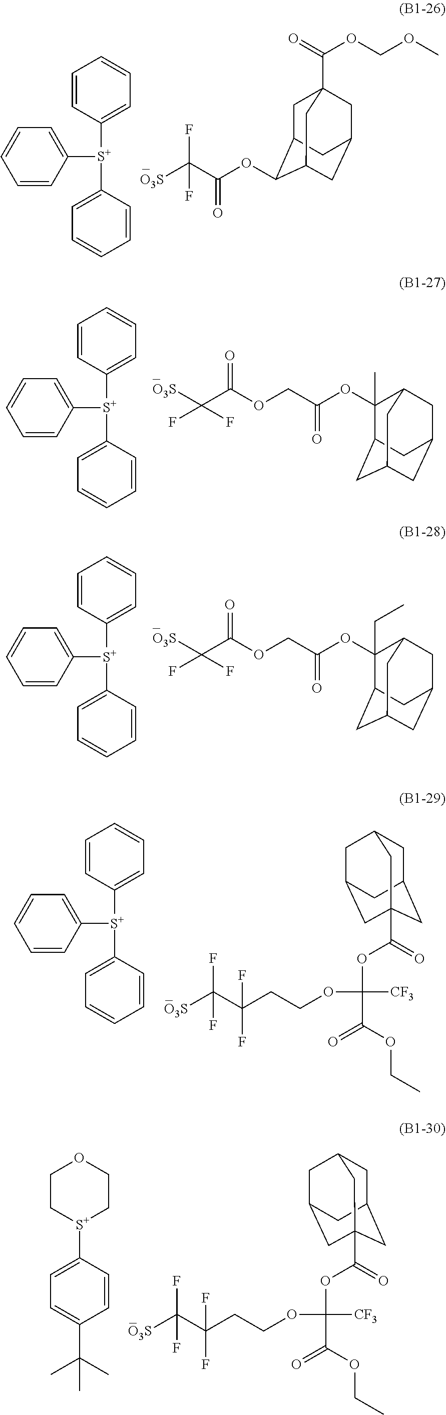

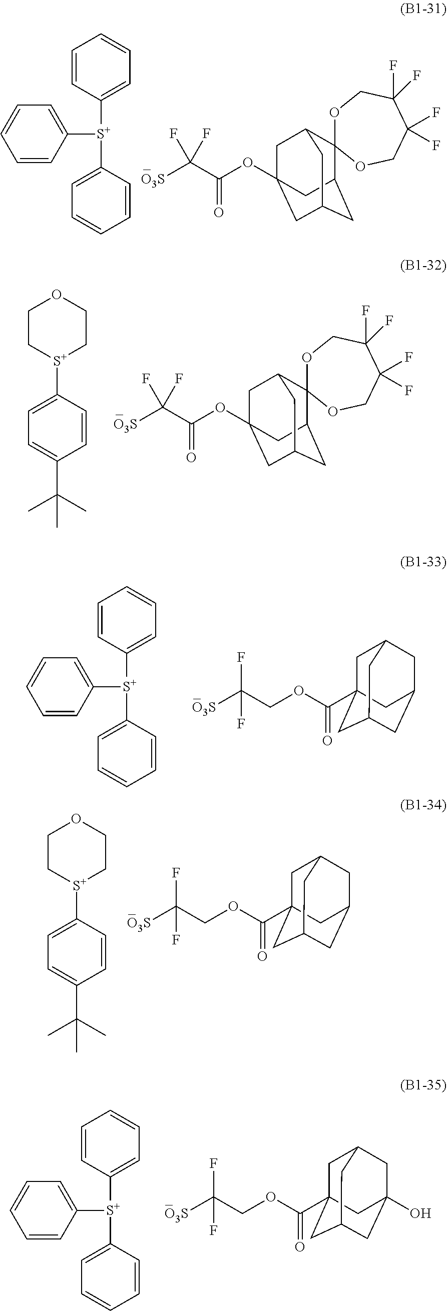

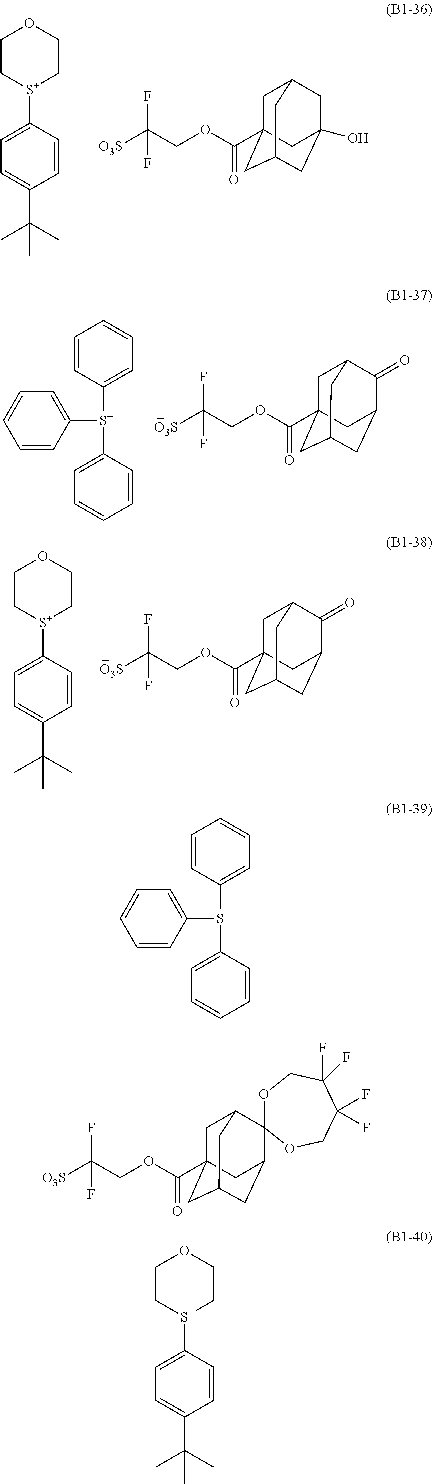

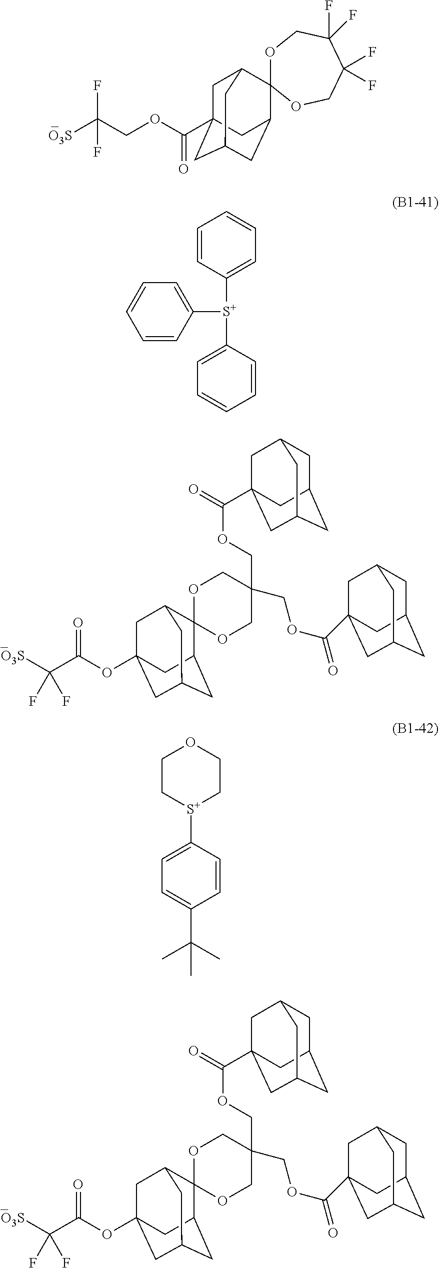

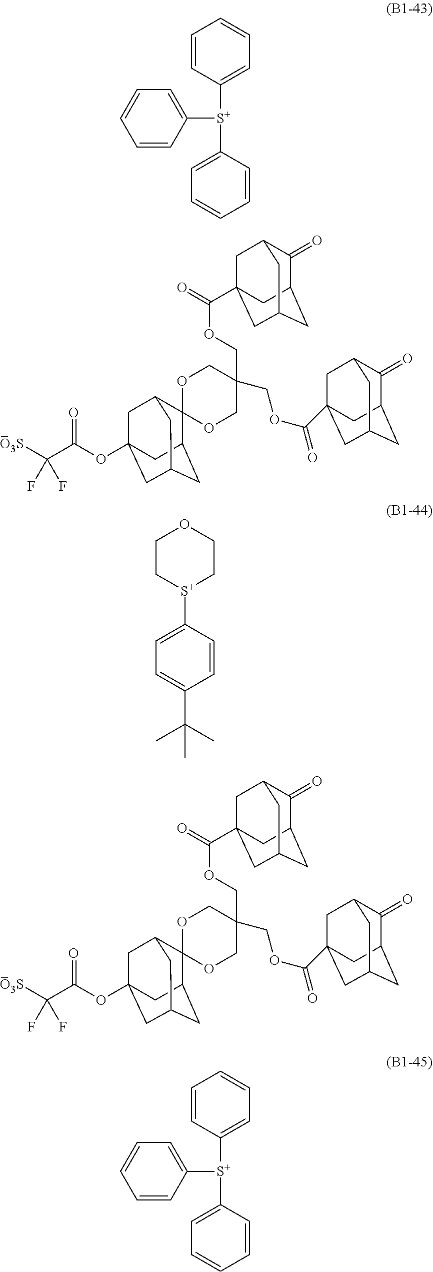

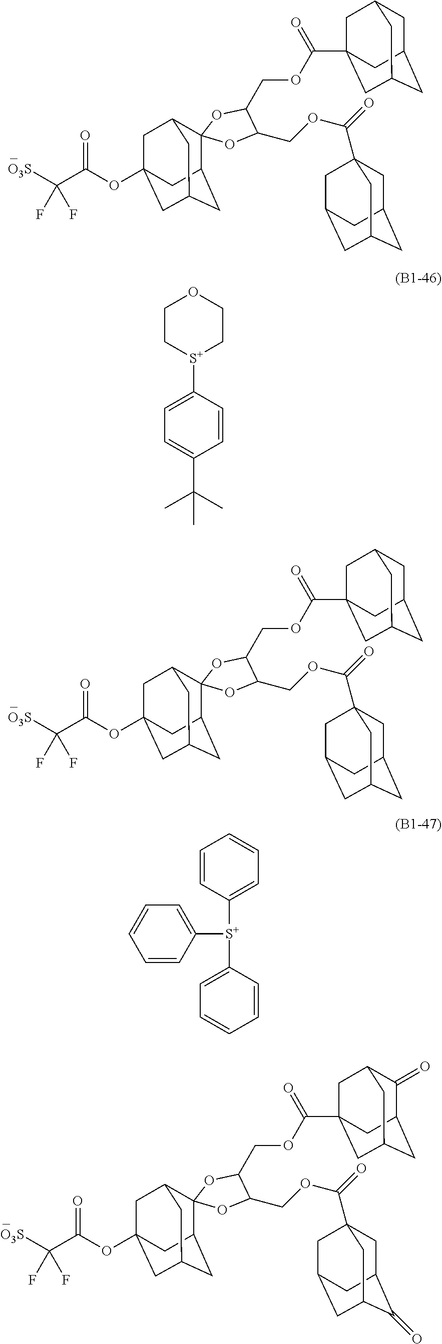

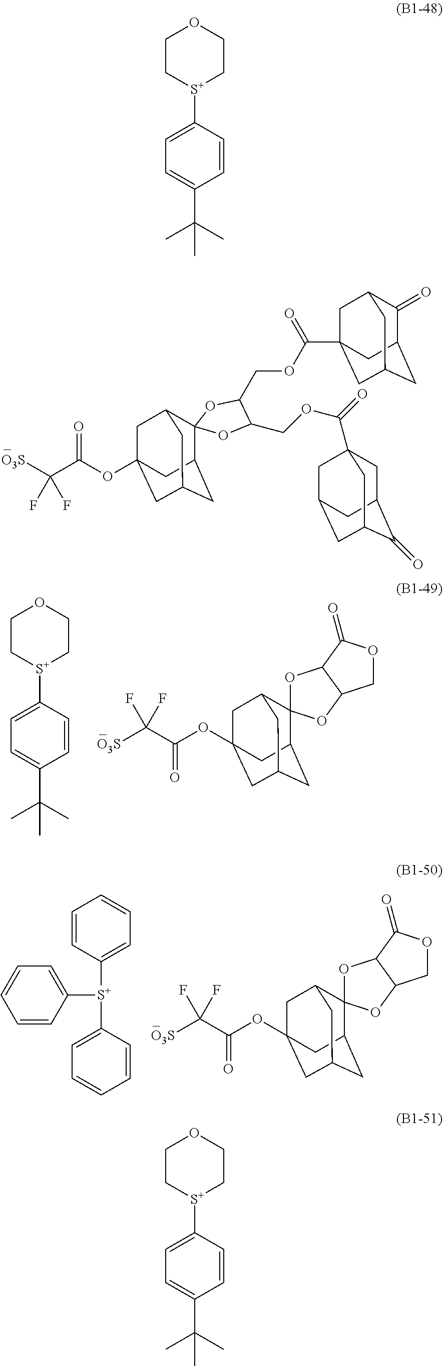

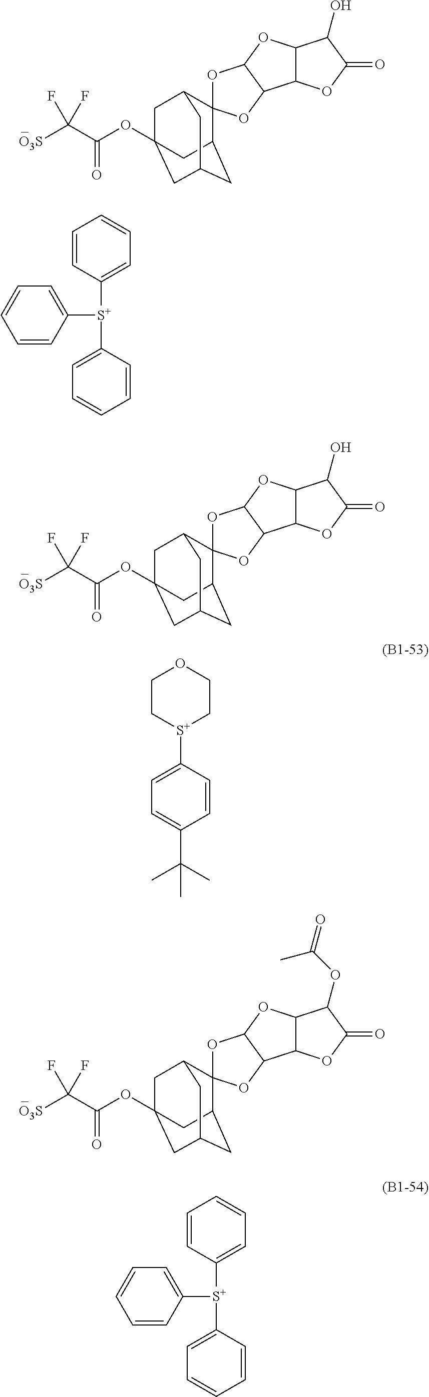

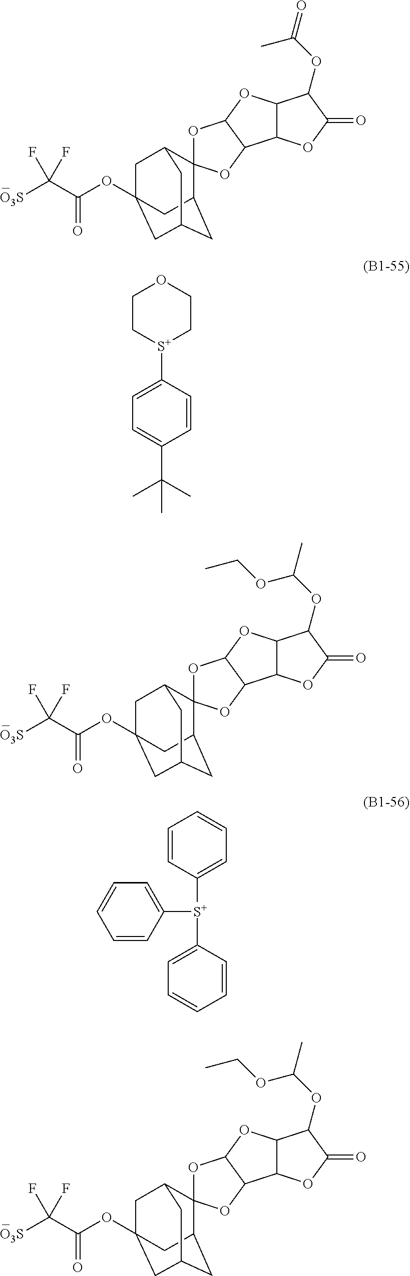

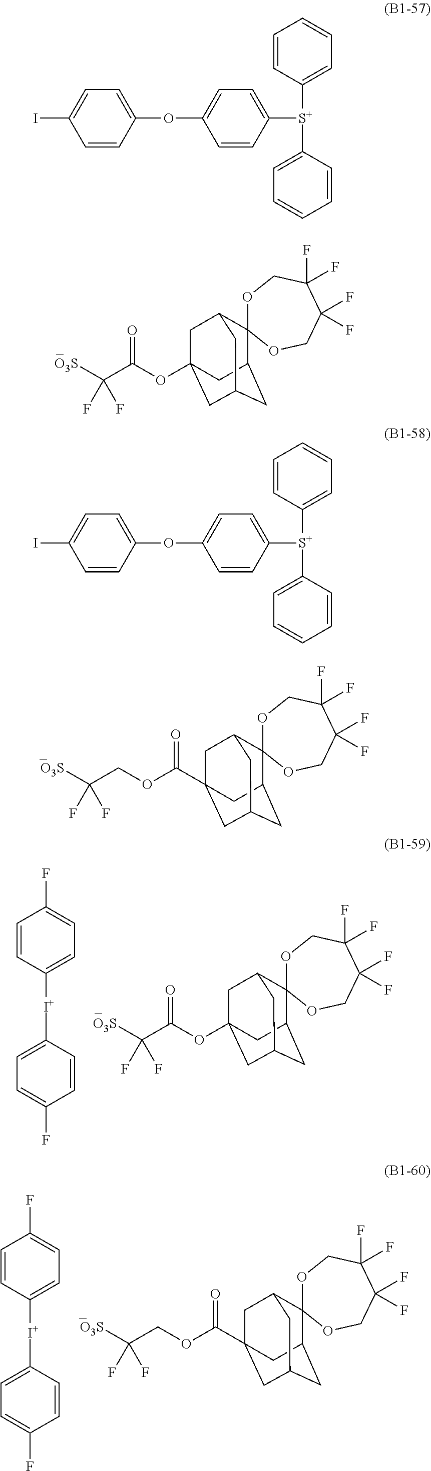

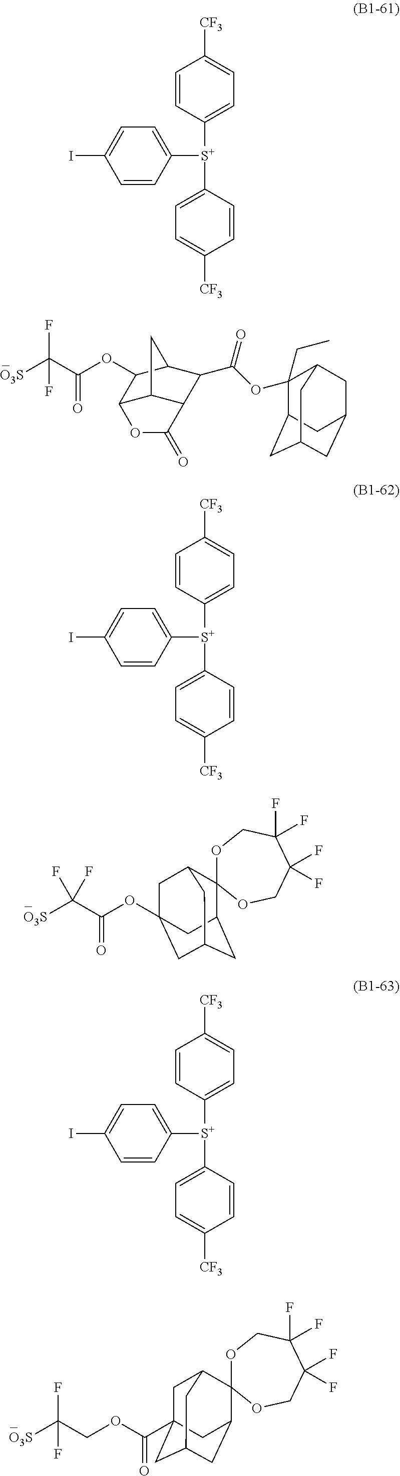

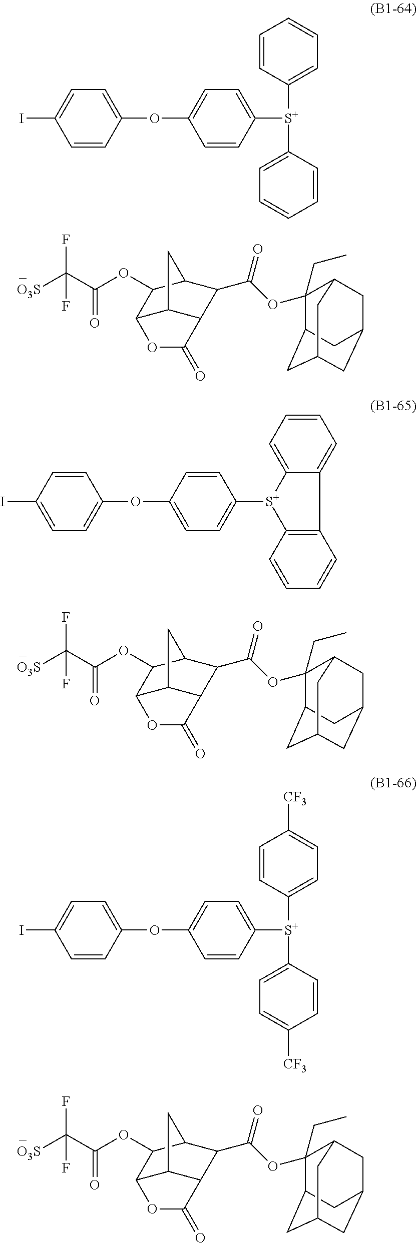

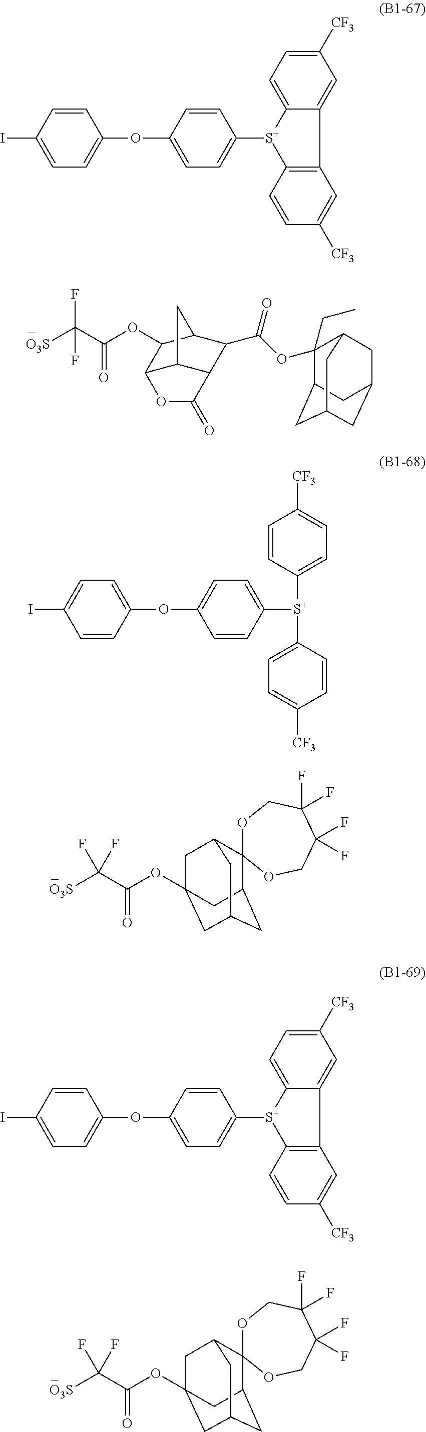

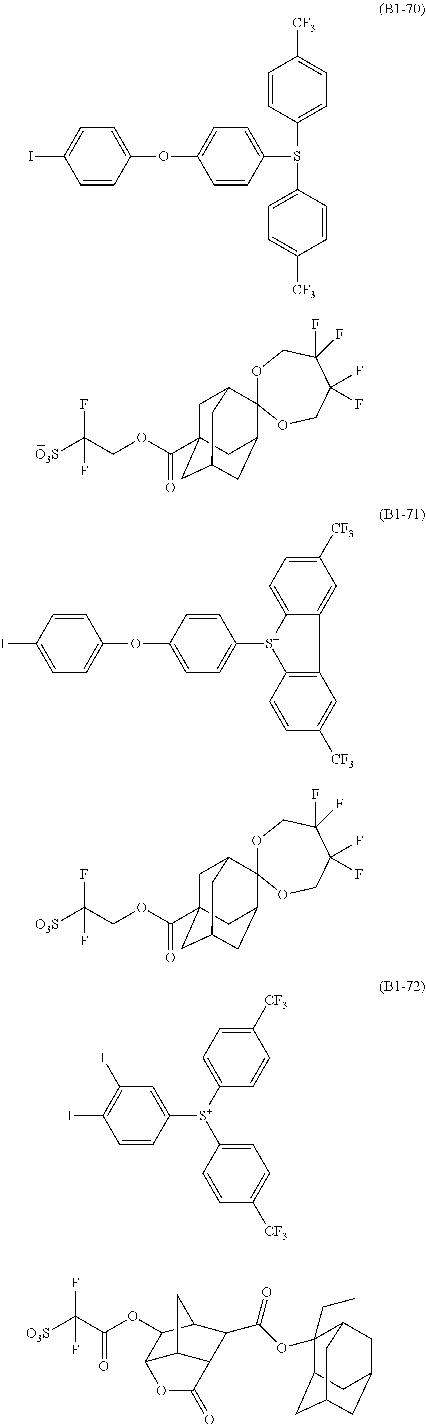

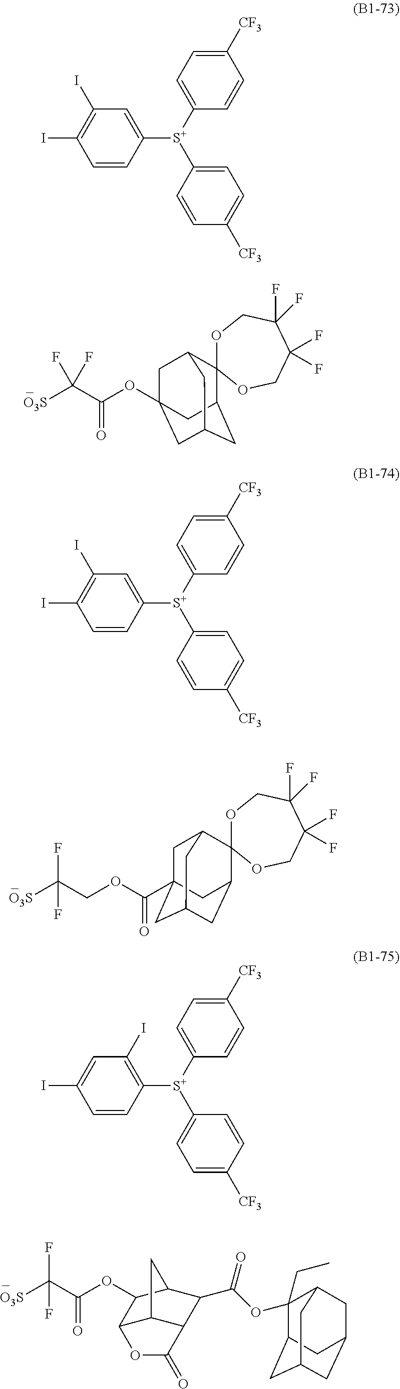

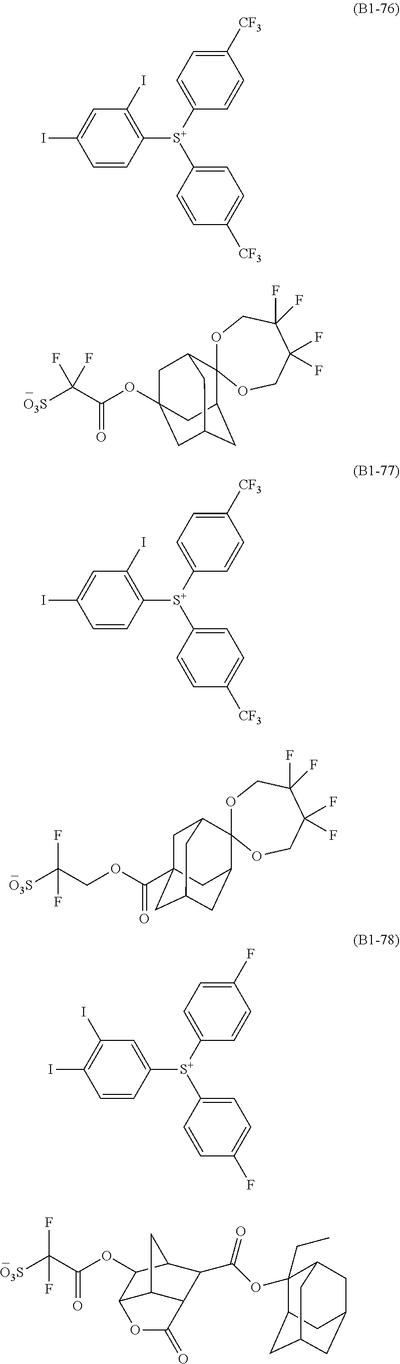

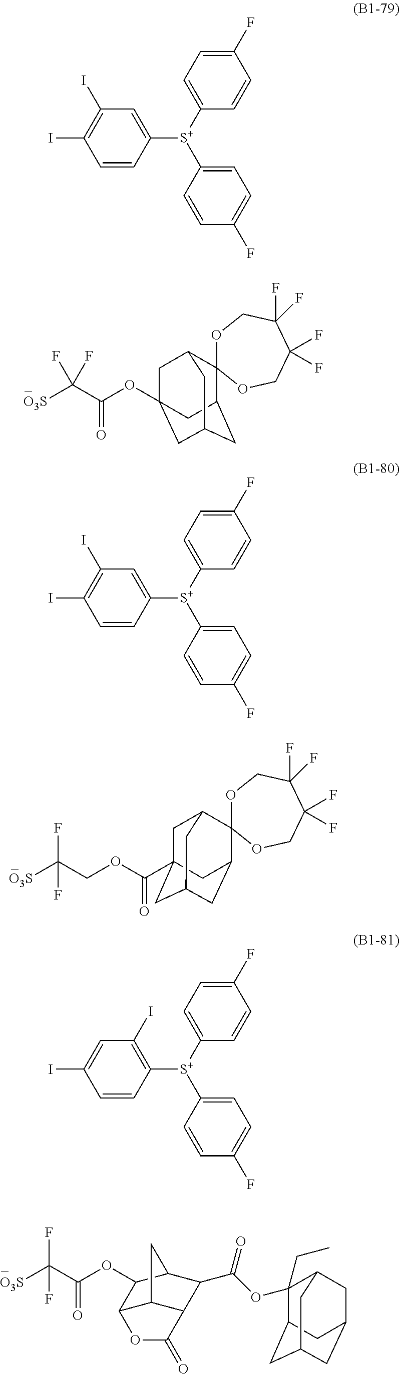

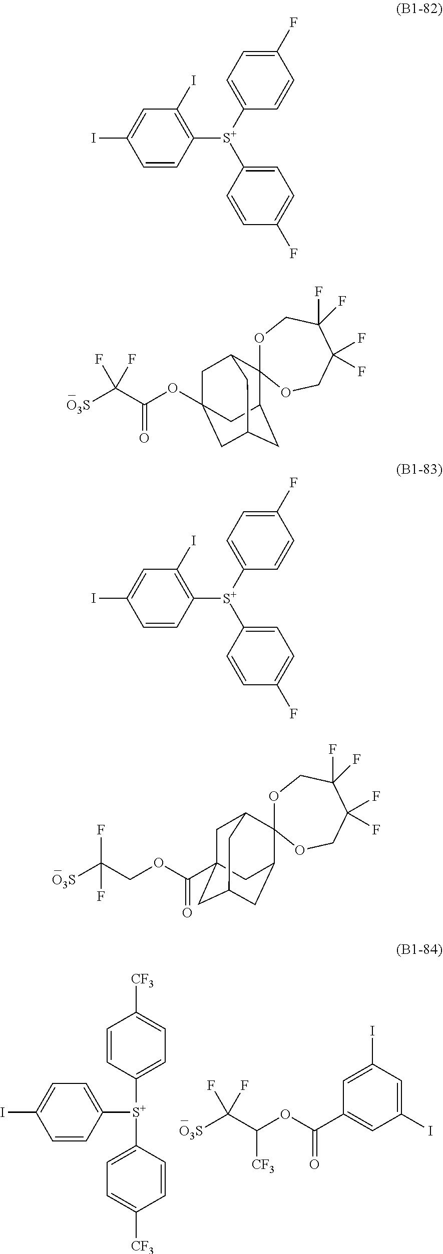

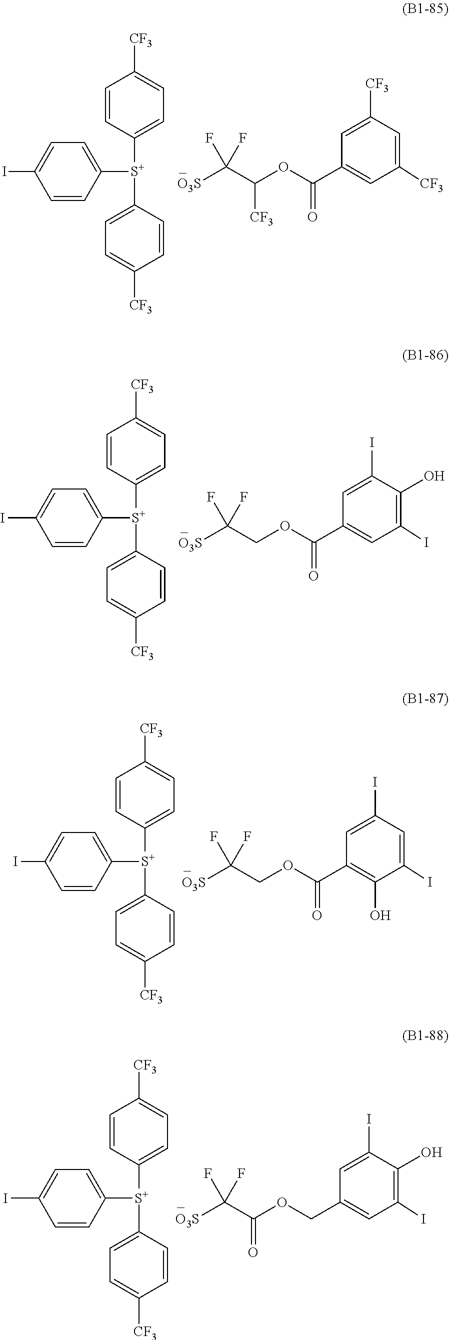

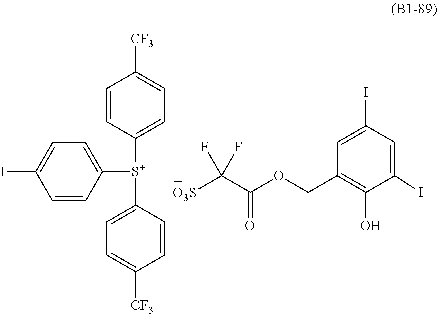

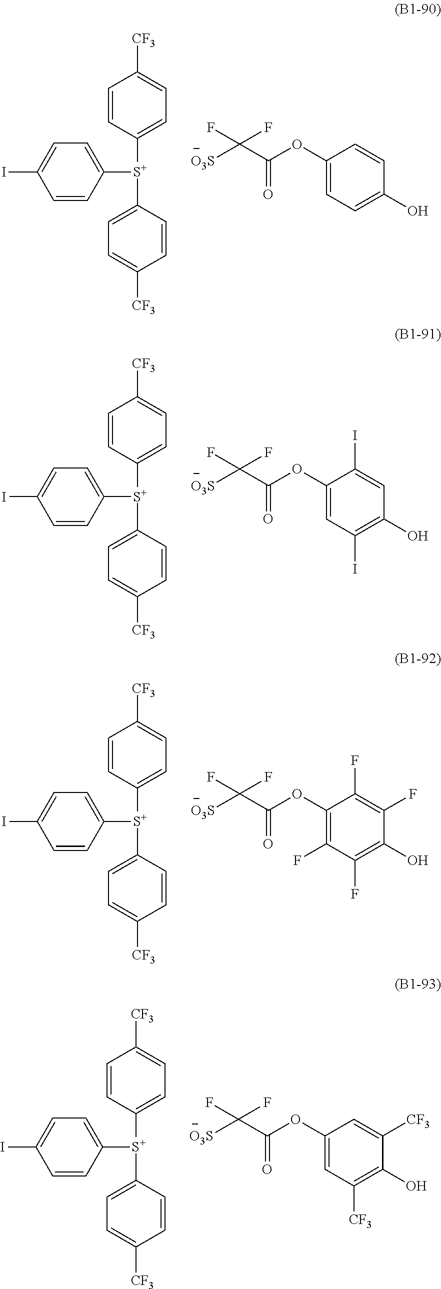

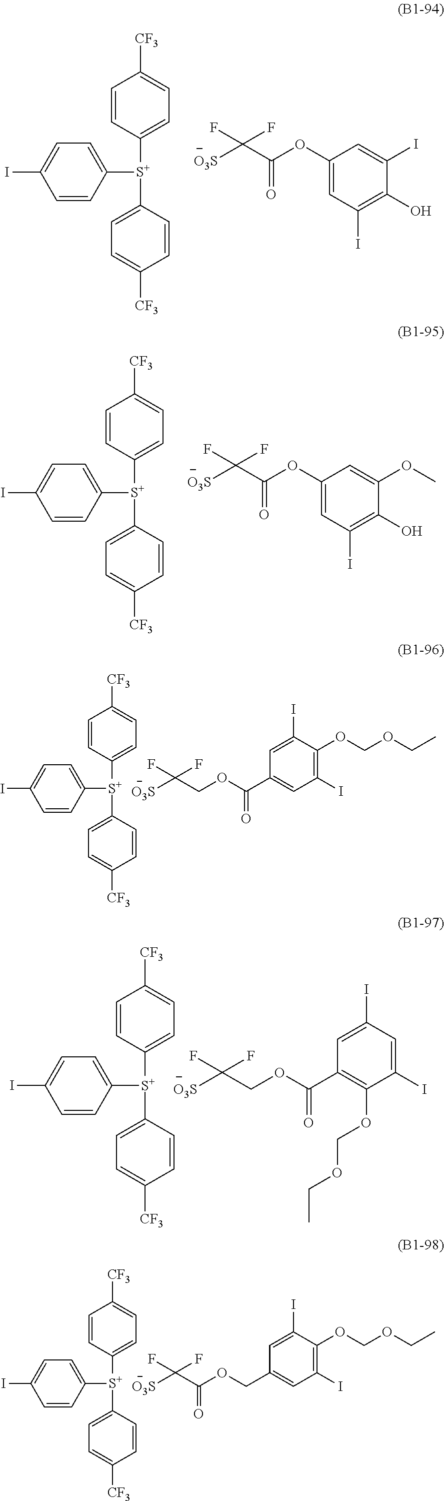

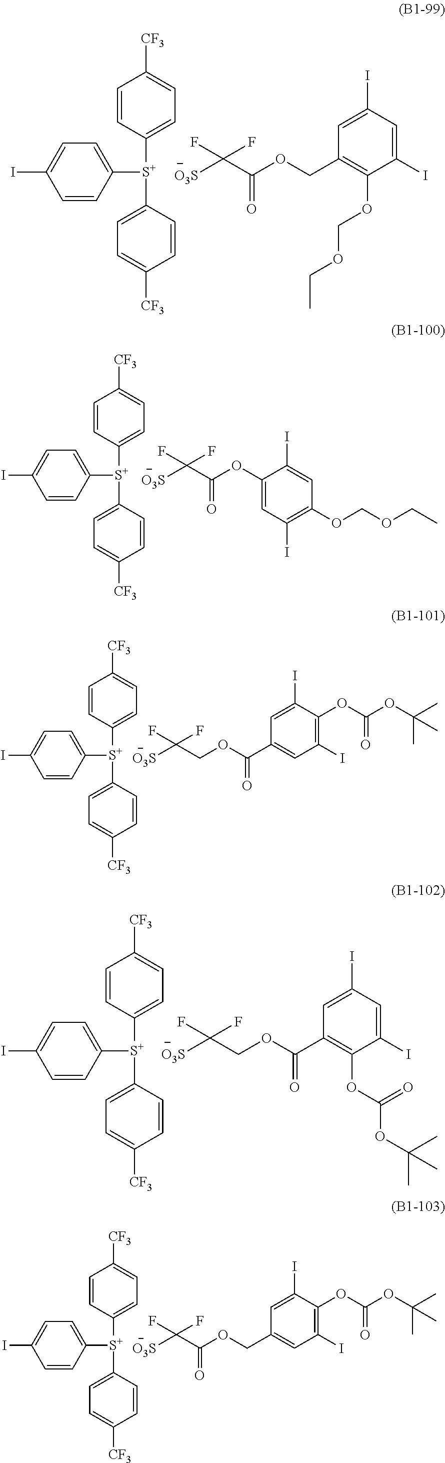

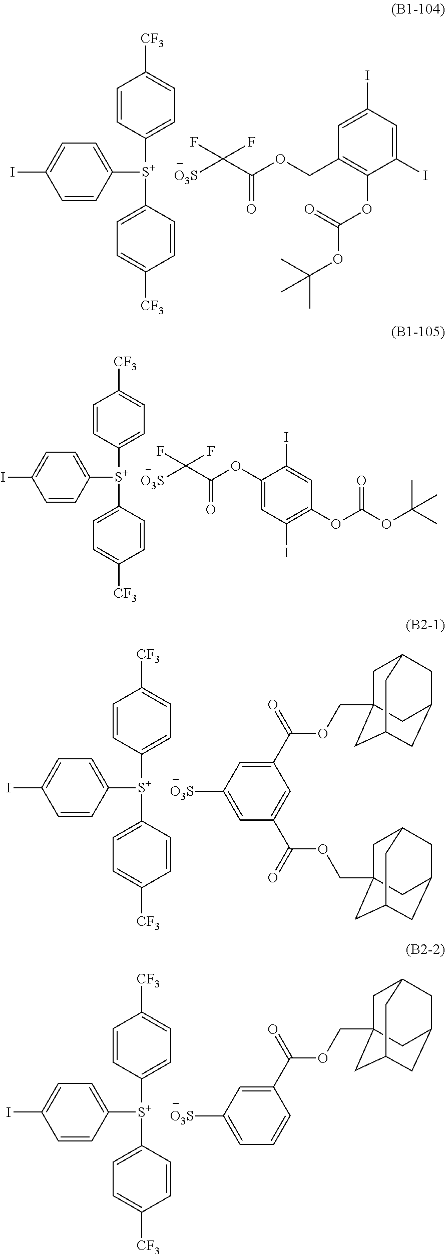

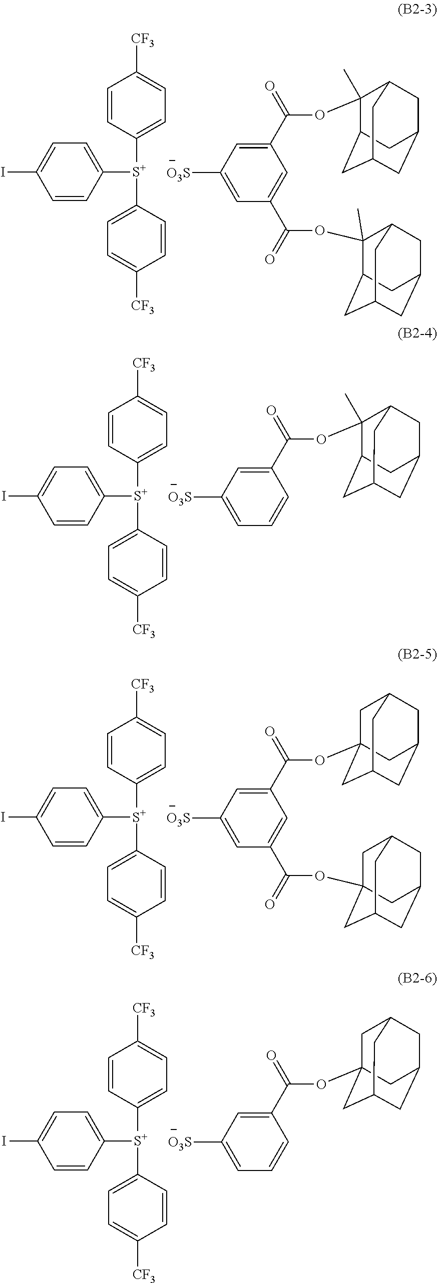

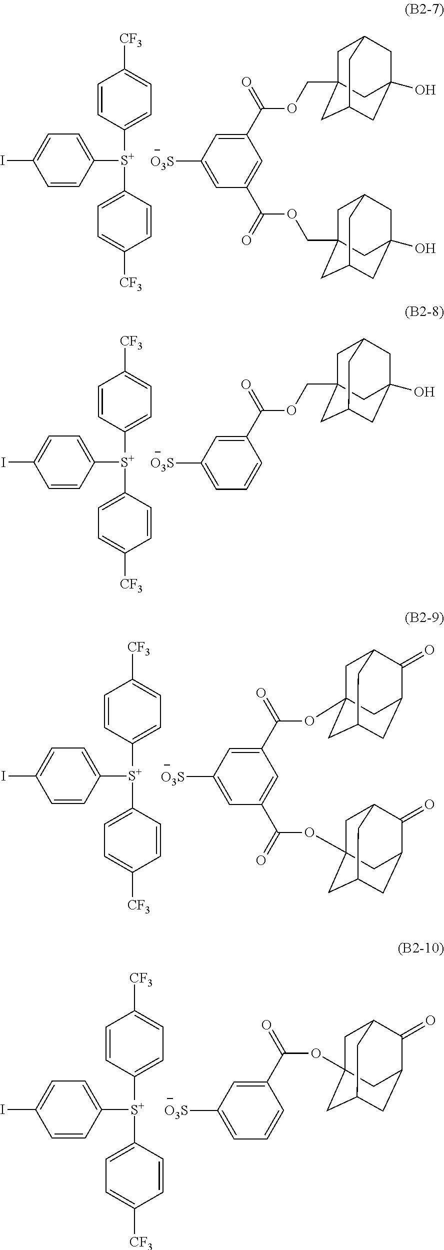

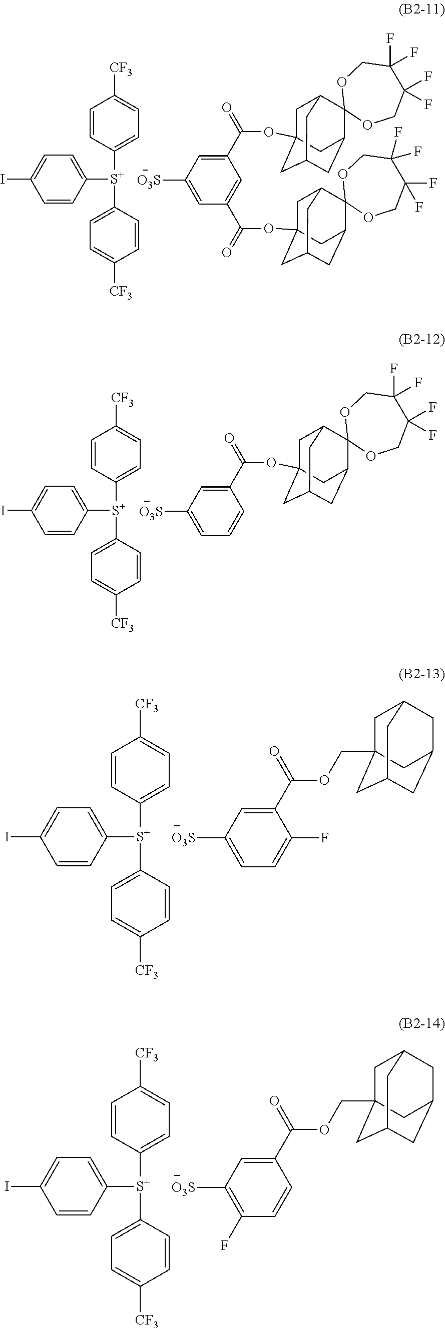

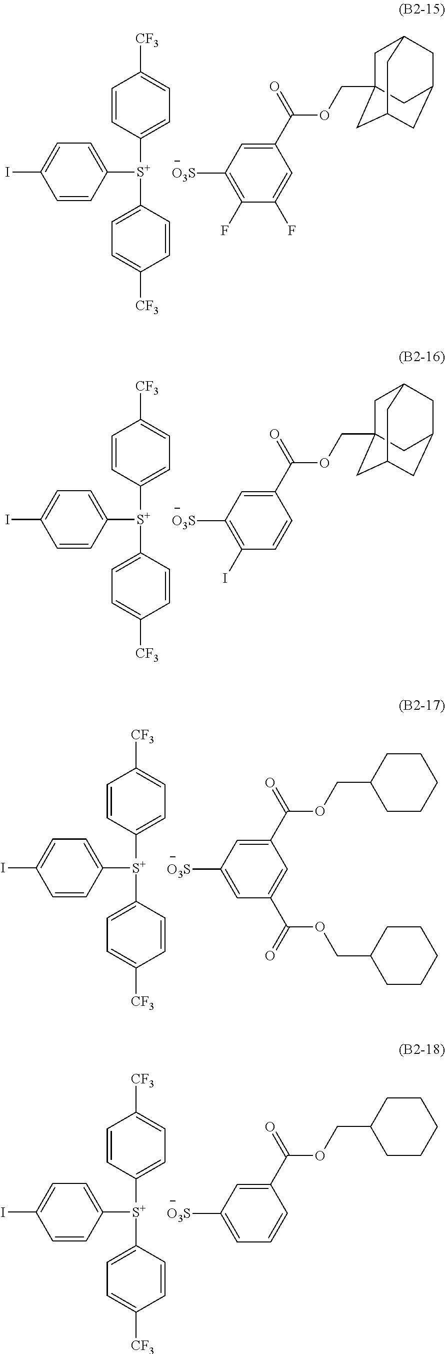

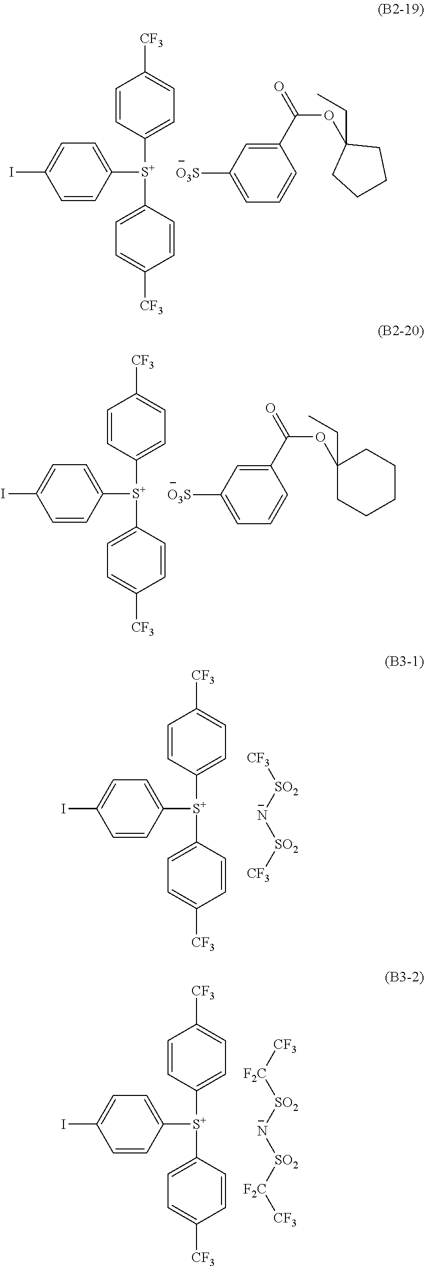

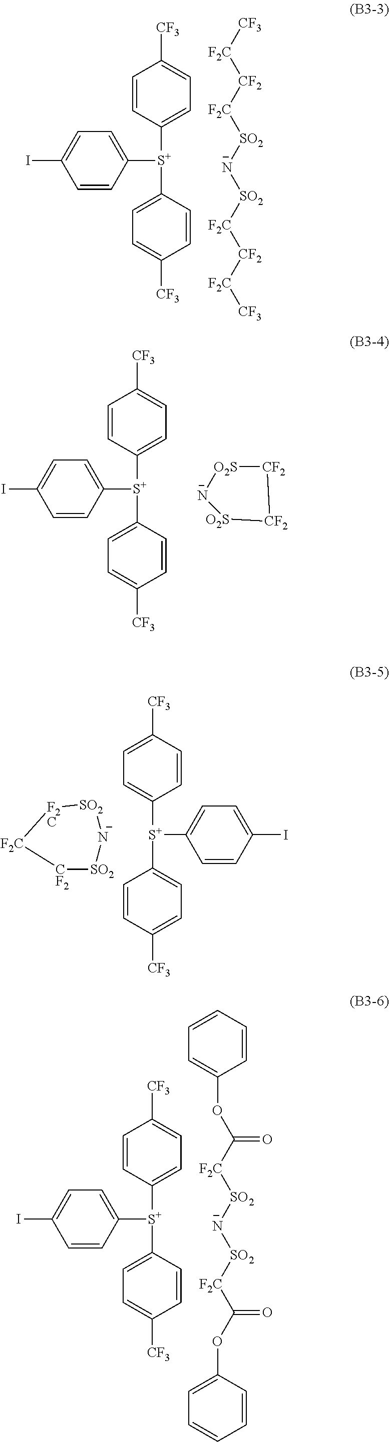

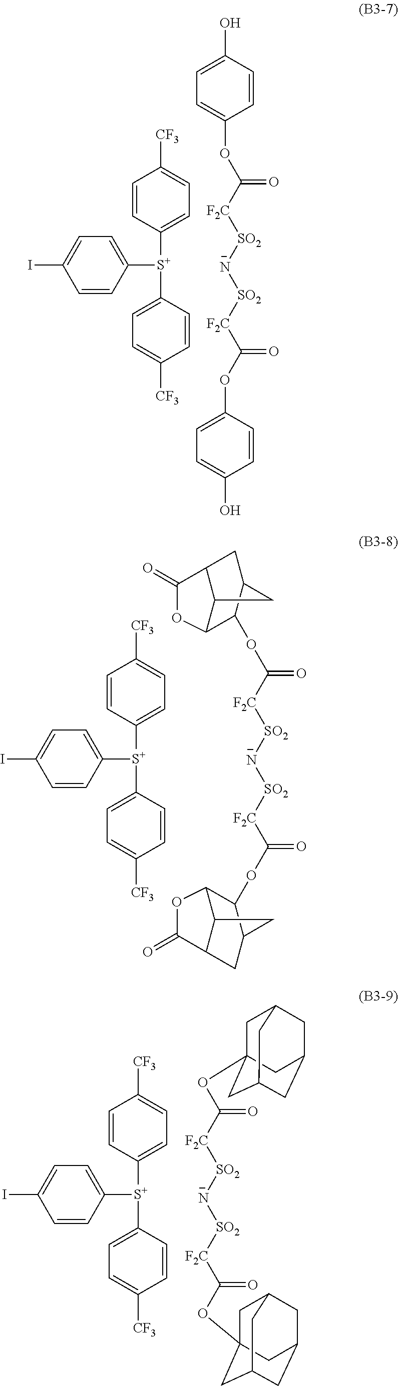 [SMILES: FC(F)(F)c1ccc([S+](c2ccc(I)cc2)c2ccc(C(F)(F)F)cc2)cc1 ; O=C(Oc1ccc(O)cc1)C(F)(F)S(=O)(=O)[N-]S(=O)(=O)C(F)(F)C(=O)Oc1ccc(O)cc1 ; FC(F)(F)c1ccc([S+](c2ccc(I)cc2)c2ccc(C(F)(F)F)cc2)cc1 ; O=C1OC2C3CC(CC13)C2OC(=O)C(F)(F)S(=O)(=O)[N-]S(=O)(=O)C(F)(F)C(=O)OC1C2CC3C(=O)OC1C3C2 ; FC(F)(F)c1ccc([S+](c2ccc(I)cc2)c2ccc(C(F)(F)F)cc2)cc1 ; O=C(OC12CC3CC(CC(C3)C1)C2)C(F)(F)S(=O)(=O)[N-]S(=O)(=O)C(F)(F)C(=O)OC12CC3CC(CC(C3)C1)C2]

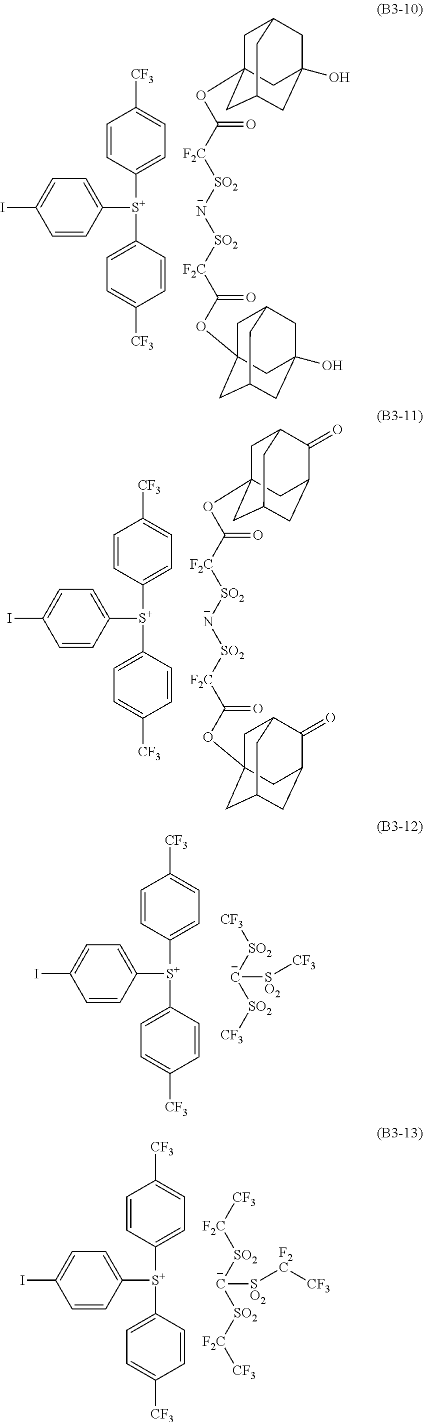

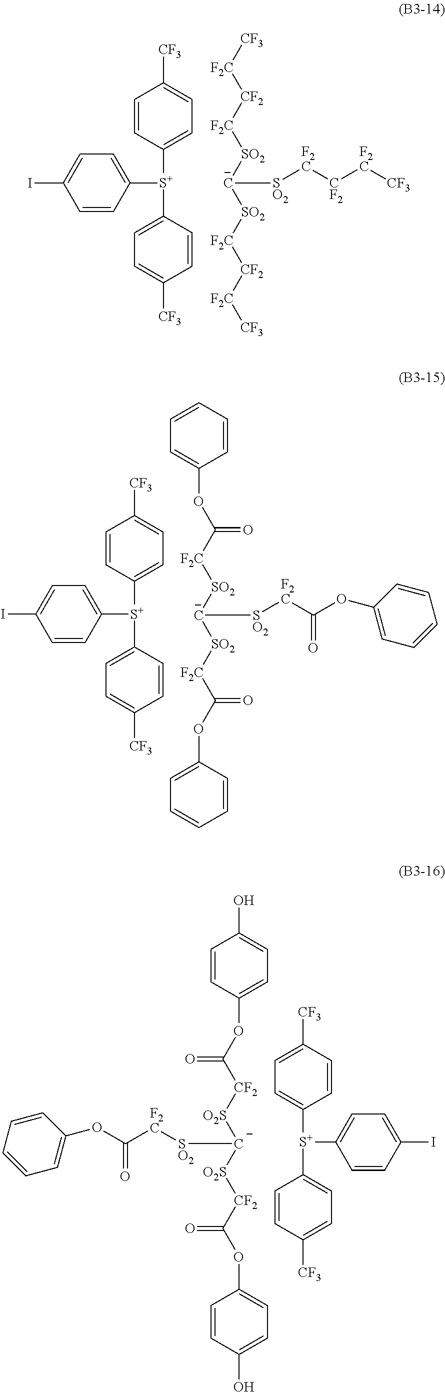 [SMILES: FC(F)(F)c1ccc([S+](c2ccc(I)cc2)c2ccc(C(F)(F)F)cc2)cc1 ; O=S(=O)([C-](S(=O)(=O)C(F)(F)C(F)(F)C(F)(F)C(F)(F)F)S(=O)(=O)C(F)(F)C(F)(F)C(F)(F)C(F)(F)F)C(F)(F)C(F)(F)C(F)(F)C(F)(F)F ; FC(F)(F)c1ccc([S+](c2ccc(I)cc2)c2ccc(C(F)(F)F)cc2)cc1 ; O=C(Oc1ccccc1)C(F)(F)S(=O)(=O)[C-](S(=O)(=O)C(F)(F)C(=O)Oc1ccccc1)S(=O)(=O)C(F)(F)C(=O)Oc1ccccc1 ; O=C(Oc1ccccc1)C(F)(F)S(=O)(=O)[C-](S(=O)(=O)C(F)(F)C(=O)Oc1ccc(O)cc1)S(=O)(=O)C(F)(F)C(=O)Oc1ccc(O)cc1 ; FC(F)(F)c1ccc([S+](c2ccc(I)cc2)c2ccc(C(F)(F)F)cc2)cc1]

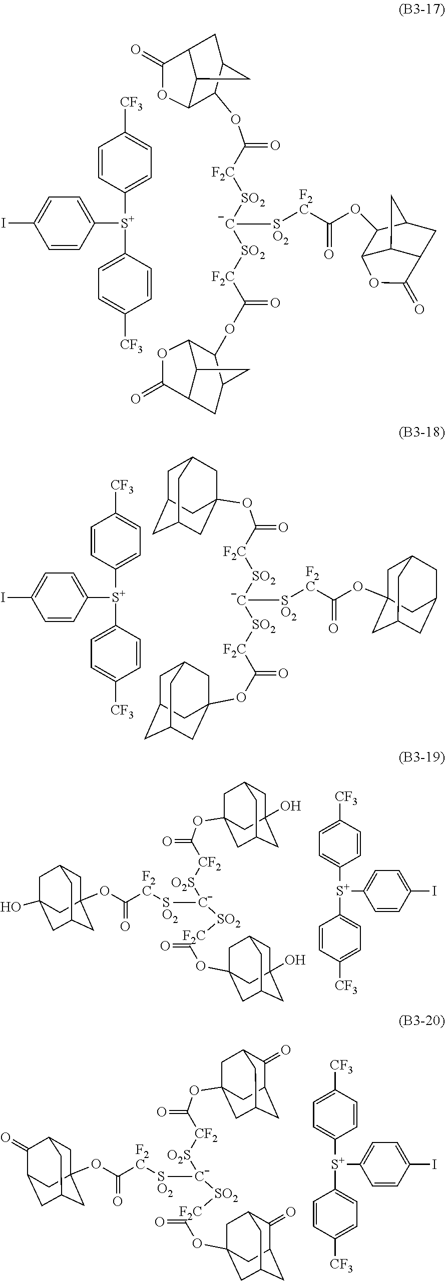

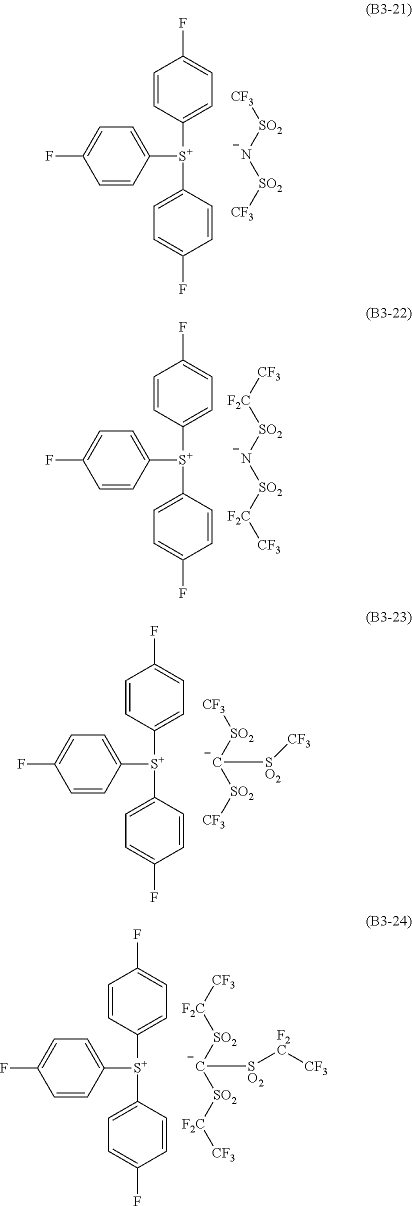

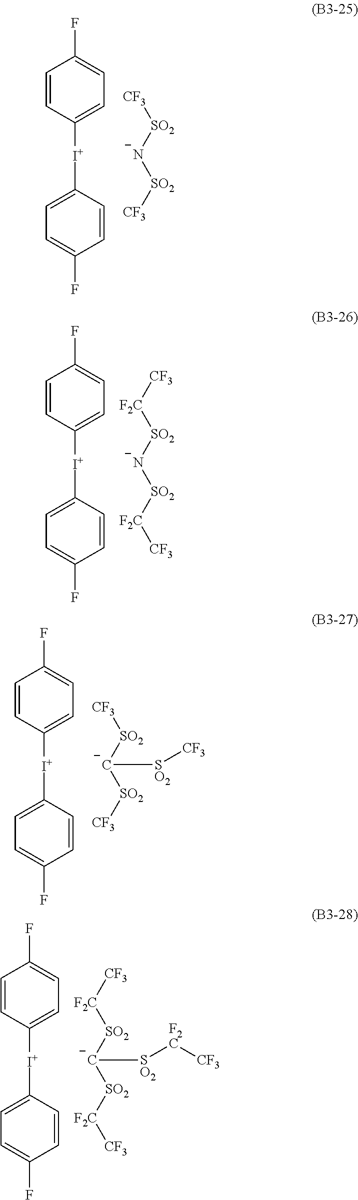

In the resist composition of the present invention, the content of the acid generator is, for example, 0.1% by mass or more and 99.9% by mass or less, preferably 1% by mass or more and 45% by mass or less, more preferably 1% by mass or more and 40% by mass or less, and still more preferably 3% by mass or more and 35% by mass or less, based on the solid content of the resist composition. When including the resin (A) or the like, the content of the acid generator is preferably 1 part by mass or more and 45 parts by mass or less, more preferably 1 part by mass or more and 40 parts by mass or less, and still more preferably 3 parts by mass or more and 35 parts by mass or less, based on 100 parts by mass of the resin (A) or the like. The resist composition of the present invention may include an acid generator alone, or a plurality thereof.

### <Solvent (E)>

The content of the solvent (E) in the resist composition is usually 90% by mass or more and 99.9% by mass or less, preferably 92% by mass or more and 99% by mass or less, and more preferably 94% by mass or more and 99% by mass or less. The content of the solvent (E) can be measured, for example, by a known analysis means such as liquid chromatography or gas chromatography.

Examples of the solvent (E) include glycol ether esters such as ethylcellosolve acetate, methylcellosolve acetate and propylene glycol monomethyl ether acetate; glycol ethers such as propylene glycol monomethyl ether; esters such as ethyl lactate, butyl acetate, amyl acetate and ethyl pyruvate; ketones such as acetone, methyl isobutyl ketone, 2-heptanone and cyclohexanone; and cyclic esters such as γ-butyrolactone. The solvent (E) may be used alone, or two or more solvents may be used.

### <Quencher (C)>

Examples of the quencher (C) include a basic nitrogen-containing organic compound, and a salt generating an acid having an acidity lower than that of an acid generated from an acid generator (acid generator (B)) (in which a carboxylate represented by formula (I) is excluded). When the resist composition includes the quencher (C), the content of the quencher (C) is preferably about 0.01 to 15% by mass, more preferably about 0.01 to 10% by mass, still more preferably about 0.1 to 8% by mass, and yet more preferably about 0.1 to 7% by mass, based on the amount of the solid component of the resist composition.

Examples of the basic nitrogen-containing organic compound include amine and an ammonium salt. Examples of the amine include an aliphatic amine and an aromatic amine. Examples of the aliphatic amine include a primary amine, a secondary amine and a tertiary amine.

Examples of the amine include 1-naphthylamine, 2-naphthylamine, aniline, diisopropylaniline, 2-, 3- or 4-methylaniline, 4-nitroaniline, N-methylaniline, N,N-dimethylaniline, diphenylamine, hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethyldipentylamine, ethyldihexylamine, ethyldiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane, 4,4′-diamino-3,3′-diethyldiphenylmethane, 2,2′-methylenebisaniline, imidazole, 4-methylimidazole, pyridine, 4-methylpyridine, 1,2-di(2-pyridyl)ethane, 1,2-di(4-pyridyl)ethane, 1,2-di(2-pyridyl)ethene, 1,2-di(4-pyridyl)ethene, 1,3-di(4-pyridyl)propane, 1,2-di(4-pyridyloxy)ethane, di(2-pyridyl)ketone, 4,4′-dipyridyl sulfide, 4,4′-dipyridyl disulfide, 2,2′-dipyridylamine, 2,2′-dipicolylamine, bipyridine and the like, preferably diisopropylaniline, and more preferably 2,6-diisopropylaniline.

Examples of the ammonium salt include tetramethylammonium hydroxide, tetraisopropylammonium hydroxide, tetrabutylammonium hydroxide, tetrahexylammonium hydroxide, tetraoctylammonium hydroxide, phenyltrimethylammonium hydroxide, 3-(trifluoromethyl)phenyltrimethylammonium hydroxide, tetra-n-butylammonium salicylate and choline.

<Salt Generating Acid Having Acidity Lower than that of Acid Generated from Acid Generator>

The acidity in a salt generating an acid having an acidity lower than that of an acid generated from the acid generator is indicated by the acid dissociation constant (pKa). Regarding the salt generating an acid having an acidity lower than that of an acid generated from the acid generator, the acid dissociation constant of an acid generated from the salt usually meets the following inequality: −3<pKa, preferably −1<pKa<7, and more preferably 0<pKa<5.

Examples of the salt generating an acid having an acidity lower than that of an acid generated from the acid generator include salts represented by the following formulas, a salt represented by formula (D) mentioned in JP 2015-147926 A (hereinafter sometimes referred to as “weak acid inner salt (D)”, and salts mentioned in JP 2012-229206 A, JP 2012-6908 A, JP 2012-72109 A, JP 2011-39502 A and JP 2011-191745 A. The salt generating an acid having an acidity lower than that of an acid generated from the acid generator is preferably a salt generating a carboxylic acid having an acidity lower than that of an acid generated from the acid generator (salt having a carboxylic acid anion), more preferably a weak acid inner salt (D), and still more preferably a diphenyliodonium salt containing a phenyl group substituted with a carboxylic acid anion among the weak acid inner salt (D).

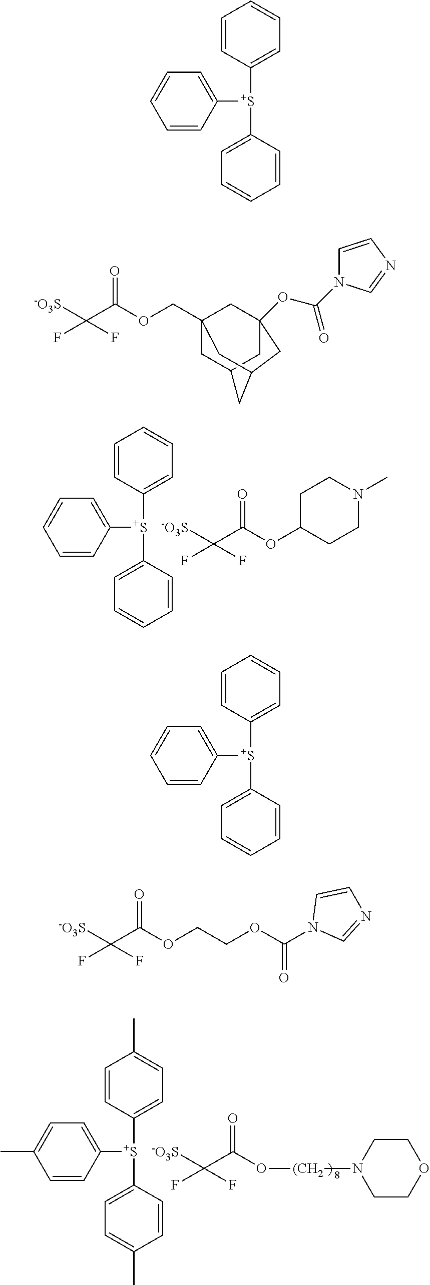

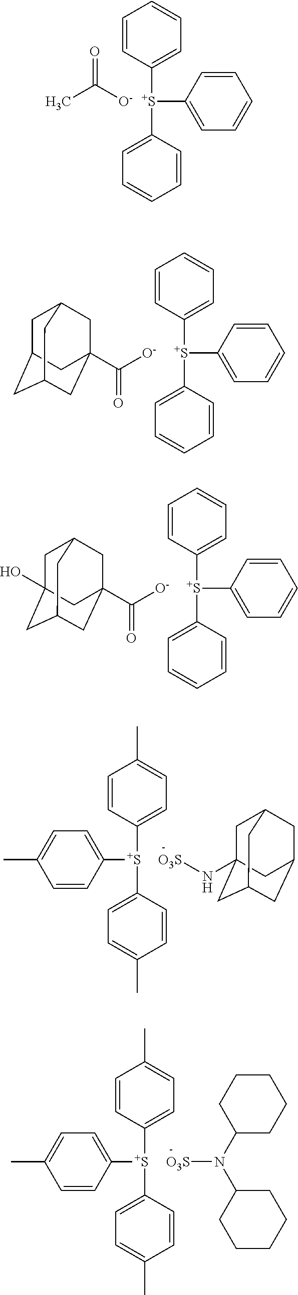

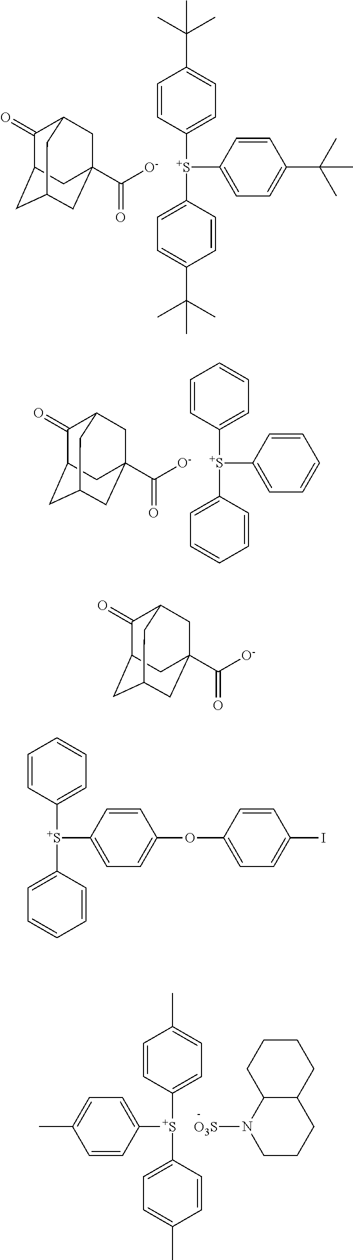

Examples of the weak acid inner salt (D) is preferably a diphenyliodonium salt having an iodonium cation to which two phenyl groups are bonded, and a carboxylic acid anion substituted with at least one phenyl group of two phenyl groups bonded to the iodonium cation, and specific examples thereof include a salt represented by the following formula:

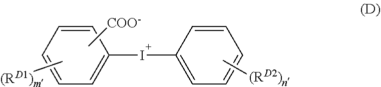

wherein, in formula (D),
R^{D1} and R^{D2} each independently represent a hydrocarbon group having 1 to 12 carbon atoms, an alkoxy group having 1 to 6 carbon atoms, an acyl group having 2 to 7 carbon atoms, an acyloxy group having 2 to 7 carbon atoms, an alkoxycarbonyl group having 2 to 7 carbon atoms, a nitro group or a halogen atom, and

m′ and n′ each independently represent an integer of 0 to 4, and when m′ is 2 or more, a plurality of R^{D1} may be the same or different, and when n′ is 2 or more, a plurality of R^{D2} may be the same or different.

Examples of the hydrocarbon group as for R^{D1} and R^{D2} include a chain hydrocarbon group, an alicyclic hydrocarbon group, an aromatic hydrocarbon group, and groups formed by combining these groups.

Examples of the chain hydrocarbon group include alkyl groups such as a methyl group, an ethyl group, a propyl group, an isopropyl group, a butyl group, an isobutyl group, a tert-butyl group, a pentyl group, a hexyl group, a nonyl group and the like.

The alicyclic hydrocarbon group may be either monocyclic or polycyclic, or may be either saturated or unsaturated. Examples thereof include cycloalkyl groups such as a cyclopropyl group, a cyclobutyl group, a cyclopentyl group, a cyclohexyl group, a cyclononyl group and a cyclododecyl group, a norbornyl group, an adamantyl group and the like.

Examples of the aromatic hydrocarbon group include aryl groups such as a phenyl group, a 1-naphthyl group, a 2-naphthyl group, a 2-methylphenyl group, a 3-methylphenyl group, a 4-methylphenyl group, a 4-ethylphenyl group, a 4-propylphenyl group, a 4-isopropylphenyl group, a 4-butylphenyl group, a 4-t-butylphenyl group, a 4-hexylphenyl group, a 4-cyclohexylphenyl group, an anthryl group, a p-adamantylphenyl group, a tolyl group, a xylyl group, a cumenyl group, a mesityl group, a biphenyl group, a phenanthryl group, a 2,6-diethylphenyl group and a 2-methyl-6-ethylphenyl group.

Examples of the groups formed by combining these groups include an alkyl-cycloalkyl group, a cycloalkyl-alkyl group, an aralkyl group (e.g., a phenylmethyl group, a 1-phenylethyl group, a 2-phenylethyl group, a 1-phenyl-1-propyl group, a 1-phenyl-2-propyl group, a 2-phenyl-2-propyl group, a 3-phenyl-1-propyl group, a 4-phenyl-1-butyl group, a 5-phenyl-1-pentyl group, a 6-phenyl-1-hexyl group, etc.) and the like.

Examples of the alkoxy group include a methoxy group, an ethoxy group and the like.

Examples of the acyl group include an acetyl group, a propanoyl group, a benzoyl group, a cyclohexanecarbonyl group and the like.

Examples of the acyloxy group include groups obtained by bonding an oxy group (—O—) to the above acyl group.

Examples of the alkoxycarbonyl group include groups obtained by bonding a carbonyl group (—CO—) to the above alkoxy group.

Examples of the halogen atom include a fluorine atom, a chlorine atom, a bromine atom and the like.

Preferably, R^{D1} and R^{D2} each independently represent an alkyl group having 1 to 8 carbon atoms, a cycloalkyl group having 3 to 10 carbon atoms, an alkoxy group having 1 to 6 carbon atoms, an acyl group having 2 to 4 carbon atoms, an acyloxy group having 2 to 4 carbon atoms, an alkoxycarbonyl group having 2 to 4 carbon atoms, a nitro group or a halogen atom.

Preferably, m′ and n′ are each independently an integer of 0 to 2, and more preferably 0, and when m′ is 2 or more, a plurality of R^{D1} may be the same or different, and when n′ is 2 or more, a plurality of R^{D2} may be the same or different.

More specifically, the following salts are exemplified.

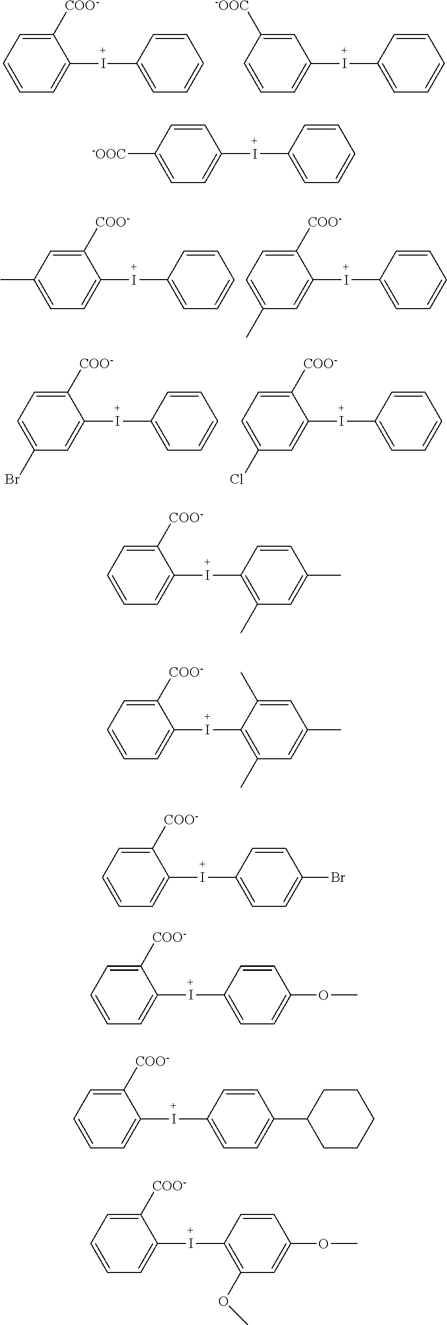

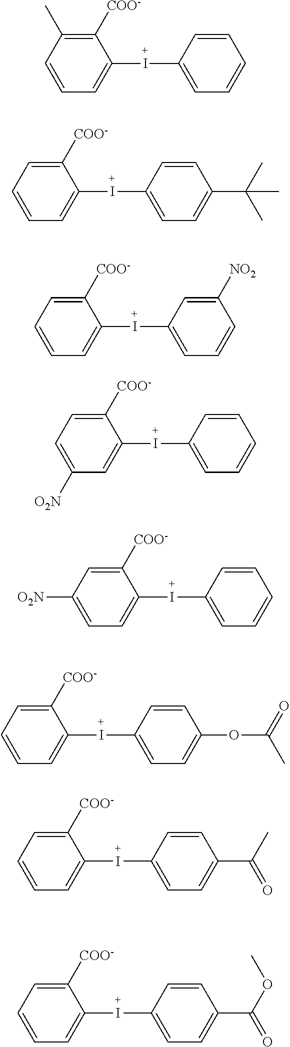

### <Other Components>

The resist composition of the present invention may also include components other than the components mentioned above (hereinafter sometimes referred to as “other components (F)”). The other components (F) are not particularly limited and it is possible to use various additives known in the resist field, for example, sensitizers, dissolution inhibitors, surfactants, stabilizers and dyes.

### <Preparation of Resist Composition>

The resist composition of the present invention can be prepared by mixing a carboxylate (I), or a compound or resin including a structural unit (IP), and if necessary, a resin (A) or the like, an acid generator (B), a resin other than the resin (A) or the like, a solvent (E), a quencher (C) and other components (F). The order of mixing these components is any order and is not particularly limited. It is possible to select, as the temperature during mixing, appropriate temperature from 10 to 40° C., according to the type of the resin, the solubility in the solvent (E) of the resin and the like. It is possible to select, as the mixing time, appropriate time from 0.5 to 24 hours according to the mixing temperature. The mixing means is not particularly limited and it is possible to use mixing with stirring.

After mixing the respective components, the mixture is preferably filtered through a filter having a pore diameter of about 0.003 to 0.2 μm.

### <Method for Producing Resist Pattern>

The method for producing a resist pattern of the present invention include:
(1) a step of applying the resist composition of the present invention on a substrate,
(2) a step of drying the applied composition to form a composition layer,
(3) a step of exposing the composition layer,
(4) a step of heating the exposed composition layer, and
(5) a step of developing the heated composition layer.

The resist composition can be usually applied on a substrate using a conventionally used apparatus, such as a spin coater. Examples of the substrate include inorganic substrates such as a silicon wafer, and organic substrates and the like in which a resist film or the like is formed on the surface. Before applying the resist composition, the substrate may be washed, and an organic antireflection film may be formed on the substrate.

The solvent is removed by drying the applied composition to form a composition layer. Drying is performed by evaporating the solvent using a heating device such as a hot plate (so-called “prebake”), or a decompression device. The heating temperature is preferably 50 to 200° C. and the heating time is preferably 10 to 180 seconds. The pressure during drying under reduced pressure is preferably about 1 to 1.0×10⁵ Pa.

The composition layer thus obtained is usually exposed using an aligner. The aligner may be a liquid immersion aligner. It is possible to use, as an exposure source, various exposure sources, for example, exposure sources capable of emitting laser beam in an ultraviolet region such as KrF excimer laser (wavelength of 248 nm), ArF excimer laser (wavelength of 193 nm) and F₂ excimer laser (wavelength of 157 nm), an exposure source capable of emitting harmonic laser beam in a far-ultraviolet or vacuum ultra violet region by wavelength-converting laser beam from a solid-state laser source (YAG or semiconductor laser), an exposure source capable of emitting electron beam or extreme ultraviolet light (EUV) and the like. As used herein, such exposure to radiation is sometimes collectively referred to as “exposure”. The exposure is usually performed through a mask corresponding to a pattern to be required. When electron beam is used as the exposure source, exposure may be performed by direct writing without using the mask.

The exposed composition layer is subjected to a heat treatment (so-called “post-exposure bake”) to promote the deprotection reaction in an acid-labile group. The heating temperature is usually about 50 to 200° C., and preferably about 70 to 150° C. It is also possible to perform a chemical treatment (silylation) which adjusts the hydrophilicity or hydrophobicity of the resin on a surface side of the composition after heating. Before performing the development, the steps of application of the resist composition, drying, exposure and heating may be repeatedly performed on the exposed composition layer.

The heated composition layer is usually developed with a developing solution using a development apparatus. Examples of the developing method include a dipping method, a paddle method, a spraying method, a dynamic dispensing method and the like. The developing temperature is preferably, for example, 5 to 60° C. and the developing time is preferably, for example, 5 to 300 seconds. It is possible to produce a positive resist pattern or negative resist pattern by selecting the type of the developing solution as follows.

When the positive resist pattern is produced from the resist composition of the present invention, an alkaline developing solution is used as the developing solution. The alkaline developing solution may be various aqueous alkaline solutions used in this field. Examples thereof include aqueous solutions of tetramethylammonium hydroxide and (2-hydroxyethyl)trimethylammonium hydroxide (commonly known as choline). The surfactant may be contained in the alkaline developing solution.

It is preferable that the developed resist pattern is washed with ultrapure water and then water remaining on the substrate and the pattern is removed.

When the negative resist pattern is produced from the resist composition of the present invention, a developing solution containing an organic solvent (hereinafter sometimes referred to as “organic developing solution”) is used as the developing solution.

Examples of the organic solvent contained in the organic developing solution include ketone solvents such as 2-hexanone and 2-heptanone; glycol ether ester solvents such as propylene glycol monomethyl ether acetate; ester solvents such as butyl acetate; glycol ether solvents such as propylene glycol monomethyl ether; amide solvents such as N,N-dimethylacetamide; and aromatic hydrocarbon solvents such as anisole.

The content of the organic solvent in the organic developing solution is preferably 90% by mass or more and 100% by mass or less, more preferably 95% by mass or more and 100% by mass or less, and still more preferably the organic developing solution is substantially composed of the organic solvent.

Particularly, the organic developing solution is preferably a developing solution containing butyl acetate and/or 2-heptanone. The total content of butyl acetate and 2-heptanone in the organic developing solution is preferably 50% by mass or more and 100% by mass or less, more preferably 90% by mass or more and 100% by mass or less, and still more preferably the organic developing solution is substantially composed of butyl acetate and/or 2-heptanone.

The surfactant may be contained in the organic developing solution. A trace amount of water may be contained in the organic developing solution.

During development, the development may be stopped by replacing by a solvent with the type different from that of the organic developing solution.

The developed resist pattern is preferably washed with a rinsing solution. The rinsing solution is not particularly limited as long as it does not dissolve the resist pattern, and it is possible to use a solution containing an ordinary organic solvent which is preferably an alcohol solvent or an ester solvent.

After washing, the rinsing solution remaining on the substrate and the pattern is preferably removed.

### <Applications>

The resist composition of the present invention is suitable as a resist composition for exposure of KrF excimer laser, a resist composition for exposure of ArF excimer laser, a resist composition for exposure of electron beam (EB) or a resist composition for exposure of EUV, particularly a resist composition for exposure of electron beam (EB) or a resist composition for exposure of EUV, and the resist composition is useful for fine processing of semiconductors.

### EXAMPLES

The present invention will be described more specifically by way of Examples. Percentages and parts expressing the contents or amounts used in the Examples are by mass unless otherwise specified.

The weight-average molecular weight is a value determined by gel permeation chromatography. Analysis conditions of gel permeation chromatography are as follows.
Column: TSKgel Multipore HXL-M×3+guardcolumn (manufactured by TOSOH CORPORATION)
Eluent: tetrahydrofuran
Flow rate: 1.0 mL/min
Detector: RI detector
Column temperature: 40° C.

Injection amount: 100 μl
Molecular weight standards: polystyrene standard (manufactured by TOSOH CORPORATION)

Structures of compounds were confirmed by measuring a molecular ion peak using mass spectrometry (LC: Model 1100, manufactured by Agilent Technologies, Inc., and MASS: Model LC/MSD, manufactured by Agilent Technologies, Inc.). The value of this molecular ion peak in the following Examples is indicated by “MASS”.

### Example 1: Synthesis of Carboxylate Represented by Formula (I-1)

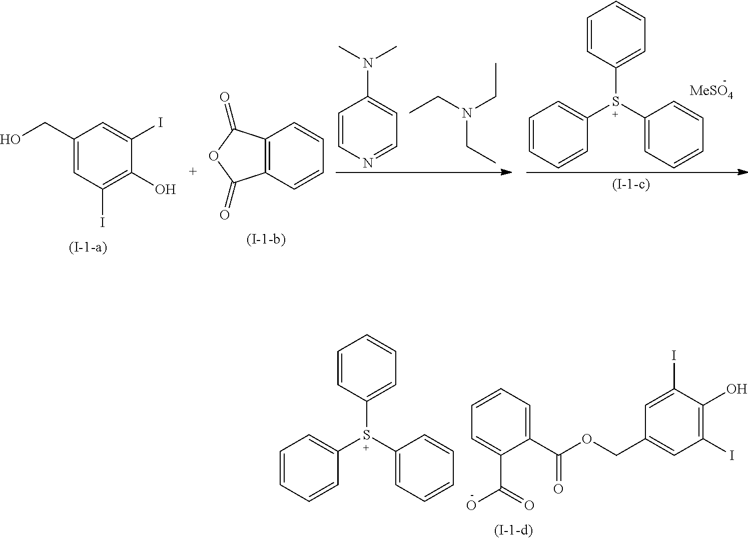

3.76 Parts of a compound represented by formula (I-1-a), 1.28 parts of dimethylaminopyridine, 1.06 parts of triethylamine and 20 parts of dimethylformamide were mixed and, after stirring at 23° C. for 30 minutes, 1.48 parts of a compound represented by formula (I-1-b) and 30 parts of chloroform were added, followed by stirring at 50° C. for 5 hours. The mixture thus obtained was cooled to 23° C. and then 3.74 parts of a salt represented by formula (I-1-c) was added, followed by stirring at 23° C. for 3 hours. The mixture thus obtained was cooled to 23° C. and then 100 parts of chloroform and 50 parts of ion-exchanged water were added and, stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 6.79 parts of a carboxylate represented by formula (I-1-d).

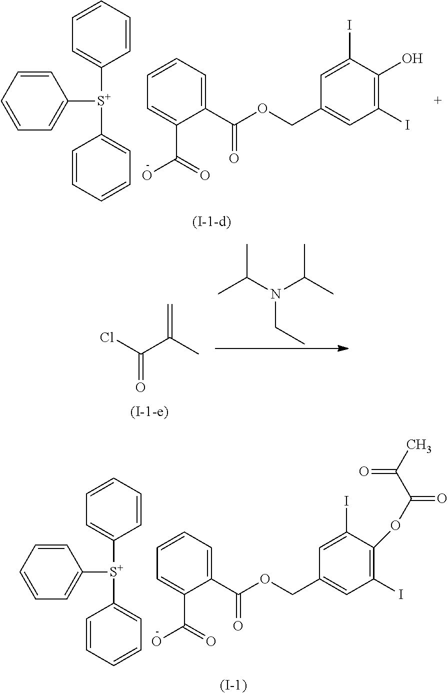

3.84 Parts of a carboxylate represented by formula (I-1-d), 50 parts of acetonitrile and 2.17 parts of a compound represented by formula (I-1-e) were mixed, followed by stirring at 23° C. for 30 minutes and further cooling to 5° C. To the mixture thus obtained, 1.33 parts of diisopropylethylamine was added dropwise, followed by temperature rise to 70° C., stirring at 70° C. for 4 hours and further cooling to 23° C. To the mixture thus obtained, 100 parts of chloroform and 50 parts of ion-exchanged water were added and, after stirring at 23° C. for 30 minutes, the organic layer was isolated through separation. To the organic layer thus obtained, 50 parts of an aqueous 5% oxalic acid solution was added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then the concentrated mass was isolated using a column (silica gel 60N (spherical, neutral) 100-210 μm; manufactured by Kanto Chemical Co., Inc., developing solvent: methanol/chloroform=1/1) to obtain 2.14 parts of a carboxylate represented by formula (I-1).
MASS (ESI (+) Spectrum): M⁺263.1
MASS (ESI (−) Spectrum): M⁻590.9

### Example 2: Synthesis of Carboxylate Represented by Formula (1-221)

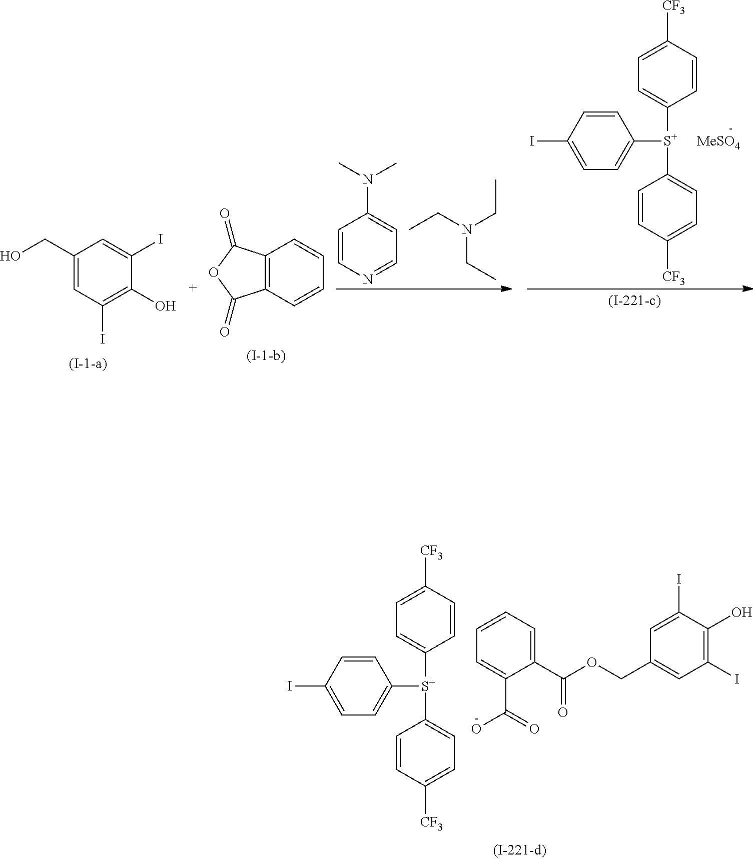

3.76 parts of a compound represented by formula (I-1-a), 1.28 parts of dimethylaminopyridine, 1.06 parts of triethylamine and 20 parts of dimethylformamide were mixed and, after stirring at 23° C. for 30 minutes, 1.48 parts of a compound represented by formula (I-1-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 8.57 parts of a carboxylate represented by formula (I-221-d).

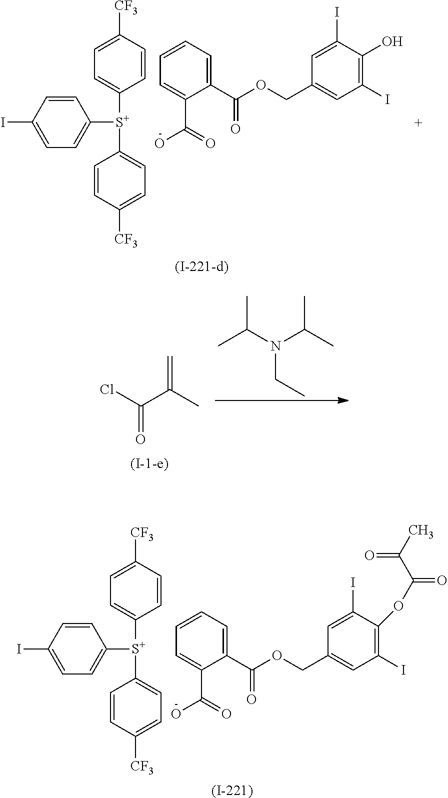

5.12 Parts of a carboxylate represented by formula (I-221-d), 50 parts of acetonitrile and 2.17 parts of a compound represented by formula (I-1-e) were mixed, followed by stirring at 23° C. for 30 minutes and further cooling to 5° C. To the mixture thus obtained, 1.33 parts of diisopropylethylamine was added dropwise, followed by temperature rise to 70° C., stirring at 70° C. for 4 hours and further cooling to 23° C. To the mixture thus obtained, 100 parts of chloroform and 50 parts of ion-exchanged water were added and, after stirring at 23° C. for 30 minutes, the organic layer was isolated through separation. To the organic layer thus obtained, 50 parts of an aqueous 5% oxalic acid solution was added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then the concentrated mass was isolated using a column (silica gel 60N (spherical, neutral) 100-210 μm; manufactured by Kanto Chemical Co., Inc., developing solvent: methanol/chloroform=1/1) to obtain 3.52 parts of a carboxylate represented by formula (I-221).
MASS (ESI (+) Spectrum): M⁺525.0
MASS (ESI (−) Spectrum): M⁻ 590.9

### Example 3: Synthesis of Carboxylate Represented by Formula (I-224)

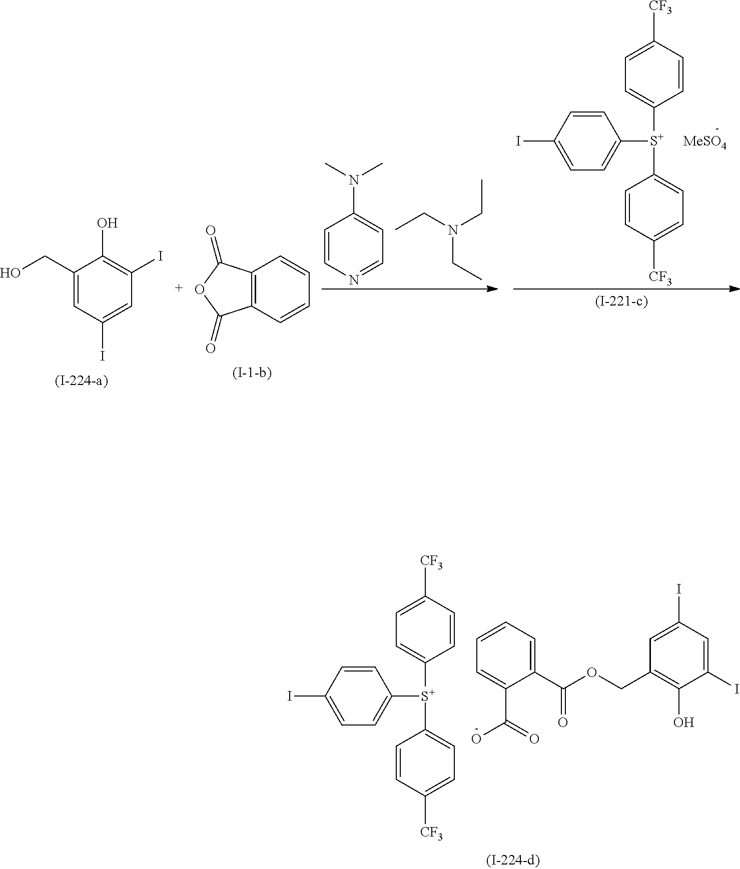

3.76 Parts of a compound represented by formula (I-224-a), 1.28 parts of dimethylaminopyridine, 1.06 parts of triethylamine and 20 parts of dimethylformamide were mixed and, after stirring at 23° C. for 30 minutes, 1.48 parts of a compound represented by formula (I-1-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 8.93 parts of a carboxylate represented by formula (I-224-d).

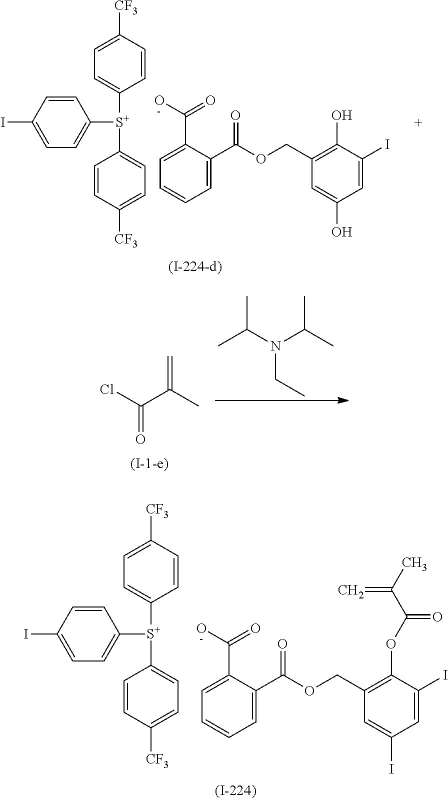

5.12 Parts of a carboxylate represented by formula (I-224-d), 50 parts of acetonitrile and 2.17 parts of a compound represented by formula (I-1-e) were mixed, followed by stirring at 23° C. for 30 minutes and further cooling to 5° C. To the mixture thus obtained, 1.33 parts of diisopropylethylamine was added dropwise, followed by temperature rise to 70° C., stirring at 70° C. for 4 hours and further cooling to 23° C. To the mixture thus obtained, 100 parts of chloroform and 50 parts of ion-exchanged water were added and, after stirring at 23° C. for 30 minutes, the organic layer was isolated through separation. To the organic layer thus obtained, 50 parts of an aqueous 5% oxalic acid solution was added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 4.38 parts of a carboxylate represented by formula (I-224).
MASS (ESI (+) Spectrum): M⁺525.0
MASS (ESI (−) Spectrum): M⁻590.9

### Example 4: Synthesis of Carboxylate Represented by Formula (I-239)

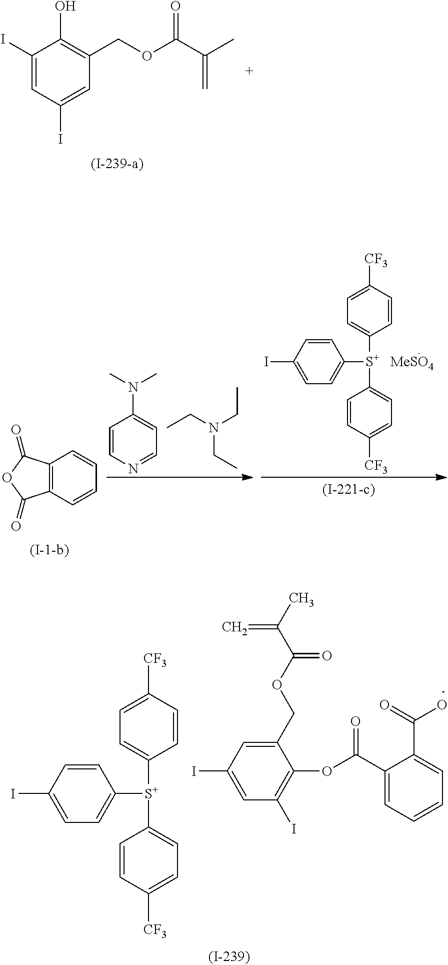

4.44 Parts of a compound represented by formula (I-239-a), 1.28 parts of dimethylaminopyridine, 1.06 parts of triethylamine and 20 parts of dimethylformamide were mixed and, after stirring at 23° C. for 30 minutes, 1.48 parts of a compound represented by formula (I-1-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 8.19 parts of a carboxylate represented by formula (I-239).
MASS (ESI (+) Spectrum): M⁺525.0
MASS (ESI (−) Spectrum): M⁻590.9

### Example 5: Synthesis of Carboxylate Represented by Formula (1-240)

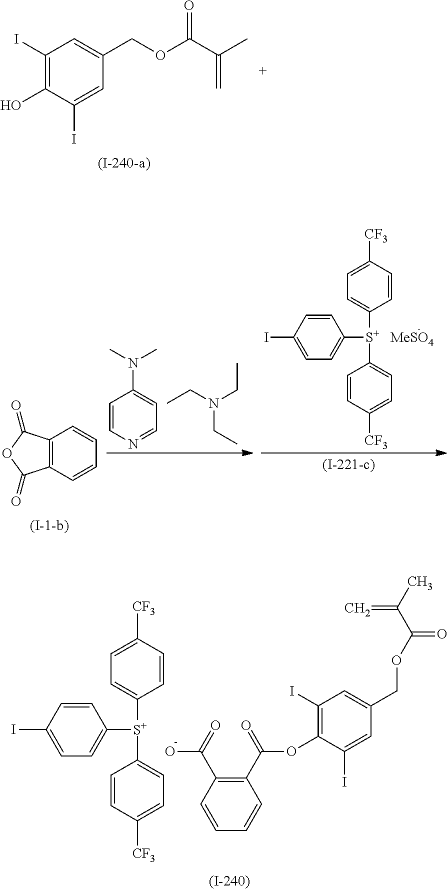

4.44 Parts of a compound represented by formula (I-240-a), 1.28 parts of dimethylaminopyridine, 1.06 parts of triethylamine and 20 parts of dimethylformamide were mixed and, after stirring at 23° C. for 30 minutes, 1.48 parts of a compound represented by formula (I-1-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then the concentrated mass was isolated using a column (silica gel 60N (spherical, neutral) 100-210 μm; manufactured by Kanto Chemical Co., Inc., developing solvent: methanol/chloroform=1/1) to obtain 3.89 parts of a carboxylate represented by formula (I-240).
MASS (ESI (+) Spectrum): M⁺525.0
MASS (ESI (−) Spectrum): M⁻590.9

### Example 6: Synthesis of Carboxylate Represented by Formula (1-238)

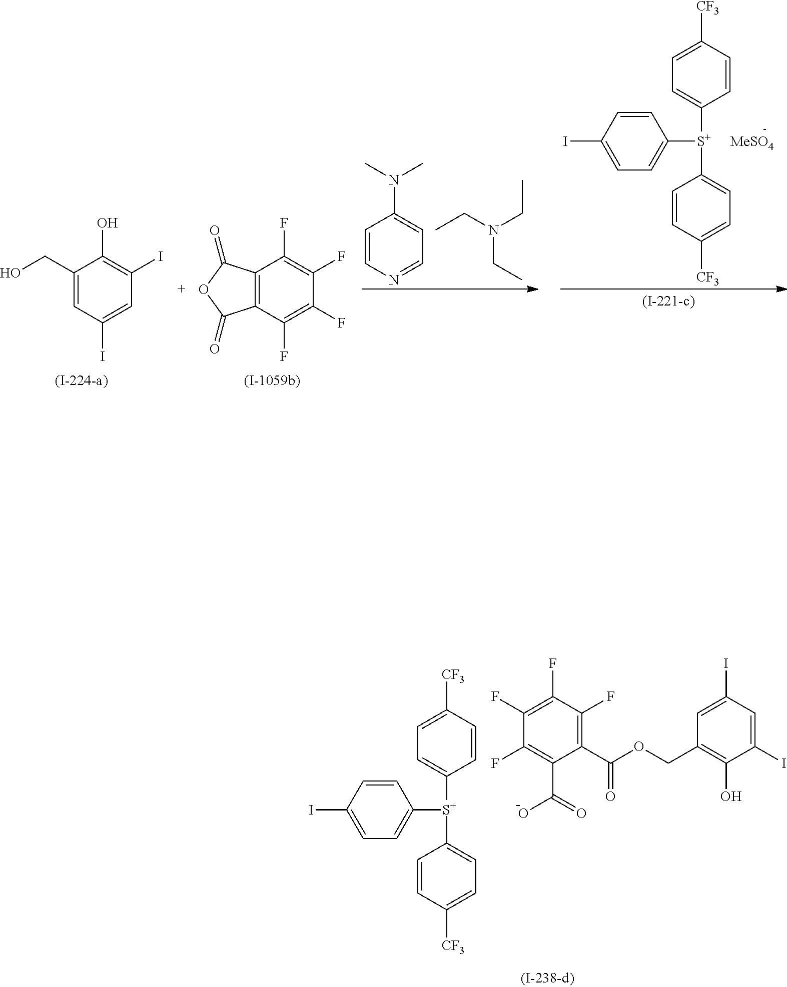

3.76 Parts of a compound represented by formula (I-224-a), 1.28 parts of dimethylaminopyridine, 1.06 parts of triethylamine and 20 parts of dimethylformamide were mixed and, after stirring at 23° C. for 30 minutes, 2.20 parts of a compound represented by formula (I-238-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 10.02 parts of a carboxylate represented by formula (I-238-d).

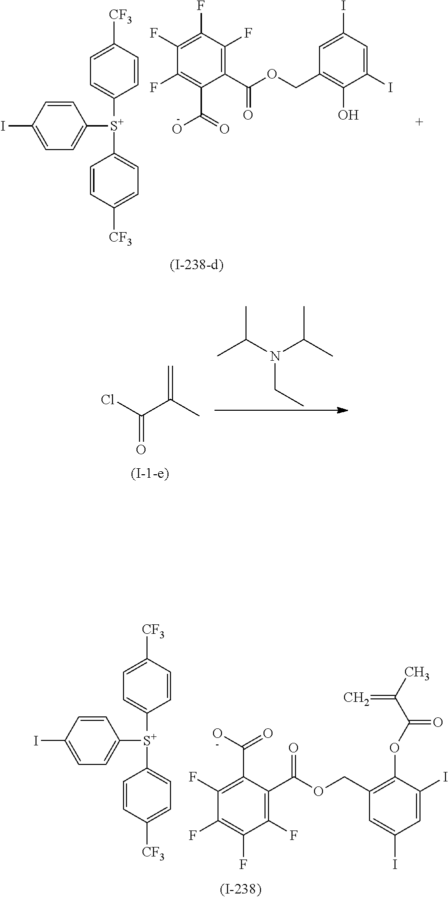

5.47 Parts of a carboxylate represented by formula (I-238-d), 50 parts of acetonitrile and 2.17 parts of a compound represented by formula (I-1-e) were mixed, followed by stirring at 23° C. for 30 minutes and further cooling to 5° C. To the mixture thus obtained, 1.33 parts of diisopropylethylamine was added dropwise, followed by temperature rise to 70° C., stirring at 70° C. for 4 hours and further cooling to 23° C. To the mixture thus obtained, 100 parts of chloroform and 50 parts of ion-exchanged water were added and, after stirring at 23° C. for 30 minutes, the organic layer was isolated through separation. To the organic layer thus obtained, 50 parts of an aqueous 5% oxalic acid solution was added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 4.88 parts of a carboxylate represented by formula (I-238).
MASS (ESI (+) Spectrum): M⁺525.0
MASS (ESI (−) Spectrum): M⁻662.8

### Example 7: Synthesis of Carboxylate Represented by Formula (1-1058)

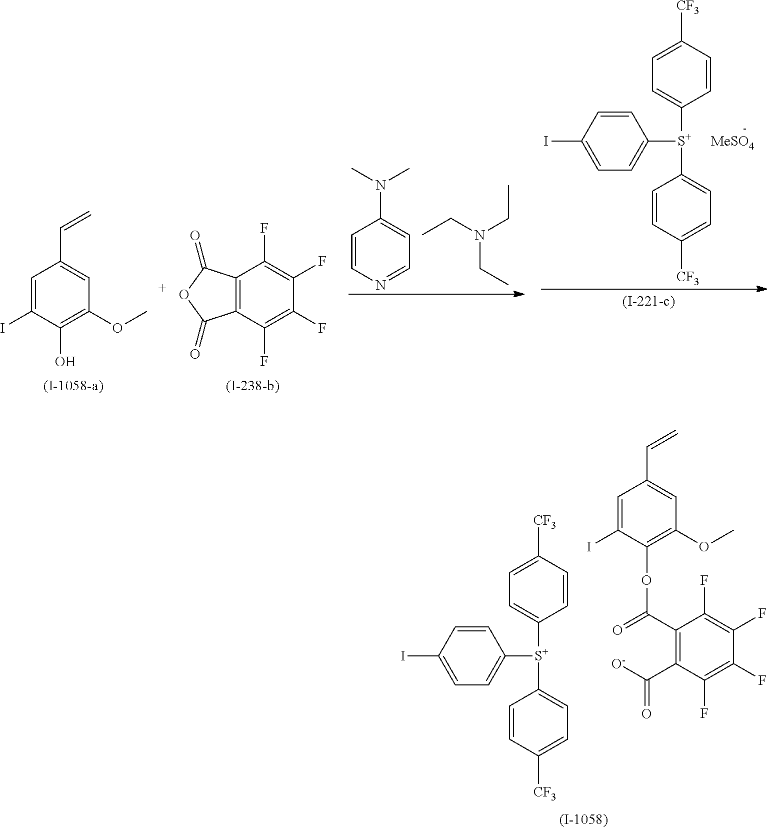

2.76 Parts of a compound represented by formula (I-1058-a), 1.28 parts of dimethylaminopyridine, 1.06 parts of triethylamine and 20 parts of dimethylformamide were mixed and, after stirring at 23° C. for 30 minutes, 2.20 parts of a compound represented by formula (I-238-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 9.15 parts of a carboxylate represented by formula (I-1058).
MASS (ESI (+) Spectrum): M⁺525.0
MASS (ESI (−) Spectrum): M⁻494.9

### Example 8: Synthesis of Carboxylate Represented by Formula (1-1059)

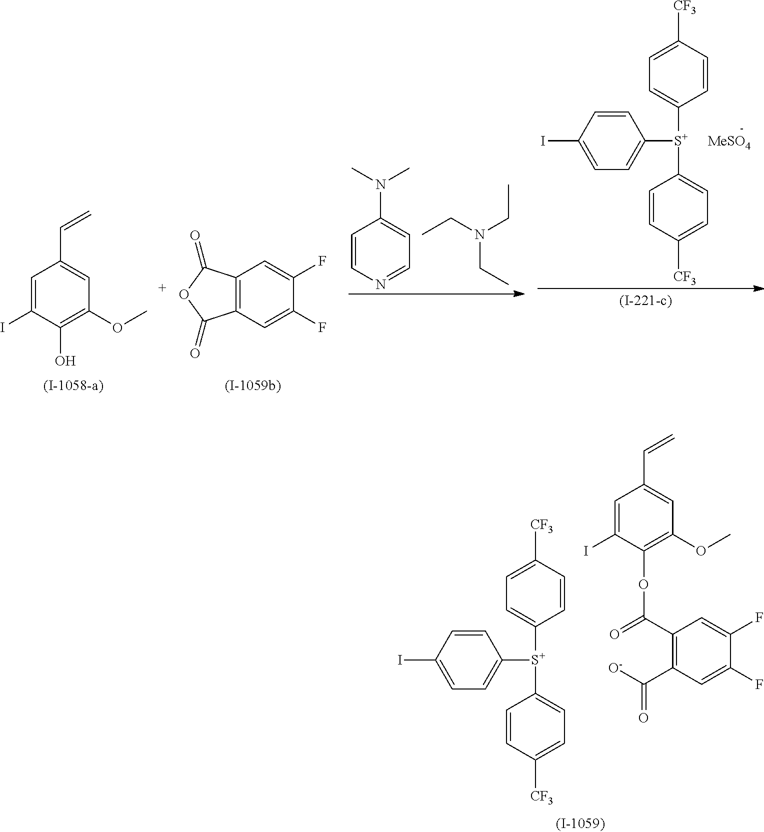

2.76 Parts of a compound represented by formula (I-1058-a), 1.28 parts of dimethylaminopyridine, 1.06 parts of triethylamine and 20 parts of dimethylformamide were mixed and, after stirring at 23° C. for 30 minutes, 1.84 parts of a compound represented by formula (I-1059-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 8.86 parts of a carboxylate represented by formula (I-1059).
MASS (ESI (+) Spectrum): M⁺525.0
MASS (ESI (−) Spectrum): M⁻459.0

### Example 9: Synthesis of Carboxylate Represented by Formula (1-1060)

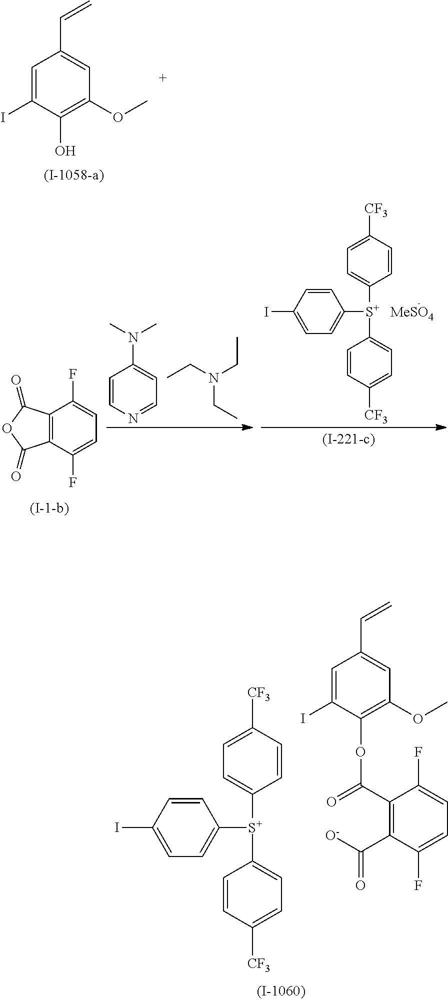

2.76 Parts of a compound represented by formula (I-1058-a), 1.28 parts of dimethylaminopyridine, 1.06 parts of triethylamine and 20 parts of dimethylformamide were mixed and, after stirring at 23° C. for 30 minutes, 1.84 parts of a compound represented by formula (I-1060-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 8.23 parts of a carboxylate represented by formula (I-1060).
MASS (ESI (+) Spectrum): M⁺525.0
MASS (ESI (−) Spectrum): M⁻459.0

### Example 10: Synthesis of Carboxylate Represented by Formula (1-1062)

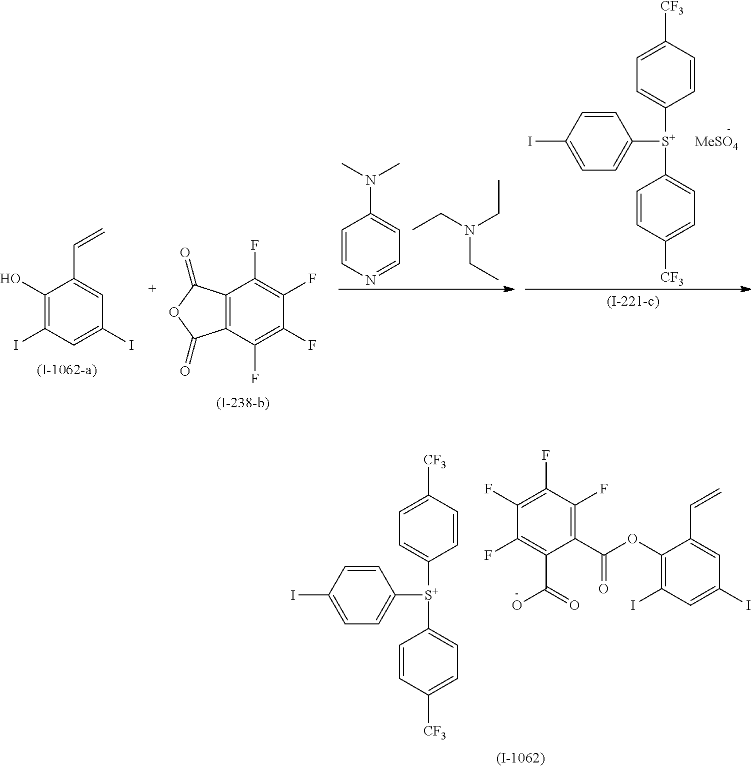

3.72 Parts of a compound represented by formula (I-1062-a), 1.28 parts of dimethylaminopyridine, 1.06 parts of triethylamine and 20 parts of dimethylformamide were mixed and, after stirring at 23° C. for 30 minutes, 2.20 parts of a compound represented by formula (I-238-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 10.09 parts of a carboxylate represented by formula (I-1062).
MASS (ESI (+) Spectrum): M⁺525.0
MASS (ESI (−) Spectrum): M⁻590.8

### Example 11: Synthesis of Salt Represented by Formula (I-1066)

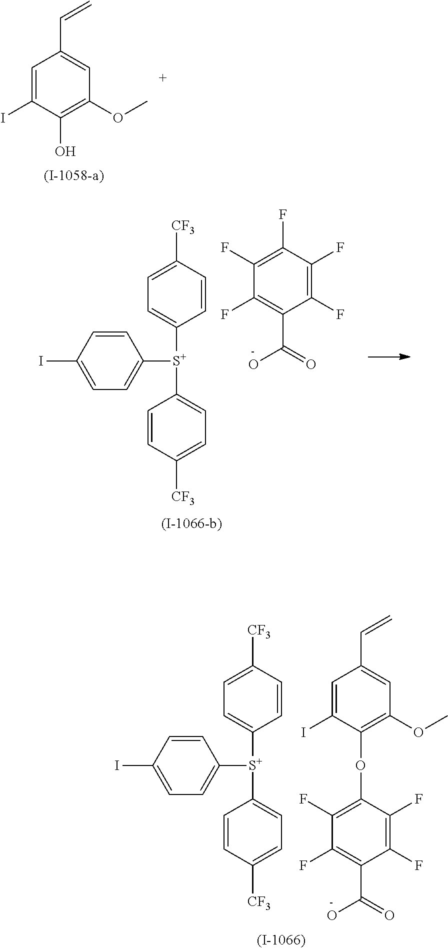 [SMILES: C=Cc1cc(I)c(O)c(OC)c1 ; FC(F)(F)c1ccc([S+](c2ccc(I)cc2)c2ccc(C(F)(F)F)cc2)cc1 ; O=C(O)c1c(F)c(F)c(F)c(F)c1F ; FC(F)(F)c1ccc([S+](c2ccc(I)cc2)c2ccc(C(F)(F)F)cc2)cc1 ; C=Cc1cc(I)c(Oc2c(F)c(F)c(C(=O)O)c(F)c2F)c(OC)c1]

2.76 Parts of a compound represented by formula (I-1058-a), 7.36 parts of a salt represented by formula (I-1066-b) and 50 parts of acetonitrile were mixed, followed by stirring at 23° C. for 30 minutes. To the mixture thus obtained, 0.88 part of potassium carbonate was added, followed by stirring at 23° C. for 30 minutes and further stirring at 60° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then the concentrated mixture was isolated using a column (silica gel 60N (spherical, neutral) 100-210 μm; manufactured by Kanto Chemical Co., Inc., developing solvent: chloroform/methanol=1/1) to obtain 1.89 parts of a salt represented by formula (I-1066).
MASS (ESI (+) Spectrum): M⁺525.0
MASS (ESI (−) Spectrum): M⁻466.9

### Example 12: Synthesis of Salt Represented by Formula (I-1067)

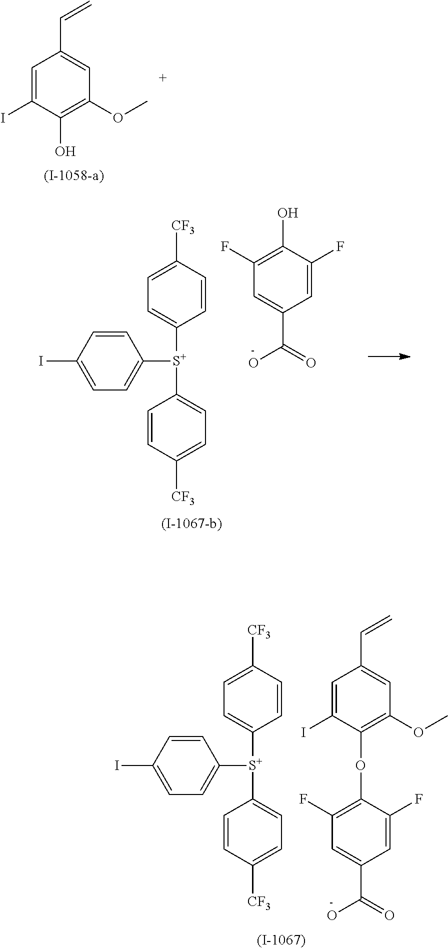 [SMILES: C=Cc1cc(I)c(O)c(OC)c1 ; FC(F)(F)c1ccc([S+](c2ccc(I)cc2)c2ccc(C(F)(F)F)cc2)cc1 ; O=C(O)c1cc(F)c(O)c(F)c1 ; FC(F)(F)c1ccc([S+](c2ccc(I)cc2)c2ccc(C(F)(F)F)cc2)cc1 ; C=Cc1cc(I)c(Oc2c(F)cc(C(=O)O)cc2F)c(OC)c1]

2.76 Parts of a compound represented by formula (I-1058-a), 6.98 parts of a salt represented by formula (I-1067-b) and 50 parts of acetonitrile were mixed, followed by stirring at 23° C. for 30 minutes. To the mixture thus obtained, 0.80 part of sodium hydroxide was added, followed by stirring at 23° C. for 30 minutes and further stirring at 80° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then the concentrated mixture was isolated using a column (silica gel 60N (spherical, neutral) 100-210 μm; manufactured by Kanto Chemical Co., Inc., developing solvent: chloroform/methanol=1/1) to obtain 1.22 parts of a salt represented by formula (I-1067).
MASS (ESI (+) Spectrum): M⁺525.0
MASS (ESI (−) Spectrum): M⁻431.0

### Example 13: Synthesis of Salt Represented by Formula (I-1068)

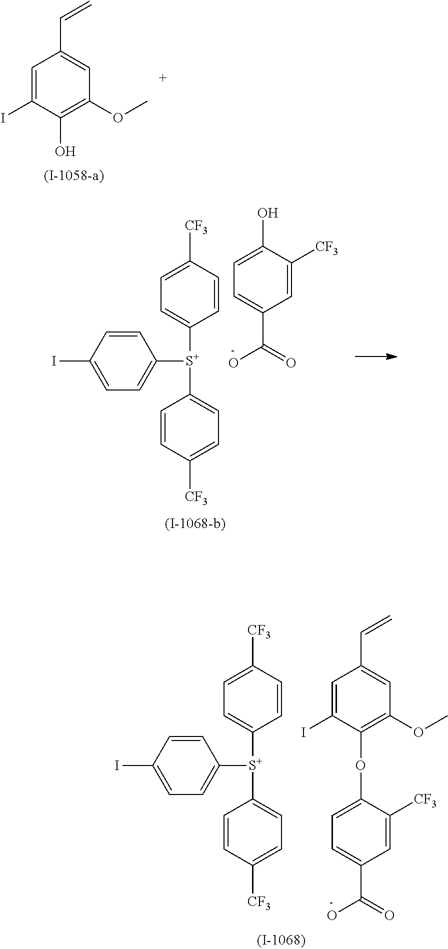 [SMILES: C=Cc1cc(I)c(O)c(OC)c1 ; FC(F)(F)c1ccc([S+](c2ccc(I)cc2)c2ccc(C(F)(F)F)cc2)cc1 ; O=C(O)c1ccc(O)c(C(F)(F)F)c1 ; FC(F)(F)c1ccc([S+](c2ccc(I)cc2)c2ccc(C(F)(F)F)cc2)cc1 ; C=Cc1cc(I)c(Oc2ccc(C(=O)O)cc2C(F)(F)F)c(OC)c1]

2.76 Parts of a compound represented by formula (I-1058-a), 7.30 parts of a salt represented by formula (I-1068-b) and 50 parts of acetonitrile were mixed, followed by stirring at 23° C. for 30 minutes. To the mixture thus obtained, 0.80 part of sodium hydroxide was added, followed by stirring at 23° C. for 30 minutes and further stirring at 80° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the organic layer thus obtained, 50 parts of ion-exchanged water was added and, after stirring at 23° C. for 30 minutes, the organic layer was isolated through separation. This water washing operation was repeated five times. The organic layer thus obtained was concentrated and then the concentrated mixture was isolated using a column (silica gel 60N (spherical, neutral) 100-210 μm; manufactured by Kanto Chemical Co., Inc., developing solvent: chloroform/methanol=1/1) to obtain 1.41 parts of a salt represented by formula (I-1068).
MASS (ESI (+) Spectrum): M⁺525.0
MASS (ESI (−) Spectrum): M⁻463.0

### Example 14: Synthesis of Salt Represented by Formula (I-1071)

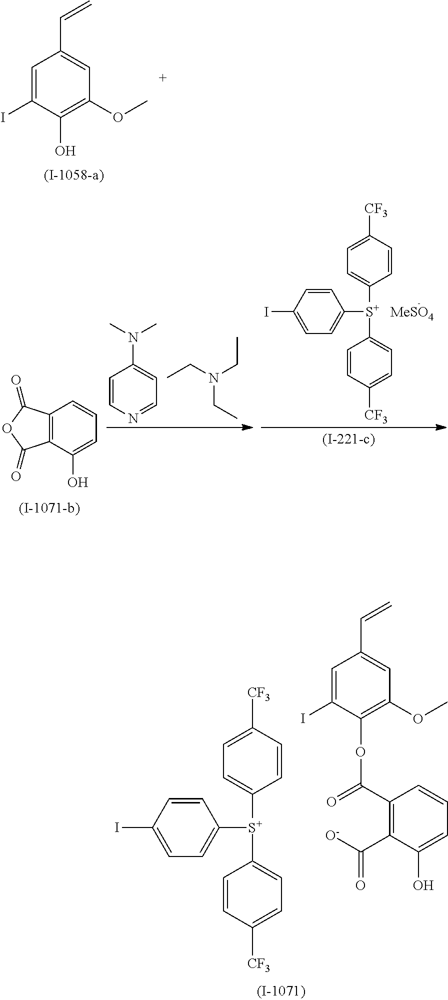

2.76 Parts of a compound represented by formula (I-1058-a), 1.28 parts of dimethylaminopyridine, 1.06 parts of triethylamine and 20 parts of dimethylformamide were mixed and, after stirring at 23° C. for 30 minutes, 1.64 parts of a compound represented by formula (I-1071-b) and 30 parts of chloroform were added, followed by stirring at 50° C. for 5 hours. The mixture thus obtained was cooled to 23° C. and then 6.36 parts of a salt represented by formula (I-221-c) was added, followed by stirring at 23° C. for 3 hours. The mixture thus obtained was cooled to 23° C. and then 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 8.51 parts of a carboxylate represented by formula (I-1071).
MASS (ESI (+) Spectrum): M⁺525.0
MASS (ESI (−) Spectrum): M⁻439.0

### Synthesis Example 1: Synthesis of Carboxylate Represented by Formula (IX-2)

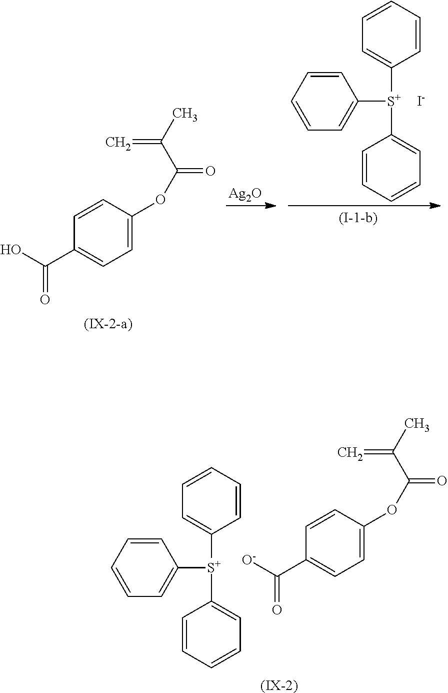

2.06 Parts of a compound represented by formula (IX-2-a), 1.16 parts of silver oxide and 30 parts of acetonitrile were added, followed by stirring at 23° C. for 4 hours. To the reaction product thus obtained, 3.90 parts of a salt represented by formula (I-1-b) and 30 parts of methanol were mixed, followed by stirring at 23° C. for 3 hours. To the mixture thus obtained, 100 parts of chloroform and 50 parts of ion-exchanged water were added and, after stirring at 23° C. for 30 minutes, the organic layer was isolated through separation. To the mixture thus obtained, 50 parts of ion-exchanged water was added and, after stirring at 23° C. for 30 minutes, the organic layer was isolated through separation. This water washing operation was repeated three times. The organic layer thus obtained was concentrated and then 30 parts of tert-butyl methyl ether was added to the concentrated residue and, after stirring at 23° C. for 30 minutes, the supernatant was removed, followed by concentration to obtain 6.89 parts of a carboxylate represented by formula (IX-2).
MASS (ESI (+) Spectrum): M⁺263.1
MASS (ESI (−) Spectrum): M⁻205.1

### Synthesis of Resin

Compounds (monomers) used in synthesis of a resin (A) are shown below. Hereinafter, these compounds are referred to as “monomer (a1-1-3)” or the like according to the formula number.

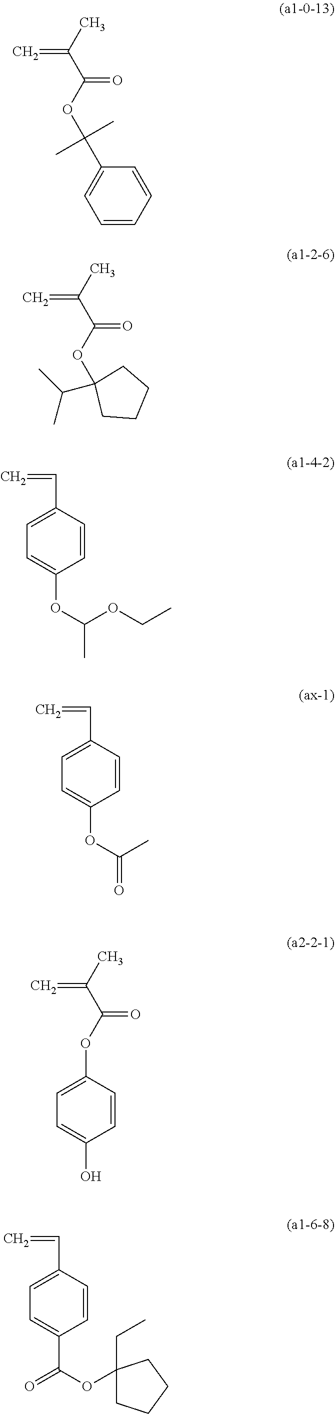

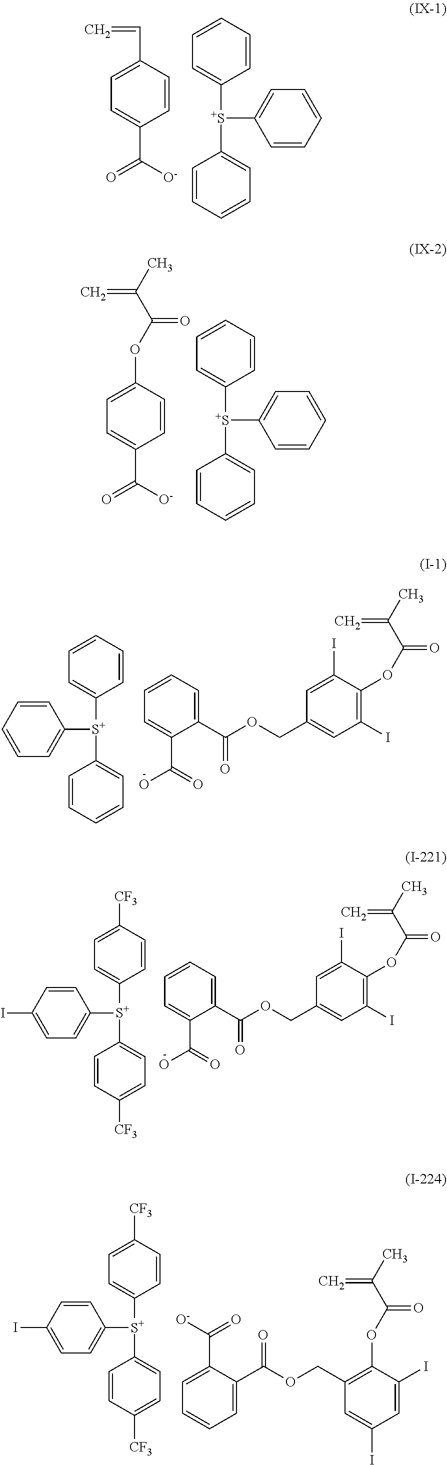

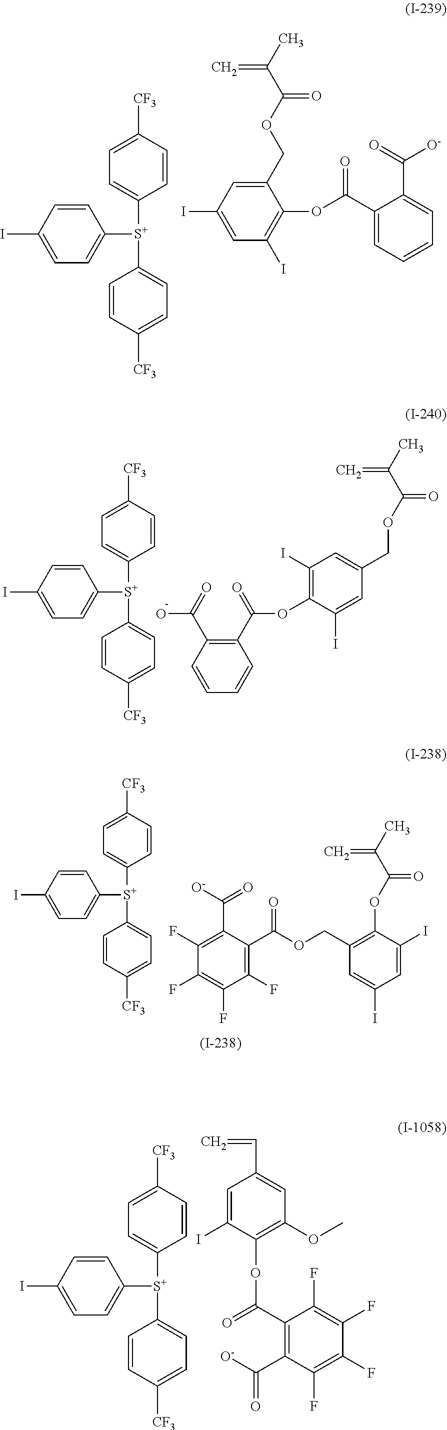

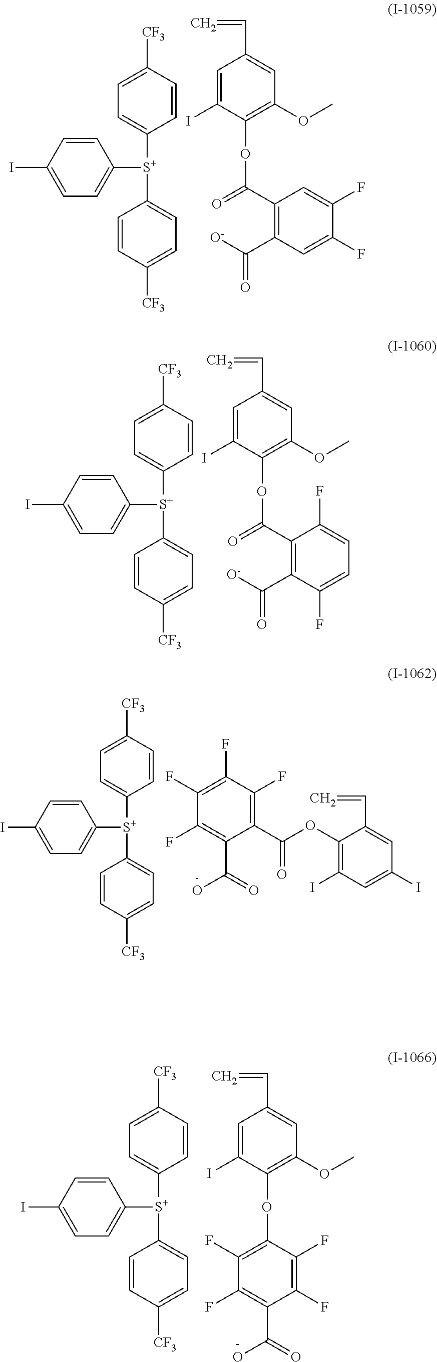

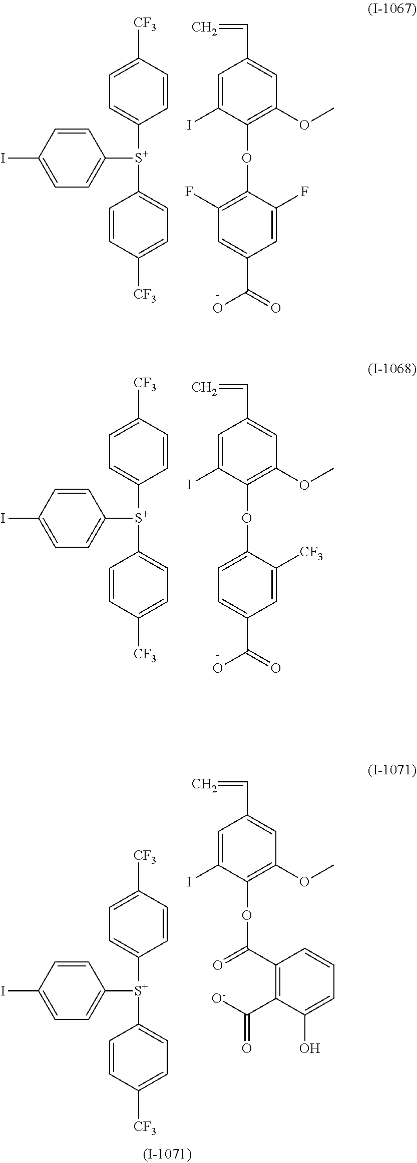 [SMILES: FC(F)(F)c1ccc([S+](c2ccc(I)cc2)c2ccc(C(F)(F)F)cc2)cc1 ; C=Cc1cc(I)c(Oc2c(F)cc(C(=O)O)cc2F)c(OC)c1 ; FC(F)(F)c1ccc([S+](c2ccc(I)cc2)c2ccc(C(F)(F)F)cc2)cc1 ; C=Cc1cc(I)c(Oc2ccc(C(=O)O)cc2C(F)(F)F)c(OC)c1 ; FC(F)(F)c1ccc([S+](c2ccc(I)cc2)c2ccc(C(F)(F)F)cc2)cc1 ; C=Cc1cc(I)c(OC(=O)c2cccc(O)c2C(=O)[O-])c(OC)c1]

### Example 15 [Synthesis of Resin A1-1]

Using a monomer (ax-1), a monomer (a1-2-6) and a monomer (I-1) as monomers, these monomers were mixed in a molar ratio of 35:62:3 [monomer (ax-1):monomer (a1-2-6):monomer (I-1)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-1 (copolymer) having a weight-average molecular weight of about 5.4×10³ in a yield of 77%. This resin A1-1 includes the following structural units.

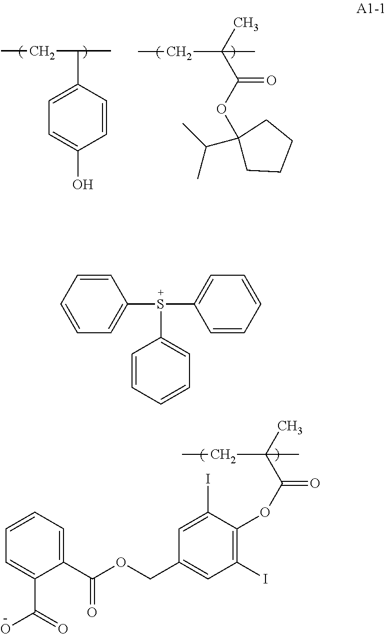

### Example 16 [Synthesis of Resin A1-2]

Using a monomer (ax-1), a monomer (a1-2-6) and a monomer (I-221) as monomers, these monomers were mixed in a molar ratio of 35:62:3 [monomer (ax-1):monomer (a1-2-6):monomer (I-221)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-2 (copolymer) having a weight-average molecular weight of about 5.3×10³ in a yield of 75%. This resin A1-2 includes the following structural units.

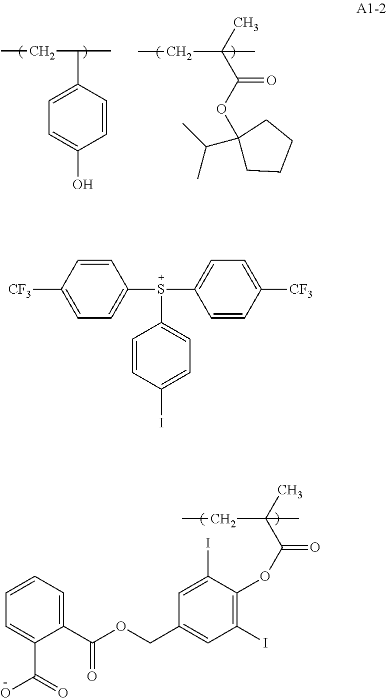

### Example 17 [Synthesis of Resin A1-3]

Using a monomer (ax-1), a monomer (a1-2-6) and a monomer (I-224) as monomers, these monomers were mixed in a molar ratio of 35:62:3 [monomer (ax-1):monomer (a1-2-6):monomer (I-224)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A¹-3 (copolymer) having a weight-average molecular weight of about 5.5×10³ in a yield of 83%. This resin A¹-3 includes the following structural units.

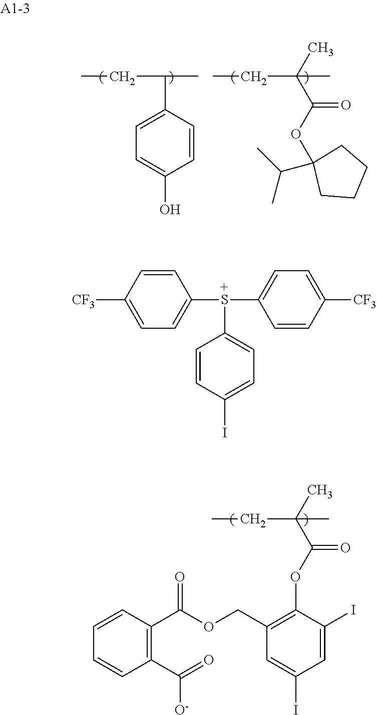

### Example 18 [Synthesis of Resin A1-4]

Using a monomer (ax-1), a monomer (a1-2-6) and a monomer (I-239) as monomers, these monomers were mixed in a molar ratio of 35:62:3 [monomer (ax-1):monomer (a1-2-6):monomer (I-239)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-4 (copolymer) having a weight-average molecular weight of about 5.6×10³ in a yield of 85%. This resin A1-4 includes the following structural units.

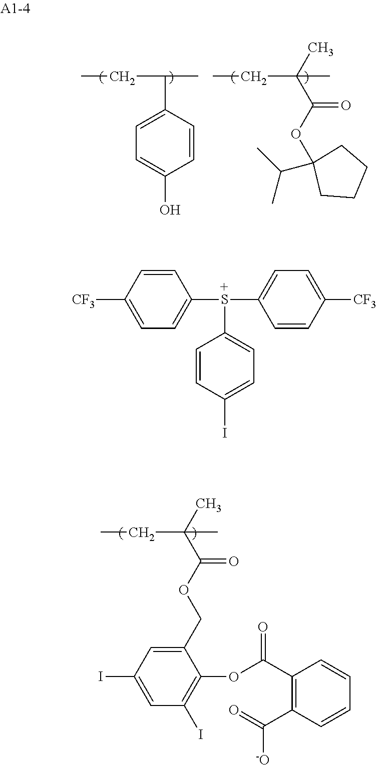

### Example 19 [Synthesis of Resin A1-5]

Using a monomer (ax-1), a monomer (a1-2-6) and a monomer (I-240) as monomers, these monomers were mixed in a molar ratio of 35:62:3 [monomer (ax-1):monomer (a1-2-6):monomer (I-240)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-5 (copolymer) having a weight-average molecular weight of about 5.3×10³ in a yield of 73%. This resin A1-5 includes the following structural units.

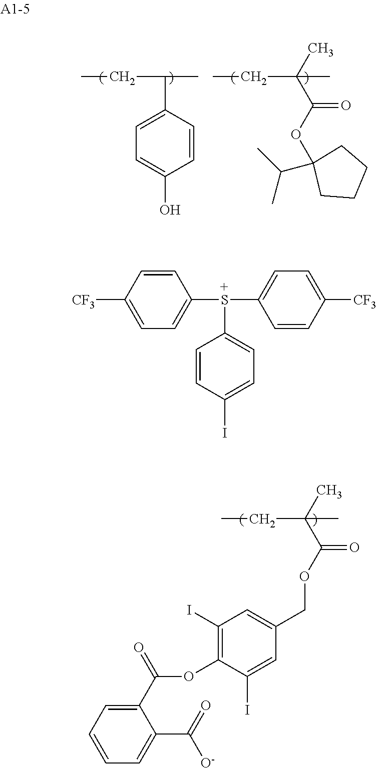

### Example 20 [Synthesis of Resin A1-6]

Using a monomer (ax-1), a monomer (a1-2-6) and a monomer (I-238) as monomers, these monomers were mixed in a molar ratio of 35:62:3 [monomer (ax-1):monomer (a1-2-6):monomer (I-238)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-6 (copolymer) having a weight-average molecular weight of about 5.4×10³ in a yield of 86%. This resin A1-6 includes the following structural units.

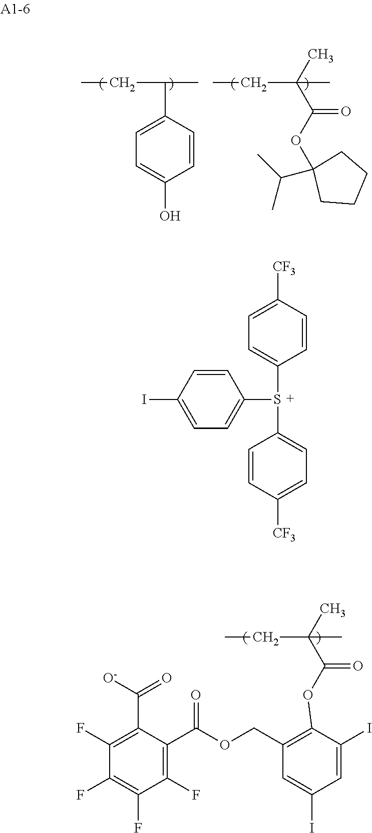

### Example 21 [Synthesis of Resin A1-7]

Using a monomer (ax-1), a monomer (a1-2-6) and a monomer (I-1058) as monomers, these monomers were mixed in a molar ratio of 35:62:3 [monomer (ax-1):monomer (a1-2-6):monomer (I-1058)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-7 (copolymer) having a weight-average molecular weight of about 5.5×10³ in a yield of 81%. This resin A1-7 includes the following structural units.

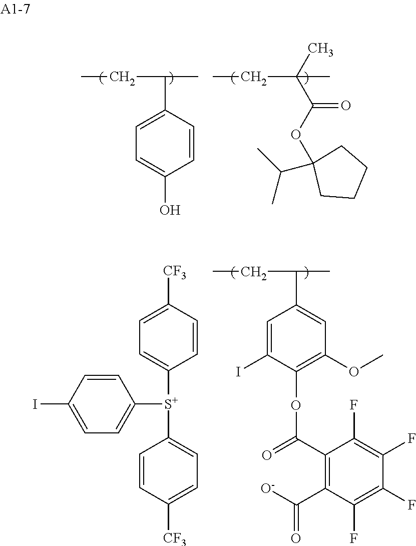

### Example 22 [Synthesis of Resin A1-8]

Using a monomer (ax-1), a monomer (a1-2-6) and a monomer (I-1059) as monomers, these monomers were mixed in a molar ratio of 35:62:3 [monomer (ax-1):monomer (a1-2-6):monomer (I-1059)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-8 (copolymer) having a weight-average molecular weight of about 5.6×10³ in a yield of 82%. This resin A1-8 includes the following structural units.

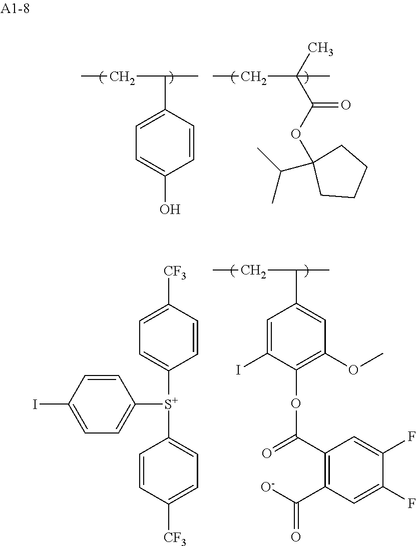

### Example 23 [Synthesis of Resin A1-9]

Using a monomer (ax-1), a monomer (a1-2-6) and a monomer (I-1060) as monomers, these monomers were mixed in a molar ratio of 35:62:3 [monomer (ax-1):monomer (a1-2-6):monomer (I-1060)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-9 (copolymer) having a weight-average molecular weight of about 5.7×10³ in a yield of 83%. This resin A1-9 includes the following structural units.

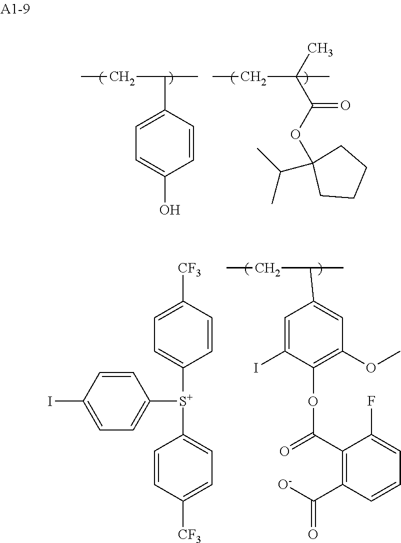

### Example 24 [Synthesis of Resin A1-10]

Using a monomer (ax-1), a monomer (a1-2-6) and a monomer (I-1062) as monomers, these monomers were mixed in a molar ratio of 35:62:3 [monomer (ax-1):monomer (a1-2-6):monomer (I-1062)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-10 (copolymer) having a weight-average molecular weight of about 5.5×10³ in a yield of 80%. This resin A1-10 includes the following structural units.

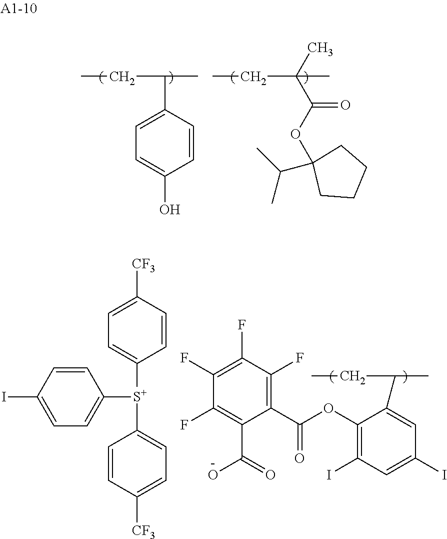

### Example 25 [Synthesis of Resin A1-11]

Using a monomer (ax-1), a monomer (a1-2-6) and a monomer (I-1066) as monomers, these monomers were mixed in a molar ratio of 35:62:3 [monomer (ax-1):monomer (a1-2-6):monomer (I-1066)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-11 (copolymer) having a weight-average molecular weight of about 5.5×10³ in a yield of 84%. This resin A1-11 includes the following structural units.

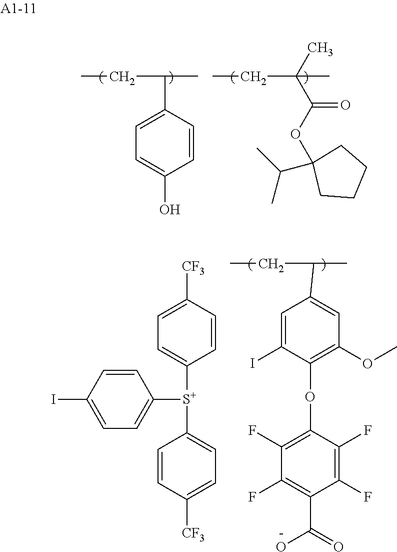

### Example 26 [Synthesis of Resin A1-12]

Using a monomer (ax-1), a monomer (a1-2-6) and a monomer (I-1067) as monomers, these monomers were mixed in a molar ratio of 35:62:3 [monomer (ax-1):monomer (a1-2-6):monomer (I-1067)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-12 (copolymer) having a weight-average molecular weight of about 5.7×10³ in a yield of 77%. This resin A1-12 includes the following structural units.

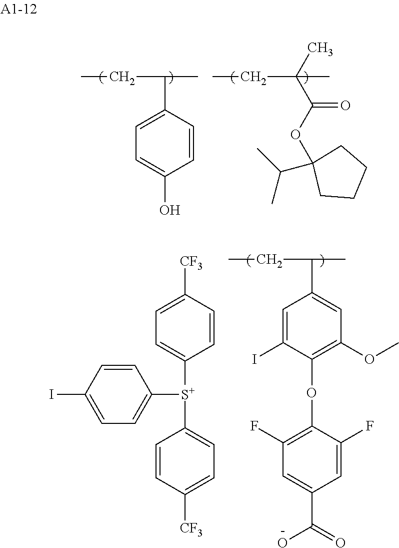

### Example 27 [Synthesis of Resin A1-13]

Using a monomer (ax-1), a monomer (a1-2-6) and a monomer (I-1068) as monomers, these monomers were mixed in a molar ratio of 35:62:3 [monomer (ax-1):monomer (a1-2-6):monomer (I-1068)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-13 (copolymer) having a weight-average molecular weight of about 5.8×10³ in a yield of 75%. This resin A1-13 includes the following structural units.

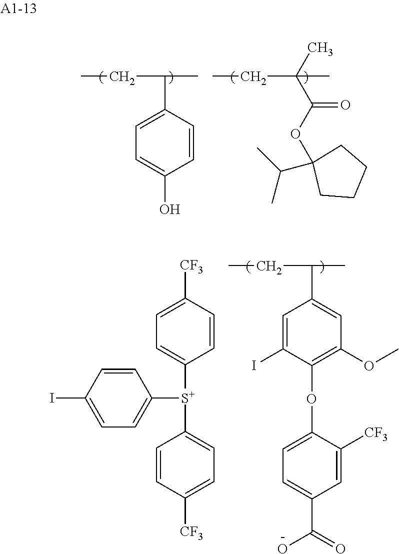

### Example 28 [Synthesis of Resin A1-14]

Using a monomer (a2-2-1), a monomer (a1-2-6) and a monomer (I-238) as monomers, these monomers were mixed in a molar ratio of 35:62:3 [monomer (a2-2-1):monomer (a1-2-6):monomer (I-238)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, the polymerization reaction solution was poured into a large amount of n-heptane to precipitate a resin, followed by filtration and recovery to obtain a resin A1-14 (copolymer) having a weight-average molecular weight of about 5.5×10³ in a yield of 88%. This resin A1-14 includes the following structural units.

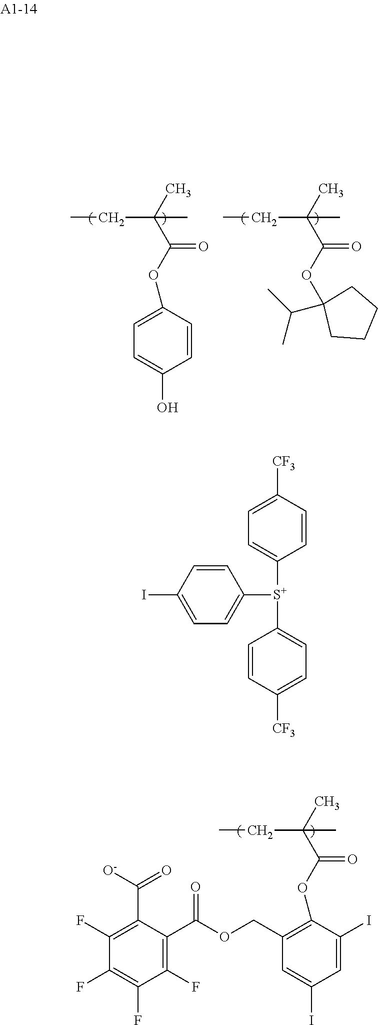

### Example 29 [Synthesis of Resin A1-15]

Using a monomer (a2-2-1), a monomer (a1-0-13) and a monomer (I-238) as monomers, these monomers were mixed in a molar ratio of 35:62:3 [monomer (a2-2-1):monomer (a1-0-13):monomer (I-238)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, the polymerization reaction solution was poured into a large amount of n-heptane to precipitate a resin, followed by filtration and recovery to obtain a resin A1-15 (copolymer) having a weight-average molecular weight of about 5.8×10³ in a yield of 88%. This resin A1-15 includes the following structural units.

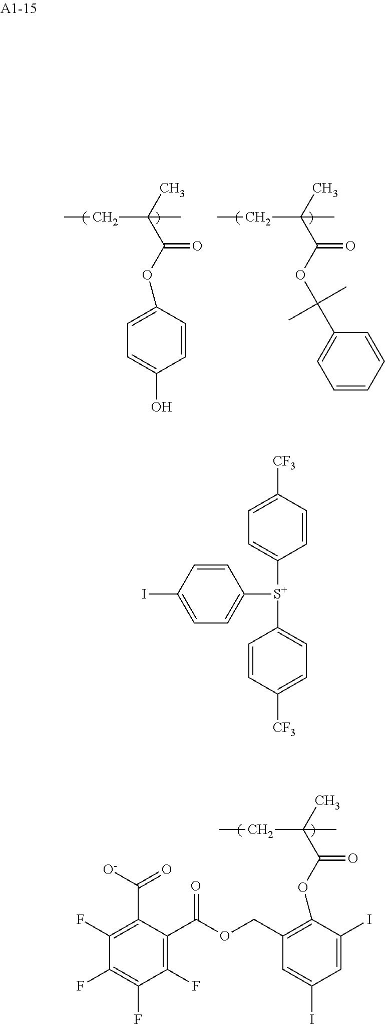

### Example 30 [Synthesis of Resin A1-16]

Using a monomer (ax-1), a monomer (a1-4-2) and a monomer (I-1058) as monomers, these monomers were mixed in a molar ratio of 35:62:3 [monomer (ax-1):monomer (a1-4-2):monomer (I-1058)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-16 (copolymer) having a weight-average molecular weight of about 5.8×10³ in a yield of 89%. This resin A1-16 includes the following structural units.

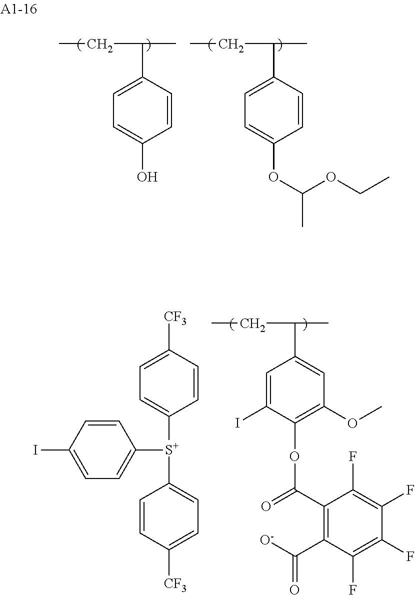

### Example 31 [Synthesis of Resin A1-17]

Using a monomer (ax-1), a monomer (a1-6-8) and a monomer (I-1058) as monomers, these monomers were mixed in a molar ratio of 35:62:3 [monomer (ax-1):monomer (a1-6-8):monomer (I-1058)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-17 (copolymer) having a weight-average molecular weight of about 5.5×10³ in a yield of 82%. This resin A1-17 includes the following structural units.

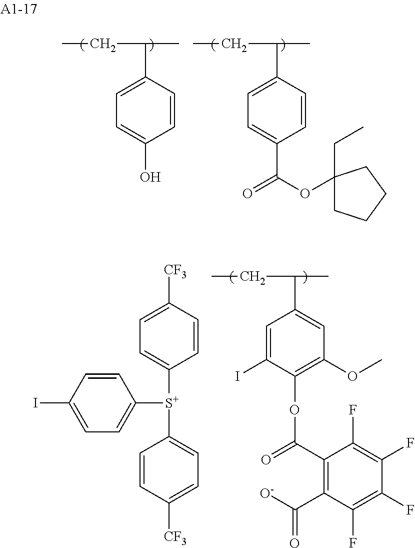

### Example 32 [Synthesis of Resin A1-18]

Using a monomer (ax-1), a monomer (a1-2-6) and a monomer (I-1071) as monomers, these monomers were mixed in a molar ratio of 35:62:3 [monomer (ax-1):monomer (a1-2-6):monomer (I-1071)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-18 (copolymer) having a weight-average molecular weight of about 5.4×10³ in a yield of 86%. This resin A1-18 includes the following structural units.

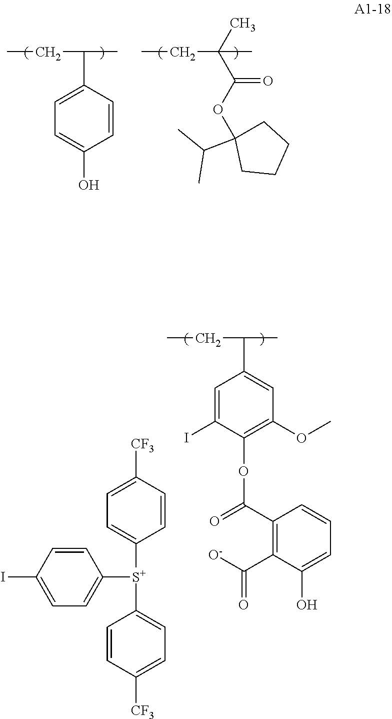

### Example 33 [Synthesis of Resin A1-19]

Using a monomer (ax-1), a monomer (a1-6-8) and a monomer (I-1071) as monomers, these monomers were mixed in a molar ratio of 35:62:3 [monomer (ax-1):monomer (a1-6-8):monomer (I-1071)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin A1-19 (copolymer) having a weight-average molecular weight of about 5.4×10³ in a yield of 85%. This resin A1-19 includes the following structural units.

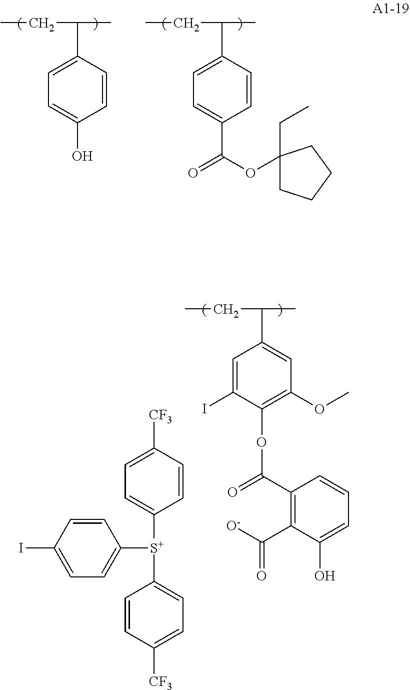

### Synthesis Example 2 [Synthesis of Resin A2-1]

Using a monomer (a1-4-2) and a monomer (a1-2-6) as monomers, these monomers were mixed in a molar ratio of 38:62 [monomer (a1-4-2):monomer (a1-2-6)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous p-toluenesulfonic acid solution was added, followed by stirring for 6 hours and further isolation through separation. The organic layer thus recovered was poured into a large amount of n-heptane to precipitate a resin, followed by filtration and recovery to obtain a resin A2-1 (copolymer) having a weight-average molecular weight of about 5.5×10³ in a yield of 89%. This resin A2-1 includes the following structural units.

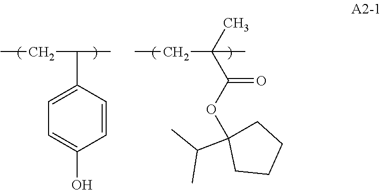

### Synthesis Example 3 [Synthesis of Resin AX1-1]

Using a monomer (ax-1), a monomer (a1-2-6) and a monomer (IX-1) as monomers, these monomers were mixed in a molar ratio of 35:62:3 [monomer (ax-1):monomer (a1-2-6):monomer (IX-1)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin AX1-1 (copolymer) having a weight-average molecular weight of about 5.5×10³ in a yield of 82%. This resin AX1-1 includes the following structural units.

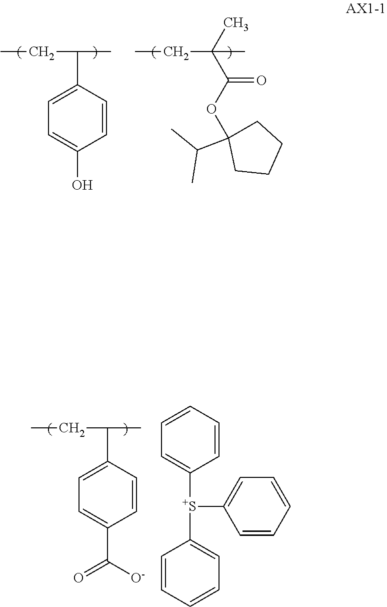

### Synthesis Example 4 [Synthesis of Resin AX1-2]

Using a monomer (ax-1), a monomer (a1-2-6) and a monomer (IX-2) as monomers, these monomers were mixed in a molar ratio of 35:62:3 [monomer (ax-1):monomer (a1-2-6):monomer (IX-2)], and then this monomer mixture was mixed with propylene glycol monomethyl ether in the amount of 1.5 mass times the total mass of all monomers. To the mixture thus obtained, azobisisobutyronitrile as an initiator was added in the amount of 7 mol % based on the total molar number of all monomers, and then the mixture was polymerized by heating at 83° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin AX1-2 (copolymer) having a weight-average molecular weight of about 5.6×10³ in a yield of 86%. This resin AX1-2 includes the following structural units.

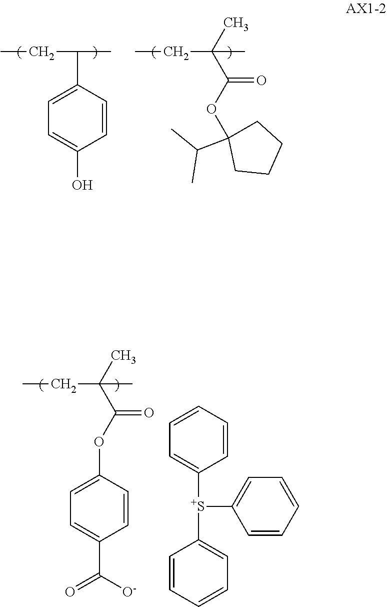

### <Preparation of Resist Composition>

The respective components shown in Table 2 and the following solvents were mixed and the mixture thus obtained was filtered through a fluororesin filter having a pore diameter of 0.2 μm to prepare resist compositions.

TABLE 2

| Resist | | Acid | Carboxylate | Quencher | |
|---|---|---|---|---|---|
| composition | Resin | generator | (I) | (C) | PB/PEB |
| Composition | A1-3 = | B1-25 = | I-224 = | — | 100° C./ |
| 1 | 10 parts | 2.5 parts | 0.1 part | | 130° C. |
| Composition | A2-1 = | B1-25 = | I-224 = | — | 100° C./ |
| 2 | 10 parts | 2.5 parts | 0.4 part | | 130° C. |
| Composition | A1-1 = | B1-25 = | — | C1 = | 100° C./ |
| 3 | 10 parts | 2.5 parts | | 0.1 part | 130° C. |
| Composition | A1-2 = | B1-25 = | — | C1 = | 100° C./ |
| 4 | 10 parts | 2.5 parts | | 0.1 part | 130° C. |
| Composition | A1-3 = | B1-25 = | — | C1 = | 100° C./ |
| 5 | 10 parts | 2.5 parts | | 0.1 part | 130° C. |
| Composition | A1-4 = | B1-25 = | — | C1 = | 100° C./ |
| 6 | 10 parts | 2.5 parts | | 0.1 part | 130° C. |
| Composition | A1-5 = | B1-25 = | — | C1 = | 100° C./ |
| 7 | 10 parts | 2.5 parts | | 0.1 part | 130° C. |
| Composition | A1-1 = | B1-25 = | — | — | 100° C./ |
| 8 | 10 parts | 2.5 parts | | | 130° C. |
| Composition | A1-2 = | B1-25 = | — | — | 100° C./ |
| 9 | 10 parts | 2.5 parts | | | 130° C. |
| Composition | A1-3 = | B1-25 | — | — | 100° C./ |
| 10 | 10 parts | 2.5 parts | | | 130° C. |
| Composition | A1-4 = | B1-25 = | — | — | 100° C./ |
| 11 | 10 parts | 2.5 parts | | | 130° C. |
| Composition | A1-5 = | B1-25 = | — | — | 100° C./ |
| 12 | 10 parts | 2.5 parts | | | 130° C. |
| Composition | A1-6 = | B1-25 = | — | — | 100° C./ |
| 13 | 10 parts | 2.5 parts | | | 130° C. |
| Composition | A1-7 = | B1-25 = | — | — | 100° C./ |
| 14 | 10 parts | 2.5 parts | | | 130° C. |
| Composition | A1-8 = | B1-25 = | — | — | 100° C./ |
| 15 | 10 parts | 2.5 parts | | | 130° C. |
| Composition | A1-9 = | B1-25 = | — | — | 100° C./ |
| 16 | 10 parts | 2.5 parts | | | 130° C. |
| Composition | A1-10 = | B1-25 = | — | — | 100° C./ |
| 17 | 10 parts | 2.5 parts | | | 130° C. |
| Composition | A1-11 = | B1-25 = | — | — | 100° C./ |
| 18 | 10 parts | 2.5 parts | | | 130° C. |
| Composition | A1-12 = | B1-25 | — | — | 100° C./ |
| 19 | 10 parts | 2.5 parts | | | 130° C. |
| Composition | A1-13 = | B1-25 = | — | — | 100° C./ |
| 20 | 10 parts | 2.5 parts | | | 130° C. |
| Composition | A1-14 = | B1-25 = | — | — | 100° C./ |
| 21 | 10 parts | 2.5 parts | | | 130° C. |
| Composition | A1-15 = | B1-25 = | — | — | 100° C./ |
| 22 | 10 parts | 2.5 parts | | | 130° C. |
| Composition | A1-16 = | B1-25 = | — | — | 100° C./ |
| 23 | 10 parts | 2.5 parts | | | 130° C. |
| Composition | A1-17 = | B1-25 = | — | — | 100° C./ |
| 24 | 10 parts | 2.5 parts | | | 130° C. |
| Composition | A1-18 = | B1-25 = | — | — | 100° C./ |
| 25 | 10 parts | 2.5 parts | | | 130° C. |
| Composition | A1-19 = | B1-25 = | — | — | 100° C./ |
| 26 | 10 parts | 2.5 parts | | | 130° C. |
| Comparative | AX1-1 = | B1-25 = | — | — | 100° C./ |
| Composition 1 | 10 parts | 2.5 parts | | | 130° C. |
| Comparative | AX1-2 = | B1-25 = | — | — | 100° C./ |
| Composition 2 | 10 parts | 2.5 parts | | | 130° C. |

### <Resin (A)>

A1-1 to A1-19, A2-1, AX1-1, AX1-2: Resin A1-1 to Resin A1-19, Resin A2-1, Resin AX1-1, Resin AX1-2

### <Carboxylate (I)>

I-224: Salt represented by Formula (I-224)

### <Acid Generator (B)>

B1-25: Salt represented by Formula (B1-25); synthesized by the method mentioned in JP 2011-126869 A

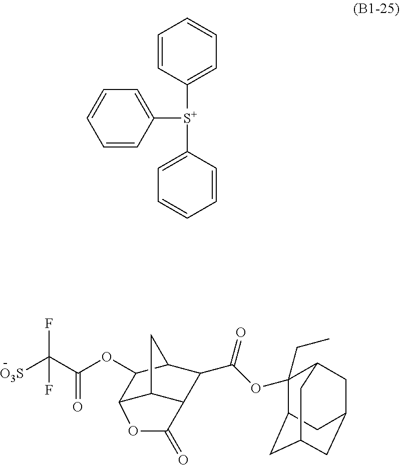

### <Quencher (C)>

C1: synthesized by the method mentioned in JP 2011-39502 A

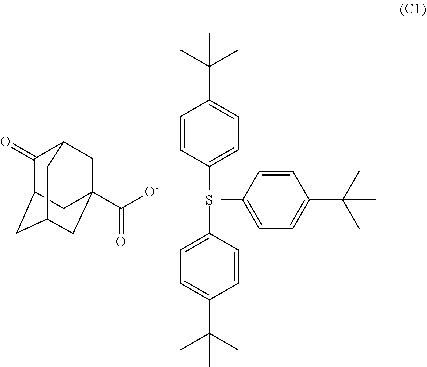 [SMILES: O=C1C2CC3CC1CC(C(=O)[O-])(C3)C2 ; CC(C)(C)c1ccc([S+](c2ccc(C(C)(C)C)cc2)c2ccc(C(C)(C)C)cc2)cc1]

### <Solvent (E)>

| | |
|---|---|
| Propylene glycol monomethyl ether acetate | 400 parts |
| Propylene glycol monomethyl ether | 100 parts |
| γ-Butyrolactone | 5 parts |

(Evaluation of Exposure of Resist Composition with Electron Beam: Alkali Development)

Each 6 inch-diameter silicon wafer was treated with hexamethyldisilazane on a direct hot plate at 90° C. for 60 seconds. A resist composition was spin-coated on the silicon wafer in such a manner that the thickness of the composition layer became 0.04 μm. Then, the coated silicon wafer was prebaked on the direct hot plate at the temperature shown in the column “PB” of Table 2 for 60 seconds to form a composition layer. Using an electron-beam direct-write system (“ELS-F125 125 keV”, manufactured by ELIONIX INC.), contact hole patterns (hole pitch of 40 nm/hole diameter of 17 nm) were directly written on the composition layer formed on the wafer while changing the exposure dose stepwise.

After exposure, post-exposure baking was performed on the hot plate at the temperature shown in the column “PEB” of Table 2 for 60 seconds. Next, the composition layer on this silicon wafer was puddle-developed with an aqueous 2.38% by mass tetramethylammonium hydroxide solution as a developer at 23° C. for 60 seconds to obtain resist patterns.

In the resist pattern obtained after development, the exposure dose at which the diameter of holes formed became 17 nm was regarded as effective sensitivity.

### <Evaluation of CD Uniformity (CDU)>

In the effective sensitivity, the hole diameter of the pattern formed with a hole dimeter of 17 nm was determined by measuring 24 times per one hole and the average of the measured values was regarded as the average hole diameter per one hole. The standard deviation was determined under the conditions that the average diameter of 400 holes about the patterns formed with a hole dimeter of 17 nm in the same wafer was regarded to as population.

The results are shown in Table 3. The numerical value in the table represents the standard deviation (nm).

TABLE 3

| | Resist composition | CDU |
|---|---|---|
| Example 34 | Composition 1 | 2.35 |
| Example 35 | Composition 2 | 2.49 |
| Example 36 | Composition 3 | 2.53 |
| Example 37 | Composition 4 | 2.42 |
| Example 38 | Composition 5 | 2.38 |
| Example 39 | Composition 6 | 2.34 |
| Example 40 | Composition 7 | 2.39 |
| Example 41 | Composition 8 | 2.55 |
| Example 42 | Composition 9 | 2.45 |
| Example 43 | Composition 10 | 2.39 |
| Example 44 | Composition 11 | 2.35 |
| Example 45 | Composition 12 | 2.43 |
| Example 46 | Composition 13 | 2.26 |
| Example 47 | Composition 14 | 2.24 |
| Example 48 | Composition 15 | 2.30 |
| Example 49 | Composition 16 | 2.32 |
| Example 50 | Composition 17 | 2.23 |
| Example 51 | Composition 18 | 2.26 |
| Example 52 | Composition 19 | 2.33 |
| Example 53 | Composition 20 | 2.35 |
| Example 54 | Composition 21 | 2.22 |
| Example 55 | Composition 22 | 2.18 |
| Example 56 | Composition 23 | 2.20 |
| Example 57 | Composition 24 | 2.12 |
| Example 58 | Composition 25 | 2.23 |
| Example 59 | Composition 26 | 2.14 |
| Comparative Example 1 | Comparative Composition 1 | 2.83 |
| Comparative Example 2 | Comparative Composition 2 | 2.85 |

As compared with Comparative Compositions 1 and 2, Compositions 1 to 26 exhibited satisfactory evaluation of CD uniformity (CDU).

The resist composition including a carboxylate of the present invention is capable of obtaining a resist pattern with satisfactory CD uniformity (CDU), and is therefore useful for fine processing of semiconductors and is industrially extremely useful.

What is claimed is:

1. A carboxylate represented by formula (I): 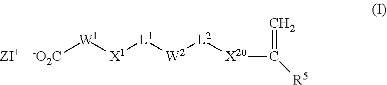
wherein, in formula (I),
W¹ and W² each independently represent a cyclic hydrocarbon group having 3 to 36 carbon atoms which may have a substituent, and —CH₂— included in the cyclic hydrocarbon group may be replaced by —O—, —S—, —CO— or —SO₂—,
X¹ represents —CO—O—, —O—CO—, —O—CO—O— or —O—,
L¹ and L² each independently represent a single bond or a hydrocarbon group having 1 to 28 carbon atoms which may have a substituent, and —CH₂— included in the hydrocarbon group may be replaced by —O—, —S—, —SO₂— or —CO—,
X²⁰ represents a single bond, *—O—**, *—CO—O—**, *—O—CO—O—** or *-Ax-Ph-Ay-**,
Ph represents a phenylene group which may have a substituent,
Ax and Ay each independently represent one or more bond species selected from the group consisting of a single bond, an ether bond, a thioether bond, an ester bond, an amide bond and a carbonic acid ester bond,
* and ** represent a bonding site, and * represents a bonding site to carbon atoms to which R⁵ is bonded,
R⁵ represents a hydrogen atom, a halogen atom, or an alkyl group having 1 to 6 carbon atoms which may have a halogen atom, and
ZI⁺ represents an organic cation.

2. The carboxylate according to claim 1, wherein X²⁰ is a single bond or a group represented by any one of formula (X²⁰-1) to formula (X²⁰-10) 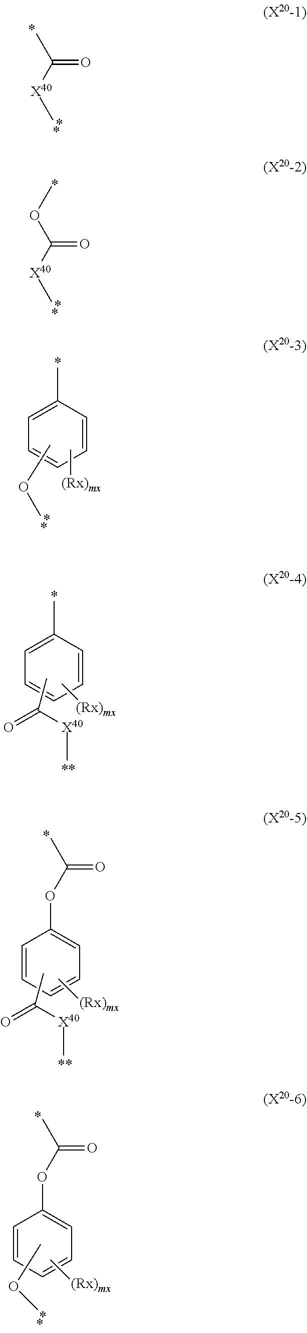 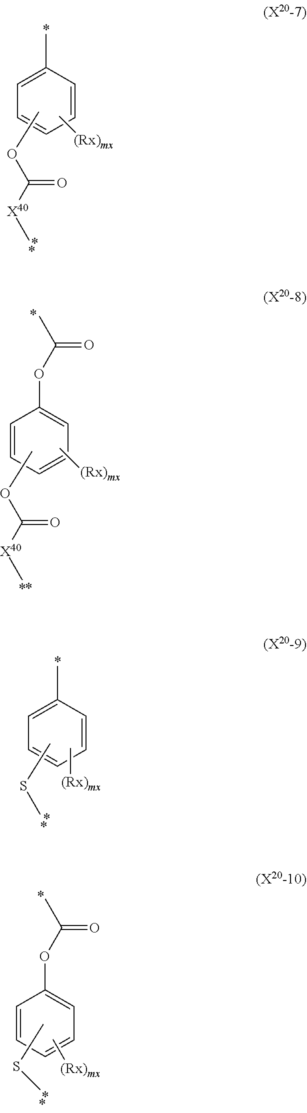
wherein, in formula (X²⁰-1) to formula (X²⁰-10),
* and ** are bonding sites, and * represents a bonding site to carbon atoms to which R⁵ is bonded,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms,
mx represents an integer of 0 to 4,
X⁴⁰ represents —O— or —NR³—, and
R³ represents a hydrogen atom or an alkyl group having 1 to 6 carbon atoms.

3. The carboxylate according to claim 1, wherein W¹ is a cyclic hydrocarbon group represented by formula (W-1): 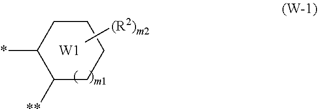
wherein, in formula (W-1),
W¹ represents a cyclic hydrocarbon group having (5+m1) carbon atoms, a carbon atom included in the cyclic hydrocarbon group may form a double bond between two adjacent carbon atoms, —CH₂— included in the cyclic hydrocarbon group may be replaced by —O—, —S—, —CO— or —SO₂—, and a hydrogen atom included in the cyclic hydrocarbon group may be substituted with a substituent,
R² represents a halogen atom, a haloalkyl group having 1 to 6 carbon atoms or an alkyl group having 1 to 12 carbon atoms, —CH₂— included in the alkyl group may be replaced by —O—, —CO—, —S— or —SO₂—, the alkyl group may be bonded to two carbon atoms included in W¹ to form an alkanediyl bridge, —CH₂— included in the alkanediyl bridge may be replaced by —O—, —CO—, —S— or —SO₂—, and the alkanediyl bridge may form a double bond between two adjacent carbon atoms,
m1 represents an integer of 0 to 3,
m2 represents an integer of 0 to 3, and when m2 is 2 or more, a plurality of R² may be the same or different from each other, and
* represents a bonding site to carbon atoms included in COO—, and ** represents a bonding site to X¹.

4. The carboxylate according to claim 1, wherein X¹ is *—CO—O— or *—O— (in which * represents a bonding site to W¹).

5. The carboxylate according to claim 1, wherein L¹ and L² each independently represent a single bond or an alkanediyl group having 1 to 8 carbon atoms (—CH₂— included in the alkanediyl group may be replaced by —O— or —CO—).

6. The carboxylate according to claim 1, wherein W² is an aromatic hydrocarbon group having 6 to 10 carbon atoms which may have a halogen atom, a haloalkyl group having 1 to 6 carbon atoms or an alkyl group having 1 to 12 carbon atoms (—CH₂— included in the alkyl group may be replaced by —O—, —CO—, —S— or —SO₂—).

7. A carboxylic acid generator comprising the carboxylate according to claim 1 or a structural unit derived from the carboxylate according to claim 1.

8. A resin including a structural unit derived from the carboxylate according to claim 1.

9. A resist composition comprising the carboxylic acid generator according to claim 7, and an acid generator other than the carboxylic acid generator.

10. The resist composition according to claim 9, wherein
the carboxylic acid generator is a carboxylate represented by formula (I), and
a resin including a structural unit having an acid-labile group is further included, or
the carboxylic acid generator is a resin including a structural unit derived from a carboxylate represented by formula (I), and
the resin further includes a structural unit having an acid-labile group.

11. The resist composition according to claim 10, wherein the structural unit having an acid-labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5) and a structural unit represented by formula (a1-6): 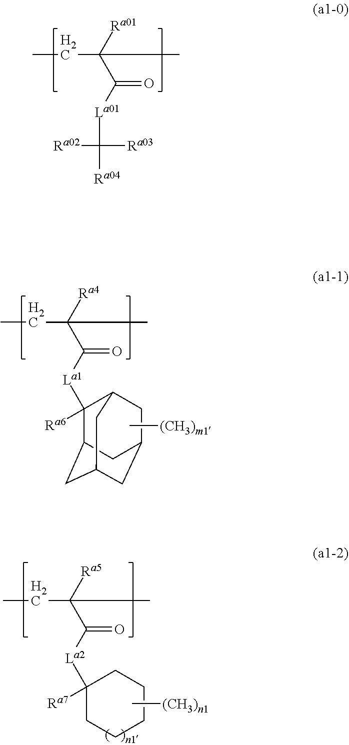
wherein, in formula (a1-0), formula (a1-1) and formula (a1-2),
L^{a01}, L^{a1} and L^{a2} each independently represent —O— or *—O—(CH₂)ₖ₁—CO—O—, k1 represents an integer of 1 to 7, and * represents a bonding site to —CO—,
R^{a01}, R^{a4} and R^{a5} each independently represent a hydrogen atom, a halogen atom, or an alkyl group having 1 to 6 carbon atoms which may have a halogen atom,
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups formed by combining these groups, and the alkyl group, the alkenyl group, the alicyclic hydrocarbon group and the aromatic hydrocarbon group may have a halogen atom,
m1′ represents an integer of 0 to 14,
n1 represents an integer of 0 to 10, and
n1′ represents an integer of 0 to 3: 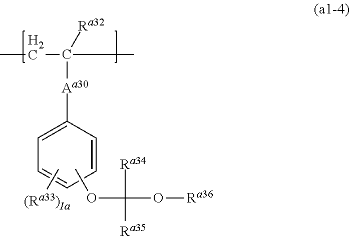
wherein, in formula (a1-4),
R^{a32} represents a hydrogen atom, a halogen atom, or an alkyl group having 1 to 6 carbon atoms which may have a halogen atom,
R^{a33} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a30} represents a single bond or *—X^{a31}-(A^{a32}-X^{a32}) and * represents a bonding site to carbon atoms to which —R^{a32} is bonded,
A^{a32} represents an alkanediyl group having 1 to 8 carbon atoms,
X^{a31} and X^{a32} each independently represent —O—, —CO—O— or —O—CO—,
nc represents 0 or 1,
1a represents an integer of 0 to 4, and when 1a is an integer of 2 or more, a plurality of R^{a33} may be the same or different from each other, and
R^{a34} and R^{a35} each independently represent a hydrogen atom or a hydrocarbon group having 1 to 12 carbon atoms, R^{a36} represents a hydrocarbon group having 1 to 20 carbon atoms, or R^{a35} and R^{a36} may be bonded to each other to form a divalent hydrocarbon group having 2 to 20 carbon atoms together with —C—O— to which R^{a35} and R^{a36} are bonded, and —CH₂— included in the hydrocarbon group and the divalent hydrocarbon group may be replaced by —O— or —S—: 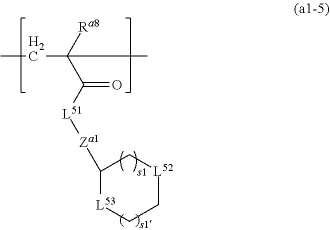
wherein, in formula (a1-5),
R^{a8} represents an alkyl group having 1 to 6 carbon atoms which may have a halogen atom, a hydrogen atom or a halogen atom,
Z^{a1} represents a single bond or *—(CH₂)ₕ₃—CO-L⁵⁴-, h3 represents an integer of 1 to 4, and * represents a bonding site to L⁵¹,
L⁵¹, L⁵², L⁵³ and L⁵⁴ each independently represent —O— or —S—,
s1 represents an integer of 1 to 3, and
s1′ represents an integer of 0 to 3, and: 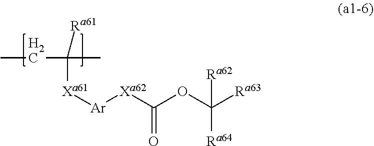
wherein, in formula (a1-6),
R^{a61} represents a hydrogen atom, a halogen atom, or an alkyl group having 1 to 6 carbon atoms which may have a halogen atom,
R^{a62}, R^{a63} and R^{a64} each independently represent an alkyl group having 1 to 6 carbon atoms or a cyclic hydrocarbon group having 3 to 18 carbon atoms which may have a substituent, or R^{a62} and R^{a63} may be bonded to each other to form a ring having 3 to 20 carbon atoms together with carbon atoms to which R^{a62} and R^{a63} are bonded,
X^{a61} represents a single bond, —CO—O—* or —CO—NR⁵—*, * represents a bonding site to —Ar, and R⁵ represents a hydrogen atom or an alkyl group having 1 to 4 carbon atoms,
X^{a62} represents a single bond, *—O-L^{a61}- or *—CO—O— L^{a62}-, * represents a bonding site to —Ar, and L^{a61} and L^{a62} each independently represent an alkanediyl group having 1 to 4 carbon atoms, and
Ar represents an aromatic hydrocarbon group having 6 to 20 carbon atoms which may have a substituent.

12. The resist composition according to claim 10, wherein the resin including a structural unit having an acid-labile group includes a structural unit represented by formula (a2-A): 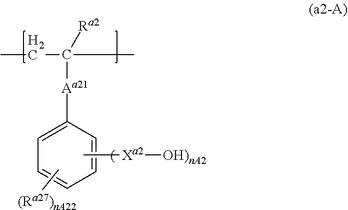
wherein, in formula (a2-A),
R^{a2} represents a hydrogen atom, a halogen atom, or an alkyl group having 1 to 6 carbon atoms which may have a halogen atom,
A^{a21} represents a single bond or an alkanediyl group having 1 to 12 carbon atoms, and —CH₂— included in the alkanediyl group may be replaced by —O—, —CO— or —NR^{a28},
R^{a28} represents a hydrogen atom or an alkyl group having 1 to 4 carbon atoms,
X^{a2} represents a single bond or —CO—,
R^{a27} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group,
nA2 represents an integer of 1 to 5, and when nA2 is 2 or more, a plurality of X^{a2} may be the same or different from each other, and
nA22 represents an integer of 0 to 4, and when nA22 is 2 or more, a plurality of R^{a27} may be the same or different from each other.

13. The resist composition according to claim 9, wherein the acid generator includes a salt represented by formula (B1): 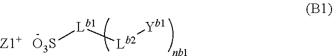
wherein, in formula (B1),
L^{b1} represents a single bond or a (nb1⁺1)-valent hydrocarbon group which may have a substituent, and —CH₂— included in the hydrocarbon group may be replaced by —O—, —S—, —CO— or —SO₂—,
L^{b2} represents a single bond or a divalent hydrocarbon group having 1 to 24 carbon atoms which may have a substituent, and —CH₂— included in the hydrocarbon group may be replaced by —O—, —S—, —CO— or —SO₂—,
Y^{b1} represents a methyl group which may have a substituent, or a cyclic hydrocarbon group having 3 to 24 carbon atoms which may have a substituent, and —CH₂— included in the cyclic hydrocarbon group may be replaced by —O—, —S—, —CO— or —SO₂—,
nb1 represents an integer of 1 to 6, and when nb1 is 2 or more, a plurality of groups in parentheses may be the same or different from each other, and
Z1⁺ represents an organic cation.

14. A method for producing a resist pattern, which comprises:
(1) a step of applying the resist composition according to claim 9 on a substrate,
(2) a step of drying the applied composition to form a composition layer,
(3) a step of exposing the composition layer,
(4) a step of heating the exposed composition layer, and
(5) a step of developing the heated composition layer.
